SEQUENCE LISTING (546 sequences)

SEQ ID NO: 1 - protein, 121 aa, synthetic construct
  source 1..121: note = 1A7[CD28]_H1L1 Variable heavy (vh) domain and 1A7[CD28]_H1_L1.71 Variable Heavy (vh) Domain
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSS

SEQ ID NO: 2 - protein, 5 aa, synthetic construct
  source 1..5: note = 1A7[CD28]_H1L1, 1A7[CD28]_H1_L1.71, 1A7[CD28]_H1sp_L1sp, and 1A7[CD28]_H1sp_L1.71sp vhCDR1
SYAMS

SEQ ID NO: 3 - protein, 17 aa, synthetic construct
  source 1..17: note = 1A7[CD28]_H1L1, 1A7[CD28]_H1.1_L1, 1A7[CD28]_H1_L1.71, 1A7[CD28]_H1.1_L1.71, 1A7[CD28]_H1sp_L1sp, 1A7[CD28]_H1.1sp_L1sp, 1A7[CD28]_H1sp_L1.71sp, and 1A7[CD28]_H1.1sp_L1.71sp vhCDR2
TISGSGDSTYYADSVKG

SEQ ID NO: 4 - protein, 12 aa, synthetic construct
  source 1..12: note = 1A7[CD28]_H1L1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, 1A7[CD28]_H1sp_L1sp, 1A7[CD28]_H1.1sp_L1sp, 1A7[CD28]_H1sp_L1.71sp, 1A7[CD28]_H1.1sp_L1.71sp, 1A7[CD28]_H1.14sp_L1sp, 1A7[CD28]_H1.14sp_L1.71sp, 1A7 VH HCDR3 , and 2F10A3.140[CD28]_H1L1 vhCDR3
SGPGLRQVGFDY

SEQ ID NO: 5 - protein, 107 aa, synthetic construct
  source 1..107: note = 1A7[CD28]_H1L1, 1A7[CD28]_H1.1_L1, 1A7[CD28]_H1.14_L1, and 2F10A3.140[CD28]_H1L1 Variable light (vl) domain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIK

SEQ ID NO: 6 - protein, 11 aa, synthetic construct
  source 1..11: note = 1A7[CD28]_H1L1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, 1A7[CD28]_H1sp_L1sp, 1A7[CD28]_H1.1sp_L1sp, 1A7[CD28]_H1sp_L1.71sp, 1A7[CD28]_H1.1sp_L1.71sp, 1A7[CD28]_H1.14sp_L1sp, 1A7[CD28]_H1.14sp_L1.71sp, and 2F10A3.140[CD28]_H1L1 vlCDR1
RASQSISSYLN

SEQ ID NO: 7 - protein, 7 aa, synthetic construct
  source 1..7: note = 1A7[CD28]_H1L1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, 1A7[CD28]_H1sp_L1sp, 1A7[CD28]_H1.1sp_L1sp, 1A7[CD28]_H1sp_L1.71sp, 1A7[CD28]_H1.1sp_L1.71sp, 1A7[CD28]_H1.14sp_L1sp, 1A7[CD28]_H1.14sp_L1.71sp, and 2F10A3.140[CD28]_H1L1 vlCDR2
AASSLQS

SEQ ID NO: 8 - protein, 9 aa, synthetic construct
  source 1..9: note = 1A7[CD28]_H1L1, 1A7[CD28]_H1.1_L1, 1A7[CD28]_H1.14_L1, 1A7[CD28]_H1sp_L1sp, 1A7[CD28]_H1.1sp_L1sp, 1A7[CD28]_H1.14sp_L1sp, and 2F10A3.140[CD28]_H1L1 vlCDR3
QQSYSTPFT

SEQ ID NO: 9 - protein, 450 aa, synthetic construct
  source 1..450: note = XENP28428 Heavy Chain
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
  source 1..214: note = XENP28428 Light Chain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 121 aa, synthetic construct
  source 1..121: note = 1A7_H1.1, 1A7[CD28]_H1.1_L1, 1A7[CD28]_H1.1_L1.71, and 1A7[CD28]_H1.1 Variable Heavy (vh) Domain
EVQLLESGGGLVQPGGSLRLSCAASGFTFSSYYMSWVRQAPGKGLEWVSTISGSGDSTYYADSVKGRFTISRDNSKNTLYLQMNSLRAEDTAVYYCAKSGPGLRQVGFDYWGQGTLVTVSS

SEQ ID NO: 12 - protein, 5 aa, synthetic construct
  source 1..5: note = 1A7[CD28]_H1.1_L1, 1A7[CD28]_H1.1_L1.71, 1A7[CD28]_H1.14_L1, 1A7[CD28]_H1.14_L1.71, 1A7[CD28]_H1.1sp_L1sp, 1A7[CD28]_H1.1sp_L1.71sp, 1A7[CD28]_H1.14sp_L1sp, and 1A7[CD28]_H1.14sp_L1.71sp vhCDR1
SYYMS

SEQ ID NO: 13 - protein, 7 aa, synthetic construct
  source 1..7: note = CD28.3[CD28]_H0L0 and hCD28.3[CD28]_H1L1 vlCDR2
AATHLVE

SEQ ID NO: 14 - protein, 714 aa, synthetic construct
  source 1..714: note = XENP42162 Chain 1 - CD20-B_H1_IgG1_L234A/L235A/D265S/T366S/L368A/Y407V
QVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPGGGGGSGGGGSGGGGSEVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 15 - protein, 121 aa, synthetic construct
  source 1..121: note = 1A7_H1.14, 1A7[CD28]_H1.14_L1, 1A7[CD28]_H1.14_L1.71, and 1A7[CD28]_H1.14 Variable Heavy (vh) Domain
EVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSS

SEQ ID NO: 16 - protein, 220 aa, Homo sapiens
  source 1..220: note = sp|P10747|CD28_HUMAN T-cell-specific surface glycoprotein CD28 OS=Homo sapiens OX=9606 GN=CD28 PE=1; SV = 1
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 17 - protein, 17 aa, synthetic construct
  source 1..17: note = 1A7[CD28]_H1.14_L1, 1A7[CD28]_H1.14_L1.71, 1A7[CD28]_H1.14sp_L1sp, and 1A7[CD28]_H1.14sp_L1.71sp vhCDR2
TISESGDSTYYADSVKG

SEQ ID NO: 18 - protein, 134 aa, Homo sapiens
  source 1..134: note = sp|P10747|19-152
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 19 - protein, 107 aa, synthetic construct
  source 1..107: note = 1A7_L1.71, 1A7[CD28]_H1_L1.71, 1A7[CD28]_H1.1_L1.71, 1A7[CD28]_H1.14_L1.71, and 1A7[CD28]_L1.71 Variable Light (vl) Domain
DIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 20 - protein, 218 aa, Mus musculus
  source 1..218: note = sp|P31041|CD28_MOUSE T-cell-specific surface glycoprotein CD28 OS=Mus musculus OX=10090 GN=Cd28 PE=1; SV = 2
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 21 - protein, 131 aa, Mus sp.
  source 1..131: note = sp|P31041|20-150
NKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKL

SEQ ID NO: 22 - protein, 9 aa, synthetic construct
  source 1..9: note = 1A7[CD28]_H1_L1.71, 1A7[CD28]_H1.1_L1.71, 1A7[CD28]_H1.14_L1.71, 1A7[CD28]_H1sp_L1.71sp, 1A7[CD28]_H1.1sp_L1.71sp, and 1A7[CD28]_H1.14sp_L1.71sp vlCDR3
QQVYSTPFT

SEQ ID NO: 23 - protein, 220 aa, Macaca fascicularis
  source 1..220: note = tr|Q0PDN3|Q0PDN3_MACFA CD28 OS=Macaca fascicularis; OX = 9541 GN=CD28 PE=2 SV=1
MLRLLLALNLLPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWALVVVGGVLACYSLLVTVAFCIFWMRSKRSRLLHSDYMNMTPRRPGPTRKHYQPYAPPRDFAAYRS

SEQ ID NO: 24 - protein, 584 aa, Macaca fascicularis
  source 1..584: note = XP_015308533.1 PREDICTED: CD276 antigen isoform X1 [Macaca fascicularis]
MKLSSDHVFPLFRKLQWLPAAFRIQFTPVSPSAGAAFHHGEPSCQLPHSKMLHRRGSPGMGVHVGAALGALWFCLTGALEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSITITPQRSPTGAVEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSVTITGQPMTFPPEALWVTVGLSVCLVALLVALAFVCWRKIKQSCEEENAGAEDQDGEGEGSKTALQPLKHSDSKEDDGQELA

SEQ ID NO: 25 - protein, 438 aa, Macaca fascicularis
  source 1..438: note = XP_015308533.1|79-516
LEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSITITPQRSPTGAVEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSVTITGQPMTFPPEA

SEQ ID NO: 26 - protein, 297 aa, Homo sapiens
  source 1..297: note = sp|P11836|CD20_HUMAN B-lymphocyte antigen CD20; OS = Homo sapiens OX=9606 GN=MS4A1 PE=1 SV=1
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 27 - protein, 6 aa, Homo sapiens
  source 1..6: note = sp|P11836|CD20_HUMAN|79-84 OS=Homo sapiens OX=9606; GN = MS4A1 PE=1 SV=1
PICVTV

SEQ ID NO: 28 - protein, 47 aa, Homo sapiens
  source 1..47: note = sp|P11836|CD20_HUMAN|142-188 OS=Homo sapiens; OX = 9606 GN=MS4A1 PE=1 SV=1
KISHFLKMESLNFIRAHTPYINIYNCEPANPSEKNSPSTQYCYSIQS

SEQ ID NO: 29 - protein, 291 aa, Mus musculus
  source 1..291: note = sp|P19437|CD20_MOUSE B-lymphocyte antigen CD20; OS = Mus musculus OX=10090 GN=Ms4a1 PE=1 SV=1
MSGPFPAEPTKGPLAMQPAPKVNLKRTSSLVGPTQSFFMRESKALGAVQIMNGLFHITLGGLLMIPTGVFAPICLSVWYPLWGGIMYIISGSLLAAAAEKTSRKSLVKAKVIMSSLSLFAAISGIILSIMDILNMTLSHFLKMRRLELIQTSKPYVDIYDCEPSNSSEKNSPSTQYCNSIQSVFLGILSAMLISAFFQKLVTAGIVENEWKRMCTRSKSNVVLLSAGEKNEQTIKMKEEIIELSGVSSQPKNEEEIEIIPVQEEEEEEAEINFPAPPQEQESLPVENEIAP

SEQ ID NO: 30 - protein, 50 aa, Mus musculus
  source 1..50: note = sp|P19437|CD20_MOUSE|133-182 OS=Mus musculus; OX = 10090 GN=Ms4a1 PE=1 SV=1
LNMTLSHFLKMRRLELIQTSKPYVDIYDCEPSNSSEKNSPSTQYCNSIQS

SEQ ID NO: 31 - protein, 297 aa, Macaca fascicularis
  source 1..297: note = XP_015289654.1 B-lymphocyte antigen CD20 [Macaca fascicularis]
MTTPRNSVNGTFPAEPMKGPIAMQPGPKPLLRRMSSLVGPTQSFFMRESKALGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRVHTPYINIYNCEPANPSEKNSPSTQYCYSIQSLFLGILSVMLIFAFFQELVIAGIVENEWRRTCSRPKSSVVLLSAEEKKEQVIEIKEEVVGLTETSSQPKNEEDIEIIPIQEEEEEETETNFPEPPQDQESSPIENDSSP

SEQ ID NO: 32 - protein, 47 aa, Macaca fascicularis
  source 1..47: note = XP_015289654.1 B-lymphocyte antigen CD20 [Macaca fascicularis]|142-188
KISHFLKMESLNFIRVHTPYINIYNCEPANPSEKNSPSTQYCYSIQS

SEQ ID NO: 33 - protein, 15 aa, synthetic construct
GGGGSGGGGSGGGGS

SEQ ID NO: 34 - protein, 18 aa, synthetic construct
GSTSGSGKPGSGEGSTKG

SEQ ID NO: 35 - protein, 14 aa, synthetic construct
IRPRAIGGSKPRVA

SEQ ID NO: 36 - protein, 15 aa, synthetic construct
GKGGSGKGGSGKGGS

SEQ ID NO: 37 - protein, 15 aa, synthetic construct
GGKGSGGKGSGGKGS

SEQ ID NO: 38 - protein, 15 aa, synthetic construct
GGGKSGGGKSGGGKS

SEQ ID NO: 39 - protein, 15 aa, synthetic construct
GKGKSGKGKSGKGKS

SEQ ID NO: 40 - protein, 15 aa, synthetic construct
GGGKSGGKGSGKGGS

SEQ ID NO: 41 - protein, 15 aa, synthetic construct
GKPGSGKPGSGKPGS

SEQ ID NO: 42 - protein, 20 aa, synthetic construct
GKPGSGKPGSGKPGSGKPGS

SEQ ID NO: 43 - protein, 20 aa, synthetic construct
GKGKSGKGKSGKGKSGKGKS

SEQ ID NO: 44 - protein, 20 aa, synthetic construct
GGGGSGGGGSGGGGSGGGGS

SEQ ID NO: 45 - protein, 14 aa, synthetic construct
STAGDTHLGGEDFD

SEQ ID NO: 46 - protein, 15 aa, synthetic construct
GEGGSGEGGSGEGGS

SEQ ID NO: 47 - protein, 15 aa, synthetic construct
GGEGSGGEGSGGEGS

SEQ ID NO: 48 - protein, 15 aa, synthetic construct
GGGESGGGESGGGES

SEQ ID NO: 49 - protein, 15 aa, synthetic construct
GEGESGEGESGEGES

SEQ ID NO: 50 - protein, 15 aa, synthetic construct
GGGESGGEGSGEGGS

SEQ ID NO: 51 - protein, 20 aa, synthetic construct
GEGESGEGESGEGESGEGES

SEQ ID NO: 52 - protein, 19 aa, synthetic construct
PRGASKSGSASQTGSAPGS

SEQ ID NO: 53 - protein, 19 aa, synthetic construct
GTAAAGAGAAGGAAAGAAG

SEQ ID NO: 54 - protein, 19 aa, synthetic construct
GTSGSSGSGSGGSGSGGGG

SEQ ID NO: 55 - protein, 20 aa, synthetic construct
GGSEGKSSGSGSESKSTGGS

SEQ ID NO: 56 - protein, 18 aa, synthetic construct
GGGSGGSGGCPPCGGSGG

SEQ ID NO: 57 - protein, 5 aa, synthetic construct
GGGGS

SEQ ID NO: 58 - protein, 10 aa, synthetic construct
GGGGSGGGGS

SEQ ID NO: 59 - protein, 25 aa, synthetic construct
GGGGSGGGGSGGGGSGGGGSGGGGS

SEQ ID NO: 60 - protein, 30 aa, synthetic construct
GGGGSGGGGSGGGGSGGGGSGGGGSGGGGS

SEQ ID NO: 61 - protein, 35 aa, synthetic construct
GGGGSGGGGSGGGGSGGGGSGGGGSGGGGSGGGGS

SEQ ID NO: 62 - protein, 5 aa, synthetic construct
GGGGA

SEQ ID NO: 63 - protein, 121 aa, synthetic construct
  source 1..121: note = 1A7_H1sp, 1A7[CD28]_H1sp_L1sp, and 1A7[CD28]_H1sp_L1.71sp Variable Heavy (vh) Domain
EVQLLESGGGLVQPGGSLRLSCAASGFTFSSYAMSWVRQAPGCGLEWVSTISGSGDSTYYADSVKGRFTISRDNSKNTLYLQMNSLRAEDTAVYYCAKSGPGLRQVGFDYWGQGTLVTVSS

SEQ ID NO: 64 - protein, 10 aa, synthetic construct
GGGGAGGGGA

SEQ ID NO: 65 - protein, 15 aa, synthetic construct
GGGGAGGGGAGGGGA

SEQ ID NO: 66 - protein, 20 aa, synthetic construct
GGGGAGGGGAGGGGAGGGGA

SEQ ID NO: 67 - protein, 121 aa, synthetic construct
  source 1..121: note = 1A7_H1.1sp, 1A7[CD28]_H1.1sp_L1sp, and 1A7[CD28]_H1.1sp_L1.71sp Variable Heavy (vh) Domain
EVQLLESGGGLVQPGGSLRLSCAASGFTFSSYYMSWVRQAPGCGLEWVSTISGSGDSTYYADSVKGRFTISRDNSKNTLYLQMNSLRAEDTAVYYCAKSGPGLRQVGFDYWGQGTLVTVSS

SEQ ID NO: 68 - protein, 25 aa, synthetic construct
GGGGAGGGGAGGGGAGGGGAGGGGA

SEQ ID NO: 69 - protein, 30 aa, synthetic construct
GGGGAGGGGAGGGGAGGGGAGGGGAGGGGA

SEQ ID NO: 70 - protein, 35 aa, synthetic construct
GGGGAGGGGAGGGGAGGGGAGGGGAGGGGAGGGGA

SEQ ID NO: 71 - protein, 121 aa, synthetic construct
  source 1..121: note = 1A7_H1.14sp, 1A7[CD28]_H1.14sp_L1sp, and 1A7[CD28]_H1.14sp_L1.71sp Variable Heavy (vh) Domain
EVQLLESGGGLVQPGGSLRLSCAASGFTFSSYYMSWVRQAPGCGLEWVSTISESGDSTYYADSVKGRFTISRDNSKNTLYLQMNSLRAEDTAVYYCAKSGPGLRQVGFDYWGQGTLVTVSS

SEQ ID NO: 72 - protein, 30 aa, synthetic construct
DPALVHQRPAPPGGGGSGGGGSGGGGSGGG

SEQ ID NO: 73 - protein, 5 aa, synthetic construct
GKPGS

SEQ ID NO: 74 - protein, 25 aa, synthetic construct
GKPGSGKPGSGKPGSGKPGSGKPGS

SEQ ID NO: 75 - protein, 107 aa, synthetic construct
  source 1..107: note = 1A7_L1sp, 1A7[CD28]_H1sp_L1sp, 1A7[CD28]_H1.1sp_L1sp, and 1A7[CD28]_H1.14sp_L1sp Variable Light (vl) Domain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGCGTKLEIK

SEQ ID NO: 76 - protein, 30 aa, synthetic construct
GKPGSGKPGSGKPGSGKPGSGKPGSGKPGS

SEQ ID NO: 77 - protein, 5 aa, synthetic construct
GGGES

SEQ ID NO: 78 - protein, 15 aa, synthetic construct
  source 1..15: note = IgG1 hinge
EPKSCDKTHTCPPCP

SEQ ID NO: 79 - protein, 107 aa, synthetic construct
  source 1..107: note = 1A7_L1.71sp, 1A7[CD28]_H1sp_L1.71sp, 1A7[CD28]_H1.1sp_L1.71sp, and 1A7[CD28]_H1.14sp_L1.71sp Variable Light (vl) Domain
DIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGCGTKLEIK

SEQ ID NO: 80 - protein, 9 aa, synthetic construct
KTHTCPPCP

SEQ ID NO: 81 - protein, 15 aa, synthetic construct
EPKSSDKTHTCPPCP

SEQ ID NO: 82 - protein, 19 aa, synthetic construct
GGGGSGGGGSKTHTCPPCP

SEQ ID NO: 83 - protein, 19 aa, synthetic construct
GKPGSGKPGSKTHTCPPCP

SEQ ID NO: 84 - protein, 14 aa, synthetic construct
GKPGSKTHTCPPCP

SEQ ID NO: 85 - protein, 5 aa, synthetic construct
EPKSC

SEQ ID NO: 86 - protein, 15 aa, synthetic construct
EPKSCGGGGSGGGGS

SEQ ID NO: 87 - protein, 15 aa, synthetic construct
EPKSCGKPGSGKPGS

SEQ ID NO: 88 - protein, 10 aa, synthetic construct
EPKSCGKPGS

SEQ ID NO: 89 - protein, 216 aa, synthetic construct
  source 1..216: note = Heterodimeric Fc Backbone 1 monomer 1 (-), Heterodimeric Fc Backbone 2 monomer 1 (-), Heterodimeric Fc Backbone 12 monomer 2 (+), and 2 + 1 mAb-scFv Heterodimeric Fc Backbone monomer 1
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 90 - protein, 216 aa, synthetic construct
  source 1..216: note = Heterodimeric Fc Backbone 1 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 216 aa, synthetic construct
  source 1..216: note = Heterodimeric Fc Backbone monomer 2 (+) and Heterodimeric Fc Backbone 3 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 216 aa, synthetic construct
  source 1..216: note = Heterodimeric Fc Backbone 3 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCEVSGFYPSDIAVEWESDGQPENNYKTTPPVLDSDGSFFLYSKLTVDKSRWEQGDVFSCSVMHEALHNHYTQKSLSLSPGK

SEQ ID NO: 93 - protein, 216 aa, synthetic construct
  source 1..216: note = Heterodimeric Fc Backbone 4 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTENEVSLTCLVKGFYPSDIAVEWESDGQPENNYKTTPPVLDSDGSFFLYSKLEVDKSRWEQGDVFSCSVMHEALHNHYTQKSLSLSPGK

SEQ ID NO: 94 - protein, 216 aa, synthetic construct
  source 1..216: note = Heterodimeric Fc Backbone 4 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSKGSFFLYSKLTVDKSRWQQGNVFSCSVMHEALHNHYTQKSLSLSPGK

SEQ ID NO: 95 - protein, 216 aa, synthetic construct
  source 1..216: note = Heterodimeric Fc Backbone 5 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 96 - protein, 216 aa, synthetic construct
  source 1..216: note = Heterodimeric Fc Backbone 5 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 216 aa, synthetic construct
  source 1..216: note = Heterodimeric Fc Backbone 6 monomer 1 (-)
APPVAGPSVFLFPPKPKDTLMISRTPEVTCVVVDVKHEDPEVKFNWYVDGVEVHNAKTKPREEEYASTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 98 - protein, 216 aa, synthetic construct
  source 1..216: note = Heterodimeric Fc Backbone 6 monomer 2 (+)
APPVAGPSVFLFPPKPKDTLMISRTPEVTCVVVDVKHEDPEVKFNWYVDGVEVHNAKTKPREEQYASTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 216 aa, synthetic construct
  source 1..216: note = Heterodimeric Fc Backbone 7 monomer 1 (-)
APPVAGPSVFLFPPKPKDTLMISRTPEVTCVVVDVKHEDPEVKFNWYVDGVEVHNAKTKPREEEYSSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 100 - protein, 216 aa, synthetic construct
  source 1..216: note = Heterodimeric Fc Backbone 7 monomer 2 (+)
APPVAGPSVFLFPPKPKDTLMISRTPEVTCVVVDVKHEDPEVKFNWYVDGVEVHNAKTKPREEQYSSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 217 aa, synthetic construct
  source 1..217: note = Heterodimeric Fc Backbone 8 monomer 1 (-)
APEFLGGPSVFLFPPKPKDTLMISRTPEVTCVVVDVSQEDPEVQFNWYVDGVEVHNAKTKPREEEFNSTYRVVSVLTVLHQDWLNGKEYKCKVSNKGLPSSIEKTISKAKGQPREPQVYTLPPSQEEMTKNQVSLTCDVSGFYPSDIAVEWESDGQPENNYKTTPPVLDSDGSFFLYSRLTVDKSRWEEGDVFSCSVMHEALHNHYTQKSLSLSLGK

SEQ ID NO: 102 - protein, 217 aa, synthetic construct
  source 1..217: note = Heterodimeric Fc Backbone 8 monomer 2 (+)
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEQMTKNQVKLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 103 - protein, 216 aa, synthetic construct
  source 1..216: note = Heterodimeric Fc Backbone 9 monomer 1 (-)
APPVAGPSVFLFPPKPKDTLMISRTPEVTCVVVDVSHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 104 - protein, 216 aa, synthetic construct
  source 1..216: note = Heterodimeric Fc Backbone 9 monomer 2 (+)
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 105 - protein, 216 aa, synthetic construct
  source 1..216: note = Heterodimeric Fc Backbone 10 monomer 1 (-)
APPVAGPSVFLFPPKPKDTLMISRTPEVTCVVVDVKHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 106 - protein, 216 aa, synthetic construct
  source 1..216: note = Heterodimeric Fc Backbone 10 monomer 2 (+)
APPVAGPSVFLFPPKPKDTLMISRTPEVTCVVVDVKHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 107 - protein, 216 aa, synthetic construct
  source 1..216: note = Heterodimeric Fc Backbone 11 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 108 - protein, 216 aa, synthetic construct
  source 1..216: note = Heterodimeric Fc Backbone 11 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 109 - protein, 216 aa, synthetic construct
  source 1..216: note = Heterodimeric Fc Backbone 12 monomer 1 (-)
APPVAGPSVFLFPPKPKDTLMISRTPEVTCVVVDVKHEDPEVKFKWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 217 aa, synthetic construct
  source 1..217: note = Heterodimeric Fc Backbone 13 monomer 1 and 2 + 1 mAb-scFv Heterodimeric Fc Backbone monomer 1
APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 217 aa, synthetic construct
  source 1..217: note = Heterodimeric Fc Backbone 13 monomer 2
APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 112 - protein, 217 aa, synthetic construct
  source 1..217: note = Heterodimeric Fc Backbone 14 monomer 1
APEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 217 aa, synthetic construct
  source 1..217: note = Heterodimeric Fc Backbone 14 monomer 2
APEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 114 - protein, 216 aa, synthetic construct
  source 1..216: note = Heterodimeric Fc Backbone 15 monomer 1
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 115 - protein, 216 aa, synthetic construct
  source 1..216: note = Heterodimeric Fc Backbone 15 monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 116 - protein, 216 aa, synthetic construct
  source 1..216: note = Heterodimeric Fc Backbone 16 monomer 1
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 117 - protein, 216 aa, synthetic construct
  source 1..216: note = Heterodimeric Fc Backbone 16 monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 118 - protein, 216 aa, synthetic construct
  source 1..216: note = Heterodimeric Fc Backbone 17 monomer 1
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 119 - protein, 216 aa, synthetic construct
  source 1..216: note = Heterodimeric Fc Backbone 17 monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 120 - protein, 215 aa, synthetic construct
  source 1..215: note = 2 + 1 mAb-scFv Heterodimeric Fc Backbone monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 216 aa, synthetic construct
  source 1..216: note = 2 + 1 mAb-scFv Heterodimeric Fc Backbone monomer 2
APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 122 - protein, 98 aa, synthetic construct
  source 1..98: note = IgG1 CH1(+)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 123 - protein, 98 aa, synthetic construct
  source 1..98: note = IgG1 CH1(-)
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKV

SEQ ID NO: 124 - protein, 98 aa, synthetic construct
  source 1..98: note = IgG2 CH1(+)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 125 - protein, 98 aa, synthetic construct
  source 1..98: note = IgG2 CH1(-)
ASTKGPSVFPLAPCSRSTSESTAALGCLVKDYFPEPVTVSWNSGALTSGVHTFPAVLQSSGLYSLSSVVTVPSSNFGTQTYTCNVDHKPSDTKVDKTV

SEQ ID NO: 126 - protein, 98 aa, synthetic construct
  source 1..98: note = IgG4 CH1(+)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 127 - protein, 98 aa, synthetic construct
  source 1..98: note = IgG4 CH1(-)
ASTKGPSVFPLAPCSRSTSESTAALGCLVKDYFPEPVTVSWNSGALTSGVHTFPAVLQSSGLYSLSSVVTVPSSSLGTKTYTCNVDHKPSDTKVDKRV

SEQ ID NO: 128 - protein, 12 aa, synthetic construct
  source 1..12: note = IgG2 hinge
ERKCCVECPPCP

SEQ ID NO: 129 - protein, 12 aa, synthetic construct
  source 1..12: note = IgG4 hinge
ESKYGPPCPSCP

SEQ ID NO: 130 - protein, 107 aa, synthetic construct [sequence omitted]

SEQ ID NO: 131 - protein, 106 aa, synthetic construct
  source 1..106: note = Light Chain Constant Domain - Lambda
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 132 - protein, 121 aa, synthetic construct
  source 1..121: note = 1A7[CD28]_H1.2
EVQLLESGGGLVQPGGSLRLSCAASGFTFSSYAMSWVRQAPGKGLEWVSTIDGSGDSTYYADSVKGRFTISRDNSKNTLYLQMNSLRAEDTAVYYCAKSGPGLRQVGFDYWGQGTLVTVSS

SEQ ID NO: 133 - protein, 121 aa, synthetic construct
  source 1..121: note = 1A7[CD28]_H1.3
EVQLLESGGGLVQPGGSLRLSCAASGFTFSSYAMSWVRQAPGKGLEWVSTIEGSGDSTYYADSVKGRFTISRDNSKNTLYLQMNSLRAEDTAVYYCAKSGPGLRQVGFDYWGQGTLVTVSS

SEQ ID NO: 134 - protein, 121 aa, synthetic construct
  source 1..121: note = 1A7[CD28]_H1.4
EVQLLESGGGLVQPGGSLRLSCAASGFTFSSYAMSWVRQAPGKGLEWVSTIYGSGDSTYYADSVKGRFTISRDNSKNTLYLQMNSLRAEDTAVYYCAKSGPGLRQVGFDYWGQGTLVTVSS

SEQ ID NO: 135 - protein, 121 aa, synthetic construct
  source 1..121: note = 1A7[CD28]_H1.5
EVQLLESGGGLVQPGGSLRLSCAASGFTFSSYAMSWVRQAPGKGLEWVSTISDSGDSTYYADSVKGRFTISRDNSKNTLYLQMNSLRAEDTAVYYCAKSGPGLRQVGFDYWGQGTLVTVSS

SEQ ID NO: 136 - protein, 121 aa, synthetic construct
  source 1..121: note = 1A7[CD28]_H1.6
EVQLLESGGGLVQPGGSLRLSCAASGFTFSSYAMSWVRQAPGKGLEWVSTISESGDSTYYADSVKGRFTISRDNSKNTLYLQMNSLRAEDTAVYYCAKSGPGLRQVGFDYWGQGTLVTVSS

SEQ ID NO: 137 - protein, 121 aa, synthetic construct
  source 1..121: note = 1A7[CD28]_H1.7
EVQLLESGGGLVQPGGSLRLSCAASGFTFSSYAMSWVRQAPGKGLEWVSTISGSGTSTYYADSVKGRFTISRDNSKNTLYLQMNSLRAEDTAVYYCAKSGPGLRQVGFDYWGQGTLVTVSS

SEQ ID NO: 138 - protein, 121 aa, synthetic construct
  source 1..121: note = 1A7[CD28]_H1.8
EVQLLESGGGLVQPGGSLRLSCAASGFTFSSYAMSWVRQAPGKGLEWVSTISGSGYSTYYADSVKGRFTISRDNSKNTLYLQMNSLRAEDTAVYYCAKSGPGLRQVGFDYWGQGTLVTVSS

SEQ ID NO: 139 - protein, 121 aa, synthetic construct
  source 1..121: note = 1A7[CD28]_H1.9
EVQLLESGGGLVQPGGSLRLSCAASGFTFSSYAMSWVRQAPGKGLEWVSTISGSGDYTYYADSVKGRFTISRDNSKNTLYLQMNSLRAEDTAVYYCAKSGPGLRQVGFDYWGQGTLVTVSS

SEQ ID NO: 140 - protein, 121 aa, synthetic construct
  source 1..121: note = 1A7[CD28]_H1.10
EVQLLESGGGLVQPGGSLRLSCAASGFTFSSYYMSWVRQAPGKGLEWVSTIDGSGDSTYYADSVKGRFTISRDNSKNTLYLQMNSLRAEDTAVYYCAKSGPGLRQVGFDYWGQGTLVTVSS

SEQ ID NO: 141 - protein, 121 aa, synthetic construct
  source 1..121: note = 1A7[CD28]_H1.11
EVQLLESGGGLVQPGGSLRLSCAASGFTFSSYYMSWVRQAPGKGLEWVSTIEGSGDSTYYADSVKGRFTISRDNSKNTLYLQMNSLRAEDTAVYYCAKSGPGLRQVGFDYWGQGTLVTVSS

SEQ ID NO: 142 - protein, 121 aa, synthetic construct
  source 1..121: note = 1A7[CD28]_H1.12
EVQLLESGGGLVQPGGSLRLSCAASGFTFSSYYMSWVRQAPGKGLEWVSTIYGSGDSTYYADSVKGRFTISRDNSKNTLYLQMNSLRAEDTAVYYCAKSGPGLRQVGFDYWGQGTLVTVSS

SEQ ID NO: 143 - protein, 121 aa, synthetic construct
  source 1..121: note = 1A7[CD28]_H1.13
EVQLLESGGGLVQPGGSLRLSCAASGFTFSSYYMSWVRQAPGKGLEWVSTISDSGDSTYYADSVKGRFTISRDNSKNTLYLQMNSLRAEDTAVYYCAKSGPGLRQVGFDYWGQGTLVTVSS

SEQ ID NO: 144 - protein, 450 aa, synthetic construct
  source 1..450: note = XENP27181 Heavy Chain
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 121 aa, synthetic construct
  source 1..121: note = 1A7[CD28]_H1.15
EVQLLESGGGLVQPGGSLRLSCAASGFTFSSYYMSWVRQAPGKGLEWVSTISGSGTSTYYADSVKGRFTISRDNSKNTLYLQMNSLRAEDTAVYYCAKSGPGLRQVGFDYWGQGTLVTVSS

SEQ ID NO: 146 - protein, 121 aa, synthetic construct
  source 1..121: note = 1A7[CD28]_H1.16
EVQLLESGGGLVQPGGSLRLSCAASGFTFSSYYMSWVRQAPGKGLEWVSTISGSGYSTYYADSVKGRFTISRDNSKNTLYLQMNSLRAEDTAVYYCAKSGPGLRQVGFDYWGQGTLVTVSS

SEQ ID NO: 147 - protein, 121 aa, synthetic construct
  source 1..121: note = 1A7[CD28]_H1.17
EVQLLESGGGLVQPGGSLRLSCAASGFTFSSYYMSWVRQAPGKGLEWVSTISGSGDYTYYADSVKGRFTISRDNSKNTLYLQMNSLRAEDTAVYYCAKSGPGLRQVGFDYWGQGTLVTVSS

SEQ ID NO: 148 - protein, 121 aa, synthetic construct
  source 1..121: note = 1A7[CD28]_H1.18
EVQLLESGGGLVQPGGSLRLSCAASGFTFSSYAMSWVRQAPGKGLEWVSTIDDSGDSTYYADSVKGRFTISRDNSKNTLYLQMNSLRAEDTAVYYCAKSGPGLRQVGFDYWGQGTLVTVSS

SEQ ID NO: 149 - protein, 121 aa, synthetic construct
  source 1..121: note = 1A7[CD28]_H1.19
EVQLLESGGGLVQPGGSLRLSCAASGFTFSSYAMSWVRQAPGKGLEWVSTIDESGDSTYYADSVKGRFTISRDNSKNTLYLQMNSLRAEDTAVYYCAKSGPGLRQVGFDYWGQGTLVTVSS

SEQ ID NO: 150 - protein, 121 aa, synthetic construct
  source 1..121: note = 1A7[CD28]_H1.20
EVQLLESGGGLVQPGGSLRLSCAASGFTFSSYAMSWVRQAPGKGLEWVSTIDGSGTSTYYADSVKGRFTISRDNSKNTLYLQMNSLRAEDTAVYYCAKSGPGLRQVGFDYWGQGTLVTVSS

SEQ ID NO: 151 - protein, 121 aa, synthetic construct
  source 1..121: note = 1A7[CD28]_H1.21
EVQLLESGGGLVQPGGSLRLSCAASGFTFSSYAMSWVRQAPGKGLEWVSTIDGSGYSTYYADSVKGRFTISRDNSKNTLYLQMNSLRAEDTAVYYCAKSGPGLRQVGFDYWGQGTLVTVSS

SEQ ID NO: 152 - protein, 121 aa, synthetic construct
  source 1..121: note = 1A7[CD28]_H1.22
EVQLLESGGGLVQPGGSLRLSCAASGFTFSSYAMSWVRQAPGKGLEWVSTIDGSGDYTYYADSVKGRFTISRDNSKNTLYLQMNSLRAEDTAVYYCAKSGPGLRQVGFDYWGQGTLVTVSS

SEQ ID NO: 153 - protein, 121 aa, synthetic construct
  source 1..121: note = 1A7[CD28]_H1.23
EVQLLESGGGLVQPGGSLRLSCAASGFTFSSYAMSWVRQAPGKGLEWVSTIEDSGDSTYYADSVKGRFTISRDNSKNTLYLQMNSLRAEDTAVYYCAKSGPGLRQVGFDYWGQGTLVTVSS

SEQ ID NO: 154 - protein, 121 aa, synthetic construct
  source 1..121: note = 1A7[CD28]_H1.24
EVQLLESGGGLVQPGGSLRLSCAASGFTFSSYAMSWVRQAPGKGLEWVSTIEESGDSTYYADSVKGRFTISRDNSKNTLYLQMNSLRAEDTAVYYCAKSGPGLRQVGFDYWGQGTLVTVSS

SEQ ID NO: 155 - protein, 121 aa, synthetic construct
  source 1..121: note = 1A7[CD28]_H1.25
EVQLLESGGGLVQPGGSLRLSCAASGFTFSSYAMSWVRQAPGKGLEWVSTIEGSGTSTYYADSVKGRFTISRDNSKNTLYLQMNSLRAEDTAVYYCAKSGPGLRQVGFDYWGQGTLVTVSS

SEQ ID NO: 156 - protein, 121 aa, synthetic construct
  source 1..121: note = 1A7[CD28]_H1.26
EVQLLESGGGLVQPGGSLRLSCAASGFTFSSYAMSWVRQAPGKGLEWVSTIEGSGYSTYYADSVKGRFTISRDNSKNTLYLQMNSLRAEDTAVYYCAKSGPGLRQVGFDYWGQGTLVTVSS

SEQ ID NO: 157 - protein, 121 aa, synthetic construct
  source 1..121: note = 1A7[CD28]_H1.27
EVQLLESGGGLVQPGGSLRLSCAASGFTFSSYAMSWVRQAPGKGLEWVSTIEGSGDYTYYADSVKGRFTISRDNSKNTLYLQMNSLRAEDTAVYYCAKSGPGLRQVGFDYWGQGTLVTVSS

SEQ ID NO: 158 - protein, 121 aa, synthetic construct
  source 1..121: note = 1A7[CD28]_H1.28
EVQLLESGGGLVQPGGSLRLSCAASGFTFSSYAMSWVRQAPGKGLEWVSTIYDSGDSTYYADSVKGRFTISRDNSKNTLYLQMNSLRAEDTAVYYCAKSGPGLRQVGFDYWGQGTLVTVSS

SEQ ID NO: 159 - protein, 121 aa, synthetic construct
  source 1..121: note = 1A7[CD28]_H1.29
EVQLLESGGGLVQPGGSLRLSCAASGFTFSSYAMSWVRQAPGKGLEWVSTIYESGDSTYYADSVKGRFTISRDNSKNTLYLQMNSLRAEDTAVYYCAKSGPGLRQVGFDYWGQGTLVTVSS

SEQ ID NO: 160 - protein, 121 aa, synthetic construct
  source 1..121: note = 1A7[CD28]_H1.30
EVQLLESGGGLVQPGGSLRLSCAASGFTFSSYAMSWVRQAPGKGLEWVSTIYGSGTSTYYADSVKGRFTISRDNSKNTLYLQMNSLRAEDTAVYYCAKSGPGLRQVGFDYWGQGTLVTVSS

SEQ ID NO: 161 - protein, 121 aa, synthetic construct
  source 1..121: note = 1A7[CD28]_H1.31
EVQLLESGGGLVQPGGSLRLSCAASGFTFSSYAMSWVRQAPGKGLEWVSTIYGSGYSTYYADSVKGRFTISRDNSKNTLYLQMNSLRAEDTAVYYCAKSGPGLRQVGFDYWGQGTLVTVSS

SEQ ID NO: 162 - protein, 121 aa, synthetic construct
  source 1..121: note = 1A7[CD28]_H1.32
EVQLLESGGGLVQPGGSLRLSCAASGFTFSSYAMSWVRQAPGKGLEWVSTIYGSGDYTYYADSVKGRFTISRDNSKNTLYLQMNSLRAEDTAVYYCAKSGPGLRQVGFDYWGQGTLVTVSS

SEQ ID NO: 163 - protein, 121 aa, synthetic construct
  source 1..121: note = 1A7[CD28]_H1.33
EVQLLESGGGLVQPGGSLRLSCAASGFTFSSYAMSWVRQAPGKGLEWVSTISDSGTSTYYADSVKGRFTISRDNSKNTLYLQMNSLRAEDTAVYYCAKSGPGLRQVGFDYWGQGTLVTVSS

SEQ ID NO: 164 - protein, 121 aa, synthetic construct
  source 1..121: note = 1A7[CD28]_H1.34
EVQLLESGGGLVQPGGSLRLSCAASGFTFSSYAMSWVRQAPGKGLEWVSTISDSGYSTYYADSVKGRFTISRDNSKNTLYLQMNSLRAEDTAVYYCAKSGPGLRQVGFDYWGQGTLVTVSS

SEQ ID NO: 165 - protein, 121 aa, synthetic construct
  source 1..121: note = 1A7[CD28]_H1.35
EVQLLESGGGLVQPGGSLRLSCAASGFTFSSYAMSWVRQAPGKGLEWVSTISDSGDYTYYADSVKGRFTISRDNSKNTLYLQMNSLRAEDTAVYYCAKSGPGLRQVGFDYWGQGTLVTVSS

SEQ ID NO: 166 - protein, 121 aa, synthetic construct
  source 1..121: note = 1A7[CD28]_H1.36
EVQLLESGGGLVQPGGSLRLSCAASGFTFSSYAMSWVRQAPGKGLEWVSTISESGTSTYYADSVKGRFTISRDNSKNTLYLQMNSLRAEDTAVYYCAKSGPGLRQVGFDYWGQGTLVTVSS

SEQ ID NO: 167 - protein, 121 aa, synthetic construct
  source 1..121: note = 1A7[CD28]_H1.37
EVQLLESGGGLVQPGGSLRLSCAASGFTFSSYAMSWVRQAPGKGLEWVSTISESGYSTYYADSVKGRFTISRDNSKNTLYLQMNSLRAEDTAVYYCAKSGPGLRQVGFDYWGQGTLVTVSS

SEQ ID NO: 168 - protein, 121 aa, synthetic construct
  source 1..121: note = 1A7[CD28]_H1.38
EVQLLESGGGLVQPGGSLRLSCAASGFTFSSYAMSWVRQAPGKGLEWVSTISESGDYTYYADSVKGRFTISRDNSKNTLYLQMNSLRAEDTAVYYCAKSGPGLRQVGFDYWGQGTLVTVSS

SEQ ID NO: 169 - protein, 121 aa, synthetic construct
  source 1..121: note = 1A7[CD28]_H1.39
EVQLLESGGGLVQPGGSLRLSCAASGFTFSSYAMSWVRQAPGKGLEWVSTISGSGTYTYYADSVKGRFTISRDNSKNTLYLQMNSLRAEDTAVYYCAKSGPGLRQVGFDYWGQGTLVTVSS

SEQ ID NO: 170 - protein, 121 aa, synthetic construct
  source 1..121: note = 1A7[CD28]_H1.40
EVQLLESGGGLVQPGGSLRLSCAASGFTFSSYAMSWVRQAPGKGLEWVSTISGSGYYTYYADSVKGRFTISRDNSKNTLYLQMNSLRAEDTAVYYCAKSGPGLRQVGFDYWGQGTLVTVSS

SEQ ID NO: 171 - protein, 121 aa, synthetic construct
  source 1..121: note = 1A7[CD28]_H1.41
EVQLLESGGGLVQPGGSLRLSCAASGFTFSSYYMSWVRQAPGKGLEWVSTIEGSGDYTYYADSVKGRFTISRDNSKNTLYLQMNSLRAEDTAVYYCAKSGPGLRQVGFDYWGQGTLVTVSS

SEQ ID NO: 172 - protein, 121 aa, synthetic construct
  source 1..121: note = 1A7[CD28]_H1.42
EVQLLESGGGLVQPGGSLRLSCAASGFTFSSYYMSWVRQAPGKGLEWVSTISESGDYTYYADSVKGRFTISRDNSKNTLYLQMNSLRAEDTAVYYCAKSGPGLRQVGFDYWGQGTLVTVSS

SEQ ID NO: 173 - protein, 121 aa, synthetic construct
  source 1..121: note = 1A7[CD28]_H1.43
EVQLLESGGGLVQPGGSLRLSCAASGFSFSGNYMTWVRQAPGKGLEWVATITADSDATYYADSVKGRFTISRDNSKNTLYLQMNSLRAEDTAVYYCAKSGPGLRQVGFDYWGQGTLVTVSS

SEQ ID NO: 174 - protein, 121 aa, synthetic construct
  source 1..121: note = 1A7[CD28]_H1.44
EVQLLESGGGLVQPGGSLRLSCAASGFTFSGYSMNWVRQAPGKGLEWVATIYANGSYTYYADSVKGRFTISRDNSKNTLYLQMNSLRAEDTAVYYCAKSGPGLRQVGFDYWGQGTLVTVSS

SEQ ID NO: 175 - protein, 121 aa, synthetic construct
  source 1..121: note = 1A7[CD28]_H1.45
EVQLLESGGGLVQPGGSLRLSCAASGFNFEEYSMNWVRQAPGKGLEWVATITYNGDYTYYADSVKGRFTISRDNSKNTLYLQMNSLRAEDTAVYYCAKSGPGLRQVGFDYWGQGTLVTVSS

SEQ ID NO: 176 - protein, 121 aa, synthetic construct
  source 1..121: note = 1A7[CD28]_H1.46
EVQLLESGGGLVQPGGSLRLSCAASGFSFRTYYMTWVRQAPGKGLEWVATITSDGDYTYYADSVKGRFTISRDNSKNTLYLQMNSLRAEDTAVYYCAKSGPGLRQVGFDYWGQGTLVTVSS

SEQ ID NO: 177 - protein, 121 aa, synthetic construct
  source 1..121: note = 1A7[CD28]_H1.47
EVQLLESGGGLVQPGGSLRLSCAASGFSFKGYSMNWVRQAPGKGLEWVATIYASSDSTYYADSVKGRFTISRDNSKNTLYLQMNSLRAEDTAVYYCAKSGPGLRQVGFDYWGQGTLVTVSS

SEQ ID NO: 178 - protein, 121 aa, synthetic construct
  source 1..121: note = 1A7[CD28]_H1.48
EVQLLESGGGLVQPGGSLRLSCAASGFSFGEYSMNWVRQAPGKGLEWVSTIYADGDYTYYADSVKGRFTISRDNSKNTLYLQMNSLRAEDTAVYYCAKSGPGLRQVGFDYWGQGTLVTVSS

SEQ ID NO: 179 - protein, 121 aa, synthetic construct
  source 1..121: note = 1A7[CD28]_H1.49
EVQLLESGGGLVQPGGSLRLSCAASGFTFTDYSMNWVRQAPGKGLEWVATIYADSSSTYYADSVKGRFTISRDNSKNTLYLQMNSLRAEDTAVYYCAKSGPGLRQVGFDYWGQGTLVTVSS

SEQ ID NO: 180 - protein, 121 aa, synthetic construct
  source 1..121: note = 1A7[CD28]_H1.50
EVQLLESGGGLVQPGGSLRLSCAASGFTFGGYSMNWVRQAPGKGLEWVATIYYDSTTTYYADSVKGRFTISRDNSKNTLYLQMNSLRAEDTAVYYCAKSGPGLRQVGFDYWGQGTLVTVSS

SEQ ID NO: 181 - protein, 121 aa, synthetic construct
  source 1..121: note = 1A7[CD28]_H1.51
EVQLLESGGGLVQPGGSLRLSCAASGFTFGAYSMNWVRQAPGKGLEWVSTIYNDGATTYYADSVKGRFTISRDNSKNTLYLQMNSLRAEDTAVYYCAKSGPGLRQVGFDYWGQGTLVTVSS

SEQ ID NO: 182 - protein, 121 aa, synthetic construct
  source 1..121: note = 1A7[CD28]_H1.52
EVQLLESGGGLVQPGGSLRLSCAASGFSFEAYSMNWVRQAPGKGLEWVATIYYDSSYTYYADSVKGRFTISRDNSKNTLYLQMNSLRAEDTAVYYCAKSGPGLRQVGFDYWGQGTLVTVSS

SEQ ID NO: 183 - protein, 121 aa, synthetic construct
  source 1..121: note = 1A7[CD28]_H1.53
EVQLLESGGGLVQPGGSLRLSCAASGFSFGSYYMSWVRQAPGKGLEWVASIYYGGYDTYYADSVKGRFTISRDNSKNTLYLQMNSLRAEDTAVYYCAKSGPGLRQVGFDYWGQGTLVTVSS

SEQ ID NO: 184 - protein, 121 aa, synthetic construct
  source 1..121: note = 1A7[CD28]_H1.54
EVQLLESGGGLVQPGGSLRLSCAASGFNFAEYSMSWVRQAPGKGLEWVATIYAGSDYTYYADSVKGRFTISRDNSKNTLYLQMNSLRAEDTAVYYCAKSGPGLRQVGFDYWGQGTLVTVSS

SEQ ID NO: 185 - protein, 121 aa, synthetic construct
  source 1..121: note = 1A7[CD28]_H1.55
EVQLLESGGGLVQPGGSLRLSCAASGFTFGAYSINWVRQAPGKGLEWVATIYYDGSYTYYADSVKGRFTISRDNSKNTLYLQMNSLRAEDTAVYYCAKSGPGLRQVGFDYWGQGTLVTVSS

SEQ ID NO: 186 - protein, 121 aa, synthetic construct
  source 1..121: note = 1A7[CD28]_H1.56
EVQLLESGGGLVQPGGSLRLSCAASGFTFSRYYINWVRQAPGKGLEWVASIYDGGADTYYADSVKGRFTISRDNSKNTLYLQMNSLRAEDTAVYYCAKSGPGLRQVGFDYWGQGTLVTVSS

SEQ ID NO: 187 - protein, 121 aa, synthetic construct
  source 1..121: note = 1A7[CD28]_H1.57
EVQLLESGGGLVQPGGSLRLSCAASGFSFGTYSINWVRQAPGKGLEWVSTIYYDGASTYYADSVKGRFTISRDNSKNTLYLQMNSLRAEDTAVYYCAKSGPGLRQVGFDYWGQGTLVTVSS

SEQ ID NO: 188 - protein, 121 aa, synthetic construct
  source 1..121: note = 1A7[CD28]_H1.58
EVQLLESGGGLVQPGGSLRLSCAASGFTFGKYSISWVRQAPGKGLEWVATIYNDGYYTYYADSVKGRFTISRDNSKNTLYLQMNSLRAEDTAVYYCAKSGPGLRQVGFDYWGQGTLVTVSS

SEQ ID NO: 189 - protein, 121 aa, synthetic construct
  source 1..121: note = 1A7[CD28]_H1.59
EVQLLESGGGLVQPGGSLRLSCAASGFSFEKYYISWVRQAPGKGLEWVASIYDGSYDTYYADSVKGRFTISRDNSKNTLYLQMNSLRAEDTAVYYCAKSGPGLRQVGFDYWGQGTLVTVSS

SEQ ID NO: 190 - protein, 121 aa, synthetic construct
  source 1..121: note = 1A7[CD28]_H1.60
EVQLLESGGGLVQPGGSLRLSCAASGFSFGTYSMNWVRQAPGKGLEWVATIDYDGSNTYYADSVKGRFTISRDNSKNTLYLQMNSLRAEDTAVYYCAKSGPGLRQVGFDYWGQGTLVTVSS

SEQ ID NO: 191 - protein, 121 aa, synthetic construct
  source 1..121: note = 1A7[CD28]_H1.61
EVQLLESGGGLVQPGGSLRLSCAASGFSFAAYSMSWVRQAPGKGLEWVATIYAGSDYTYYADSVKGRFTISRDNSKNTLYLQMNSLRAEDTAVYYCAKSGPGLRQVGFDYWGQGTLVTVSS

SEQ ID NO: 192 - protein, 121 aa, synthetic construct
  source 1..121: note = 1A7[CD28]_H1.62
EVQLLESGGGLVQPGGSLRLSCAASGFTFTTYSMTWVRQAPGKGLEWVATIYNDGYYTYYADSVKGRFTISRDNSKNTLYLQMNSLRAEDTAVYYCAKSGPGLRQVGFDYWGQGTLVTVSS

SEQ ID NO: 193 - protein, 121 aa, synthetic construct
  source 1..121: note = 1A7[CD28]_H1.63
EVQLLESGGGLVQPGGSLRLSCAASGFSFKDYSMNWVRQAPGKGLEWVATIYYDGTYTYYADSVKGRFTISRDNSKNTLYLQMNSLRAEDTAVYYCAKSGPGLRQVGFDYWGQGTLVTVSS

SEQ ID NO: 194 - protein, 121 aa, synthetic construct
  source 1..121: note = 1A7[CD28]_H1.64
EVQLLESGGGLVQPGGSLRLSCAASGFSFAAYSMNWVRQAPGKGLEWVATIYAGGAYTYYADSVKGRFTISRDNSKNTLYLQMNSLRAEDTAVYYCAKSGPGLRQVGFDYWGQGTLVTVSS

SEQ ID NO: 195 - protein, 121 aa, synthetic construct
  source 1..121: note = 1A7[CD28]_H1.65
EVQLLESGGGLVQPGGSLRLSCAASGFNFAEYYISWVRQAPGKGLEWVASIYNGGSDTYYADSVKGRFTISRDNSKNTLYLQMNSLRAEDTAVYYCAKSGPGLRQVGFDYWGQGTLVTVSS

SEQ ID NO: 196 - protein, 121 aa, synthetic construct
  source 1..121: note = 1A7[CD28]_H1.66
EVQLLESGGGLVQPGGSLRLSCAASGFSLGEYYMNWVRQAPGKGLEWVSTIDYDGTYTYYADSVKGRFTISRDNSKNTLYLQMNSLRAEDTAVYYCAKSGPGLRQVGFDYWGQGTLVTVSS

SEQ ID NO: 197 - protein, 121 aa, synthetic construct
  source 1..121: note = 1A7[CD28]_H1.67
EVQLLESGGGLVQPGGSLRLSCAASGFTFNEYYITWVRQAPGKGLEWVSSIYSSSYDTYYADSVKGRFTISRDNSKNTLYLQMNSLRAEDTAVYYCAKSGPGLRQVGFDYWGQGTLVTVSS

SEQ ID NO: 198 - protein, 121 aa, synthetic construct
  source 1..121: note = 1A7[CD28]_H1.68
EVQLLESGGGLVQPGGSLRLSCAASGFTFKDYYITWVRQAPGKGLEWVSSIYTSGYDTYYADSVKGRFTISRDNSKNTLYLQMNSLRAEDTAVYYCAKSGPGLRQVGFDYWGQGTLVTVSS

SEQ ID NO: 199 - protein, 121 aa, synthetic construct
  source 1..121: note = 1A7[CD28]_H1.69
EVQLLESGGGLVQPGGSLRLSCAASGFSFGKYSINWVRQAPGKGLEWVATIYSDGTDTYYADSVKGRFTISRDNSKNTLYLQMNSLRAEDTAVYYCAKSGPGLRQVGFDYWGQGTLVTVSS

SEQ ID NO: 200 - protein, 107 aa, synthetic construct
  source 1..107: note = 1A7[CD28]_L1.1
DIQMTQSPSSLSASVGDRVTITCRASQSISAYLNWYQQKPGKAPKLLIYAASSLQSGVPSRFSGSGSGTDFTLTISSLQPEDFATYYCQQSYSTPFTFGQGTKLEIK

SEQ ID NO: 201 - protein, 107 aa, synthetic construct
  source 1..107: note = 1A7[CD28]_L1.2
DIQMTQSPSSLSASVGDRVTITCRASQSISDYLNWYQQKPGKAPKLLIYAASSLQSGVPSRFSGSGSGTDFTLTISSLQPEDFATYYCQQSYSTPFTFGQGTKLEIK

SEQ ID NO: 202 - protein, 107 aa, synthetic construct
  source 1..107: note = 1A7[CD28]_L1.3
DIQMTQSPSSLSASVGDRVTITCRASQSISGYLNWYQQKPGKAPKLLIYAASSLQSGVPSRFSGSGSGTDFTLTISSLQPEDFATYYCQQSYSTPFTFGQGTKLEIK

SEQ ID NO: 203 - protein, 107 aa, synthetic construct
  source 1..107: note = 1A7[CD28]_L1.4
DIQMTQSPSSLSASVGDRVTITCRASQSISHYLNWYQQKPGKAPKLLIYAASSLQSGVPSRFSGSGSGTDFTLTISSLQPEDFATYYCQQSYSTPFTFGQGTKLEIK

SEQ ID NO: 204 - protein, 107 aa, synthetic construct
  source 1..107: note = 1A7[CD28]_L1.5
DIQMTQSPSSLSASVGDRVTITCRASQSISKYLNWYQQKPGKAPKLLIYAASSLQSGVPSRFSGSGSGTDFTLTISSLQPEDFATYYCQQSYSTPFTFGQGTKLEIK

SEQ ID NO: 205 - protein, 107 aa, synthetic construct
  source 1..107: note = 1A7[CD28]_L1.6
DIQMTQSPSSLSASVGDRVTITCRASQSISNYLNWYQQKPGKAPKLLIYAASSLQSGVPSRFSGSGSGTDFTLTISSLQPEDFATYYCQQSYSTPFTFGQGTKLEIK

SEQ ID NO: 206 - protein, 107 aa, synthetic construct
  source 1..107: note = 1A7[CD28]_L1.7
DIQMTQSPSSLSASVGDRVTITCRASQSISQYLNWYQQKPGKAPKLLIYAASSLQSGVPSRFSGSGSGTDFTLTISSLQPEDFATYYCQQSYSTPFTFGQGTKLEIK

SEQ ID NO: 207 - protein, 107 aa, synthetic construct
  source 1..107: note = 1A7[CD28]_L1.8
DIQMTQSPSSLSASVGDRVTITCRASQSISTYLNWYQQKPGKAPKLLIYAASSLQSGVPSRFSGSGSGTDFTLTISSLQPEDFATYYCQQSYSTPFTFGQGTKLEIK

SEQ ID NO: 208 - protein, 107 aa, synthetic construct
  source 1..107: note = 1A7[CD28]_L1.9
DIQMTQSPSSLSASVGDRVTITCRASQSISVYLNWYQQKPGKAPKLLIYAASSLQSGVPSRFSGSGSGTDFTLTISSLQPEDFATYYCQQSYSTPFTFGQGTKLEIK

SEQ ID NO: 209 - protein, 107 aa, synthetic construct
  source 1..107: note = 1A7[CD28]_L1.10
DIQMTQSPSSLSASVGDRVTITCRASQSISYYLNWYQQKPGKAPKLLIYAASSLQSGVPSRFSGSGSGTDFTLTISSLQPEDFATYYCQQSYSTPFTFGQGTKLEIK

SEQ ID NO: 210 - protein, 107 aa, synthetic construct
  source 1..107: note = 1A7[CD28]_L1.11
DIQMTQSPSSLSASVGDRVTITCRASQSISSALNWYQQKPGKAPKLLIYAASSLQSGVPSRFSGSGSGTDFTLTISSLQPEDFATYYCQQSYSTPFTFGQGTKLEIK

SEQ ID NO: 211 - protein, 107 aa, synthetic construct
  source 1..107: note = 1A7[CD28]_L1.12
DIQMTQSPSSLSASVGDRVTITCRASQSISSDLNWYQQKPGKAPKLLIYAASSLQSGVPSRFSGSGSGTDFTLTISSLQPEDFATYYCQQSYSTPFTFGQGTKLEIK

SEQ ID NO: 212 - protein, 107 aa, synthetic construct
  source 1..107: note = 1A7[CD28]_L1.13
DIQMTQSPSSLSASVGDRVTITCRASQSISSFLNWYQQKPGKAPKLLIYAASSLQSGVPSRFSGSGSGTDFTLTISSLQPEDFATYYCQQSYSTPFTFGQGTKLEIK

SEQ ID NO: 213 - protein, 107 aa, synthetic construct
  source 1..107: note = 1A7[CD28]_L1.14
DIQMTQSPSSLSASVGDRVTITCRASQSISSHLNWYQQKPGKAPKLLIYAASSLQSGVPSRFSGSGSGTDFTLTISSLQPEDFATYYCQQSYSTPFTFGQGTKLEIK

SEQ ID NO: 214 - protein, 107 aa, synthetic construct
  source 1..107: note = 1A7[CD28]_L1.15
DIQMTQSPSSLSASVGDRVTITCRASQSISSKLNWYQQKPGKAPKLLIYAASSLQSGVPSRFSGSGSGTDFTLTISSLQPEDFATYYCQQSYSTPFTFGQGTKLEIK

SEQ ID NO: 215 - protein, 107 aa, synthetic construct
  source 1..107: note = 1A7[CD28]_L1.16
DIQMTQSPSSLSASVGDRVTITCRASQSISSLLNWYQQKPGKAPKLLIYAASSLQSGVPSRFSGSGSGTDFTLTISSLQPEDFATYYCQQSYSTPFTFGQGTKLEIK

SEQ ID NO: 216 - protein, 107 aa, synthetic construct
  source 1..107: note = 1A7[CD28]_L1.17
DIQMTQSPSSLSASVGDRVTITCRASQSISSNLNWYQQKPGKAPKLLIYAASSLQSGVPSRFSGSGSGTDFTLTISSLQPEDFATYYCQQSYSTPFTFGQGTKLEIK

SEQ ID NO: 217 - protein, 107 aa, synthetic construct
  source 1..107: note = 1A7[CD28]_L1.18
DIQMTQSPSSLSASVGDRVTITCRASQSISSQLNWYQQKPGKAPKLLIYAASSLQSGVPSRFSGSGSGTDFTLTISSLQPEDFATYYCQQSYSTPFTFGQGTKLEIK

SEQ ID NO: 218 - protein, 107 aa, synthetic construct
  source 1..107: note = 1A7[CD28]_L1.19
DIQMTQSPSSLSASVGDRVTITCRASQSISSSLNWYQQKPGKAPKLLIYAASSLQSGVPSRFSGSGSGTDFTLTISSLQPEDFATYYCQQSYSTPFTFGQGTKLEIK

SEQ ID NO: 219 - protein, 107 aa, synthetic construct
  source 1..107: note = 1A7[CD28]_L1.20
DIQMTQSPSSLSASVGDRVTITCRASQSISSWLNWYQQKPGKAPKLLIYAASSLQSGVPSRFSGSGSGTDFTLTISSLQPEDFATYYCQQSYSTPFTFGQGTKLEIK

SEQ ID NO: 220 - protein, 107 aa, synthetic construct
  source 1..107: note = 1A7[CD28]_L1.21
DIQMTQSPSSLSASVGDRVTITCRASQSISSYLAWYQQKPGKAPKLLIYAASSLQSGVPSRFSGSGSGTDFTLTISSLQPEDFATYYCQQSYSTPFTFGQGTKLEIK

SEQ ID NO: 221 - protein, 107 aa, synthetic construct
  source 1..107: note = 1A7[CD28]_L1.22
DIQMTQSPSSLSASVGDRVTITCRASQSISSYLDWYQQKPGKAPKLLIYAASSLQSGVPSRFSGSGSGTDFTLTISSLQPEDFATYYCQQSYSTPFTFGQGTKLEIK

SEQ ID NO: 222 - protein, 107 aa, synthetic construct
  source 1..107: note = 1A7[CD28]_L1.23
DIQMTQSPSSLSASVGDRVTITCRASQSISSYLGWYQQKPGKAPKLLIYAASSLQSGVPSRFSGSGSGTDFTLTISSLQPEDFATYYCQQSYSTPFTFGQGTKLEIK

SEQ ID NO: 223 - protein, 107 aa, synthetic construct
  source 1..107: note = 1A7[CD28]_L1.24
DIQMTQSPSSLSASVGDRVTITCRASQSISSYLHWYQQKPGKAPKLLIYAASSLQSGVPSRFSGSGSGTDFTLTISSLQPEDFATYYCQQSYSTPFTFGQGTKLEIK

SEQ ID NO: 224 - protein, 107 aa, synthetic construct
  source 1..107: note = 1A7[CD28]_L1.25
DIQMTQSPSSLSASVGDRVTITCRASQSISSYLQWYQQKPGKAPKLLIYAASSLQSGVPSRFSGSGSGTDFTLTISSLQPEDFATYYCQQSYSTPFTFGQGTKLEIK

SEQ ID NO: 225 - protein, 107 aa, synthetic construct
  source 1..107: note = 1A7[CD28]_L1.26
DIQMTQSPSSLSASVGDRVTITCRASQSISSYLSWYQQKPGKAPKLLIYAASSLQSGVPSRFSGSGSGTDFTLTISSLQPEDFATYYCQQSYSTPFTFGQGTKLEIK

SEQ ID NO: 226 - protein, 107 aa, synthetic construct
  source 1..107: note = 1A7[CD28]_L1.27
DIQMTQSPSSLSASVGDRVTITCRASQSISSYLTWYQQKPGKAPKLLIYAASSLQSGVPSRFSGSGSGTDFTLTISSLQPEDFATYYCQQSYSTPFTFGQGTKLEIK

SEQ ID NO: 227 - protein, 107 aa, synthetic construct
  source 1..107: note = 1A7[CD28]_L1.28
DIQMTQSPSSLSASVGDRVTITCRASQSISSYLYWYQQKPGKAPKLLIYAASSLQSGVPSRFSGSGSGTDFTLTISSLQPEDFATYYCQQSYSTPFTFGQGTKLEIK

SEQ ID NO: 228 - protein, 107 aa, synthetic construct
  source 1..107: note = 1A7[CD28]_L1.29
DIQMTQSPSSLSASVGDRVTITCRASQSISSYLNWYQQKPGKAPKLLIYDASSLQSGVPSRFSGSGSGTDFTLTISSLQPEDFATYYCQQSYSTPFTFGQGTKLEIK

SEQ ID NO: 229 - protein, 107 aa, synthetic construct
  source 1..107: note = 1A7[CD28]_L1.30
DIQMTQSPSSLSASVGDRVTITCRASQSISSYLNWYQQKPGKAPKLLIYGASSLQSGVPSRFSGSGSGTDFTLTISSLQPEDFATYYCQQSYSTPFTFGQGTKLEIK

SEQ ID NO: 230 - protein, 107 aa, synthetic construct
  source 1..107: note = 1A7[CD28]_L1.31
DIQMTQSPSSLSASVGDRVTITCRASQSISSYLNWYQQKPGKAPKLLIYKASSLQSGVPSRFSGSGSGTDFTLTISSLQPEDFATYYCQQSYSTPFTFGQGTKLEIK

SEQ ID NO: 231 - protein, 107 aa, synthetic construct
  source 1..107: note = 1A7[CD28]_L1.32
DIQMTQSPSSLSASVGDRVTITCRASQSISSYLNWYQQKPGKAPKLLIYLASSLQSGVPSRFSGSGSGTDFTLTISSLQPEDFATYYCQQSYSTPFTFGQGTKLEIK

SEQ ID NO: 232 - protein, 107 aa, synthetic construct
  source 1..107: note = 1A7[CD28]_L1.33
DIQMTQSPSSLSASVGDRVTITCRASQSISSYLNWYQQKPGKAPKLLIYQASSLQSGVPSRFSGSGSGTDFTLTISSLQPEDFATYYCQQSYSTPFTFGQGTKLEIK

SEQ ID NO: 233 - protein, 107 aa, synthetic construct
  source 1..107: note = 1A7[CD28]_L1.34
DIQMTQSPSSLSASVGDRVTITCRASQSISSYLNWYQQKPGKAPKLLIYSASSLQSGVPSRFSGSGSGTDFTLTISSLQPEDFATYYCQQSYSTPFTFGQGTKLEIK

SEQ ID NO: 234 - protein, 107 aa, synthetic construct
  source 1..107: note = 1A7[CD28]_L1.35
DIQMTQSPSSLSASVGDRVTITCRASQSISSYLNWYQQKPGKAPKLLIYTASSLQSGVPSRFSGSGSGTDFTLTISSLQPEDFATYYCQQSYSTPFTFGQGTKLEIK

SEQ ID NO: 235 - protein, 107 aa, synthetic construct
  source 1..107: note = 1A7[CD28]_L1.36
DIQMTQSPSSLSASVGDRVTITCRASQSISSYLNWYQQKPGKAPKLLIYWASSLQSGVPSRFSGSGSGTDFTLTISSLQPEDFATYYCQQSYSTPFTFGQGTKLEIK

SEQ ID NO: 236 - protein, 107 aa, synthetic construct
  source 1..107: note = 1A7[CD28]_L1.37
DIQMTQSPSSLSASVGDRVTITCRASQSISSYLNWYQQKPGKAPKLLIYYASSLQSGVPSRFSGSGSGTDFTLTISSLQPEDFATYYCQQSYSTPFTFGQGTKLEIK

SEQ ID NO: 237 - protein, 107 aa, synthetic construct
  source 1..107: note = 1A7[CD28]_L1.38
DIQMTQSPSSLSASVGDRVTITCRASQSISSYLNWYQQKPGKAPKLLIYAASALQSGVPSRFSGSGSGTDFTLTISSLQPEDFATYYCQQSYSTPFTFGQGTKLEIK

SEQ ID NO: 238 - protein, 107 aa, synthetic construct
  source 1..107: note = 1A7[CD28]_L1.39
DIQMTQSPSSLSASVGDRVTITCRASQSISSYLNWYQQKPGKAPKLLIYAASDLQSGVPSRFSGSGSGTDFTLTISSLQPEDFATYYCQQSYSTPFTFGQGTKLEIK

SEQ ID NO: 239 - protein, 107 aa, synthetic construct
  source 1..107: note = 1A7[CD28]_L1.40
DIQMTQSPSSLSASVGDRVTITCRASQSISSYLNWYQQKPGKAPKLLIYAASKLQSGVPSRFSGSGSGTDFTLTISSLQPEDFATYYCQQSYSTPFTFGQGTKLEIK

SEQ ID NO: 240 - protein, 107 aa, synthetic construct
  source 1..107: note = 1A7[CD28]_L1.41
DIQMTQSPSSLSASVGDRVTITCRASQSISSYLNWYQQKPGKAPKLLIYAASNLQSGVPSRFSGSGSGTDFTLTISSLQPEDFATYYCQQSYSTPFTFGQGTKLEIK

SEQ ID NO: 241 - protein, 107 aa, synthetic construct
  source 1..107: note = 1A7[CD28]_L1.42
DIQMTQSPSSLSASVGDRVTITCRASQSISSYLNWYQQKPGKAPKLLIYAASQLQSGVPSRFSGSGSGTDFTLTISSLQPEDFATYYCQQSYSTPFTFGQGTKLEIK

SEQ ID NO: 242 - protein, 107 aa, synthetic construct
  source 1..107: note = 1A7[CD28]_L1.43
DIQMTQSPSSLSASVGDRVTITCRASQSISSYLNWYQQKPGKAPKLLIYAASTLQSGVPSRFSGSGSGTDFTLTISSLQPEDFATYYCQQSYSTPFTFGQGTKLEIK

SEQ ID NO: 243 - protein, 107 aa, synthetic construct
  source 1..107: note = 1A7[CD28]_L1.44
DIQMTQSPSSLSASVGDRVTITCRASQSISSYLNWYQQKPGKAPKLLIYAASYLQSGVPSRFSGSGSGTDFTLTISSLQPEDFATYYCQQSYSTPFTFGQGTKLEIK

SEQ ID NO: 244 - protein, 107 aa, synthetic construct
  source 1..107: note = 1A7[CD28]_L1.45
DIQMTQSPSSLSASVGDRVTITCRASQSISSYLNWYQQKPGKAPKLLIYAASSLASGVPSRFSGSGSGTDFTLTISSLQPEDFATYYCQQSYSTPFTFGQGTKLEIK

SEQ ID NO: 245 - protein, 107 aa, synthetic construct
  source 1..107: note = 1A7[CD28]_L1.46
DIQMTQSPSSLSASVGDRVTITCRASQSISSYLNWYQQKPGKAPKLLIYAASSLESGVPSRFSGSGSGTDFTLTISSLQPEDFATYYCQQSYSTPFTFGQGTKLEIK

SEQ ID NO: 246 - protein, 107 aa, synthetic construct
  source 1..107: note = 1A7[CD28]_L1.47
DIQMTQSPSSLSASVGDRVTITCRASQSISSYLNWYQQKPGKAPKLLIYAASSLFSGVPSRFSGSGSGTDFTLTISSLQPEDFATYYCQQSYSTPFTFGQGTKLEIK

SEQ ID NO: 247 - protein, 107 aa, synthetic construct
  source 1..107: note = 1A7[CD28]_L1.48
DIQMTQSPSSLSASVGDRVTITCRASQSISSYLNWYQQKPGKAPKLLIYAASSLHSGVPSRFSGSGSGTDFTLTISSLQPEDFATYYCQQSYSTPFTFGQGTKLEIK

SEQ ID NO: 248 - protein, 107 aa, synthetic construct
  source 1..107: note = 1A7[CD28]_L1.49
DIQMTQSPSSLSASVGDRVTITCRASQSISSYLNWYQQKPGKAPKLLIYAASSLISGVPSRFSGSGSGTDFTLTISSLQPEDFATYYCQQSYSTPFTFGQGTKLEIK

SEQ ID NO: 249 - protein, 107 aa, synthetic construct
  source 1..107: note = 1A7[CD28]_L1.50
DIQMTQSPSSLSASVGDRVTITCRASQSISSYLNWYQQKPGKAPKLLIYAASSLKSGVPSRFSGSGSGTDFTLTISSLQPEDFATYYCQQSYSTPFTFGQGTKLEIK

SEQ ID NO: 250 - protein, 107 aa, synthetic construct
  source 1..107: note = 1A7[CD28]_L1.51
DIQMTQSPSSLSASVGDRVTITCRASQSISSYLNWYQQKPGKAPKLLIYAASSLNSGVPSRFSGSGSGTDFTLTISSLQPEDFATYYCQQSYSTPFTFGQGTKLEIK

SEQ ID NO: 251 - protein, 107 aa, synthetic construct
  source 1..107: note = 1A7[CD28]_L1.52
DIQMTQSPSSLSASVGDRVTITCRASQSISSYLNWYQQKPGKAPKLLIYAASSLSSGVPSRFSGSGSGTDFTLTISSLQPEDFATYYCQQSYSTPFTFGQGTKLEIK

SEQ ID NO: 252 - protein, 107 aa, synthetic construct
  source 1..107: note = 1A7[CD28]_L1.53
DIQMTQSPSSLSASVGDRVTITCRASQSISSYLNWYQQKPGKAPKLLIYAASSLVSGVPSRFSGSGSGTDFTLTISSLQPEDFATYYCQQSYSTPFTFGQGTKLEIK

SEQ ID NO: 253 - protein, 107 aa, synthetic construct
  source 1..107: note = 1A7[CD28]_L1.54
DIQMTQSPSSLSASVGDRVTITCRASQSISSYLNWYQQKPGKAPKLLIYAASSLYSGVPSRFSGSGSGTDFTLTISSLQPEDFATYYCQQSYSTPFTFGQGTKLEIK

SEQ ID NO: 254 - protein, 107 aa, synthetic construct
  source 1..107: note = 1A7[CD28]_L1.55
DIQMTQSPSSLSASVGDRVTITCRASQSISSYLNWYQQKPGKAPKLLIYAASSLQAGVPSRFSGSGSGTDFTLTISSLQPEDFATYYCQQSYSTPFTFGQGTKLEIK

SEQ ID NO: 255 - protein, 107 aa, synthetic construct
  source 1..107: note = 1A7[CD28]_L1.56
DIQMTQSPSSLSASVGDRVTITCRASQSISSYLNWYQQKPGKAPKLLIYAASSLQDGVPSRFSGSGSGTDFTLTISSLQPEDFATYYCQQSYSTPFTFGQGTKLEIK

SEQ ID NO: 256 - protein, 107 aa, synthetic construct
  source 1..107: note = 1A7[CD28]_L1.57
DIQMTQSPSSLSASVGDRVTITCRASQSISSYLNWYQQKPGKAPKLLIYAASSLQGGVPSRFSGSGSGTDFTLTISSLQPEDFATYYCQQSYSTPFTFGQGTKLEIK

SEQ ID NO: 257 - protein, 107 aa, synthetic construct
  source 1..107: note = 1A7[CD28]_L1.58
DIQMTQSPSSLSASVGDRVTITCRASQSISSYLNWYQQKPGKAPKLLIYAASSLQHGVPSRFSGSGSGTDFTLTISSLQPEDFATYYCQQSYSTPFTFGQGTKLEIK

SEQ ID NO: 258 - protein, 107 aa, synthetic construct
  source 1..107: note = 1A7[CD28]_L1.59
DIQMTQSPSSLSASVGDRVTITCRASQSISSYLNWYQQKPGKAPKLLIYAASSLQKGVPSRFSGSGSGTDFTLTISSLQPEDFATYYCQQSYSTPFTFGQGTKLEIK

SEQ ID NO: 259 - protein, 107 aa, synthetic construct
  source 1..107: note = 1A7[CD28]_L1.60
DIQMTQSPSSLSASVGDRVTITCRASQSISSYLNWYQQKPGKAPKLLIYAASSLQQGVPSRFSGSGSGTDFTLTISSLQPEDFATYYCQQSYSTPFTFGQGTKLEIK

SEQ ID NO: 260 - protein, 107 aa, synthetic construct
  source 1..107: note = 1A7[CD28]_L1.61
DIQMTQSPSSLSASVGDRVTITCRASQSISSYLNWYQQKPGKAPKLLIYAASSLQTGVPSRFSGSGSGTDFTLTISSLQPEDFATYYCQQSYSTPFTFGQGTKLEIK

SEQ ID NO: 261 - protein, 107 aa, synthetic construct
  source 1..107: note = 1A7[CD28]_L1.62
DIQMTQSPSSLSASVGDRVTITCRASQSISSYLNWYQQKPGKAPKLLIYAASSLQVGVPSRFSGSGSGTDFTLTISSLQPEDFATYYCQQSYSTPFTFGQGTKLEIK

SEQ ID NO: 262 - protein, 107 aa, synthetic construct
  source 1..107: note = 1A7[CD28]_L1.63
DIQMTQSPSSLSASVGDRVTITCRASQSISSYLNWYQQKPGKAPKLLIYAASSLQYGVPSRFSGSGSGTDFTLTISSLQPEDFATYYCQQSYSTPFTFGQGTKLEIK

SEQ ID NO: 263 - protein, 107 aa, synthetic construct
  source 1..107: note = 1A7[CD28]_L1.64
DIQMTQSPSSLSASVGDRVTITCRASQSISSYLNWYQQKPGKAPKLLIYAASSLQSGVPSRFSGSGSGTDFTLTISSLQPEDFATYYCQQAYSTPFTFGQGTKLEIK

SEQ ID NO: 264 - protein, 107 aa, synthetic construct
  source 1..107: note = 1A7[CD28]_L1.65
DIQMTQSPSSLSASVGDRVTITCRASQSISSYLNWYQQKPGKAPKLLIYAASSLQSGVPSRFSGSGSGTDFTLTISSLQPEDFATYYCQQDYSTPFTFGQGTKLEIK

SEQ ID NO: 265 - protein, 107 aa, synthetic construct
  source 1..107: note = 1A7[CD28]_L1.66
DIQMTQSPSSLSASVGDRVTITCRASQSISSYLNWYQQKPGKAPKLLIYAASSLQSGVPSRFSGSGSGTDFTLTISSLQPEDFATYYCQQFYSTPFTFGQGTKLEIK

SEQ ID NO: 266 - protein, 107 aa, synthetic construct
  source 1..107: note = 1A7[CD28]_L1.67
DIQMTQSPSSLSASVGDRVTITCRASQSISSYLNWYQQKPGKAPKLLIYAASSLQSGVPSRFSGSGSGTDFTLTISSLQPEDFATYYCQQHYSTPFTFGQGTKLEIK

SEQ ID NO: 267 - protein, 107 aa, synthetic construct
  source 1..107: note = 1A7[CD28]_L1.68
DIQMTQSPSSLSASVGDRVTITCRASQSISSYLNWYQQKPGKAPKLLIYAASSLQSGVPSRFSGSGSGTDFTLTISSLQPEDFATYYCQQKYSTPFTFGQGTKLEIK

SEQ ID NO: 268 - protein, 107 aa, synthetic construct
  source 1..107: note = 1A7[CD28]_L1.69
DIQMTQSPSSLSASVGDRVTITCRASQSISSYLNWYQQKPGKAPKLLIYAASSLQSGVPSRFSGSGSGTDFTLTISSLQPEDFATYYCQQLYSTPFTFGQGTKLEIK

SEQ ID NO: 269 - protein, 107 aa, synthetic construct
  source 1..107: note = 1A7[CD28]_L1.70
DIQMTQSPSSLSASVGDRVTITCRASQSISSYLNWYQQKPGKAPKLLIYAASSLQSGVPSRFSGSGSGTDFTLTISSLQPEDFATYYCQQTYSTPFTFGQGTKLEIK

SEQ ID NO: 270 - protein, 218 aa, synthetic construct
  source 1..218: note = XENP27181 Light Chain
DIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - protein, 107 aa, synthetic construct
  source 1..107: note = 1A7[CD28]_L1.72
DIQMTQSPSSLSASVGDRVTITCRASQSISSYLNWYQQKPGKAPKLLIYAASSLQSGVPSRFSGSGSGTDFTLTISSLQPEDFATYYCQQYYSTPFTFGQGTKLEIK

SEQ ID NO: 272 - protein, 107 aa, synthetic construct
  source 1..107: note = 1A7[CD28]_L1.73
DIQMTQSPSSLSASVGDRVTITCRASQSISSYLNWYQQKPGKAPKLLIYAASSLQSGVPSRFSGSGSGTDFTLTISSLQPEDFATYYCQQSASTPFTFGQGTKLEIK

SEQ ID NO: 273 - protein, 107 aa, synthetic construct
  source 1..107: note = 1A7[CD28]_L1.74
DIQMTQSPSSLSASVGDRVTITCRASQSISSYLNWYQQKPGKAPKLLIYAASSLQSGVPSRFSGSGSGTDFTLTISSLQPEDFATYYCQQSDSTPFTFGQGTKLEIK

SEQ ID NO: 274 - protein, 107 aa, synthetic construct
  source 1..107: note = 1A7[CD28]_L1.75
DIQMTQSPSSLSASVGDRVTITCRASQSISSYLNWYQQKPGKAPKLLIYAASSLQSGVPSRFSGSGSGTDFTLTISSLQPEDFATYYCQQSFSTPFTFGQGTKLEIK

SEQ ID NO: 275 - protein, 107 aa, synthetic construct
  source 1..107: note = 1A7[CD28]_L1.76
DIQMTQSPSSLSASVGDRVTITCRASQSISSYLNWYQQKPGKAPKLLIYAASSLQSGVPSRFSGSGSGTDFTLTISSLQPEDFATYYCQQSHSTPFTFGQGTKLEIK

SEQ ID NO: 276 - protein, 107 aa, synthetic construct
  source 1..107: note = 1A7[CD28]_L1.77
DIQMTQSPSSLSASVGDRVTITCRASQSISSYLNWYQQKPGKAPKLLIYAASSLQSGVPSRFSGSGSGTDFTLTISSLQPEDFATYYCQQSKSTPFTFGQGTKLEIK

SEQ ID NO: 277 - protein, 107 aa, synthetic construct
  source 1..107: note = 1A7[CD28]_L1.78
DIQMTQSPSSLSASVGDRVTITCRASQSISSYLNWYQQKPGKAPKLLIYAASSLQSGVPSRFSGSGSGTDFTLTISSLQPEDFATYYCQQSLSTPFTFGQGTKLEIK

SEQ ID NO: 278 - protein, 107 aa, synthetic construct
  source 1..107: note = 1A7[CD28]_L1.79
DIQMTQSPSSLSASVGDRVTITCRASQSISSYLNWYQQKPGKAPKLLIYAASSLQSGVPSRFSGSGSGTDFTLTISSLQPEDFATYYCQQSQSTPFTFGQGTKLEIK

SEQ ID NO: 279 - protein, 107 aa, synthetic construct
  source 1..107: note = 1A7[CD28]_L1.80
DIQMTQSPSSLSASVGDRVTITCRASQSISSYLNWYQQKPGKAPKLLIYAASSLQSGVPSRFSGSGSGTDFTLTISSLQPEDFATYYCQQSVSTPFTFGQGTKLEIK

SEQ ID NO: 280 - protein, 107 aa, synthetic construct
  source 1..107: note = 1A7[CD28]_L1.81
DIQMTQSPSSLSASVGDRVTITCRASQSISSYLNWYQQKPGKAPKLLIYAASSLQSGVPSRFSGSGSGTDFTLTISSLQPEDFATYYCQQSWSTPFTFGQGTKLEIK

SEQ ID NO: 281 - protein, 107 aa, synthetic construct
  source 1..107: note = 1A7[CD28]_L1.82
DIQMTQSPSSLSASVGDRVTITCRASQSISSYLNWYQQKPGKAPKLLIYAASSLQSGVPSRFSGSGSGTDFTLTISSLQPEDFATYYCQQSYATPFTFGQGTKLEIK

SEQ ID NO: 282 - protein, 107 aa, synthetic construct
  source 1..107: note = 1A7[CD28]_L1.83
DIQMTQSPSSLSASVGDRVTITCRASQSISSYLNWYQQKPGKAPKLLIYAASSLQSGVPSRFSGSGSGTDFTLTISSLQPEDFATYYCQQSYDTPFTFGQGTKLEIK

SEQ ID NO: 283 - protein, 107 aa, synthetic construct
  source 1..107: note = 1A7[CD28]_L1.84
DIQMTQSPSSLSASVGDRVTITCRASQSISSYLNWYQQKPGKAPKLLIYAASSLQSGVPSRFSGSGSGTDFTLTISSLQPEDFATYYCQQSYGTPFTFGQGTKLEIK

SEQ ID NO: 284 - protein, 107 aa, synthetic construct
  source 1..107: note = 1A7[CD28]_L1.85
DIQMTQSPSSLSASVGDRVTITCRASQSISSYLNWYQQKPGKAPKLLIYAASSLQSGVPSRFSGSGSGTDFTLTISSLQPEDFATYYCQQSYHTPFTFGQGTKLEIK

SEQ ID NO: 285 - protein, 107 aa, synthetic construct
  source 1..107: note = 1A7[CD28]_L1.86
DIQMTQSPSSLSASVGDRVTITCRASQSISSYLNWYQQKPGKAPKLLIYAASSLQSGVPSRFSGSGSGTDFTLTISSLQPEDFATYYCQQSYKTPFTFGQGTKLEIK

SEQ ID NO: 286 - protein, 107 aa, synthetic construct
  source 1..107: note = 1A7[CD28]_L1.87
DIQMTQSPSSLSASVGDRVTITCRASQSISSYLNWYQQKPGKAPKLLIYAASSLQSGVPSRFSGSGSGTDFTLTISSLQPEDFATYYCQQSYNTPFTFGQGTKLEIK

SEQ ID NO: 287 - protein, 107 aa, synthetic construct
  source 1..107: note = 1A7[CD28]_L1.88
DIQMTQSPSSLSASVGDRVTITCRASQSISSYLNWYQQKPGKAPKLLIYAASSLQSGVPSRFSGSGSGTDFTLTISSLQPEDFATYYCQQSYQTPFTFGQGTKLEIK

SEQ ID NO: 288 - protein, 107 aa, synthetic construct
  source 1..107: note = 1A7[CD28]_L1.89
DIQMTQSPSSLSASVGDRVTITCRASQSISSYLNWYQQKPGKAPKLLIYAASSLQSGVPSRFSGSGSGTDFTLTISSLQPEDFATYYCQQSYTTPFTFGQGTKLEIK

SEQ ID NO: 289 - protein, 107 aa, synthetic construct
  source 1..107: note = 1A7[CD28]_L1.90
DIQMTQSPSSLSASVGDRVTITCRASQSISSYLNWYQQKPGKAPKLLIYAASSLQSGVPSRFSGSGSGTDFTLTISSLQPEDFATYYCQQSYVTPFTFGQGTKLEIK

SEQ ID NO: 290 - protein, 107 aa, synthetic construct
  source 1..107: note = 1A7[CD28]_L1.91
DIQMTQSPSSLSASVGDRVTITCRASQSISSYLNWYQQKPGKAPKLLIYAASSLQSGVPSRFSGSGSGTDFTLTISSLQPEDFATYYCQQSYYTPFTFGQGTKLEIK

SEQ ID NO: 291 - protein, 107 aa, synthetic construct
  source 1..107: note = 1A7[CD28]_L1.92
DIQMTQSPSSLSASVGDRVTITCRASQSISSYLNWYQQKPGKAPKLLIYAASSLQSGVPSRFSGSGSGTDFTLTISSLQPEDFATYYCQQSYSAPFTFGQGTKLEIK

SEQ ID NO: 292 - protein, 107 aa, synthetic construct
  source 1..107: note = 1A7[CD28]_L1.93
DIQMTQSPSSLSASVGDRVTITCRASQSISSYLNWYQQKPGKAPKLLIYAASSLQSGVPSRFSGSGSGTDFTLTISSLQPEDFATYYCQQSYSDPFTFGQGTKLEIK

SEQ ID NO: 293 - protein, 107 aa, synthetic construct
  source 1..107: note = 1A7[CD28]_L1.94
DIQMTQSPSSLSASVGDRVTITCRASQSISSYLNWYQQKPGKAPKLLIYAASSLQSGVPSRFSGSGSGTDFTLTISSLQPEDFATYYCQQSYSFPFTFGQGTKLEIK

SEQ ID NO: 294 - protein, 107 aa, synthetic construct
  source 1..107: note = 1A7[CD28]_L1.95
DIQMTQSPSSLSASVGDRVTITCRASQSISSYLNWYQQKPGKAPKLLIYAASSLQSGVPSRFSGSGSGTDFTLTISSLQPEDFATYYCQQSYSIPFTFGQGTKLEIK

SEQ ID NO: 295 - protein, 107 aa, synthetic construct
  source 1..107: note = 1A7[CD28]_L1.96
DIQMTQSPSSLSASVGDRVTITCRASQSISSYLNWYQQKPGKAPKLLIYAASSLQSGVPSRFSGSGSGTDFTLTISSLQPEDFATYYCQQSYSKPFTFGQGTKLEIK

SEQ ID NO: 296 - protein, 107 aa, synthetic construct
  source 1..107: note = 1A7[CD28]_L1.97
DIQMTQSPSSLSASVGDRVTITCRASQSISSYLNWYQQKPGKAPKLLIYAASSLQSGVPSRFSGSGSGTDFTLTISSLQPEDFATYYCQQSYSLPFTFGQGTKLEIK

SEQ ID NO: 297 - protein, 107 aa, synthetic construct
  source 1..107: note = 1A7[CD28]_L1.98
DIQMTQSPSSLSASVGDRVTITCRASQSISSYLNWYQQKPGKAPKLLIYAASSLQSGVPSRFSGSGSGTDFTLTISSLQPEDFATYYCQQSYSQPFTFGQGTKLEIK

SEQ ID NO: 298 - protein, 107 aa, synthetic construct
  source 1..107: note = 1A7[CD28]_L1.99
DIQMTQSPSSLSASVGDRVTITCRASQSISSYLNWYQQKPGKAPKLLIYAASSLQSGVPSRFSGSGSGTDFTLTISSLQPEDFATYYCQQSYSSPFTFGQGTKLEIK

SEQ ID NO: 299 - protein, 107 aa, synthetic construct
  source 1..107: note = 1A7[CD28]_L1.100
DIQMTQSPSSLSASVGDRVTITCRASQSISSYLNWYQQKPGKAPKLLIYAASSLQSGVPSRFSGSGSGTDFTLTISSLQPEDFATYYCQQSYSVPFTFGQGTKLEIK

SEQ ID NO: 300 - protein, 107 aa, synthetic construct
  source 1..107: note = 1A7[CD28]_L1.101
DIQMTQSPSSLSASVGDRVTITCRASQSISSYLNWYQQKPGKAPKLLIYAASSLQSGVPSRFSGSGSGTDFTLTISSLQPEDFATYYCQQSYSYPFTFGQGTKLEIK

SEQ ID NO: 301 - protein, 107 aa, synthetic construct
  source 1..107: note = 1A7[CD28]_L1.102
DIQMTQSPSSLSASVGDRVTITCRASQSISSYLNWYQQKPGKAPKLLIYAASSLQSGVPSRFSGSGSGTDFTLTISSLQPEDFATYYCQQSYSTPITFGQGTKLEIK

SEQ ID NO: 302 - protein, 107 aa, synthetic construct
  source 1..107: note = 1A7[CD28]_L1.103
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKLEIK

SEQ ID NO: 303 - protein, 107 aa, synthetic construct
  source 1..107: note = 1A7[CD28]_L1.104
DIQMTQSPSSLSASVGDRVTITCRASQSISSYLNWYQQKPGKAPKLLIYAASSLQSGVPSRFSGSGSGTDFTLTISSLQPEDFATYYCQQSYSTPWTFGQGTKLEIK

SEQ ID NO: 304 - protein, 107 aa, synthetic construct
  source 1..107: note = 1A7[CD28]_L1.105
DIQMTQSPSSLSASVGDRVTITCRASQSISSYLTWYQQKPGKAPKLLIYAASSLQSGVPSRFSGSGSGTDFTLTISSLQPEDFATYYCQQVYSTPFTFGQGTKLEIK

SEQ ID NO: 305 - protein, 107 aa, synthetic construct
  source 1..107: note = 1A7[CD28]_L1.106
DIQMTQSPSSLSASVGDRVTITCRASQSISSYLTWYQQKPGKAPKLLIYAASSLQSGVPSRFSGSGSGTDFTLTISSLQPEDFATYYCQQSYNTPFTFGQGTKLEIK

SEQ ID NO: 306 - protein, 30 aa, synthetic construct
  source 1..30: note = 1A7 VH HFR1
  VARIANT 28: note = T, S, or N
  VARIANT 29: note = F or L
  VARIANT 30: note = S, E, R, K, G, T, A, or N
EVQLLESGGGLVQPGGSLRLSCAASGFXXX

SEQ ID NO: 307 - skipped

SEQ ID NO: 308 - protein, 14 aa, synthetic construct
  source 1..14: note = 1A7 VH HFR2
  VARIANT 14: note = S or A
WVRQAPGKGLEWVX

SEQ ID NO: 309 - protein, 17 aa, synthetic construct
  source 1..17: note = 1A7 VH HCDR2
  VARIANT 1: note = T or S
  VARIANT 3: note = S, D, E, Y, or T
  VARIANT 4: note = G, D, E, A, Y, S, N, or T
  VARIANT 5: note = S, D, N, or G
  VARIANT 6: note = D, T, Y, S, or A
  VARIANT 7: note = D, T, Y, S, or A
  VARIANT 8: note = S, Y, A, T, D, or N
XIXXXXXXTYYADSVKG

SEQ ID NO: 310 - protein, 32 aa, synthetic construct
  source 1..32: note = 1A7 VH HFR3
RFTISRDNSKNTLYLQMNSLRAEDTAVYYCAK

SEQ ID NO: 311 - protein, 446 aa, synthetic construct
  source 1..446: note = XENP29154 Heavy Chain - TGN1412 HC
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 312 - protein, 11 aa, synthetic construct
  source 1..11: note = 1A7 VH HFR4
WGQGTLVTVSS

SEQ ID NO: 313 - protein, 23 aa, synthetic construct
  source 1..23: note = 1A7 VL LFR1
DIQMTQSPSSLSASVGDRVTITC

SEQ ID NO: 314 - protein, 11 aa, synthetic construct
  source 1..11: note = 1A7 VL LCDR1
  VARIANT 8: note = S, A, D, G, H, K, N, Q, T, V, or Y
  VARIANT 9: note = Y, A, D, F, H, K, L, N, Q, S, or W
  VARIANT 11: note = N, A, D, G, H, Q, S, T, or Y
RASQSISXXLX

SEQ ID NO: 315 - protein, 15 aa, synthetic construct
  source 1..15: note = 1A7 VL LFR2
WYQQKPGKAPKLLIY

SEQ ID NO: 316 - skipped

SEQ ID NO: 317 - protein, 32 aa, synthetic construct
  source 1..32: note = 1A7 VL LFR3
GVPSRFSGSGSGTDFTLTISSLQPEDFATYYC

SEQ ID NO: 318 - protein, 9 aa, synthetic construct
  source 1..9: note = 1A7 VL LCDR3
  VARIANT 3: note = S, A, D, F, H, K, L, T, V, or Y
  VARIANT 4: note = Y, A, D, F, H, K, L, Q, V, or W
  VARIANT 5: note = S, A, D, G, H, K, N, Q, T, V, or Y
  VARIANT 6: note = T, A, D, F, I, K, L, Q, S, V, or Y
  VARIANT 8: note = F, I, L, or W
QQXXXXPXT

SEQ ID NO: 319 - protein, 10 aa, synthetic construct
  source 1..10: note = 1A7 VL LFR4
FGQGTKLEIK

SEQ ID NO: 320 - protein, 121 aa, synthetic construct
  source 1..121: note = CD28.3[CD28]_H0L0 Variable heavy (vh) domain
EVKLQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTVSS

SEQ ID NO: 321 - protein, 5 aa, synthetic construct
  source 1..5: note = CD28.3[CD28]_H0L0 and hCD28.3[CD28]_H1L1 vhCDR1
EYIIH

SEQ ID NO: 322 - protein, 17 aa, synthetic construct
  source 1..17: note = CD28.3[CD28]_H0L0 vhCDR2
WFYPGSNDIQYNAKFKG

SEQ ID NO: 323 - protein, 12 aa, synthetic construct
  source 1..12: note = CD28.3[CD28]_H0L0 and hCD28.3[CD28]_H1L1 vhCDR3
RDDFSGYDALPY

SEQ ID NO: 324 - protein, 107 aa, synthetic construct
  source 1..107: note = CD28.3[CD28]_H0L0 Variable light (vl) domain
DIQMTQSPASLSVSVGETVTITCRTNENIYSNLAWYQQKQGKSPQLLIYAATHLVEGVPSRFSGSGSGTQYSLKITSLQSEDFGNYYCQHFWGTPCTFGGGTKLEIK

SEQ ID NO: 325 - protein, 11 aa, synthetic construct
  source 1..11: note = CD28.3[CD28]_H0L0 vlCDR1
RTNENIYSNLA

SEQ ID NO: 326 - protein, 9 aa, synthetic construct
  source 1..9: note = CD28.3[CD28]_H0L0 and hCD28.3[CD28]_H1L1 vlCDR3
QHFWGTPCT

SEQ ID NO: 327 - protein, 121 aa, synthetic construct
  source 1..121: note = hCD28.3[CD28]_H1L1 Variable heavy (vh) domain
QVQLQQSGAELKKPGASVKVSCKASGYTFTEYIIHWIKLRSGQGLEWIGWFYPGSNDIQYNAQFKGKATLTADKSSSTVYMELTGLTPEDSAVYFCARRDDFSGYDALPYWGQGTLVTVSA

SEQ ID NO: 328 - protein, 214 aa, synthetic construct
  source 1..214: note = XENP29154 Light Chain - TGN1412 LC
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 329 - protein, 17 aa, synthetic construct
  source 1..17: note = hCD28.3[CD28]_H1L1 vhCDR2
WFYPGSNDIQYNAQFKG

SEQ ID NO: 330 - skipped

SEQ ID NO: 331 - protein, 107 aa, synthetic construct
  source 1..107: note = hCD28.3[CD28]_H1L1 Variable light (vl) domain
DIQMTQSPSSLSASVGDRVTITCKTNENIYSNLAWYQQKDGKSPQLLIYAATHLVEGVPSRFSGSGSGTQYSLTISSLQPEDFGNYYCQHFWGTPCTFGGGTKLEIK

SEQ ID NO: 332 - protein, 11 aa, synthetic construct
  source 1..11: note = hCD28.3[CD28]_H1L1 vlCDR1
KTNENIYSNLA

SEQ ID NO: 333 - skipped

SEQ ID NO: 334 - protein, 213 aa, synthetic construct
  source 1..213: note = XENP39575 Chain 3 - CD20-A_L1, XENP39580 Chain 3 - CD20-A_L1, XENP39581 Chain 3 - CD20-A_L1, XENP39582 Chain 3 - CD20-A_L1, XENP39583 Chain 3 - CD20-A_L1, XENP40384 Chain 3 - CD20-A_L1, XENP40385 Chain 3 - CD20-A_L1, XENP39572 Chain 3 - CD20-A_L1, and XENP39573 Chain 3 - CD20-A_L1, XENP39574 Chain 3 - CD20-A_L1
QIVLTQSPSSLSASVGDRVTITCRASSSVSYIHWFQQKPGKSPKPLIYATSNLASGVPVRFSGSGSGTDYTLTISSLQPEDFATYYCQQWTSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 335 - protein, 120 aa, synthetic construct
  source 1..120: note = 5.11A1[CD28]_H0L0 Variable heavy (vh) domain
QVQLQQSGPELVKPGTSVRISCEASGYTFTSYYIHWVKQRPGQGLEWIGCIYPGNVNTNYNEKFKDKATLIVDTSSNTAYMQLSRMTSEDSAVYFCTRSHYGLDWNFDVWGAGTTVTVSS

SEQ ID NO: 336 - protein, 5 aa, synthetic construct
  source 1..5: note = 5.11A1[CD28]_H0L0 and TGN1412_H1L1 vhCDR1
SYYIH

SEQ ID NO: 337 - protein, 17 aa, synthetic construct
  source 1..17: note = 5.11A1[CD28]_H0L0 and TGN1412_H1L1 vhCDR2
CIYPGNVNTNYNEKFKD

SEQ ID NO: 338 - protein, 11 aa, synthetic construct
  source 1..11: note = 5.11A1[CD28]_H0L0 and TGN1412_H1L1 vhCDR3
SHYGLDWNFDV

SEQ ID NO: 339 - protein, 107 aa, synthetic construct
  source 1..107: note = 5.11A1[CD28]_H0L0 Variable light (vl) domain
DIQMNQSPSSLSASLGDTITITCHASQNIYVWLNWYQQKPGNIPKLLIYKASNLHTGVPSRFSGSGSGTGFTLTISSLQPEDIATYYCQQGQTYPYTFGGGTKLEIK

SEQ ID NO: 340 - protein, 11 aa, synthetic construct
  source 1..11: note = 5.11A1[CD28]_H0L0 and TGN1412_H1L1 vlCDR1
HASQNIYVWLN

SEQ ID NO: 341 - protein, 7 aa, synthetic construct
  source 1..7: note = 5.11A1[CD28]_H0L0 and TGN1412_H1L1 vlCDR2
KASNLHT

SEQ ID NO: 342 - protein, 9 aa, synthetic construct
  source 1..9: note = 5.11A1[CD28]_H0L0 and TGN1412_H1L1 vlCDR3
QQGQTYPYT

SEQ ID NO: 343 - protein, 120 aa, synthetic construct
  source 1..120: note = TGN1412_H1L1 Variable heavy (vh) domain
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 344-346 - skipped (3 entries)

SEQ ID NO: 347 - protein, 107 aa, synthetic construct
  source 1..107: note = TGN1412_H1L1 Variable light (vl) domain
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 348-350 - skipped (3 entries)

SEQ ID NO: 351 - protein, 127 aa, synthetic construct
  source 1..127: note = 341VL34[CD28]_H1L1 Variable heavy (vh) domain
EVQLVESGGGLVKPGGSLRLSCGGSGFTFNNAWMNWVRQAPGKGLEWVGRIKGKTDGGTADYAAPVKGRFTISRDYSKNTLYLQMNSLTTEDTAVYYCNTDLPYYYGSGRYSGMDVWGQGTTVTVSS

SEQ ID NO: 352 - protein, 5 aa, synthetic construct
  source 1..5: note = 341VL34[CD28]_H1L1 and 341VL36[CD28]_H1L1 vhCDR1
NAWMN

SEQ ID NO: 353 - protein, 19 aa, synthetic construct
  source 1..19: note = 341VL34[CD28]_H1L1 and 341VL36[CD28]_H1L1 vhCDR2
RIKGKTDGGTADYAAPVKG

SEQ ID NO: 354 - protein, 16 aa, synthetic construct
  source 1..16: note = 341VL34[CD28]_H1L1 vhCDR3
DLPYYYGSGRYSGMDV

SEQ ID NO: 355 - protein, 104 aa, synthetic construct
  source 1..104: note = 341VL34[CD28]_H1L1 and 341VL36[CD28]_H1L1 Variable light (vl) domain
EIVLTQSPATLSLSPGERATLSCRASQSVSSYLAWYQQKPGQAPRLLIYDASNRATGIPARFSGSGSGTDFTLTISSLEPEDFAVYYCQQRSNTFGPGTKVDIK

SEQ ID NO: 356 - protein, 11 aa, synthetic construct
  source 1..11: note = 341VL34[CD28]_H1L1, 341VL36[CD28]_H1L1, and CD20-C vlCDR1
RASQSVSSYLA

SEQ ID NO: 357 - protein, 7 aa, synthetic construct
  source 1..7: note = 341VL34[CD28]_H1L1, 341VL36[CD28]_H1L1, and CD20-C vlCDR2
DASNRAT

SEQ ID NO: 358 - protein, 6 aa, synthetic construct
  source 1..6: note = 341VL34[CD28]_H1L1 and 341VL36[CD28]_H1L1 vlCDR3
QQRSNT

SEQ ID NO: 359 - protein, 127 aa, synthetic construct
  source 1..127: note = 341VL36[CD28]_H1L1 Variable heavy (vh) domain
EVQLVESGGGLVKPGGSLRLSCGGSGFTFNNAWMNWVRQAPGKGLEWVGRIKGKTDGGTADYAAPVKGRFTISRDYSKNTLYLQMNSLKTEDTGVYYCTTYLPYYYGSERWSGMDVWGQGTTVTVSS

SEQ ID NO: 360-361 - skipped (2 entries)

SEQ ID NO: 362 - protein, 16 aa, synthetic construct
  source 1..16: note = 341VL36[CD28]_H1L1 vhCDR3
YLPYYYGSERWSGMDV

SEQ ID NO: 363-365 - skipped (3 entries)

SEQ ID NO: 366 - protein, 214 aa, synthetic construct
  source 1..214: note = XENP39956 Chain 3 - CD20-C_L1, XENP39957 Chain 3 - CD20-C_L1, XENP39958 Chain 3 - CD20-C_L1, XENP39959 Chain 3 - CD20-C_L1, XENP40367 Chain 3 - CD20-C_L1, XENP40368 Chain 3 - CD20-C_L1, XENP42159 Chain 3 - CD20-C_L1, XENP42160 Chain 3 - CD20-C_L1, XENP42161 Chain 3 - CD20-C_L1, XENP39968 Chain 3 - CD20-C_L1, XENP39969 Chain 3 - CD20-C_L1, XENP39970 Chain 3 - CD20-C_L1, and XENP39971 Chain 3 - CD20-C_L1
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 367 - protein, 126 aa, synthetic construct
  source 1..126: note = 281VL4[CD28]_H1L1 Variable heavy (vh) domain
EVQLVQSGSELKKPGSSVKVSCKASGGTSRSFAISWVRQAPGQGLEWMGGIIPIFGPANYAQKFQGRVTITADESTSTAYMELSSLRSEDTAVYYCARHAIAMGWGVITTNYFDSWGQGTMVTVSS

SEQ ID NO: 368 - protein, 5 aa, synthetic construct
  source 1..5: note = 281VL4[CD28]_H1L1 vhCDR1
SFAIS

SEQ ID NO: 369 - protein, 17 aa, synthetic construct
  source 1..17: note = 281VL4[CD28]_H1L1 vhCDR2
GIIPIFGPANYAQKFQG

SEQ ID NO: 370 - protein, 17 aa, synthetic construct
  source 1..17: note = 281VL4[CD28]_H1L1 vhCDR3
HAIAMGWGVITTNYFDS

SEQ ID NO: 371 - protein, 108 aa, synthetic construct
  source 1..108: note = 281VL4[CD28]_H1L1 Variable light (vl) domain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGQGTRLEIK

SEQ ID NO: 372 - protein, 12 aa, synthetic construct
  source 1..12: note = 281VL4[CD28]_H1L1 vlCDR1
RASQSVSSSYLA

SEQ ID NO: 373 - protein, 7 aa, synthetic construct
  source 1..7: note = 281VL4[CD28]_H1L1 vlCDR2
GASSRAT

SEQ ID NO: 374 - protein, 9 aa, synthetic construct
  source 1..9: note = 281VL4[CD28]_H1L1 vlCDR3
QQYGSSPIT

SEQ ID NO: 375 - protein, 121 aa, synthetic construct
  source 1..121: note = HuTN228[CD28]_H1L1 Variable heavy (vh) domain
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSS

SEQ ID NO: 376 - protein, 5 aa, synthetic construct
  source 1..5: note = HuTN228[CD28]_H1L1 and TN228[CD28]_H4L2 vhCDR1
SYGVH

SEQ ID NO: 377 - protein, 16 aa, synthetic construct
  source 1..16: note = HuTN228[CD28]_H1L1 vhCDR2
VIWPGGGTNFNSALMS

SEQ ID NO: 378 - protein, 13 aa, synthetic construct
  source 1..13: note = HuTN228[CD28]_H1L1 and TN228[CD28]_H4L2 vhCDR3
DRAYGNYLYAMDY

SEQ ID NO: 379 - protein, 111 aa, synthetic construct
  source 1..111: note = HuTN228[CD28]_H1L1 Variable light (vl) domain
DIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIK

SEQ ID NO: 380 - protein, 15 aa, synthetic construct
  source 1..15: note = HuTN228[CD28]_H1L1, m9.3[CD28]_H0L0, and TN228[CD28]_H4L2 vlCDR1
RASESVEYYVTSLMQ

SEQ ID NO: 381 - protein, 7 aa, synthetic construct
  source 1..7: note = HuTN228[CD28]_H1L1 and TN228[CD28]_H4L2 vlCDR2
AASNVDS

SEQ ID NO: 382 - protein, 9 aa, synthetic construct
  source 1..9: note = HuTN228[CD28]_H1L1 and TN228[CD28]_H4L2 vlCDR3
QQSRKVPFT

SEQ ID NO: 383 - protein, 124 aa, synthetic construct
  source 1..124: note = PV1[CD28]_H0L0 Variable heavy (vh) domain
QVQLKQSGAELVKPGASVKISCKTSGYTFTDGYMNWVEQKPGQGLEWIGRIDPDSGNTRYNQKFQGKATLTRDKSSSTVYMDLRSLTSEDSAVYYCARDGTFYGTYGYWYFDFWGQGTQVTVSS

SEQ ID NO: 384 - protein, 5 aa, synthetic construct
  source 1..5: note = PV1[CD28]_H0L0 vhCDR1
DGYMN

SEQ ID NO: 385 - protein, 17 aa, synthetic construct
  source 1..17: note = PV1[CD28]_H0L0 vhCDR2
RIDPDSGNTRYNQKFQG

SEQ ID NO: 386 - protein, 15 aa, synthetic construct
  source 1..15: note = PV1[CD28]_H0L0 vhCDR3
DGTFYGTYGYWYFDF

SEQ ID NO: 387 - protein, 113 aa, synthetic construct
  source 1..113: note = PV1[CD28]_H0L0 Variable light (vl) domain
DIVMTQSPYSLAVSAGEKVTMSCRSSQSLYYSGIKKNLLAWYQQKPGQSPKLLIYFTSTRLPGVPDRFTGSGSGTDYTLTITSVQAEDMGHYFCQQGISTPLTFGDGTKLEIR

SEQ ID NO: 388 - protein, 17 aa, synthetic construct
  source 1..17: note = PV1[CD28]_H0L0 vlCDR1
RSSQSLYYSGIKKNLLA

SEQ ID NO: 389 - protein, 7 aa, synthetic construct
  source 1..7: note = PV1[CD28]_H0L0 vlCDR2
FTSTRLP

SEQ ID NO: 390 - protein, 9 aa, synthetic construct
  source 1..9: note = PV1[CD28]_H0L0 vlCDR3
QQGISTPLT

SEQ ID NO: 391 - protein, 120 aa, synthetic construct
  source 1..120: note = m9.3[CD28]_H0L0 Variable heavy (vh) domain
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSS

SEQ ID NO: 392 - protein, 5 aa, synthetic construct
  source 1..5: note = m9.3[CD28]_H0L0 and hu9.3[CD28]_H1L1 vhCDR1
DYGVH

SEQ ID NO: 393 - protein, 16 aa, synthetic construct
  source 1..16: note = m9.3[CD28]_H0L0 vhCDR2
VIWAGGGTNYNSALMS

SEQ ID NO: 394 - protein, 12 aa, synthetic construct
  source 1..12: note = m9.3[CD28]_H0L0 and hu9.3[CD28]_H1L1 vhCDR3
DKGYSYYYSMDY

SEQ ID NO: 395 - protein, 111 aa, synthetic construct
  source 1..111: note = m9.3[CD28]_H0L0 Variable light (vl) domain
DIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIK

SEQ ID NO: 396 - skipped

SEQ ID NO: 397 - protein, 7 aa, synthetic construct
  source 1..7: note = m9.3[CD28]_H0L0 and hu9.3[CD28]_H1L1 vlCDR2
AASNVES

SEQ ID NO: 398 - protein, 9 aa, synthetic construct
  source 1..9: note = m9.3[CD28]_H0L0 and hu9.3[CD28]_H1L1 vlCDR3
QQSRKVPYT

SEQ ID NO: 399 - protein, 120 aa, synthetic construct
  source 1..120: note = hu9.3[CD28]_H1L1 Variable heavy (vh) domain
EVQLVQSGGGLVQPGGSLRLSCAGSGFTFSDYGVHWVRQAPGKGLEWVSAIWAGGGTNYASSVMGRFTISRDNAKNSLYLQMNSLRAEDMAVYYCARDKGYSYYYSMDYWGQGTLVTVSS

SEQ ID NO: 400 - skipped

SEQ ID NO: 401 - protein, 16 aa, synthetic construct
  source 1..16: note = hu9.3[CD28]_H1L1 vhCDR2
AIWAGGGTNYASSVMG

SEQ ID NO: 402 - skipped

SEQ ID NO: 403 - protein, 111 aa, synthetic construct
  source 1..111: note = hu9.3[CD28]_H1L1 Variable light (vl) domain
DIVMTQSPDSLAVSLGERATINCRASESVEYYVTSLMAWYQQKPGQPPKLLIYAASNVESGVPDRFSGSGSGTNFSLTISSLQAEDVAVYYCQQSRKVPYTFGQGTKLEIK

SEQ ID NO: 404 - protein, 15 aa, synthetic construct
  source 1..15: note = hu9.3[CD28]_H1L1 vlCDR1
RASESVEYYVTSLMA

SEQ ID NO: 405 - protein, 450 aa, synthetic construct
  source 1..450: note = XENP39580 Chain 1 - CD20-A_H1_IgG1_pI(-)_Isosteric_ A_PVA_/S267K/L368D/K370S/M428L/N434S, XENP39581 Chain 1 - CD20-A_H1_IgG1_pI(-)_Isosteric_A_PVA_/S267K/L368D/K370S/M4 28L/N434S, XENP39582 Chain 1 - CD20-A_H1_IgG1_pI(-)_Isoste ric_A_PVA_/S267K/L368D/K370S/M428L/N434S, XENP39583 Chain 1 - CD20-A_H1_IgG1_pI(-)_Isosteric_A_PVA_/S267K/L368D/K370 S/M428L/N434S, XENP39572 Chain 1 - CD20-A_H1_IgG1_pI(-)_Is osteric_A_PVA_/S267K/L368D/K370S/M428L/N434S, XENP39573 Chain 1 - CD20-A_H1_IgG1_pI(-)_Isosteric_A_PVA_/S267K/L368 D/K370S/M428L/N434S, XENP39574 Chain 1 - CD20-A_H1_IgG1_pI (-)_Isosteric_A_PVA_/S267K/L368D/K370S/M428L/N434S, and XENP39575 Chain 1 - CD20-A_H1_IgG1_pI(-)_Isosteric_A_PVA_/ S267K/L368D/K370S/M428L/N434S
QVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 406 - protein, 479 aa, synthetic construct
  source 1..479: note = XENP39580 Chain 2 - 1A7[CD28]_H1.14_L1_scFv(GKPGS)4 _Fc(216)_IgG1_C220S/PVA_/S267K/S364K/E357Q/M428L/N434S and XENP39584 Chain 2 - 1A7[CD28]_H1.14_L1_scFv(GKPGS)4_Fc(216 )_IgG1_C220S/PVA_/S267K/S364K/E357Q/M428L/N434S
EVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 407 - protein, 127 aa, synthetic construct
  source 1..127: note = 9G2[CD28]_H0L0 Variable heavy (vh) domain
EVQLVESGGGLVQPGRSLKLSCAASGFTFSNYYMAWVRQAPTKGLEWVASITNTGGSTYYRDSVKGRFTISRDNAKSTLYLQMDSLRSEDTATYYCTRGLIYYYDGRNYYDYVMDAWGQGASVTVSS

SEQ ID NO: 408 - protein, 5 aa, synthetic construct
  source 1..5: note = 9G2[CD28]_H0L0 and 9G2[CD28]_H1L1 vhCDR1
NYYMA

SEQ ID NO: 409 - protein, 17 aa, synthetic construct
  source 1..17: note = 9G2[CD28]_H0L0 and 9G2[CD28]_H1L1 vhCDR2
SITNTGGSTYYRDSVKG

SEQ ID NO: 410 - protein, 18 aa, synthetic construct
  source 1..18: note = 9G2[CD28]_H0L0 and 9G2[CD28]_H1L1 vhCDR3
GLIYYYDGRNYYDYVMDA

SEQ ID NO: 411 - protein, 107 aa, synthetic construct
  source 1..107: note = 9G2[CD28]_H0L0 Variable light (vl) domain
DIQMTQSPASLSASLGETVSIECLASEGISNSLAWYQQKPGKSPQLLIYGASSLQDGVPSRFSGSGSGTQYSLKISGMQPEDEGVYYCQQGYKYPLTFGSGTKLEIK

SEQ ID NO: 412 - protein, 11 aa, synthetic construct
  source 1..11: note = 9G2[CD28]_H0L0 vlCDR1
LASEGISNSLA

SEQ ID NO: 413 - protein, 7 aa, synthetic construct
  source 1..7: note = 9G2[CD28]_H0L0 and 9G2[CD28]_H1L1 vlCDR2
GASSLQD

SEQ ID NO: 414 - protein, 9 aa, synthetic construct
  source 1..9: note = 9G2[CD28]_H0L0 and 9G2[CD28]_H1L1 vlCDR3
QQGYKYPLT

SEQ ID NO: 415 - protein, 127 aa, synthetic construct
  source 1..127: note = 9G2[CD28]_H1L1 Variable heavy (vh) domain
EVQLLESGGGLVQPGGSLRLSCAASGFTFSNYYMAWVRQAPGKGLEWVSSITNTGGSTYYRDSVKGRFTISRDNSKSTLYLQMNSLRAEDTAVYYCTRGLIYYYDGRNYYDYVMDAWGQGTTVTVSS

SEQ ID NO: 416 - protein, 712 aa, synthetic construct
  source 1..712: note = XENP42163 Chain 2 - CD20-B_H1_IgG1_L234A/L235A/D265 S/T366W/H435R/Y436F/K447del_(G4S)3_1A7[CD28]_H1sp_L1.71sp_ scFv(Staple Linker))
QVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPGGGGGSGGGGSGGGGSEVQLLESGGGLVQPGGSLRLSCAASGFTFSSYAMSWVRQAPGCGLEWVSTISGSGDSTYYADSVKGRFTISRDNSKNTLYLQMNSLRAEDTAVYYCAKSGPGLRQVGFDYWGQGTLVTVSSGGGSGGSGGCPPCGGSGGDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGCGTKLEIK

SEQ ID NO: 417 - protein, 479 aa, synthetic construct
  source 1..479: note = XENP39581 Chain 2 - 1A7[CD28]_H1.14_L1.71_scFv(GKPG S)4_Fc(216)_IgG1_C220S/PVA_/S267K/S364K/E357Q/M428L/N434S and XENP39585 Chain 2 - 1A7[CD28]_H1.14_L1.71_scFv(GKPGS)4 _Fc(216)_IgG1_C220S/PVA_/S267K/S364K/E357Q/M428L/N434S
EVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 418 - protein, 479 aa, synthetic construct
  source 1..479: note = XENP39582 Chain 2 - 1A7[CD28]_H1_L1.71_scFv(GKPGS)4 _Fc(216)_IgG1_C220S/PVA_/S267K/S364K/E357Q/M428L/N434S and XENP39586 Chain 2 - 1A7[CD28]_H1_L1.71_scFv(GKPGS)4_Fc(216 )_IgG1_C220S/PVA_/S267K/S364K/E357Q/M428L/N434S
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 419 - protein, 107 aa, synthetic construct
  source 1..107: note = 9G2[CD28]_H1L1 Variable light (vl) domain
DIQMTQSPSSLSASVGDRVTITCRASEGISNSLAWYQQKPGKSPKLLIYGASSLQDGVPSRFSGSGSGTDYTLTISSLQPEDFATYYCQQGYKYPLTFGSGTKVEIK

SEQ ID NO: 420 - protein, 11 aa, synthetic construct
  source 1..11: note = 9G2[CD28]_H1L1 vlCDR1
RASEGISNSLA

SEQ ID NO: 421 - protein, 479 aa, synthetic construct
  source 1..479: note = XENP39583 Chain 2 - 1A7[CD28]_H1.1_L1.71_scFv(GKPGS )4_Fc(216)_IgG1_C220S/PVA_/S267K/S364K/E357Q/M428L/N434S and XENP39587 Chain 2 - 1A7[CD28]_H1.1_L1.71_scFv(GKPGS)4_ Fc(216)_IgG1_C220S/PVA_/S267K/S364K/E357Q/M428L/N434S
EVQLLESGGGLVQPGGSLRLSCAASGFTFSSYYMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 422 - protein, 450 aa, synthetic construct
  source 1..450: note = XENP39584 Chain 1 - CD20-A_H1.202_IgG1_pI(-)_Isoste ric_A_PVA_/S267K/L368D/K370S/M428L/N434S, XENP39585 Chain 1 - CD20-A_H1.202_IgG1_pI(-)_Isosteric_A_PVA_/S267K/L368D/ K370S/M428L/N434S, XENP39586 Chain 1 - CD20-A_H1.202_IgG1_ pI(-)_Isosteric_A_PVA_/S267K/L368D/K370S/M428L/N434S, XENP39587 Chain 1 - CD20-A_H1.202_IgG1_pI(-)_Isosteric_A_P VA_/S267K/L368D/K370S/M428L/N434S, XENP39576 Chain 1 - CD20-A_H1.202_IgG1_pI(-)_Isosteric_A_PVA_/S267K/L368D/K370 S/M428L/N434S, XENP39577 Chain 1 - CD20-A_H1.202_IgG1_pI(- )_Isosteric_A_PVA_/S267K/L368D/K370S/M428L/N434S, XENP39578 Chain 1 - CD20-A_H1.202_IgG1_pI(-)_Isosteric_A_P VA_/S267K/L368D/K370S/M428L/N434S, and XENP39579 Chain 1 - CD20-A_H1.202_IgG1_pI(-)_Isosteric_A_PVA_/S267K/L368D/K370 S/M428L/N434S
QVQLVQSGAEVKKPGASVKVSCKASGYTFTSYNMHWVRQAPGQRLEWMGAIYPGNGATSYSQKFQGRVTITADTSASTAYMELSSLRSEDTAVYYCARSYYMGGDWYFDVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 423 - protein, 121 aa, synthetic construct
  source 1..121: note = 2F10A3.140[CD28]_H1L1 Variable heavy (vh) domain
EVQLLESGGGLVQPGGSLRLSCAASGFSFGGNSMSWVRQAPGKGLEWVATISDNSYSTYYADSVKGRFTISRDNSKNTLYLQMNSLRAEDTAVYYCAKSGPGLRQVGFDYWGQGTLVTVSS

SEQ ID NO: 424 - protein, 5 aa, synthetic construct
  source 1..5: note = 2F10A3.140[CD28]_H1L1 vhCDR1
GNSMS

SEQ ID NO: 425 - protein, 17 aa, synthetic construct
  source 1..17: note = 2F10A3.140[CD28]_H1L1 vhCDR2
TISDNSYSTYYADSVKG

SEQ ID NO: 426 - protein, 213 aa, synthetic construct
  source 1..213: note = XENP39584 Chain 3 - CD20-A_L1.113, XENP39585 Chain 3 - CD20-A_L1.113, XENP39586 Chain 3 - CD20-A_L1.113, XENP39587 Chain 3 - CD20-A_L1.113, XENP40386 Chain 3 - CD20-A_L1.113, XENP40387 Chain 3 - CD20-A_L1.113, XENP39576 Chain 3 - CD20-A_L1.113, XENP39577 Chain 3 - CD20-A_L1.113, XENP39578 Chain 3 - CD20-A_L1.113, and XENP39579 Chain 3 - CD20-A_L1.113
QIVLTQSPSSLSASVGDRVTITCRASWSVSYIHWFQQKPGKSPKPLIYATSNLASGVPVRFSGSGSGTDYTLTISSLQPEDFATYYCQQWTHNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 427 - protein, 451 aa, synthetic construct
  source 1..451: note = XENP39952 Chain 1 - CD20-B_H1_IgG1_pI(-)_Isosteric_A_/PVA_/S267K/L368D/K370S, XENP39953 Chain 1 - CD20-B_H1_IgG1_pI(-)_Isosteric_A_/PVA_/S267K/L368D/K370S, XENP39954 Chain 1 - CD20-B_H1_IgG1_pI(-)_Isosteric_A_/PVA_/S267K/L368D/K370S, XENP39955 Chain 1 - CD20-B_H1_IgG1_pI(-)_Isosteric_A_/PVA_/S267K/L368D/K370S, XENP40365 Chain 1 - CD20-B_H1_IgG1_pI(-)_Isosteric_A_/PVA_/S267K/L368D/K370S, XENP40366 Chain 1 - CD20-B_H1_IgG1_pI(-)_Isosteric_A_/PVA_/S267K/L368D/K370S, XENP41432 Chain 1 - CD20-B_H1_IgG1_pI(-)_Isosteric_A_/PVA_/S267K/L368D/K370S, XENP41433 Chain 1 - CD20-B_H1_IgG1_pI(-)_Isosteric_A_/PVA_/S267K/L368D/K370S, XENP39964 Chain 1 - CD20-B_H1_IgG1_pI(-)_Isosteric_A_/PVA_/S267K/L368D/K370S, XENP39965 Chain 1 - CD20-B_H1_IgG1_pI(-)_Isosteric_A_/PVA_/S267K/L368D/K370S, XENP39966 Chain 1 - CD20-B_H1_IgG1_pI(-)_Isosteric_A_/PVA_/S267K/L368D/K370S, and XENP39967 Chain 1 - CD20-B_H1_IgG1_pI(-)_Isosteric_A_/PVA_/S267K/L368D/K370S
QVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 428 - protein, 479 aa, synthetic construct
  source 1..479: note = XENP39952 Chain 2 - 1A7[CD28]_H1.14_L1_scFv(GKPGS)4 _Fc(216)_IgG1_C220S/PVA_/S267K/S364K/E357Q, XENP39956 Chain 2 - 1A7[CD28]_H1.14_L1_scFv(GKPGS)4_Fc(216)_IgG1_C22 0S/PVA_/S267K/S364K/E357Q, and XENP41436 Chain 2 - 1A7[CD28]_H1.14_L1_scFv(GKPGS)4_Fc(216)_IgG1_C220S/PVA_/S2 67K/S364K/E357Q
EVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 429 - protein, 213 aa, synthetic construct
  source 1..213: note = XENP39952 Chain 3 - CD20-B_L1, XENP39953 Chain 3 - CD20-B_L1, XENP39954 Chain 3 - CD20-B_L1, XENP39955 Chain 3 - CD20-B_L1, XENP40365 Chain 3 - CD20-B_L1, XENP40366 Chain 3 - CD20-B_L1, XENP42156 Chain 3 - CD20-B_L1, XENP42157 Chain 3 - CD20-B_L1, XENP42158 Chain 3 - CD20-B_L1, XENP41432 Chain 3 - CD20-B_L1, XENP41433 Chain 3 - CD20-B_L1, XENP41434 Chain 3 - CD20-B_L1, XENP41435 Chain 3 - CD20-B_L1, XENP41436 Chain 3 - CD20-B_L1, XENP41437 Chain 3 - CD20-B_L1, XENP41438 Chain 3 - CD20-B_L1, XENP41439 Chain 3 - CD20-B_L1, XENP39964 Chain 3 - CD20-B_L1, XENP39965 Chain 3 - CD20-B_L1, XENP39966 Chain 3 - CD20-B_L1, XENP39967 Chain 3 - CD20-B_L1, XENP42162 Chain 3 - CD20-B_L1, XENP42163 Chain 3 - CD20-B_L1, and XENP42164 Chain 3 - CD20-B_L1
EIVLTQSPATLSLSPGERATLSCRASLSVSSMHWYQQKPGQAPRLLIYATSNLASGIPARFSGSGSGTDFTLTISSLEPEDFAVYYCQQWIFNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 430 - protein, 479 aa, synthetic construct
  source 1..479: note = XENP39953 Chain 2 - 1A7[CD28]_H1_L1.71_scFv(GKPGS)4 _Fc(216)_IgG1_C220S/PVA_/S267K/S364K/E357Q, XENP39957 Chain 2 - 1A7[CD28]_H1_L1.71_scFv(GKPGS)4_Fc(216)_IgG1_C22 0S/PVA_/S267K/S364K/E357Q, and XENP41437 Chain 2 - 1A7[CD28]_H1_L1.71_scFv(GKPGS)4_Fc(216)_IgG1_C220S/PVA_/S2 67K/S364K/E357Q
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 431 - protein, 121 aa, synthetic construct
  source 1..121: note = TN228[CD28]_H4L2 Variable heavy (vh) domain
QVQLQESGPGLVKPSETLSLTCTVSGFSLTSYGVHWVRQPPGKGLEWIGVIWPGGGTNYNSALKSRVTISEDTSKSQVSLKLSSVTAADTAVYYCARDRAYGNYLYAMDYWGQGTLVTVSS

SEQ ID NO: 432 - protein, 479 aa, synthetic construct
  source 1..479: note = XENP39954 Chain 2 - 1A7[CD28]_H1.1_L1.71_scFv(GKPGS )4_Fc(216)_IgG1_C220S/PVA_/S267K/S364K/E357Q, XENP39958 Chain 2 - 1A7[CD28]_H1.1_L1.71_scFv(GKPGS)4_Fc(216)_IgG1_C 220S/PVA_/S267K/S364K/E357Q, and XENP41438 Chain 2 - 1A7[CD28]_H1.1_L1.71_scFv(GKPGS)4_Fc(216)_IgG1_C220S/PVA_/ S267K/S364K/E357Q
EVQLLESGGGLVQPGGSLRLSCAASGFTFSSYYMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 433 - protein, 16 aa, synthetic construct
  source 1..16: note = TN228[CD28]_H4L2 vhCDR2
VIWPGGGTNYNSALKS

SEQ ID NO: 434 - protein, 479 aa, synthetic construct
  source 1..479: note = XENP39955 Chain 2 - 1A7[CD28]_H1.14_L1.71_scFv(GKPG S)4_Fc(216)_IgG1_C220S/PVA_/S267K/S364K/E357Q, XENP39959 Chain 2 - 1A7[CD28]_H1.14_L1.71_scFv(GKPGS)4_Fc(216)_IgG1_ C220S/PVA_/S267K/S364K/E357Q, and XENP41439 Chain 2 - 1A7[CD28]_H1.14_L1.71_scFv(GKPGS)4_Fc(216)_IgG1_C220S/PVA_ /S267K/S364K/E357Q
EVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 435 - protein, 111 aa, synthetic construct
  source 1..111: note = TN228[CD28]_H4L2 Variable light (vl) domain
EIVLTQSPATLSLSPGERATLSCRASESVEYYVTSLMQWYQQKPGQAPRLLIYAASNVDSGVPARFSGSGSGTDFTLTISSLEPEDFAVYYCQQSRKVPFTFGGGTKVEIK

SEQ ID NO: 436 - protein, 451 aa, synthetic construct
  source 1..451: note = XENP39956 Chain 1 - CD20-C_H1_IgG1_pI(-)_Isosteric_A_/PVA_/S267K/L368D/K370S, XENP39957 Chain 1 - CD20-C_H1_IgG1_pI(-)_Isosteric_A_/PVA_/S267K/L368D/K370S, XENP39958 Chain 1 - CD20-C_H1_IgG1_pI(-)_Isosteric_A_/PVA_/S267K/L368D/K370S, XENP39959 Chain 1 - CD20-C_H1_IgG1_pI(-)_Isosteric_A_/PVA_/S267K/L368D/K370S, XENP40367 Chain 1 - CD20-C_H1_IgG1_pI(-)_Isosteric_A_/PVA_/S267K/L368D/K370S, XENP40368 Chain 1 - CD20-C_H1_IgG1_pI(-)_Isosteric_A_/PVA_/S267K/L368D/K370S, XENP39968 Chain 1 - CD20-C_H1_IgG1_pI(-)_Isosteric_A_/PVA_/S267K/L368D/K370S, XENP39969 Chain 1 - CD20-C_H1_IgG1_pI(-)_Isosteric_A_/PVA_/S267K/L368D/K370S, and XENP39970 Chain 1 - CD20-C_H1_IgG1_pI(-)_Isosteric_A_/PVA_/S267K/L368D/K370S, XENP39971 Chain 1 - CD20-C_H1_IgG1_pI(-)_Isosteric_A_/PVA_/S267K/L368D/K370S
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 437 - protein, 134 aa, Macaca fascicularis; identical to SEQ ID NO: 18
  source 1..134: note = tr|Q0PDN3|19-152

SEQ ID NO: 438 - protein, 479 aa, synthetic construct
  source 1..479: note = XENP40365 Chain 2 - 1A7[CD28]_H1L1_scFv(GKPGS)4_Fc( 216)_IgG1_C220S/PVA_/S267K/S364K/E357Q, XENP40367 Chain 2 - 1A7[CD28]_H1L1_scFv(GKPGS)4_Fc(216)_IgG1_C220S/PVA_/S267 K/S364K/E357Q, XENP40384 Chain 2 - 1A7[CD28]_H1L1_scFv(GKP GS)4_Fc(216)_IgG1_C220S/PVA_/S267K/S364K/E357Q, XENP40386 Chain 2 - 1A7[CD28]_H1L1_scFv(GKPGS)4_Fc(216)_IgG1_C220S/P VA_/S267K/S364K/E357Q, and XENP41434 Chain 2 - 1A7[CD28]_H 1L1_scFv(GKPGS)4_Fc(216)_IgG1_C220S/PVA_/S267K/S364K/E357Q
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 439 - protein, 121 aa, synthetic construct
  source 1..121: note = CD20-A_H1L1 Variable Heavy (vh) Domain
QVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSS

SEQ ID NO: 440 - protein, 5 aa, synthetic construct
  source 1..5: note = CD20-A_H1L1, CD20-A_H1.202_L1.113, and CD20-B vhCDR1
SYNMH

SEQ ID NO: 441 - protein, 17 aa, synthetic construct
  source 1..17: note = CD20-A_H1L1 vhCDR2
AIYPGNGDTSYNQKFQG

SEQ ID NO: 442 - protein, 12 aa, synthetic construct
  source 1..12: note = CD20-A_H1L1 vhCDR3
STYYGGDWYFNV

SEQ ID NO: 443 - protein, 106 aa, synthetic construct
  source 1..106: note = CD20-A_H1L1 Variable Light (vl) Domain
QIVLTQSPSSLSASVGDRVTITCRASSSVSYIHWFQQKPGKSPKPLIYATSNLASGVPVRFSGSGSGTDYTLTISSLQPEDFATYYCQQWTSNPPTFGGGTKVEIK

SEQ ID NO: 444 - protein, 10 aa, synthetic construct
  source 1..10: note = CD20-A_H1L1 vlCDR1
RASSSVSYIH

SEQ ID NO: 445 - protein, 7 aa, synthetic construct
  source 1..7: note = CD20-A_H1L1, CD20-A_H1.202_L1.113, and CD20-B vlCDR2
ATSNLAS

SEQ ID NO: 446 - protein, 9 aa, synthetic construct
  source 1..9: note = CD20-A_H1L1 vlCDR3
QQWTSNPPT

SEQ ID NO: 447 - protein, 121 aa, synthetic construct
  source 1..121: note = CD20-A_H1.202_L1.113 Variable Heavy (vh) Domain
QVQLVQSGAEVKKPGASVKVSCKASGYTFTSYNMHWVRQAPGQRLEWMGAIYPGNGATSYSQKFQGRVTITADTSASTAYMELSSLRSEDTAVYYCARSYYMGGDWYFDVWGAGTLVTVSS

SEQ ID NO: 448 - protein, 479 aa, synthetic construct
  source 1..479: note = XENP40366 Chain 2 - 1A7[CD28]_H1.1_L1_scFv(GKPGS)4_ Fc(216)_IgG1_C220S/PVA_/S267K/S364K/E357Q, XENP40368 Chain 2 - 1A7[CD28]_H1.1_L1_scFv(GKPGS)4_Fc(216)_IgG1_C220S/PVA_ /S267K/S364K/E357Q, XENP40385 Chain 2 - 1A7[CD28]_H1.1_L1_ scFv(GKPGS)4_Fc(216)_IgG1_C220S/PVA_/S267K/S364K/E357Q, XENP40387 Chain 2 - 1A7[CD28]_H1.1_L1_scFv(GKPGS)4_Fc(216) _IgG1_C220S/PVA_/S267K/S364K/E357Q, and XENP41435 Chain 2 - 1A7[CD28]_H1.1_L1_scFv(GKPGS)4_Fc(216)_IgG1_C220S/PVA_/S 267K/S364K/E357Q
EVQLLESGGGLVQPGGSLRLSCAASGFTFSSYYMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 449 - protein, 17 aa, synthetic construct
  source 1..17: note = CD20-A_H1.202_L1.113 vhCDR2
AIYPGNGATSYSQKFQG

SEQ ID NO: 450 - protein, 12 aa, synthetic construct
  source 1..12: note = CD20-A_H1.202_L1.113 vhCDR3
SYYMGGDWYFDV

SEQ ID NO: 451 - protein, 106 aa, synthetic construct
  source 1..106: note = CD20-A_H1.202_L1.113 Variable Light (vl) Domain
QIVLTQSPSSLSASVGDRVTITCRASWSVSYIHWFQQKPGKSPKPLIYATSNLASGVPVRFSGSGSGTDYTLTISSLQPEDFATYYCQQWTHNPPTFGGGTKVEIK

SEQ ID NO: 452 - protein, 10 aa, synthetic construct
  source 1..10: note = CD20-A_H1.202_L1.113 vlCDR1
RASWSVSYIH

SEQ ID NO: 453 - protein, 450 aa, synthetic construct
  source 1..450: note = XENP40384 Chain 1 - CD20-A_H1_IgG1_pI(-)_Isosteric_A_/PVA_/S267K/L368D/K370S and XENP40385 Chain 1 - CD20-A_H1_IgG1_pI(-)_Isosteric_A_/PVA_/S267K/L368D/K370S
QVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 454 - protein, 9 aa, synthetic construct
  source 1..9: note = CD20-A_H1.202_L1.113 vlCDR3
QQWTHNPPT

SEQ ID NO: 455 - protein, 122 aa, synthetic construct
  source 1..122: note = CD20-B Variable Heavy (vh) Domain
QVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSS

SEQ ID NO: 456 - protein, 450 aa, synthetic construct
  source 1..450: note = XENP40386 Chain 1 - CD20-A_H1.202_IgG1_pI(-)_Isoste ric_A_/PVA_/S267K/L368D/K370S and XENP40387 Chain 1 - CD20-A_H1.202_IgG1_pI(-)_Isosteric_A_/PVA_/S267K/L368D/K37 0S
QVQLVQSGAEVKKPGASVKVSCKASGYTFTSYNMHWVRQAPGQRLEWMGAIYPGNGATSYSQKFQGRVTITADTSASTAYMELSSLRSEDTAVYYCARSYYMGGDWYFDVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 457 - protein, 17 aa, synthetic construct
  source 1..17: note = CD20-B vhCDR2
AIYPGAGDTSYAQKFQG

SEQ ID NO: 458 - protein, 13 aa, synthetic construct
  source 1..13: note = CD20-B vhCDR3
SNYYGSSGWYFDV

SEQ ID NO: 459 - protein, 106 aa, synthetic construct
  source 1..106: note = CD20-B Variable Light (vl) Domain
EIVLTQSPATLSLSPGERATLSCRASLSVSSMHWYQQKPGQAPRLLIYATSNLASGIPARFSGSGSGTDFTLTISSLEPEDFAVYYCQQWIFNPPTFGGGTKVEIK

SEQ ID NO: 460 - protein, 10 aa, synthetic construct
  source 1..10: note = CD20-B vlCDR1
RASLSVSSMH

SEQ ID NO: 461 - protein, 452 aa, synthetic construct
  source 1..452: note = XENP42156 Chain 1 - CD20-B_H1_IgG1_L234A/L235A/D265 S/T366S/L368A/Y407V/H435R/Y436F, XENP42157 Chain 1 - CD20-B_H1_IgG1_L234A/L235A/D265S/T366S/L368A/Y407V/H435R/Y 436F, and XENP42158 Chain 1 - CD20-B_H1_IgG1_L234A/L235A/D 265S/T366S/L368A/Y407V/H435R/Y436F
QVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 462 - protein, 9 aa, synthetic construct
  source 1..9: note = CD20-B vlCDR3
QQWIFNPPT

SEQ ID NO: 463 - protein, 122 aa, synthetic construct
  source 1..122: note = CD20-C Variable Heavy (vh) Domain
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 464 - protein, 5 aa, synthetic construct
  source 1..5: note = CD20-C vhCDR1
DYAMH

SEQ ID NO: 465 - protein, 17 aa, synthetic construct
  source 1..17: note = CD20-C vhCDR2
TISWNSGSIGYADSVKG

SEQ ID NO: 466 - protein, 13 aa, synthetic construct
  source 1..13: note = CD20-C vhCDR3
DIQYGNYYYGMDV

SEQ ID NO: 467 - protein, 107 aa, synthetic construct
  source 1..107: note = CD20-C and Epcoritamab Variable Light (vl) Domain
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 468 - protein, 480 aa, synthetic construct
  source 1..480: note = XENP42156 Chain 2 - 1A7[CD28]_H1_L1.71_scFv(EB Linker)_Fc(216)_IgG1_C220S/L234A/L235A/D265S/T366W and XENP42159 Chain 2 - 1A7[CD28]_H1_L1.71_scFv(EB Linker)_Fc(216)_IgG1_C220S/L234A/L235A/D265S/T366W
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGGSEGKSSGSGSESKSTGGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 469 - protein, 478 aa, synthetic construct
  source 1..478: note = XENP42157 Chain 2 - 1A7[CD28]_H1sp_L1.71sp_scFv(Staple Linker))_Fc(216)_IgG1_C220S/L234A/L235A/D265S/T366W and XENP42160 Chain 2 - 1A7[CD28]_H1sp_L1.71sp_scFv(Staple Linker))_Fc(216)_IgG1_C220S/L234A/L235A/D265S/T366W
EVQLLESGGGLVQPGGSLRLSCAASGFTFSSYAMSWVRQAPGCGLEWVSTISGSGDSTYYADSVKGRFTISRDNSKNTLYLQMNSLRAEDTAVYYCAKSGPGLRQVGFDYWGQGTLVTVSSGGGSGGSGGCPPCGGSGGDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGCGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 470 - protein, 9 aa, synthetic construct
  source 1..9: note = CD20-C vlCDR3
QQRSNWPIT

SEQ ID NO: 471 - protein, 121 aa, synthetic construct [sequence omitted]

SEQ ID NO: 472 - protein, 106 aa, synthetic construct [sequence omitted]

SEQ ID NO: 473 - protein, 119 aa, synthetic construct
  source 1..119: note = obinutuzumab and Glofitamab Variable Heavy (vh) Domain
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 474 - protein, 112 aa, synthetic construct
  source 1..112: note = obinutuzumab and Glofitamab Variable Light (vl) Domain
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 475 - protein, 121 aa, synthetic construct
  source 1..121: note = ibritumomab Variable Heavy (vh) Domain
QVQLQQPGAELVKAGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSA

SEQ ID NO: 476 - protein, 6 aa, Macaca fascicularis
  source 1..6: note = XP_015289654.1 B-lymphocyte antigen CD20 [Macaca fascicularis]|79-84
PICVTV

SEQ ID NO: 477 - protein, 122 aa, synthetic construct [sequence omitted]

SEQ ID NO: 478 - protein, 106 aa, synthetic construct [sequence omitted]

SEQ ID NO: 479 - protein, 122 aa, synthetic construct
  source 1..122: note = mosunetuzumab Variable Heavy (vh) Domain
EVQLVESGGGLVQPGGSLRLSCAASGYTFTSYNMHWVRQAPGKGLEWVGAIYPGNGDTSYNQKEKGRFTISVDKSKNTLYLQMNSLRAEDTAVYYCARVVYYSNSYWYFDVWGQGTLVTVSS

SEQ ID NO: 480 - protein, 106 aa, synthetic construct
  source 1..106: note = mosunetuzumab Variable Light (vl) Domain
DIQMTQSPSSLSASVGDRVTITCRASSSVSYMHWYQQKPGKAPKPLIYAPSNLASGVPSRFSGSGSGTDFTLTISSLQPEDFATYYCQQWSENPPTFGQGTKVEIK

SEQ ID NO: 481 - protein, 118 aa, synthetic construct
  source 1..118: note = ublituximab Variable Heavy (vh) Domain
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSS

SEQ ID NO: 482 - protein, 106 aa, synthetic construct
  source 1..106: note = ublituximab Variable Light (vl) Domain
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEIK

SEQ ID NO: 483 - protein, 121 aa, synthetic construct
  source 1..121: note = Veltuzumab Variable Heavy (vh) Domain
QVQLQQSGAEVKKPGSSVKVSCKASGYTFTSYNMHWVKQAPGQGLEWIGAIYPGMGDTSYNQKFKGKATLTADESTNTAYMELSSLRSEDTAFYYCARSTYYGGDWYFDVWGQGTTVTVSS

SEQ ID NO: 484 - protein, 106 aa, synthetic construct
  source 1..106: note = Veltuzumab Variable Light (vl) Domain
DIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIK

SEQ ID NO: 485 - protein, 452 aa, synthetic construct
  source 1..452: note = XENP42159 Chain 1 - CD20-C_H1_IgG1_L234A/L235A/D265 S/T366S/L368A/Y407V/H435R/Y436F, XENP42160 Chain 1 - CD20-C_H1_IgG1_L234A/L235A/D265S/T366S/L368A/Y407V/H435R/Y 436F, and XENP42161 Chain 1 - CD20-C_H1_IgG1_L234A/L235A/D 265S/T366S/L368A/Y407V/H435R/Y436F
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 486 - protein, 480 aa, synthetic construct
  source 1..480: note = XENP42158 Chain 2 - 1A7[CD28]_H1_L1.71_scFv(GKPGS)4 _Fc(216)_IgG1_C220S/L234A/L235A/D265S/T366W and XENP42161 Chain 2 - 1A7[CD28]_H1_L1.71_scFv(GKPGS)4_Fc(216)_IgG1_C22 0S/L234A/L235A/D265S/T366W
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 487 - protein, 122 aa, synthetic construct
  source 1..122: note = Epcoritamab Variable Heavy (vh) Domain
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSS

SEQ ID NO: 488 - protein, 718 aa, synthetic construct
  source 1..718: note = XENP41432 Chain 2 - CD20-B_H1_(G4S)2_1A7[CD28]_H1L1 _scFv(GKPGS)4_(G4S)2_Fc(222)_IgG1_PVA_/S267K/S364K/E357Q
QVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIKGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 489 - protein, 121 aa, synthetic construct
  source 1..121: note = TRU-015_SMIP Variable Heavy (vh) Domain
QAYLQQSGAESVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVS

SEQ ID NO: 490 - protein, 106 aa, synthetic construct [sequence omitted]

SEQ ID NO: 491 - protein, 127 aa, synthetic construct
  source 1..127: note = Odronextamab Variable Heavy (vh) Domain
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSS

SEQ ID NO: 492 - protein, 107 aa, synthetic construct
  source 1..107: note = Odronextamab Variable Light (vl) Domain
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIK

SEQ ID NO: 493 - protein, 718 aa, synthetic construct
  source 1..718: note = XENP41433 Chain 2 - CD20-B_H1_(G4S)2_1A7[CD28]_H1_L 1.71_scFv(GKPGS)4_(G4S)2_Fc(222)_IgG1_PVA_/S267K/S364K/E35 7Q
QVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 494 - protein, 714 aa, synthetic construct
  source 1..714: note = XENP42162 Chain 2 - CD20-B_H1_IgG1_L234A/L235A/D265 S/T366W/H435R/Y436F/K447del_(G4S)3_1A7[CD28]_H1_L1.71_scFv (EB Linker)
QVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPGGGGGSGGGGSGGGGSEVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGGSEGKSSGSGSESKSTGGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 495 - protein, 121 aa, synthetic construct
  source 1..121: note = Ocaratuzumab Variable Heavy (vh) Domain
EVQLVQSGAEVKKPGESLKISCKGSGRTFTSYNMHWVRQMPGKGLEWMGAIYPLTGDTSYNQKSKLQVTISADKSISTAYLQWSSLKASDTAMYYCARSTYVGGDWQFDVWGKGTTVTVSS

SEQ ID NO: 496 - protein, 106 aa, synthetic construct
  source 1..106: note = Ocaratuzumab Variable Light (vl) Domain
EIVLTQSPGTLSLSPGERATLSCRASSSVPYIHWYQQKPGQAPRLLIYATSALASGIPDRFSGSGSGTDFTLTISRLEPEDFAVYYCQQWLSNPPTFGQGTKLEIK

SEQ ID NO: 497 - protein, 121 aa, synthetic construct
  source 1..121: note = Divozilimab Variable Heavy (vh) Domain
EVQLVQPGAEVVKPGASVKVSCKASGYTFTSYNMHWVRQAPGRGLEWMGAIYPGNGDTSYNQKFKGRVTMTRDKSTSTVYMELSSLRSEDTAVYYCARSTYYGGDWYFNVWGQGTLVTVSS

SEQ ID NO: 498 - protein, 106 aa, synthetic construct
  source 1..106: note = Divozilimab Variable Light (vl) Domain
QIVLSQSPAILSASPGERVTLTCRASSSVSYIHWFQQKPGKAPKPLIYATSNLASGVPSRFSGSGSGTDFSLTISRVEPEDFAVYYCQQWTSNPPTFGGGTKVEIK

SEQ ID NO: 499 - protein, 686 aa, synthetic construct
  source 1..686: note = XENP41434 Chain 1 - CD20-B_H1_(G4S)2_CD20-B_H1_IgG1 _pI(-)_Isosteric_A_/PVA_/S267K/L368D/K370S, XENP41435 Chain 1 - CD20-B_H1_(G4S)2_CD20-B_H1_IgG1_pI(-)_Isosteric_ A_/PVA_/S267K/L368D/K370S, XENP41436 Chain 1 - CD20-B_H1_( G4S)2_CD20-B_H1_IgG1_pI(-)_Isosteric_A_/PVA_/S267K/L368D/K 370S, XENP41437 Chain 1 - CD20-B_H1_(G4S)2_CD20-B_H1_IgG1_ pI(-)_Isosteric_A_/PVA_/S267K/L368D/K370S, XENP41438 Chain 1 - CD20-B_H1_(G4S)2_CD20-B_H1_IgG1_pI(-)_Isosteric_A_/PVA _/S267K/L368D/K370S, and XENP41439 Chain 1 - CD20-B_H1_(G4 S)2_CD20-B_H1_IgG1_pI(-)_Isosteric_A_/PVA_/S267K/L368D/K37 0S
QVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCGGGGSGGGGSQVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 500 - protein, 712 aa, synthetic construct
  source 1..712: note = XENP39572 Chain 2 - CD20-A_H1_IgG1_PVA_/S267K/S364K /E357Q/M428L/N434S/K447del_(G4S)3_1A7[CD28]_H1.14_scFv(GKP GS)4
QVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIK

SEQ ID NO: 501 - protein, 712 aa, synthetic construct
  source 1..712: note = XENP39573 Chain 2 - CD20-A_H1_IgG1_PVA_/S267K/S364K /E357Q/M428L/N434S/K447del_(G4S)3_1A7[CD28]_H1.14_L1.71_sc Fv(GKPGS)4
QVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 502 - protein, 712 aa, synthetic construct
  source 1..712: note = XENP39574 Chain 2 - CD20-A_H1_IgG1_PVA_/S267K/S364K /E357Q/M428L/N434S/K447del_(G4S)3_1A7[CD28]_H1_L1.71_scFv( GKPGS)4
QVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 503 - protein, 712 aa, synthetic construct
  source 1..712: note = XENP39575 Chain 2 - CD20-A_H1_IgG1_PVA_/S267K/S364K /E357Q/M428L/N434S/K447del_(G4S)3_1A7[CD28]_H1.1_L1.71_scF v(GKPGS)4
QVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYYMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 504 - protein, 712 aa, synthetic construct
  source 1..712: note = XENP39576 Chain 2 - CD20-A_H1.202_IgG1_PVA_/S267K/S 364K/E357Q/M428L/N434S/K447del_(G4S)3_1A7[CD28]_H1.14_scFv (GKPGS)4
QVQLVQSGAEVKKPGASVKVSCKASGYTFTSYNMHWVRQAPGQRLEWMGAIYPGNGATSYSQKFQGRVTITADTSASTAYMELSSLRSEDTAVYYCARSYYMGGDWYFDVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIK

SEQ ID NO: 505 - protein, 712 aa, synthetic construct
  source 1..712: note = XENP39577 Chain 2 - CD20-A_H1.202_IgG1_PVA_/S267K/S 364K/E357Q/M428L/N434S/K447del_(G4S)3_1A7[CD28]_H1.14_L1.7 1_scFv(GKPGS)4
QVQLVQSGAEVKKPGASVKVSCKASGYTFTSYNMHWVRQAPGQRLEWMGAIYPGNGATSYSQKFQGRVTITADTSASTAYMELSSLRSEDTAVYYCARSYYMGGDWYFDVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 506 - protein, 712 aa, synthetic construct
  source 1..712: note = XENP39578 Chain 2 - CD20-A_H1.202_IgG1_PVA_/S267K/S 364K/E357Q/M428L/N434S/K447del_(G4S)3_1A7[CD28]_H1_L1.71_s cFv(GKPGS)4
QVQLVQSGAEVKKPGASVKVSCKASGYTFTSYNMHWVRQAPGQRLEWMGAIYPGNGATSYSQKFQGRVTITADTSASTAYMELSSLRSEDTAVYYCARSYYMGGDWYFDVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 507 - protein, 712 aa, synthetic construct
  source 1..712: note = XENP39579 Chain 2 - CD20-A_H1.202_IgG1_PVA_/S267K/S 364K/E357Q/M428L/N434S/K447del_(G4S)3_1A7[CD28]_H1.1_L1.71 _scFv(GKPGS)4
QVQLVQSGAEVKKPGASVKVSCKASGYTFTSYNMHWVRQAPGQRLEWMGAIYPGNGATSYSQKFQGRVTITADTSASTAYMELSSLRSEDTAVYYCARSYYMGGDWYFDVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSEVQLLESGGGLVQPGGSLRLSCAASGFTFSSYYMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 508 - protein, 713 aa, synthetic construct
  source 1..713: note = XENP39964 Chain 2 - CD20-B_H1_IgG1_PVA_/S267K/S364K /E357Q/K447del_(G4S)3_1A7[CD28]_H1.14_scFv(GKPGS)4
QVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIK

SEQ ID NO: 509 - protein, 713 aa, synthetic construct
  source 1..713: note = XENP39965 Chain 2 - CD20-B_H1_IgG1_PVA_/S267K/S364K /E357Q/K447del_(G4S)3_1A7[CD28]_H1_L1.71_scFv(GKPGS)4
QVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 510 - protein, 713 aa, synthetic construct
  source 1..713: note = XENP39966 Chain 2 - CD20-B_H1_IgG1_PVA_/S267K/S364K /E357Q/K447del_(G4S)3_1A7[CD28]_H1.1_L1.71_scFv(GKPGS)4
QVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEVQLLESGGGLVQPGGSLRLSCAASGFTFSSYYMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 511 - protein, 713 aa, synthetic construct
  source 1..713: note = XENP39967 Chain 2 - CD20-B_H1_IgG1_PVA_/S267K/S364K /E357Q/K447del_(G4S)3_1A7[CD28]_H1.14_L1.71_scFv(GKPGS)4
QVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 512 - protein, 713 aa, synthetic construct
  source 1..713: note = XENP39968 Chain 2 - CD20-C_H1_IgG1_PVA_/S267K/S364K /E357Q/K447del_(G4S)3_1A7[CD28]_H1.14_scFv(GKPGS)4
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIK

SEQ ID NO: 513 - protein, 713 aa, synthetic construct
  source 1..713: note = XENP39969 Chain 2 - CD20-C_H1_IgG1_PVA_/S267K/S364K /E357Q/K447del_(G4S)3_1A7[CD28]_H1_L1.71_scFv(GKPGS)4
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 514 - protein, 713 aa, synthetic construct
  source 1..713: note = XENP39970 Chain 2 - CD20-C_H1_IgG1_PVA_/S267K/S364K /E357Q/K447del_(G4S)3_1A7[CD28]_H1.1_L1.71_scFv(GKPGS)4
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEVQLLESGGGLVQPGGSLRLSCAASGFTFSSYYMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 515 - protein, 713 aa, synthetic construct
  source 1..713: note = XENP39971 Chain 2 - CD20-C_H1_IgG1_PVA_/S267K/S364K /E357Q/K447del_(G4S)3_1A7[CD28]_H1.14_L1.71_scFv(GKPGS)4
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 516 - protein, 452 aa, synthetic construct
  source 1..452: note = XENP42164 Chain 2 - CD20-B_IgG1_L234A/L235A/D265S/T 366W/H435R/Y436F/K447del_(G4S)3_1A7[CD28]_H1_L1.71_scFv(GK PGS)4, XENP42163 Chain 1 - CD20-B_H1_IgG1_L234A/L235A/D265S/T366S/L368A/Y407V, and XENP42164 Chain 1 - CD20-B_H1_IgG1_L234A/L235A/D265S/T366S/L368A/Y407V
QVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 517 - protein, 4 aa, synthetic construct
YMNM

SEQ ID NO: 518 - protein, 5 aa, synthetic construct
GSGGS

SEQ ID NO: 519 - protein, 4 aa, synthetic construct
GGGS

SEQ ID NO: 520 - protein, 747 aa, synthetic construct
  source 1..747: note = CD79b x CD20 x CD3 Chain 1
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPATLSLSPGERATLSCRASLSVSSMHWYQQKPGQAPRLLIYATSNLASGIPARFSGSGSGTDFTLTISSLEPEDFAVYYCQQWIFNPPTFGGGTKVEIKGGSEGKSSGSGSESKSTGGSQVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSS

SEQ ID NO: 521 - protein, 451 aa, synthetic construct
  source 1..451: note = CD79b x CD20 x CD3 Chain 2
QVQLQESGPGLVKPSETLSLTCSVSGASISSFYWSWIRQPADEGLEWIGRISPSGKTNYIPSLKSRIIMSLDASKNQFSLRLNSVTAADTAMYYCARGEYSGTYSYSFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 522 - protein, 220 aa, synthetic construct
  source 1..220: note = CD79b x CD20 x CD3 Chain 3
DIVMTQSPLSLSVTPGEPASISCRSSESLLDSEDGNTYLDWFLQKPGQSPQLLIYTLSYRASGVPDRFSGSGSDTDFTLHISSLEAEDVGLYYCMQRMEFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 523 - protein, 121 aa, synthetic construct
  source 1..121: note = Anti-CD79b VH
QVQLQESGPGLVKPSETLSLTCSVSGASISSFYWSWIRQPADEGLEWIGRISPSGKTNYIPSLKSRIIMSLDASKNQFSLRLNSVTAADTAMYYCARGEYSGTYSYSFDVWGQGTMVTVSS

SEQ ID NO: 524 - protein, 10 aa, synthetic construct
  source 1..10: note = Anti-CD79b VHCDR1
GASISSFYWS

SEQ ID NO: 525 - protein, 9 aa, synthetic construct
  source 1..9: note = Anti-CD79b VHCDR2
RISPSGKTN

SEQ ID NO: 526 - protein, 13 aa, synthetic construct
  source 1..13: note = Anti-CD79b VHCDR3
GEYSGTYSYSFDV

SEQ ID NO: 527 - protein, 113 aa, synthetic construct
  source 1..113: note = Anti-CD79b VL
DIVMTQSPLSLSVTPGEPASISCRSSESLLDSEDGNTYLDWFLQKPGQSPQLLIYTLSYRASGVPDRFSGSGSDTDFTLHISSLEAEDVGLYYCMQRMEFPLTFGQGTKVEIK

SEQ ID NO: 528 - protein, 17 aa, synthetic construct
  source 1..17: note = Anti-CD79b VLCDR1
RSSESLLDSEDGNTYLD

SEQ ID NO: 529 - protein, 7 aa, synthetic construct
  source 1..7: note = Anti-CD79b VLCDR2
TLSYRAS

SEQ ID NO: 530 - protein, 9 aa, synthetic construct
  source 1..9: note = Anti-CD79b VLCDR3
MQRMEFPLT

SEQ ID NO: 531 - protein, 121 aa, synthetic construct
  source 1..121: note = Anti-CD3 VH
QVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSS

SEQ ID NO: 532 - protein, 10 aa, synthetic construct
  source 1..10: note = Anti-CD3 VHCDR1
GYTFTRSTMH

SEQ ID NO: 533 - protein, 10 aa, synthetic construct
  source 1..10: note = Anti-CD3VHCDR2
YINPSSAYTN

SEQ ID NO: 534 - protein, 12 aa, synthetic construct
  source 1..12: note = Anti-CD3VHCDR3
PQVHYDYNGFPY

SEQ ID NO: 535 - protein, 106 aa, synthetic construct
  source 1..106: note = Anti-CD3 VL
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIK

SEQ ID NO: 536 - protein, 10 aa, synthetic construct
  source 1..10: note = Anti-CD3 VLCDR1
SASSSVSYMN

SEQ ID NO: 537 - protein, 7 aa, synthetic construct
  source 1..7: note = Anti-CD3 VLCDR2
DSSKLAS

SEQ ID NO: 538 - protein, 9 aa, synthetic construct
  source 1..9: note = Anti-CD3 VLCDR3
QQWSRNPPT

SEQ ID NO: 539 - protein, 122 aa, synthetic construct; identical to SEQ ID NO: 455
  source 1..122: note = Anti-CD20 VH

SEQ ID NO: 540 - protein, 10 aa, synthetic construct
  source 1..10: note = Anti-CD20 VHCDR1
GYTFSSYNMH

SEQ ID NO: 541 - protein, 10 aa, synthetic construct
  source 1..10: note = Anti-CD20 VHCDR2
AIYPGAGDTS

SEQ ID NO: 542 - protein, 13 aa, synthetic construct
  source 1..13: note = Anti-CD20 VHCDR3
SNYYGSSGWYFDV

SEQ ID NO: 543 - protein, 106 aa, synthetic construct; identical to SEQ ID NO: 459
  source 1..106: note = Anti-CD20 VL

SEQ ID NO: 544 - protein, 10 aa, synthetic construct
  source 1..10: note = Anti-CD20 VLCDR1
RASLSVSSMH

SEQ ID NO: 545 - protein, 7 aa, synthetic construct
  source 1..7: note = Anti-CD20 VLCDR2
ATSNLAS

SEQ ID NO: 546 - protein, 9 aa, synthetic construct
  source 1..9: note = Anti-CD20 VLCDR3
QQWIFNPPT

(19) United States
(12) Patent Application Publication
(10) Pub. No.: US 2025/0122299 A1
(43) Pub. Date: Apr. 17, 2025

(54) ANTI-CD20 x ANTI-CD28 COMBINATION THERAPIES

(71) Applicants: Xencor, Inc., Pasadena, CA (US); Janssen Biotech, Inc., Horsham, PA (US)

(72) Inventors: Gregory L. Moore, Azusa, CA (US); Michael Hedvat, Encino, CA (US); Matthew Adam Dragovich, Rancho Cucamonga, CA (US); Lorena Fontan Gabas, Antwerp (BE); Adam Zwolak, Spring House, PA (US); Ulrike Philippar, Antwerp (BE); Pankaj Seth, Audubon, PA (US); Nele Vloemans, Beerse (BE); Ivo Cornelissen, Beerse (BE)

(21) Appl. No.: 18/818,199

(22) Filed: Aug. 28, 2024

Related U.S. Application Data
(60) Provisional application No. 63/579,267, filed on Aug. 28, 2023.

(51) Int. Cl. C07K 16/28; A61K 39/00; A61P 35/00

(52) U.S. Cl. CPC C07K 16/2887; A61P 35/00; C07K 16/2803; C07K 16/2809; C07K 16/2818; A61K 2039/505; C07K 2317/24; C07K 2317/31; C07K 2317/52; C07K 2317/55; C07K 2317/565; C07K 2317/622

(57) ABSTRACT

Provided herein are novel anti-CD20 x anti-CD28 antibodies and methods of using such antibodies for the treatment of B-cell malignancies. Subject anti-CD20 x anti-CD28 antibodies are capable of agonistically binding to CD28 costimulatory molecules on T cells and CD20 on tumor cells. Thus, such antibodies enhance anti-tumor activity at tumor sites. The subject antibodies provided herein are particularly useful in combination with other anti-cancer therapies (e.g., anti-CD3 x anti-CD20 x anti-CD79b antibodies) for the treatment of B-cell malignancies.

### CROSS REFERENCE TO OTHER APPLICATIONS

This application claims priority to U.S. Provisional Application No. 63/579,267 filed on Aug. 28, 2023, titled “Anti-CD20 X Anti-CD28 Combination Therapies” which is incorporated herein by reference in its entirety.

### SEQUENCE LISTING

The instant application contains a Sequence Listing which has been submitted electronically in XML file format and is hereby incorporated by reference in its entirety. Said XML copy, created on Aug. 22, 2024, is named 067461-5318-WO_SL.xml and is 640,102 bytes in size.

### BACKGROUND

The CD20 molecule (also called human B-lymphocyte-restricted differentiation antigen or Bp35) is a hydrophobic transmembrane protein with a molecular weight of approximately 35 kD located on pre-B and mature B lymphocytes. CD20 is found on the surface of greater than 90% of B cells from peripheral blood or lymphoid organs and is expressed during early pre-B cell development and remains until plasma cell differentiation. CD20 is present on both normal B cells as well as malignant B cells, including certain types of B-cell lymphomas and leukemias. In particular, CD20 is expressed on greater than 90% of B cell non-Hodgkin's lymphomas (NHL) but is not found on hematopoietic stem cells, pro-B cells, normal plasma cells, or other normal tissues.

CD28 is a type I transmembrane protein expressed on the surface of T cells which has a single extracellular Ig-V-like domain assembled as a homodimer. CD28 is the receptor for CD80 (B7.1) and CD86 (B7.2) proteins and is activated by CD80 or CD86 expressed on APCs. The binding of CD28 to CD80 or CD86 provide co-stimulatory signals important for T cell activation and survival. T cell stimulation through CD28 in addition to the T-cell receptor (TCR) provides a potent signal for the production of various interleukins. CD28 potentiates cellular signals such as pathways controlled by the NFκB transcription factor after TCR activation. The CD28 co-signal is important for effective T-cell activation such as T cell differentiation, proliferation, cytokine release and cell-death.

Antibody-based therapeutics have been used successfully to treat a variety of diseases, including cancer. An increasingly prevalent avenue being explored is the engineering of single immunoglobulin molecules that co-engage two different antigens. Such alternate antibody formats that engage two different antigens are often referred to as bispecific antibodies. One particular approach for bispecific antibodies is to engineer a first binding domain which engages CD28 and a second binding domain which engages an antigen associated with or upregulated on cancer cells (e.g., CD20) so that the bispecific antibody redirects T cells to destroy the cancer cells. Bispecific antigen-binding molecules that bind both CD28 and a target antigen (such as CD20) would be useful in therapeutic settings in which specific targeting and T cell-mediated killing of cells that express the target antigen is desired. There is also a need for an anti-CD20 X anti-CD28 antibody that is safe for use in a pharmaceutical composition.

### SUMMARY

Provided herein are novel anti-CD20 x anti-CD28 antibodies and methods of using such antibodies for the treatment of B-cell malignancies. Subject anti-CD20 x anti-CD28 antibodies are capable of binding to CD28 costimulatory molecules on T cells and CD20 on B-cells. The subject antibodies provided herein are particularly useful in combination with other T-cell engaging antibodies (e.g., anti-CD3 x anti-CD20 x anti-CD79b antibodies) for the treatment of B-cell malignancies.

In one embodiment, provided herein is an antibody or an antibody fragment thereof comprising:
(a) a first antigen binding domain that binds to CD20, wherein the first antigen binding domain comprises:
   (i) a heavy chain complimentarity determining region 1 (vhCDR1), a vhCDR2 and a vhCDR3 of a variable heavy chain domain (VH1) having an amino acid sequence of SEQ ID NO:439, and a light chain complimentarity determining region 1 (vICDR1), a vICDR2 and a vICDR3 of a variable light chain domain (VL1) having an amino acid sequence of SEQ ID NO:443;
   (ii) a vhCDR1, a vhCDR2 and a vhCDR3 of a variable heavy chain domain (VH1) having an amino acid sequence of SEQ ID NO:447, and a light chain complimentarity determining region (vICDR1), a vICDR2 and a vICDR3 of a variable light chain domain (VL1) having an amino acid sequence of SEQ ID NO:451;
   (iii) a vhCDR1, a vhCDR2 and a vhCDR3 of a variable heavy chain domain (VH1) having an amino acid sequence of SEQ ID NO:455, and a light chain complimentarity determining region (vICDR1), a vICDR2 and a vICDR3 of a variable light chain domain (VL1) having an amino acid sequence of SEQ ID NO:459;
   (iv) a vhCDR1, a vhCDR2 and a vhCDR3 of a variable heavy chain domain (VH1) having an amino acid sequence of SEQ ID NO:463, and a light chain complimentarity determining region (vICDR1), a vICDR2 and a vICDR3 of a variable light chain domain (VL1) having an amino acid sequence of SEQ ID NO:467;
   (v) a vhCDR1, a vhCDR2 and a vhCDR3 of a variable heavy chain domain (VH1) having an amino acid sequence of SEQ ID NO:471, and a light chain complimentarity determining region (vICDR1), a vlCDR2 and a vlCDR3 of a variable light chain domain (VL1) having an amino acid sequence of SEQ ID NO:472;
   (vi) a vhCDR1, a vhCDR2 and a vhCDR3 of a variable heavy chain domain (VH1) having an amino acid sequence of SEQ ID NO:473, and a light chain complimentarity determining region (vICDR1), a vlCDR2 and a vlCDR3 of a variable light chain domain (VL1) having an amino acid sequence of SEQ ID NO:474;
   (vii) a vhCDR1, a vhCDR2 and a vhCDR3 of a variable heavy chain domain (VH1) having an amino acid sequence of SEQ ID NO:475, and a light chain complimentarity determining region (vICDR1), a vlCDR2 and a vlCDR3 of a variable light chain domain (VL1) having an amino acid sequence of SEQ ID NO:472;
   (viii) a vhCDR1, a vhCDR2 and a vhCDR3 of a variable heavy chain domain (VH1) having an amino acid sequence of SEQ ID NO:477, and a light chain complimentarity determining region (vICDR1), a vlCDR2 and a vlCDR3 of a variable light chain domain (VL1) having an amino acid sequence of SEQ ID NO:478;
   (ix) a vhCDR1, a vhCDR2 and a vhCDR3 of a variable heavy chain domain (VH1) having an amino acid sequence of SEQ ID NO:479, and a light chain complimentarity determining region (vICDR1), a vlCDR2 and a vlCDR3 of a variable light chain domain (VL1) having an amino acid sequence of SEQ ID NO:480;
   (x) a vhCDR1, a vhCDR2 and a vhCDR3 of a variable heavy chain domain (VH1) having an amino acid sequence of SEQ ID NO:481, and a light chain complimentarity determining region (vICDR1), a vlCDR2 and a vlCDR3 of a variable light chain domain (VL1) having an amino acid sequence of SEQ ID NO:482;
   (xi) a vhCDR1, a vhCDR2 and a vhCDR3 of a variable heavy chain domain (VH1) having an amino acid sequence of SEQ ID NO:483, and a light chain complimentarity determining region (vICDR1), a vlCDR2 and a vlCDR3 of a variable light chain domain (VL1) having an amino acid sequence of SEQ ID NO:484;
   (xii) a vhCDR1, a vhCDR2 and a vhCDR3 of a variable heavy chain domain (VH1) having an amino acid sequence of SEQ ID NO:487, and a light chain complimentarity determining region (vICDR1), a vlCDR2 and a vlCDR3 of a variable light chain domain (VL1) having an amino acid sequence of SEQ ID NO:467;
   (xiii) a vhCDR1, a vhCDR2 and a vhCDR3 of a variable heavy chain domain (VH1) having an amino acid sequence of SEQ ID NO:489, and a light chain complimentarity determining region (vICDR1), a vlCDR2 and a vlCDR3 of a variable light chain domain (VL1) having an amino acid sequence of SEQ ID NO:490;
   (xiv) a vhCDR1, a vhCDR2 and a vhCDR3 of a variable heavy chain domain (VH1) having an amino acid sequence of SEQ ID NO:491, and a light chain complimentarity determining region (vICDR1), a vlCDR2 and a vlCDR3 of a variable light chain domain (VL1) having an amino acid sequence of SEQ ID NO:492;
   (xv) a vhCDR1, a vhCDR2 and a vhCDR3 of a variable heavy chain domain (VH1) having an amino acid sequence of SEQ ID NO:495, and a light chain complimentarity determining region (vICDR1), a vlCDR2 and a vlCDR3 of a variable light chain domain (VL1) having an amino acid sequence of SEQ ID NO:496; or
   (xvi) a vhCDR1, a vhCDR2 and a vhCDR3 of a variable heavy chain domain (VH1) having an amino acid sequence of SEQ ID NO:497, and a light chain complimentarity determining region (vICDR1), a vlCDR2 and a vlCDR3 of a variable light chain domain (VL1) having an amino acid sequence of SEQ ID NO:498; and
(b) a second antigen binding domain that binds to CD28, wherein the second antigen binding domain comprises:
   (i) a vhCDR1, a vhCDR2 and a vhCDR3 of a variable heavy chain domain (VH2) having an amino acid sequence of SEQ ID NO:1, and a light chain complimentarity determining region (vICDR1), a vlCDR2 and a vlCDR3 of a variable light chain domain (VL2) having an amino acid sequence of SEQ ID NO:5;
   (ii) a vhCDR1, a vhCDR2 and a vhCDR3 of a variable heavy chain domain (VH2) having an amino acid sequence of SEQ ID NO:11, and a light chain complimentarity determining region (vICDR1), a vlCDR2 and a vlCDR3 of a variable light chain domain (VL2) having an amino acid sequence of SEQ ID NO:5;
   (iii) a vhCDR1, a vhCDR2 and a vhCDR3 of a variable heavy chain domain (VH2) having an amino acid sequence of SEQ ID NO:1, and a light chain complimentarity determining region (vICDR1), a vlCDR2 and a vlCDR3 of a variable light chain domain (VL2) having an amino acid sequence of SEQ ID NO:19;
   (iv) a vhCDR1, a vhCDR2 and a vhCDR3 of a variable heavy chain domain (VH2) having an amino acid sequence of SEQ ID NO:11, and a light chain complimentarity determining region (vICDR1), a vlCDR2 and a vlCDR3 of a variable light chain domain (VL2) having an amino acid sequence of SEQ ID NO:19;
   (v) a vhCDR1, a vhCDR2 and a vhCDR3 of a variable heavy chain domain (VH2) having an amino acid sequence of SEQ ID NO:15, and a light chain complimentarity determining region (vICDR1), a vlCDR2 and a vlCDR3 of a variable light chain domain (VL2) having an amino acid sequence of SEQ ID NO:5;
   (vi) a vhCDR1, a vhCDR2 and a vhCDR3 of a variable heavy chain domain (VH2) having an amino acid sequence of SEQ ID NO:15, and a light chain complimentarity determining region (vICDR1), a vlCDR2 and a vlCDR3 of a variable light chain domain (VL2) having an amino acid sequence of SEQ ID NO:19;
   (vii) a vhCDR1, a vhCDR2 and a vhCDR3 of a variable heavy chain domain (VH2) having an amino acid sequence of SEQ ID NO:63, and a light chain complimentarity determining region (vICDR1), a vlCDR2 and a vlCDR3 of a variable light chain domain (VL2) having an amino acid sequence of SEQ ID NO:75;
   (viii) a vhCDR1, a vhCDR2 and a vhCDR3 of a variable heavy chain domain (VH2) having an amino acid sequence of SEQ ID NO:67, and a light chain complimentarity determining region (vICDR1), a vlCDR2 and a vlCDR3 of a variable light chain domain (VL2) having an amino acid sequence of SEQ ID NO:75;
   (ix) a vhCDR1, a vhCDR2 and a vhCDR3 of a variable heavy chain domain (VH2) having an amino acid sequence of SEQ ID NO:63, and a light chain complimentarity determining region (vICDR1), a vlCDR2 and a vlCDR3 of a variable light chain domain (VL2) having an amino acid sequence of SEQ ID NO:79;
   (x) a vhCDR1, a vhCDR2 and a vhCDR3 of a variable heavy chain domain (VH2) having an amino acid sequence of SEQ ID NO:67, and a light chain complimentarity determining region (vICDR1), a vlCDR2 and a vlCDR3 of a variable light chain domain (VL2) having an amino acid sequence of SEQ ID NO:111;
   (xi) a vhCDR1, a vhCDR2 and a vhCDR3 of a variable heavy chain domain (VH2) having an amino acid sequence of SEQ ID NO:71, and a light chain complimentarity determining region (vICDR1), a vlCDR2 and a vlCDR3 of a variable light chain domain (VL2) having an amino acid sequence of SEQ ID NO:75;
   (xii) a vhCDR1, a vhCDR2 and a vhCDR3 of a variable heavy chain domain (VH2) having an amino acid sequence of SEQ ID NO:71, and a light chain complimentarity determining region (vICDR1), a vlCDR2 and a vlCDR3 of a variable light chain domain (VL2) having an amino acid sequence of SEQ ID NO:79;
   (xiii) a vhCDR1, a vhCDR2 and a vhCDR3 of a variable heavy chain domain (VH2) having an amino acid sequence of SEQ ID NO:320, and a light chain complimentarity determining region (vICDR1), a vlCDR2 and a vlCDR3 of a variable light chain domain (VL2) having an amino acid sequence of SEQ ID NO:324;
   (xiv) a vhCDR1, a vhCDR2 and a vhCDR3 of a variable heavy chain domain (VH2) having an amino acid sequence of SEQ ID NO:327, and a light chain complimentarity determining region (vICDR1), a vlCDR2 and a vlCDR3 of a variable light chain domain (VL2) having an amino acid sequence of SEQ ID NO:331;
   (xv) a vhCDR1, a vhCDR2 and a vhCDR3 of a variable heavy chain domain (VH2) having an amino acid sequence of SEQ ID NO:335, and a light chain complimentarity determining region (vICDR1), a vlCDR2 and a vlCDR3 of a variable light chain domain (VL2) having an amino acid sequence of SEQ ID NO:339;
   (xvi) a vhCDR1, a vhCDR2 and a vhCDR3 of a variable heavy chain domain (VH2) having an amino acid sequence of SEQ ID NO:343, and a light chain complimentarity determining region (vICDR1), a vlCDR2 and a vlCDR3 of a variable light chain domain (VL2) having an amino acid sequence of SEQ ID NO:347;
   (xvii) a vhCDR1, a vhCDR2 and a vhCDR3 of a variable heavy chain domain (VH2) having an amino acid sequence of SEQ ID NO:351, and a light chain complimentarity determining region (vICDR1), a vlCDR2 and a vlCDR3 of a variable light chain domain (VL2) having an amino acid sequence of SEQ ID NO:355;
   (xviii) a vhCDR1, a vhCDR2 and a vhCDR3 of a variable heavy chain domain (VH2) having an amino acid sequence of SEQ ID NO:359, and a light chain complimentarity determining region (vICDR1), a vlCDR2 and a vlCDR3 of a variable light chain domain (VL2) having an amino acid sequence of SEQ ID NO:355;
   (xix) a vhCDR1, a vhCDR2 and a vhCDR3 of a variable heavy chain domain (VH2) having an amino acid sequence of SEQ ID NO:367, and a light chain complimentarity determining region (vICDR1), a vlCDR2 and a vlCDR3 of a variable light chain domain (VL2) having an amino acid sequence of SEQ ID NO:371;
   (xx) a vhCDR1, a vhCDR2 and a vhCDR3 of a variable heavy chain domain (VH2) having an amino acid sequence of SEQ ID NO:375, and a light chain complimentarity determining region (vICDR1), a vlCDR2 and a vlCDR3 of a variable light chain domain (VL2) having an amino acid sequence of SEQ ID NO:380;
   (xxi) a vhCDR1, a vhCDR2 and a vhCDR3 of a variable heavy chain domain (VH2) having an amino acid sequence of SEQ ID NO:391, and a light chain complimentarity determining region (vICDR1), a vlCDR2 and a vlCDR3 of a variable light chain domain (VL2) having an amino acid sequence of SEQ ID NO:395;
   (xxii) a vhCDR1, a vhCDR2 and a vhCDR3 of a variable heavy chain domain (VH2) having an amino acid sequence of SEQ ID NO:399, and a light chain complimentarity determining region (vICDR1), a vlCDR2 and a vlCDR3 of a variable light chain domain (VL2) having an amino acid sequence of SEQ ID NO:403;
   (xxiii) a vhCDR1, a vhCDR2 and a vhCDR3 of a variable heavy chain domain (VH2) having an amino acid sequence of SEQ ID NO:407, and a light chain complimentarity determining region (vICDR1), a vICDR2 and a vICDR3 of a variable light chain domain (VL2) having an amino acid sequence of SEQ ID NO:411;
   (xxiv) a vhCDR1, a vhCDR2 and a vhCDR3 of a variable heavy chain domain (VH2) having an amino acid sequence of SEQ ID NO:415, and a light chain complimentarity determining region (vICDR1), a vICDR2 and a vICDR3 of a variable light chain domain (VL2) having an amino acid sequence of SEQ ID NO:419;
   (xxv) a vhCDR1, a vhCDR2 and a vhCDR3 of a variable heavy chain domain (VH2) having an amino acid sequence of SEQ ID NO:423, and a light chain complimentarity determining region (vICDR1), a vICDR2 and a vICDR3 of a variable light chain domain (VL2) having an amino acid sequence of SEQ ID NO:5; or
   (xxvi) a vhCDR1, a vhCDR2 and a vhCDR3 of a variable heavy chain domain (VH2) having an amino acid sequence of SEQ ID NO:431, and a light chain complimentarity determining region (vICDR1), a vICDR2 and a vICDR3 of a variable light chain domain (VL2) having an amino acid sequence of SEQ ID NO:435.

In another embodiment, provided herein is an antibody or an antibody fragment thereof comprising a first antigen binding domain that binds to CD20, and a second antigen binding domain that binds to CD28, and wherein:
(a) the first antigen binding domain comprises:
   (i) a vhCDR1 having an amino acid sequence of SEQ ID NO:440; a vhCDR2 having an amino acid sequence of SEQ ID NO:441; and a vhCDR3 having an amino acid sequence of SEQ ID NO:442; and a vICDR1 having an amino acid sequence of SEQ ID NO:444, a vICDR2 having an amino acid sequence of SEQ ID NO: 445, and a vICDR3 having an amino acid sequence of SEQ ID NO:446;
   (ii) a vhCDR1 having an amino acid sequence of SEQ ID NO:440; a vhCDR2 having an amino acid sequence of SEQ ID NO:449; and a vhCDR3 having an amino acid sequence of SEQ ID NO:450; and a vICDR1 having an amino acid sequence of SEQ ID NO:452, a vICDR2 having an amino acid sequence of SEQ ID NO:445, and a vICDR3 having an amino acid sequence of SEQ ID NO:454;
   (iii) a vhCDR1 having an amino acid sequence of SEQ ID NO:440; a vhCDR2 having an amino acid sequence of SEQ ID NO:457; and a vhCDR3 having an amino acid sequence of SEQ ID NO:458; and a vICDR1 having an amino acid sequence of SEQ ID NO:460, a vICDR2 having an amino acid sequence of SEQ ID NO:445, and a vICDR3 having an amino acid sequence of SEQ ID NO:462; or
   (iv) a vhCDR1 having an amino acid sequence of SEQ ID NO:464; a vhCDR2 having an amino acid sequence of SEQ ID NO:465; and a vhCDR3 having an amino acid sequence of SEQ ID NO:466; and a vICDR1 having an amino acid sequence of SEQ ID NO:356, a vICDR2 having an amino acid sequence of SEQ ID NO:357, and a vICDR3 having an amino acid sequence of SEQ ID NO:470; and
(b) the second antigen binding domain comprises:
   (i) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:8;
   (ii) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:8;
   (iii) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:22;
   (iv) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:22;
   (v) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:8; or
   (vi) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:22.

In another embodiment, provided herein is an antibody or an antibody fragment thereof comprising a first antigen binding domain that binds to CD20, and a second antigen binding domain that binds to CD28, and wherein:
(a) the first antibody binding domain comprises:
   (i) a VH1 having an amino acid sequence of SEQ ID NO:439; and a VL1 having an amino acid sequence of SEQ ID NO:443;
   (ii) a VH1 having an amino acid sequence of SEQ ID NO:447; and a VL1 having an amino acid sequence of SEQ ID NO:451;
   (iii) a VH1 having an amino acid sequence of SEQ ID NO:455; and a VL1 having an amino acid sequence of SEQ ID NO:459;
   (iv) a VH1 having an amino acid sequence of SEQ ID NO:463; and a VL1 having an amino acid sequence of SEQ ID NO:467;
   (v) a VH1 having an amino acid sequence of SEQ ID NO:471; and a VL1 having an amino acid sequence of SEQ ID NO:472;
   (vi) a VH1 having an amino acid sequence of SEQ ID NO:473; and a VL1 having an amino acid sequence of SEQ ID NO:474;
   (vii) a VH1 having an amino acid sequence of SEQ ID NO:475; and a VL1 having an amino acid sequence of SEQ ID NO:472;
   (viii) a VH1 having an amino acid sequence of SEQ ID NO:477; and a VL1 having an amino acid sequence of SEQ ID NO:478;
   (ix) a VH1 having an amino acid sequence of SEQ ID NO:479; and a VL1 having an amino acid sequence of SEQ ID NO:480;
   (x) a VH1 having an amino acid sequence of SEQ ID NO:481; and a VL1 having an amino acid sequence of SEQ ID NO:482;
   (xi) a VH1 having an amino acid sequence of SEQ ID NO:483; and a VL1 having an amino acid sequence of SEQ ID NO:484;
   (xii) a VH1 having an amino acid sequence of SEQ ID NO:487; and a VL1 having an amino acid sequence of SEQ ID NO:467;
   (xiii) a VH1 having an amino acid sequence of SEQ ID NO:489; and a VL1 having an amino acid sequence of SEQ ID NO:490;
   (xiv) a VH1 having an amino acid sequence of SEQ ID NO:491; and a VL1 having an amino acid sequence of SEQ ID NO:492;
   (xv) a VH1 having an amino acid sequence of SEQ ID NO:495; and a VL1 having an amino acid sequence of SEQ ID NO:496; or
   (xvi) a VH1 having an amino acid sequence of SEQ ID NO:497; and a VL1 having an amino acid sequence of SEQ ID NO:498; and
(b) the second antigen binding domain comprises:
   (i) a VH2 having an amino acid sequence of SEQ ID NO:1; and a VL2 having an amino acid sequence of SEQ ID NO:5;
   (ii) a VH2 having an amino acid sequence of SEQ ID NO:11; and a VL2 having an amino acid sequence of SEQ ID NO:5;
   (iii) a VH2 having an amino acid sequence of SEQ ID NO:1; and a VL2 having an amino acid sequence of SEQ ID NO:19;
   (iv) a VH2 having an amino acid sequence of SEQ ID NO:11; and a VL2 having an amino acid sequence of SEQ ID NO:19;
   (v) a VH2 having an amino acid sequence of SEQ ID NO:15; and a VL2 having an amino acid sequence of SEQ ID NO:5;
   (vi) a VH2 having an amino acid sequence of SEQ ID NO:15; and a VL2 having an amino acid sequence of SEQ ID NO:19;
   (vii) a VH2 having an amino acid sequence of SEQ ID NO:63; and a VL2 having an amino acid sequence of SEQ ID NO:75;
   (viii) a VH2 having an amino acid sequence of SEQ ID NO:67; and a VL2 having an amino acid sequence of SEQ ID NO:75;
   (ix) a VH2 having an amino acid sequence of SEQ ID NO:63; and a VL2 having an amino acid sequence of SEQ ID NO:79;
   (x) a VH2 having an amino acid sequence of SEQ ID NO:67; and a VL2 having an amino acid sequence of SEQ ID NO:111;
   (xi) a VH2 having an amino acid sequence of SEQ ID NO:71; and a VL2 having an amino acid sequence of SEQ ID NO:75;
   (xii) a VH2 having an amino acid sequence of SEQ ID NO:71; and a VL2 having an amino acid sequence of SEQ ID NO:79;
   (xiii) a VH2 having an amino acid sequence of SEQ ID NO:320; and a VL2 having an amino acid sequence of SEQ ID NO:324;
   (xiv) a VH2 having an amino acid sequence of SEQ ID NO:327; and a VL2 having an amino acid sequence of SEQ ID NO:331;
   (xv) a VH2 having an amino acid sequence of SEQ ID NO:335; and a VL2 having an amino acid sequence of SEQ ID NO:339;
   (xvi) a VH2 having an amino acid sequence of SEQ ID NO:343; and a VL2 having an amino acid sequence of SEQ ID NO:347;
   (xvii) a VH2 having an amino acid sequence of SEQ ID NO:351; and a VL2 having an amino acid sequence of SEQ ID NO:355;
   (xviii) a VH2 having an amino acid sequence of SEQ ID NO:359; and a VL2 having an amino acid sequence of SEQ ID NO:355;
   (xix) a VH2 having an amino acid sequence of SEQ ID NO:367; and a VL2 having an amino acid sequence of SEQ ID NO:371;
   (xx) a VH2 having an amino acid sequence of SEQ ID NO:375; and a VL2 having an amino acid sequence of SEQ ID NO:380; or
   (xxi) a VH2 having an amino acid sequence of SEQ ID NO:391; and a VL2 having an amino acid sequence of SEQ ID NO:395;
   (xxii) a VH2 having an amino acid sequence of SEQ ID NO:399; and a VL2 having an amino acid sequence of SEQ ID NO:403;
   (xxiii) a VH2 having an amino acid sequence of SEQ ID NO:407; and a VL2 having an amino acid sequence of SEQ ID NO:411;
   (xxiv) a VH2 having an amino acid sequence of SEQ ID NO:415; and a VL2 having an amino acid sequence of SEQ ID NO:419;
   (xxv) a VH2 having an amino acid sequence of SEQ ID NO:423; and a VL2 having an amino acid sequence of SEQ ID NO:5; or
   (xxvi) a VH2 having an amino acid sequence of SEQ ID NO:431; and a VL2 having an amino acid sequence of SEQ ID NO:435.

In another embodiment, provided herein is an antibody or an antibody fragment thereof comprising a first antigen binding domain that binds to CD20, and a second antigen binding domain that binds to CD28, and wherein:
(a) the first antigen binding domain comprises:
   (i) a VH1 having an amino acid sequence of SEQ ID NO:439; and a VL1 having an amino acid sequence of SEQ ID NO:443;
   (ii) a VH1 having an amino acid sequence of SEQ ID NO:447; and a VL1 having an amino acid sequence of SEQ ID NO:451;
   (iii) a VH1 having an amino acid sequence of SEQ ID NO:455; and a VL1 having an amino acid sequence of SEQ ID NO:459; or
   (iv) a VH1 having an amino acid sequence of SEQ ID NO:463; and a VL1 having an amino acid sequence of SEQ ID NO:467; and
(b) the second antigen binding domain comprises:
   (i) a VH2 having an amino acid sequence of SEQ ID NO:1; and a VL2 having an amino acid sequence of SEQ ID NO:5;
   (ii) a VH2 having an amino acid sequence of SEQ ID NO:11; and a VL2 having an amino acid sequence of SEQ ID NO:5;
   (iii) a VH2 having an amino acid sequence of SEQ ID NO:1; and a VL2 having an amino acid sequence of SEQ ID NO:19;
   (iv) a VH2 having an amino acid sequence of SEQ ID NO:11; and a VL2 having an amino acid sequence of SEQ ID NO:19;
   (v) a VH2 having an amino acid sequence of SEQ ID NO:15; and a VL2 having an amino acid sequence of SEQ ID NO:5;
   (vi) a VH2 having an amino acid sequence of SEQ ID NO:15; and a VL2 having an amino acid sequence of SEQ ID NO:19;
   (vii) a VH2 having an amino acid sequence of SEQ ID NO:63; and a VL2 having an amino acid sequence of SEQ ID NO:75;
   (viii) a VH2 having an amino acid sequence of SEQ ID NO:67; and a VL2 having an amino acid sequence of SEQ ID NO:75;
   (ix) a VH2 having an amino acid sequence of SEQ ID NO:63; and a VL2 having an amino acid sequence of SEQ ID NO:79;
   (x) a VH2 having an amino acid sequence of SEQ ID NO:67; and a VL2 having an amino acid sequence of SEQ ID NO:79;
   (xi) a VH2 having an amino acid sequence of SEQ ID NO:71; and a VL2 having an amino acid sequence of SEQ ID NO:75; or
   (xii) a VH2 having an amino acid sequence of SEQ ID NO:71; and a VL2 having an amino acid sequence of SEQ ID NO:79.

In one embodiment, provided herein is an antibody or an antibody fragment thereof comprising a first antigen binding domain that binds to CD20, and a second antigen binding domain that binds to CD28, and wherein:
(a) the first antigen binding domain comprises a vhCDR1 having an amino acid sequence of SEQ ID NO:440; a vhCDR2 having an amino acid sequence of SEQ ID NO:457; and a vhCDR3 having an amino acid sequence of SEQ ID NO:458; and a vICDR1 having an amino acid sequence of SEQ ID NO:460, a vICDR2 having an amino acid sequence of SEQ ID NO:445, and a vICDR3 having an amino acid sequence of SEQ ID NO:462; and
(b) the second antigen binding domain comprises a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:22.

In one embodiment, provided herein is an antibody or an antibody fragment thereof comprising a first antigen binding domain that binds to CD20, and a second antigen binding domain that binds to CD28, and wherein:
(a) the first antigen binding domain comprises a VH1 having an amino acid sequence of SEQ ID NO:455; and a VL1 having an amino acid sequence of SEQ ID NO:459; and
(b) the second antigen binding domain comprises a VH2 having an amino acid sequence of SEQ ID NO:63; and a VL2 having an amino acid sequence of SEQ ID NO:79.

In an another embodiment, provided herein is an antibody or an antibody fragment thereof comprising:
a) a first monomer comprising, from N-terminal to C-terminal, a VH1-CH1-hinge-CH2-CH3, wherein the VH1 is the first variable heavy chain domain and CH2-CH3 is a first Fc domain; and
b) a second monomer comprising:
   i) a single chain variable fragment (scFv) comprising a second variable heavy chain domain (VH2), a linker, and a second variable light chain domain (VL2), and
   ii) a second Fc domain, wherein the scFv is covalently attached to the N-terminus of the second Fc domain using a domain linker;
c) a light chain comprising, from N-terminal to C-terminal, VL1-CL, wherein the VL1 is the first variable light chain domain and CL is a constant light chain domain,
wherein the VH1 and the VL1 together form a first antigen binding domain and the VH2 and the VL2 together form a second binding domain, and
wherein the first antigen binding domain binds to CD20 and the second antigen binding domain binds to CD28.

Also provided herein are methods to treat a B-cell malignancy. In one embodiment, a method of treating a B-cell malignancy in a patient in need thereof, comprising administering to the patient a combination of an anti-CD20 x anti-CD28 antibody or an antibody fragment thereof and a T-cell engager, wherein:
(A) the anti-CD20 x anti-CD28 antibody or the fragment thereof comprises:
(a) a first antigen binding domain that binds to CD20 comprising:
(i) a vhCDR1 having an amino acid sequence of SEQ ID NO:440; a vhCDR2 having an amino acid sequence of SEQ ID NO:441; and a vhCDR3 having an amino acid sequence of SEQ ID NO:442; and a vICDR1 having an amino acid sequence of SEQ ID NO:444, a vICDR2 having an amino acid sequence of SEQ ID NO: 445, and a vICDR3 having an amino acid sequence of SEQ ID NO:446;
(ii) a vhCDR1 having an amino acid sequence of SEQ ID NO:440; a vhCDR2 having an amino acid sequence of SEQ ID NO:449; and a vhCDR3 having an amino acid sequence of SEQ ID NO:450; and a vICDR1 having an amino acid sequence of SEQ ID NO:452, a vICDR2 having an amino acid sequence of SEQ ID NO:445, and a vICDR3 having an amino acid sequence of SEQ ID NO:454;
(iii) a vhCDR1 having an amino acid sequence of SEQ ID NO:440; a vhCDR2 having an amino acid sequence of SEQ ID NO:457; and a vhCDR3 having an amino acid sequence of SEQ ID NO:458; and a vICDR1 having an amino acid sequence of SEQ ID NO:460, a vICDR2 having an amino acid sequence of SEQ ID NO:445, and a vICDR3 having an amino acid sequence of SEQ ID NO:462; or
(iv) a vhCDR1 having an amino acid sequence of SEQ ID NO:464; a vhCDR2 having an amino acid sequence of SEQ ID NO:465; and a vhCDR3 having an amino acid sequence of SEQ ID NO:466; and a vICDR1 having an amino acid sequence of SEQ ID NO:356, a vICDR2 having an amino acid sequence of SEQ ID NO:357, and a vICDR3 having an amino acid sequence of SEQ ID NO:470; and
(b) a second antigen binding domain that binds to CD28 comprising:
(i) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:8;
(ii) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:8;
(iii) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:22;
(iv) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:22;
(v) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:8; or
(vi) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:22; and
(B) the T cell engager is an anti-CD79b x anti-CD20 x anti-CD3 antibody or an antibody fragment thereof.

In one embodiment, provided herein is a method of treating a B cell malignancy in a patient in need thereof, comprising administering to the patient: A) anti-CD20 x anti-CD28 antibody comprising: i) a first antigen binding domain having: (a) a VH1 comprising a vhCDR1 having an amino acid sequence of SEQ ID NO:440; a vhCDR2 having an amino acid sequence of SEQ ID NO:457; and a vhCDR3 having an amino acid sequence of SEQ ID NO:458; and (b) a VL1 comprising a vICDR1 having an amino acid sequence of SEQ ID NO:460, a vICDR2 having an amino acid sequence of SEQ ID NO:445, and a vICDR3 having an amino acid sequence of SEQ ID NO:462; and ii) a second antigen binding domain having: (a) a VH2 comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (b) a VL2 comprising a vICDR1 having an amino acid sequence of SEQ ID NO:6, a vICDR2 having an amino acid sequence of SEQ ID NO:7, and a vICDR3 having an amino acid sequence of SEQ ID NO:22; and B) a T cell engager, wherein the T cell engager is an anti-CD79b x anti-CD20 x anti-CD3 antibody. In some embodiments, anti-CD79b x anti-CD20 x anti-CD3 antibody or the antibody fragment comprises: (a) an antigen binding domain that binds to CD79b comprising a vhCDR1 having an amino acid sequence of SEQ ID NO:524, a vhCDR2 having an amino acid sequence of SEQ ID NO:525, and a vhCDR3 having an amino acid sequence of SEQ ID NO:526; and a vICDR1 having an amino acid sequence of SEQ ID NO:528, a vICDR2 having an amino acid sequence of SEQ ID NO:529, and a vICDR3 having an amino acid sequence of SEQ ID NO:530; (b) an antigen binding domain that binds to CD20 comprising a vhCDR1 having an amino acid sequence of SEQ ID NO:540, a vhCDR2 having an amino acid sequence of SEQ ID NO:541, and a vhCDR3 having an amino acid sequence of SEQ ID NO:542; and a vICDR1 having an amino acid sequence of SEQ ID NO:544, a vICDR2 having an amino acid sequence of SEQ ID NO:545, and a vICDR3 having an amino acid sequence of SEQ ID NO:546; and (c) an antigen binding domain that binds to CD3 comprising a vhCDR1 having an amino acid sequence of SEQ ID NO:532, a vhCDR2 having an amino acid sequence of SEQ ID NO:533, and a vhCDR3 having an amino acid sequence of SEQ ID NO:534; and a vICDR1 having an amino acid sequence of SEQ ID NO:536, a vICDR2 having an amino acid sequence of SEQ ID NO:537, and a vICDR3 having an amino acid sequence of SEQ ID NO:538.

In another embodiment, provided herein is a method of enhancing T cell proliferation comprising contacting the cells with: A) anti-CD20 x anti-CD28 antibody comprising: i) a first antigen binding domain having: (a) a VH1 comprising a vhCDR1 having an amino acid sequence of SEQ ID NO:440; a vhCDR2 having an amino acid sequence of SEQ ID NO:457; and a vhCDR3 having an amino acid sequence of SEQ ID NO:458; and (b) a VL1 comprising a vICDR1 having an amino acid sequence of SEQ ID NO:460, a vICDR2 having an amino acid sequence of SEQ ID NO:445, and a vICDR3 having an amino acid sequence of SEQ ID NO:462; and ii) a second antigen binding domain having: (a) a VH2 comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (b) a VL2 comprising a vICDR1 having an amino acid sequence of SEQ ID NO:6, a vICDR2 having an amino acid sequence of SEQ ID NO:7, and a vICDR3 having an amino acid sequence of SEQ ID NO:22; and B) a T cell engager, wherein the T cell engager is an anti-CD79b x anti-CD20 x anti-CD3 antibody. In some embodiments, anti-CD79b x anti-CD20 x anti-CD3 antibody or the antibody fragment comprises: (a) an antigen binding domain that binds to CD79b comprising a vhCDR1 having an amino acid sequence of SEQ ID NO:524, a vhCDR2 having an amino acid sequence of SEQ ID NO:525, and a vhCDR3 having an amino acid sequence of SEQ ID NO:526; and a vICDR1 having an amino acid sequence of SEQ ID NO:528, a vICDR2 having an amino acid sequence of SEQ ID NO:529, and a vICDR3 having an amino acid sequence of SEQ ID NO:530; (b) an antigen binding domain that binds to CD20 comprising a vhCDR1 having an amino acid sequence of SEQ ID NO:540, a vhCDR2 having an amino acid sequence of SEQ ID NO:541, and a vhCDR3 having an amino acid sequence of SEQ ID NO:542; and a vICDR1 having an amino acid sequence of SEQ ID NO:544, a vICDR2 having an amino acid sequence of SEQ ID NO:545, and a vICDR3 having an amino acid sequence of SEQ ID NO:546; and (c) an antigen binding domain that binds to CD3 comprising a vhCDR1 having an amino acid sequence of SEQ ID NO:532, a vhCDR2 having an amino acid sequence of SEQ ID NO:533, and a vhCDR3 having an amino acid sequence of SEQ ID NO:534; and a vICDR1 having an amino acid sequence of SEQ ID NO:536, a vICDR2 having an amino acid sequence of SEQ ID NO:537, and a vICDR3 having an amino acid sequence of SEQ ID NO:538.

In some embodiments, the anti-CD79b x anti-CD20 x anti-CD3 antibody comprises: i) a first monomer having an amino acid sequence of SEQ ID NO:520; ii) a second monomer having an amino acid sequence of SEQ ID NO:521; and iii) a light chain having an amino acid sequence of SEQ ID NO:522.

In some embodiments, the anti-CD20 x anti-CD28 antibody is a heterodimeric antibody comprising: i) a first monomer having an amino acid sequence of SEQ ID NO:461; ii) a second monomer having an amino acid sequence of SEQ ID NO:469; and iii) a light chain having an amino acid sequence of SEQ ID NO:429.

### BRIEF DESCRIPTION OF THE DRAWINGS

FIGS. 1A and 1B depict the sequences for human, mouse, and cynomolgus CD28. Such CD28 are useful for the development of cross-reactive CD28 antigen binding domains for ease of clinical development.

FIG. 2 depicts the sequences for human, mouse, and cynomolgus CD20. Such CD20 are useful for the development of cross-reactive CD20 antigen binding domains for ease of clinical development.

FIGS. 3A-3F depict useful pairs of heterodimerization variant sets (including skew and pI variants). In FIG. 3F, there are variants for which there are no corresponding “monomer 2” variants. Such variants are pI variants that can be used alone on either monomer of a bispecific antibody (e.g., CD20 x CD28 bsAb), or included, for example, on the non-scFv side of a format that utilizes an scFv as a component and an appropriate charged scFv linker can be used on the second monomer that utilizes an scFv as the CD28 binding domain. Suitable charged linkers are shown in FIG. 6.

FIG. 4 depicts a list of isosteric variant antibody constant regions and their respective substitutions. pI_(−) indicates lower pI variants, while pI_(+) indicates higher pI variants. These variants can be optionally and independently combined with other variants, including heterodimerization variants, outlined herein.

FIG. 5 depict useful ablation variants that ablate FcγR binding (also referred to as “knockouts” or “KO” variants). In some embodiments, such ablation variants are included in the Fc domain of both monomers of the subject antibody described herein. In other embodiments, the ablation variants are only included on only one variant Fc domain.

FIG. 6 depicts a number of charged scFv linkers that find use in increasing or decreasing the pI of the subject heterodimeric bispecific antibodies that utilize one or more scFv as a component, as described herein (e.g., CD20 x CD28 bispecific antibodies (bsAbs)). The (+H) positive linker finds particular use herein, particularly with anti-CD28 VL and VH sequences shown herein. A single prior art scFv linker with a single charge is referenced as “Whitlow,” from Whitlow et al., Protein Engineering 6(8):989-995 (1993). It should be noted that this linker was used for reducing aggregation and enhancing proteolytic stability in scFvs. Such charged scFv linkers can be used in any of the subject antibody formats disclosed herein that include scFvs (e.g., 1+1 Fab-scFv-Fc and 2+1 Fab2-scFv-Fc formats).

FIGS. 7A and 7B depict a number of exemplary domain linkers. In some embodiments, these linkers find use linking a single-chain Fv to an Fc chain. In some embodiments, these linkers may be combined in any orientation. For example, a GGGGS (SEQ ID NO:57) linker may be combined with a “lower half hinge” linker at the N-terminus or at the C-terminus.

FIG. 8 shows a particularly useful bispecific antibody platforms for the CD20 x CD28 bispecific antibodies (bsAbs) of the invention. Although the platforms are described here in the context of the 1+1 Fab-scFv-Fc format, they can be adapted for use in other bispecific antibody formats.

FIG. 9 depicts various heterodimeric skew variant amino acid substitutions that can be used with the heterodimeric antibodies described herein.

FIGS. 10A-10E show the sequences of several useful heterodimeric CD20 x CD28 bsAb backbones based on human IgG1, without the variable domains. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain, and the E233P/L234V/L235A/G236del/S267K ablation variants on both chains. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297A variant that removes glycosylation on both chains. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297S variant that removes glycosylation on both chains. Heterodimeric Fc backbone 8 is based on human IgG4, and includes the L368D/K370S skew variants and the Q295E/N384D/Q418E/N421D pI variants on a first heterodimeric Fc chain, the S364K/E357Q skew variants on a second heterodimeric Fc chain, and the S228P (according to EU numbering, S241P in Kabat) variant that ablates Fab arm exchange (as is known in the art) on both chains. Heterodimeric Fc backbone 9 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 10 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain, and the S267K ablation variant on both chains. Heterodimeric Fc backbone 11 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 12 is based on human IgG1 (356E/358M allotype), and includes the L368D/K370S skew variants on a first heterodimeric Fc chain, the S364K/E357Q skew variants and P217R/P229R/N276K pI variants on a second heterodimeric Fc chain, and the E233P/L234V/L235A/G236del/S267K ablation variants on both chains. Heterodimeric Fc backbone 13 is based on human IgG1 (356E/358M allotype), and includes the T366W skew variant on a first heterodimeric Fc chain, the T366S/L368A/Y407V skew variants and H435R/Y436F purification variants on a second heterodimeric Fc chain, and the L234A/L235A/D265S ablation variants on both chains. Heterodimeric Fc backbone 14 is based on human IgG1 (356E/358M allotype), and includes the T366W skew variant on a first heterodimeric Fc chain, the T366S/L368A/Y407V skew variants and H435R/Y436F purification variants on a second heterodimeric Fc chain, and the L234A/L235A/D265S ablation variants and M252Y/S254T/T256E half-life extension variants on both chains. Heterodimeric Fc backbone 15 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 16 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains. Heterodimeric Fc backbone 17 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (G446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

FIG. 11 depicts illustrative sequences of heterodimeric CD20 x CD28 bsAb backbone for use in the 2+1 mAb-scFv format. The format depicted here is based on heterodimeric Fc backbone 1 as depicted in FIG. X, except further including G446_ on monomer 1 (−) and G446_/K447_ on monomer 2 (+). It should be noted that any of the additional backbones depicted in FIG. X may be adapted for use in the 2+1 mAb-scFv format with or without including K447_ on one or both chains. It should be noted that these sequences may further include the M428L/N434S variants or M252Y/S254T/T256E half-life extension variants.

FIG. 12 depicts sequences for “CH1” that find use in embodiments of CD20 x CD28 bsAbs.

FIG. 13 depicts sequences for “hinge” that find use in embodiments of CD20 x CD28 bsAbs.

FIG. 14 depicts the constant domain of the cognate light chains that find use in the subject CD20 x CD28 bsAbs that utilize a Fab binding domain.

FIG. 15 depicts the variable heavy and variable light chain sequences for 1A7, an exemplary phage-derived CD28 binding domain, as well as the sequences for XENP28428, an anti-CD28 mAb based on 1A7 and IgG1 backbone with E233P/L234V/L235A/G236del/S267K ablation variant. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 16 depicts the sequences for affinity-optimized variable heavy domains from anti-CD28 clone 1A7. It should be noted that the variable heavy domains can be paired with any of the other variable light domains depicted herein including, for example, SEQ ID NOs: 19, 75, 79, and 200-305 (e.g. 1A7_H1.1_L1.71 as utilized in XENP39583).

FIG. 17 depicts the sequences for affinity-optimized variable light domain from anti-CD28 clone 1A7. It should be noted that the variable light domains can be paired with any of the other variable heavy domains depicted herein including, for example, SEQ ID NOs: 1, 11, 15, 63, 67, 71, and 131-199 (e.g. 1A7_H1.1_L1.71 as utilized in XENP39583).

FIGS. 18A-18C depict the sequence for illustrative affinity-optimized 1A7 VH/VH pairs. It should be noted that these pairs may be formatted as Fabs or as scFvs. Additionally, in the scFv format, these pairs may be formatted in the VHVL orientation or the VLVH orientation.

FIG. 19 depicts the sequences for illustrative variable heavy domains from anti-CD28 clone 1A7 with an introduced cysteine to allow for “stapling” of the scFv. It should be noted that the variable heavy domains can be paired with any of the other variable light domains depicted in FIGS. 17, 20, and 23, including SEQ ID NOs: SEQ ID NOs: 19, 75, 79, and 200-305 (e.g. as utilized in XENP42157, XENP42160, and XENP42163).

FIG. 20 depicts the sequences for illustrative variable light domain from anti-CD28 clone 1A7 with an introduced cysteine to allow for “stapling” of the scFv. It should be noted that the variable light domains can be paired with any of the other variable heavy domains depicted FIGS. 15, 16, 19, and 22, including SEQ ID NOs: 1, 11, 15, 63, 67, 71, and 131-199 (e.g. as utilized in XENP42157, XENP42160, and XENP42163).

FIGS. 21A and 21B depict the sequence for illustrative stapled 1A7 VH/VH pairs. These pairs may be formatted in the VHVL orientation or the VLVH orientation.

FIGS. 22A-22F depict additional sequences for affinity-optimized variable heavy domains from anti-CD28 clone 1A7. It should be noted that the variable heavy domains can be paired with any of the other variable light domains depicted herein including SEQ ID NOs: SEQ ID NOs: 19, 75, 79, and 200-305.

FIGS. 23A-231 depict additional sequences for affinity-optimized variable light domains from anti-CD28 clone 1A7. It should be noted that the variable light domains can be paired with any of the other variable heavy domains depicted herein including SEQ ID NOs: 1, 11, 15, 63, 67, 71, and 131-199.

FIGS. 24A and 24B depict consensus framework regions (FR) and complementarity determining regions (CDRs) (as in Kabat) for anti-CD28 clone 1A7 variable heavy and variable light domain variants. These consensus sequences may further include “staple” modifications to enable use in a “stapled” scFv.

FIG. 25 depicts illustrative affinity-engineered 1A7 VH/VL pairs and their binding affinities in the context of scFvs (in the context of 1+1 Fab-scFv-Fc bsAb format).

FIGS. 26A-26H depict the variable heavy and variable light chain sequences for additional CD28 binding domains which find use in the CD20 x CD28 bsAbs of the invention. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 27 depicts the sequences for XENP27181, a bivalent anti-CD28 mAb based on HuTN228 binding domain and IgG1 backbone with E233P/L234V/L235A/G236del/S267K ablation variant; and XENP29154 which is an in-house produced version of TGN1412.

FIG. 28 depicts binding of illustrative bivalent anti-CD28 mAbs based on phage-derived clones on human PBMCs. The data show that the phage campaign generated CD28 binding domains having weaker maximum binding than prior art HuTN228 (which is related to the humanized CD28 binding domains described in Example 1A).

FIGS. 29A-29N depicts bispecific formats of the present invention. FIG. 29A depicts the “1+1 Fab-scFv-Fc” format, with a first Fab arm binding a first antigen and a second scFv arm binding second antigen. The 1+1 Fab-scFv-Fc format comprises a first monomer comprising a first heavy chain variable region (VH1) covalently attached to the N-terminus of a first heterodimeric Fc backbone (optionally via a linker), a second monomer comprising a single-chain Fv covalently attached to the N-terminus of a second corresponding heterodimeric Fc backbone (optionally via a linker), and a third monomer comprising a light chain variable region covalently to a light chain constant domain, wherein the light chain variable region is complementary to the VH1. FIG. 29B depicts the “2+1 Fab2-scFv-Fc” format, with a first Fab arm and a second Fab-scFv arm, wherein the Fab binds a first antigen and the scFv binds second antigen. The 2+1 Fab2-scFv-Fc format comprises a first monomer comprising a first heavy chain variable region (VH1) covalently attached to the N-terminus of a first heterodimeric Fc backbone (optionally via a linker), a second monomer comprising the VH1 covalently attached (optionally via a linker) to a single-chain Fv covalently attached (optionally via a linker) to the N-terminus of a second corresponding heterodimeric Fc backbone, and a third monomer comprising a light chain variable region covalently to a light chain constant domain, wherein the light chain variable region is complementary to the VH1. FIG. 29C depicts the “1+1 Common Light Chain” or “1+1 CLC” format, with a first Fc comprising a first Fab arm binding a first antigen and a second Fc comprising a second Fab arm binding second antigen. The 1+1 CLC format comprises a first monomer comprising VH1-CH1-hinge-CH2-CH3, a second monomer comprising VH2-CH1-hinge-CH2-CH3, and a third monomer comprising VL-CL. The VL pairs with the VH1 to form a binding domain with a first antigen binding specificity; and the VL pairs with the VH2 to form a binding domain with a second antigen binding specificity. FIG. 29D depicts the “2+1 Common Light Chain” or “2+1 CLC” format, with a first Fc comprising 2 Fab arms each binding a first antigen and a second Fc comprising 1 Fab arm binding a second antigen. The 2+1 CLC format comprises a first monomer comprising VH1-CH1-hinge-VH1-CH1-hinge-CH2-CH3, a second monomer comprising VH2-CH1-hinge-CH2-CH3, and a third monomer comprising VL-CL. The VL pairs with the first and second VH1 to form binding domains with a first antigen binding specificity; and the VL pairs with the VH2 to form a binding domain with a second antigen binding specificity. FIG. 29E depicts the “2+1 mAb-scFv” format, with a first Fc comprising an N-terminal Fab arm binding a first antigen and a second Fc comprising an N-terminal Fab arm binding the first antigen and a C-terminal scFv binding a second antigen. The 2+1 mAb-scFv format comprises a first monomer comprising VH1-CH1-hinge-CH2-CH3, a second monomer comprising VH1-CH1-hinge-CH2-CH3-scFv, and a third monomer comprising VL-CL. The VL pairs with the first and second VH1 to form binding domains with binding specificity for the first antigen. FIG. 29F depicts the “2+1 mAb-scFv” format which comprises a first monomer comprising from N-terminal to C-terminal VH1-CH1-linker-VH2-CH1-hinge-CH2-CH3 wherein CH2-CH3 is a first heterodimeric Fc domain; a second monomer comprising from N-terminal to C-terminal scFv-linker-CH2-CH3 wherein CH2-CH3 is a second heterodimeric Fc domain complementary to the first heterodimeric Fc domain and wherein the scFv has a first antigen specificity; and a third monomer that is a common light chain comprising from N-terminal to C-terminal VL-CL wherein the VL pairs with VH1 and VH2 of the first monomer to form two antigen binding domains each having a specificity for a second antigen binding domain. Additional bispecific formats include G) dual scFv, H) one-arm scFv-mAb, I) scFv-mAb, J) bispecific mAb, K) one-arm central-scFv, L) mAb-Fv, M) central-Fv, and N) trident.

FIGS. 30A-30E depict the variable heavy and variable light chain sequences for CD20 binding domains which find use in the CD20 x CD28 bsAbs of the invention. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 31A and 31B depicts A) classic T cell/APC interaction and B) replication of the classic T cell/APC interaction by combining CD3 bispecific antibodies with CD28 bispecific antibodies. In classic T cell/APC interaction, there is a first signal provided by TCR reactivity with peptide-MHC (Signal 1) and a second signal provided by CD28 crosslinking by CD80/CD86 being expressed on APCs (Signal 2) which together fully activate T cells. In contrast in treatment with CD3 bispecifics, only the first signal is provided. The CD28 signal may be provided by a CD28 bispecific with the idea to promote activation and proliferation through CD28 costimulation. In some embodiments, TAA1 and TAA2 may be different antigens. In some embodiments, TAA1 and TAA2 may be same antigen but different epitopes. In some embodiments, TAA1 and TAA2 may be same antigen and same epitope.

FIGS. 32A and 32B depict sequences for illustrative CD79b x CD20 x CD3 trispecific antibody that may be combined with the CD20 x CD28 bsAbs of the invention.

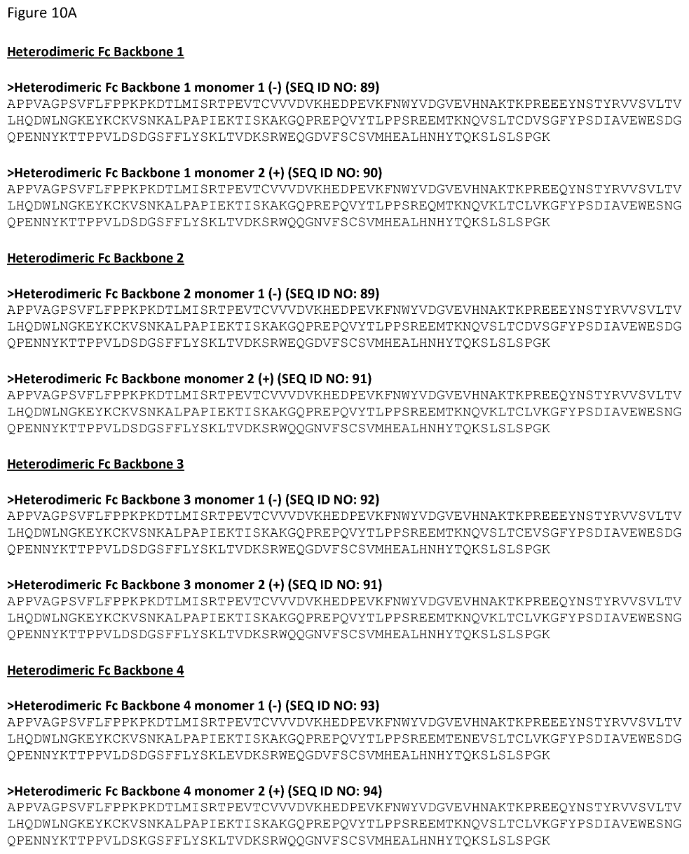

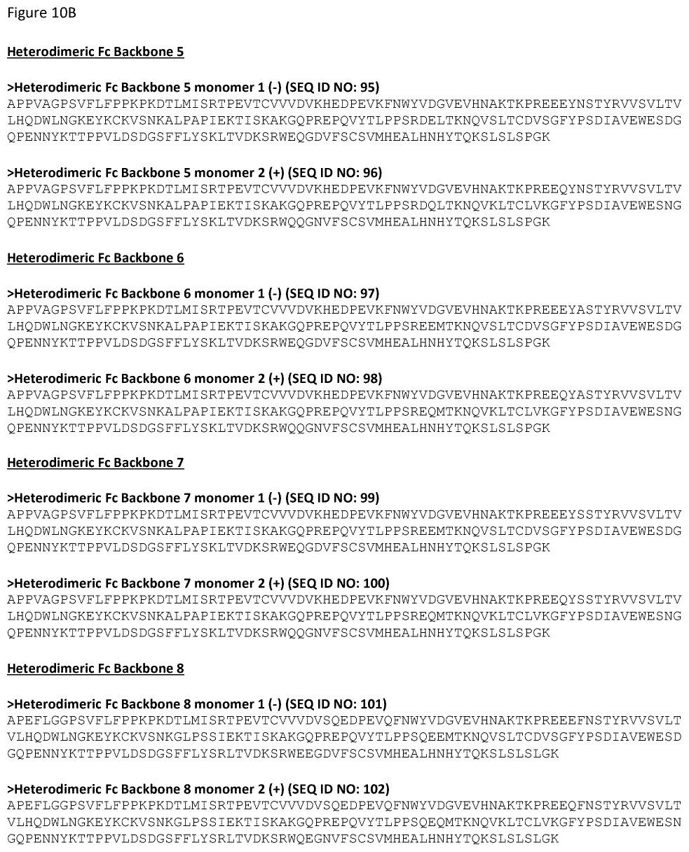

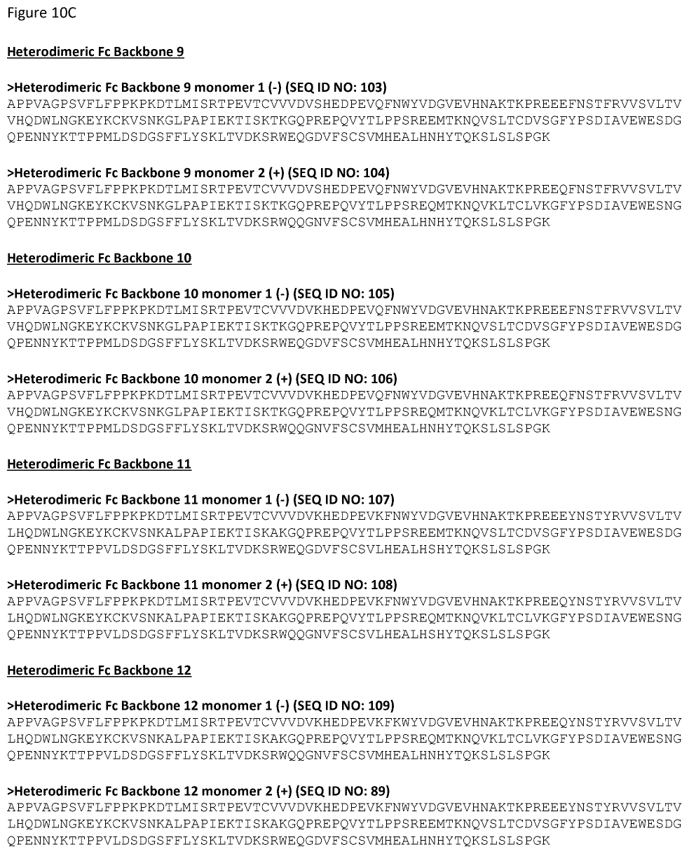

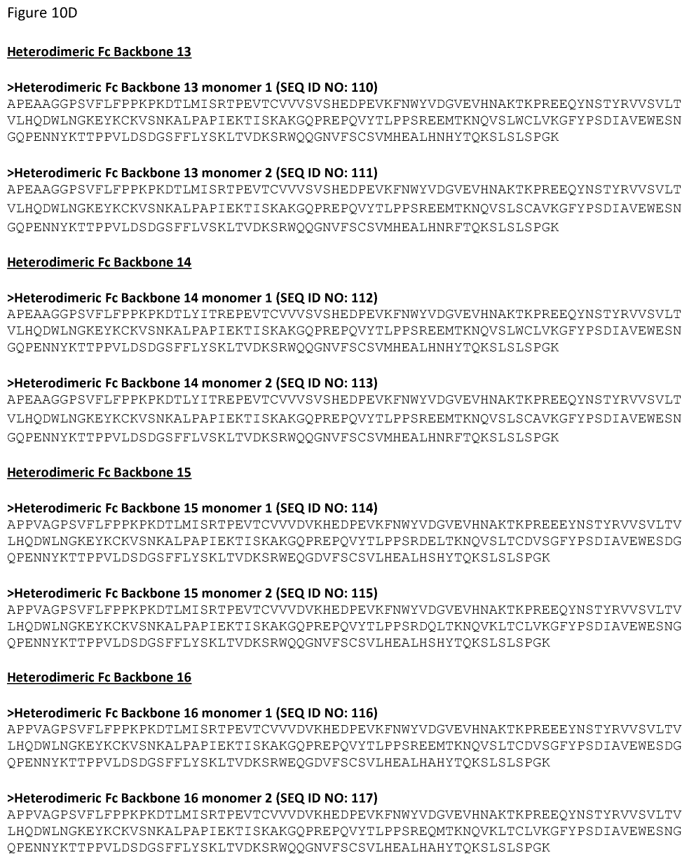

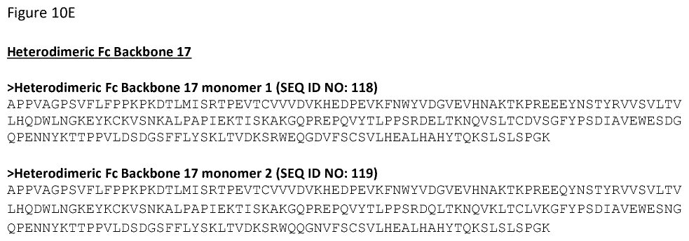

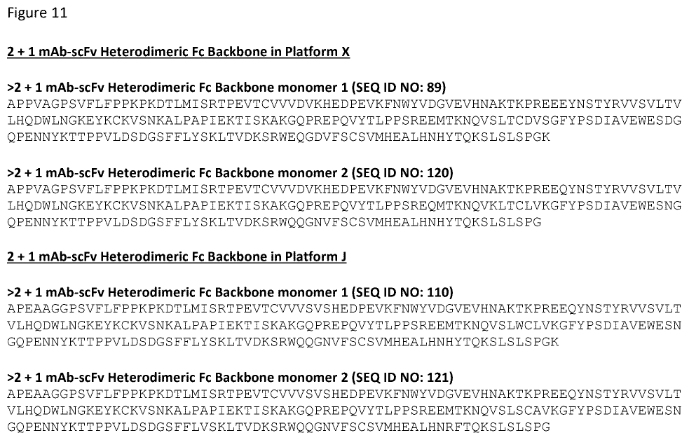

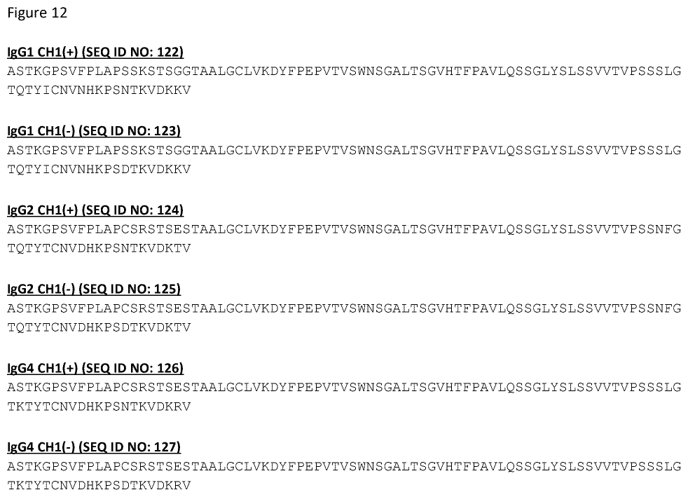

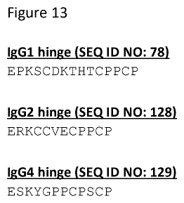

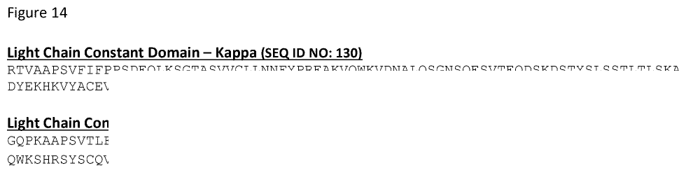

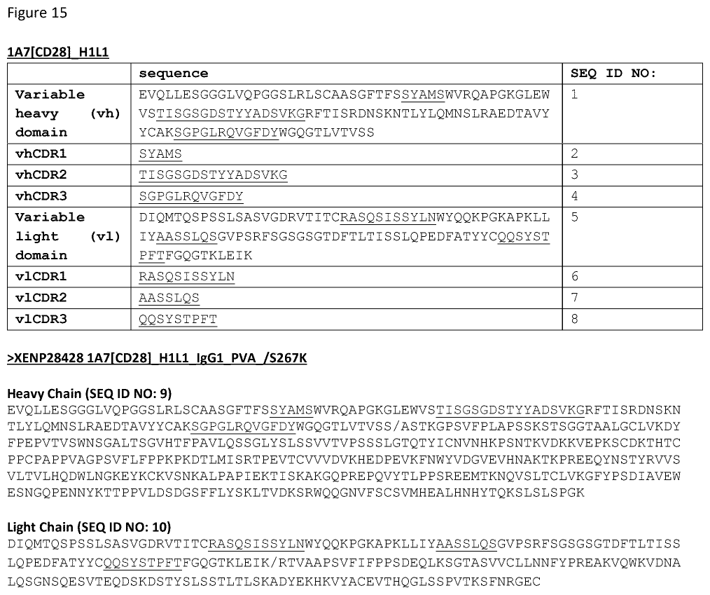

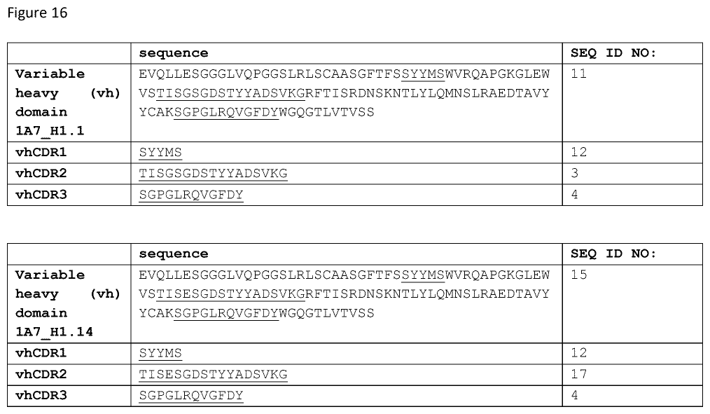

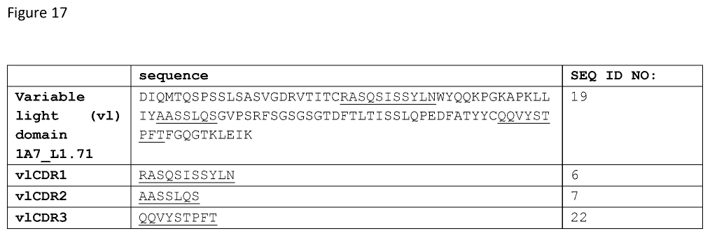

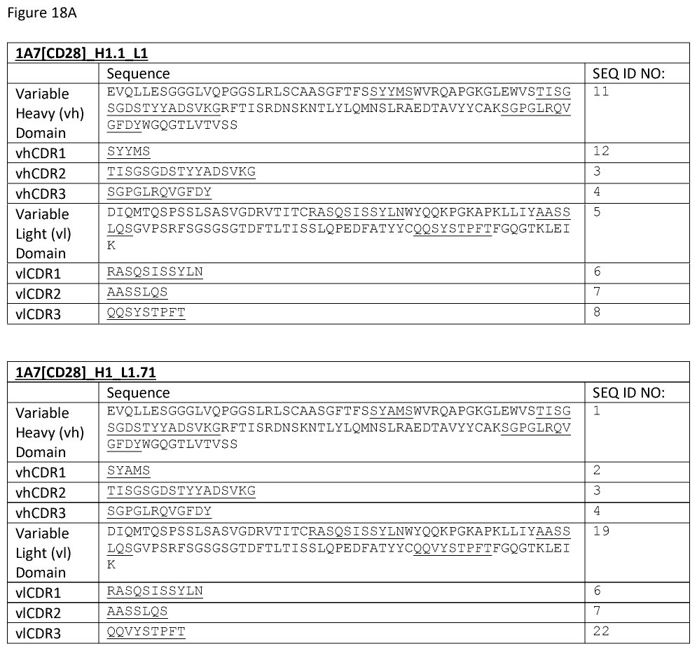

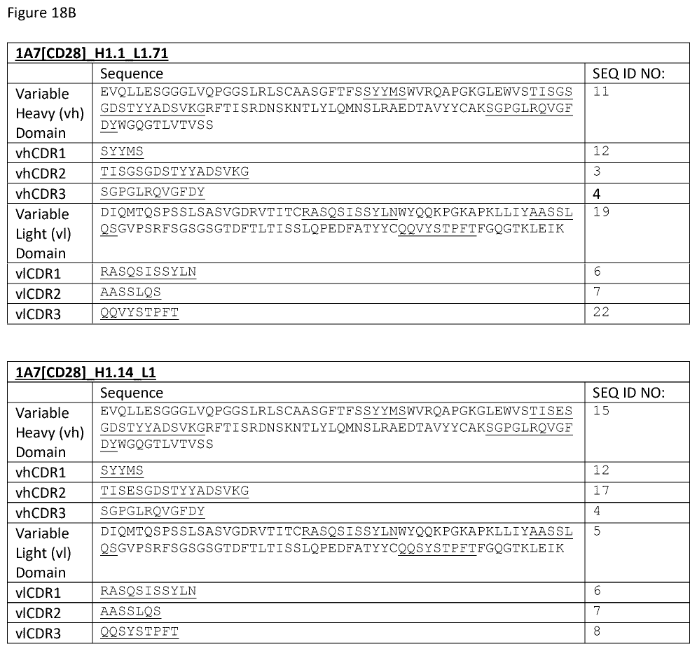

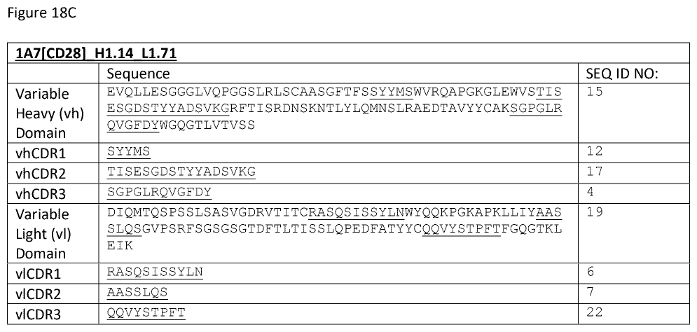

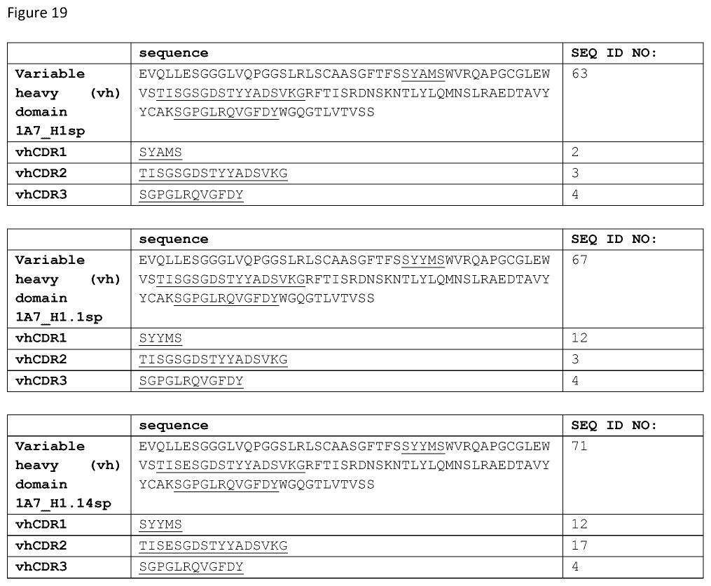

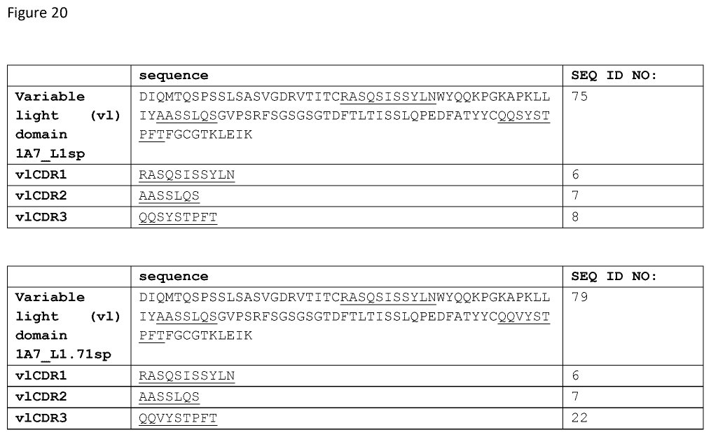

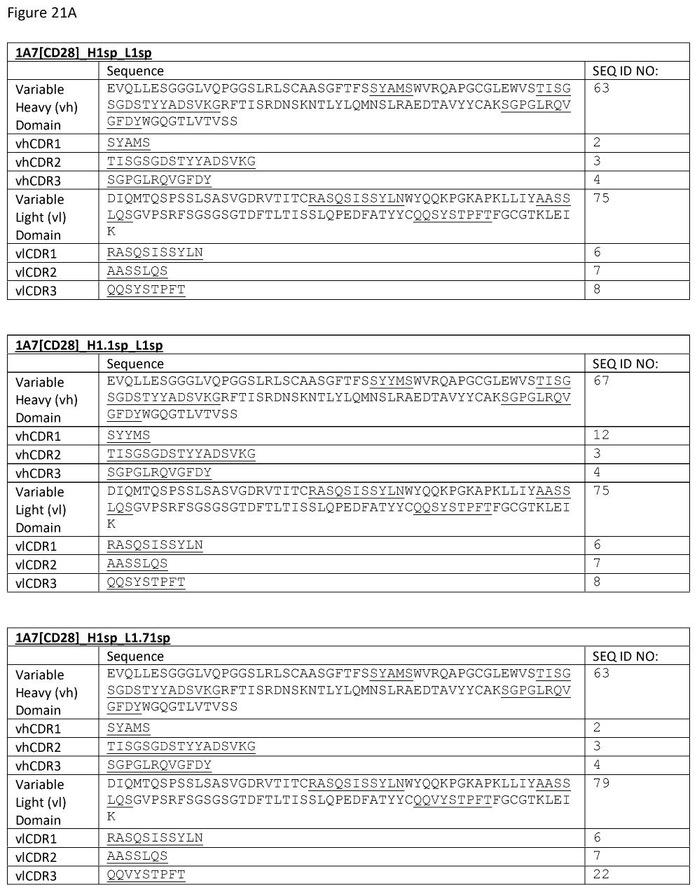

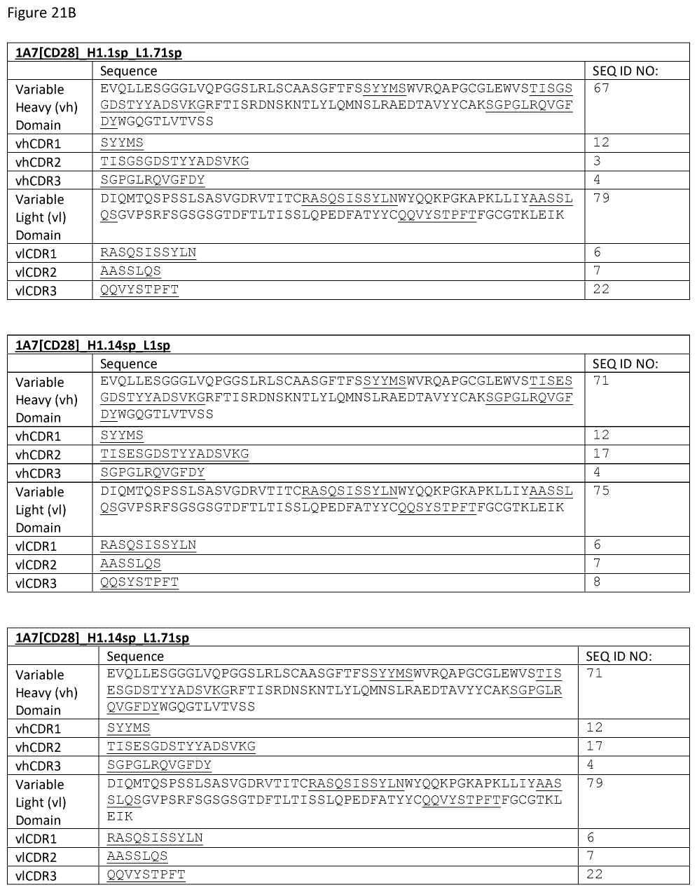

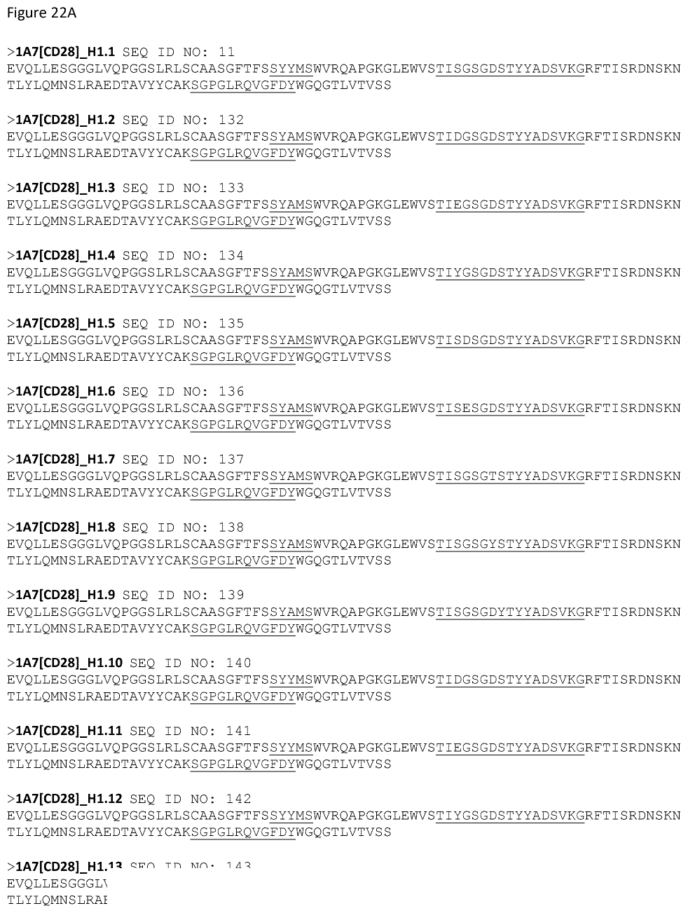

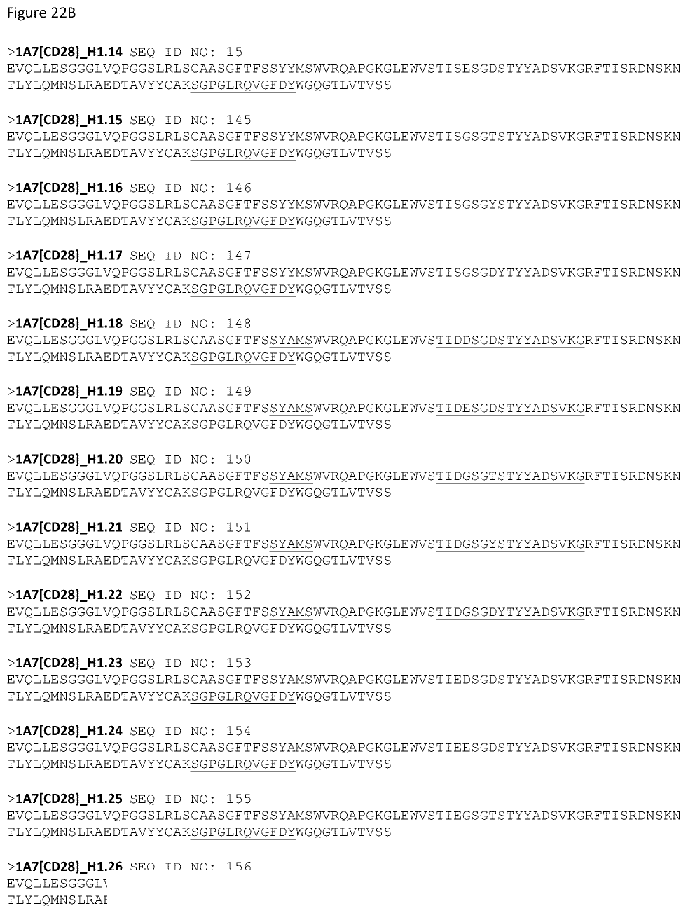

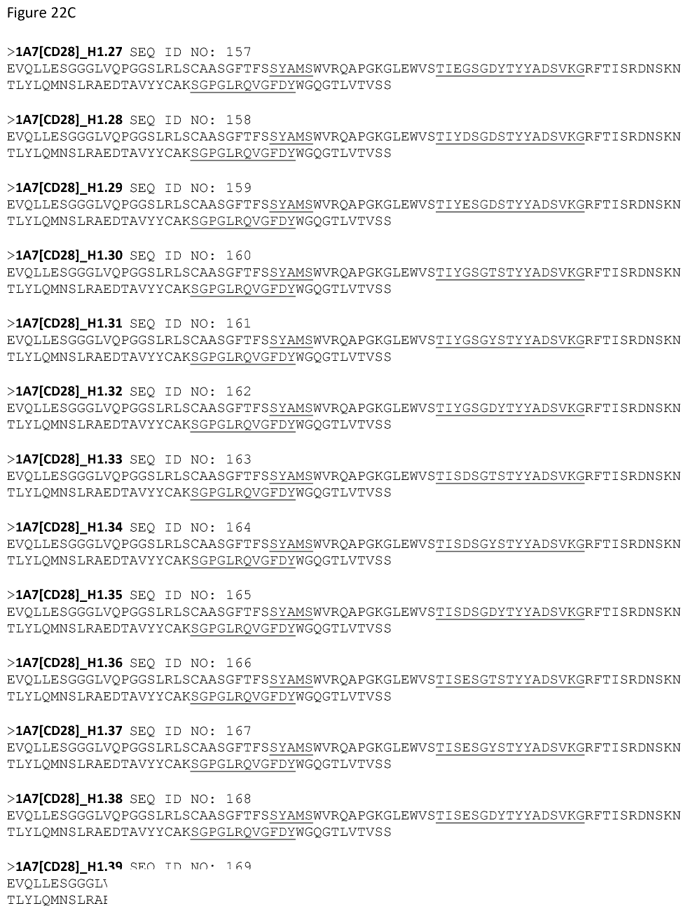

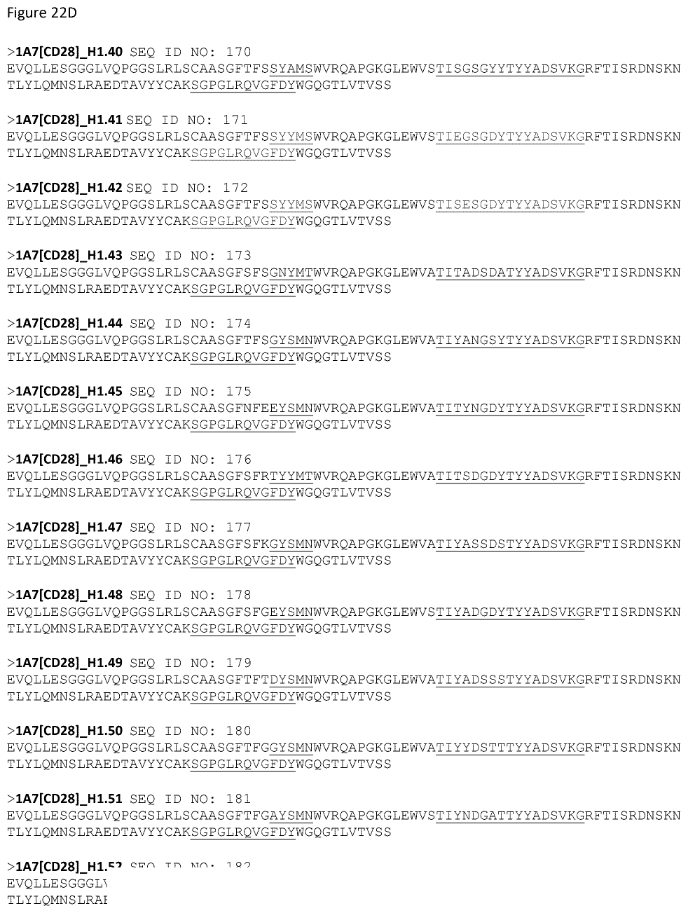

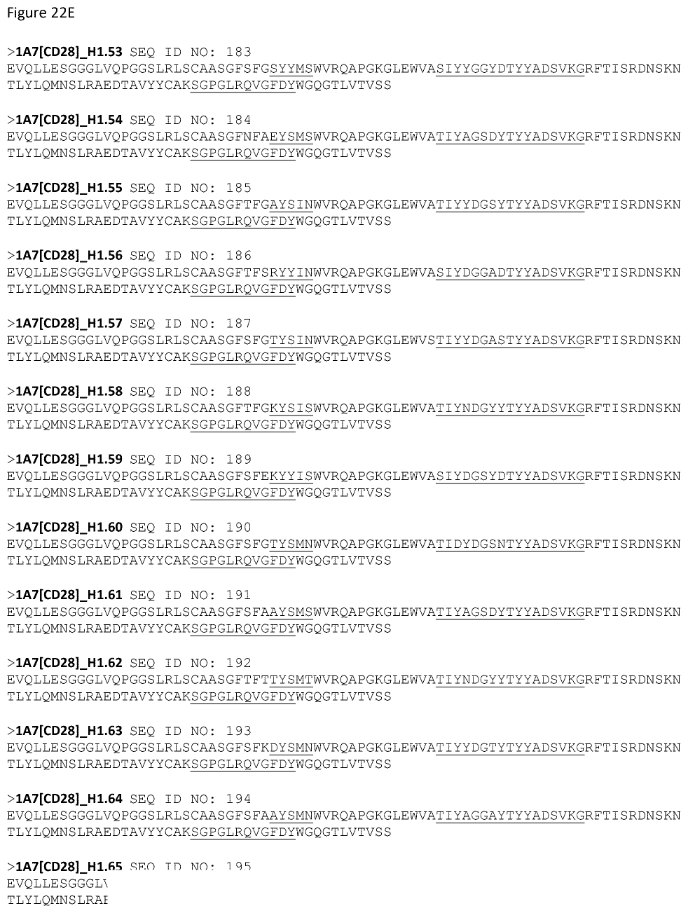

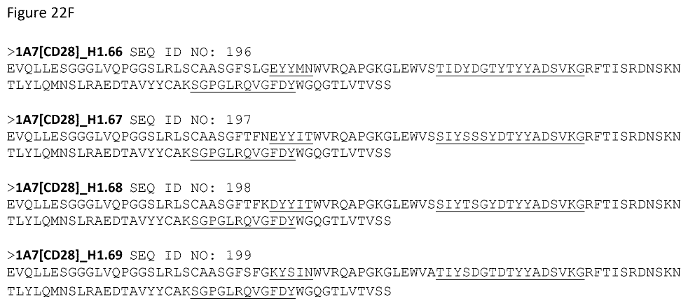

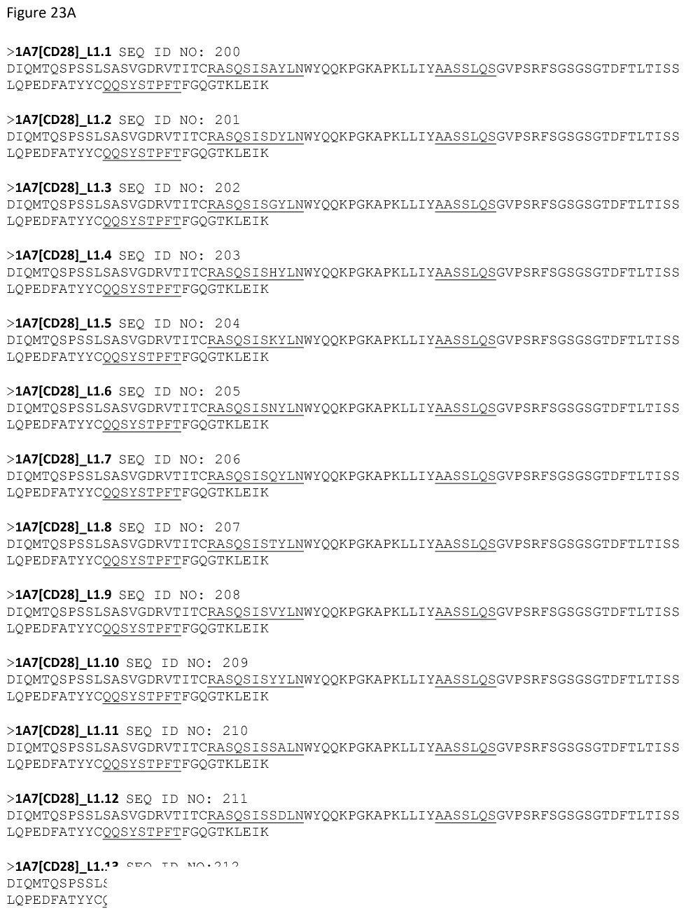

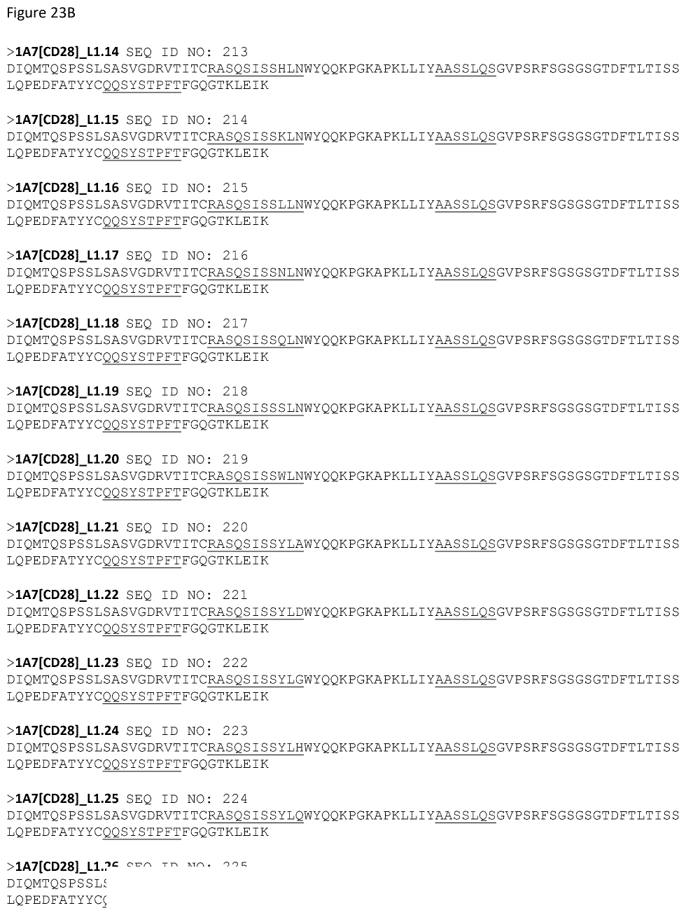

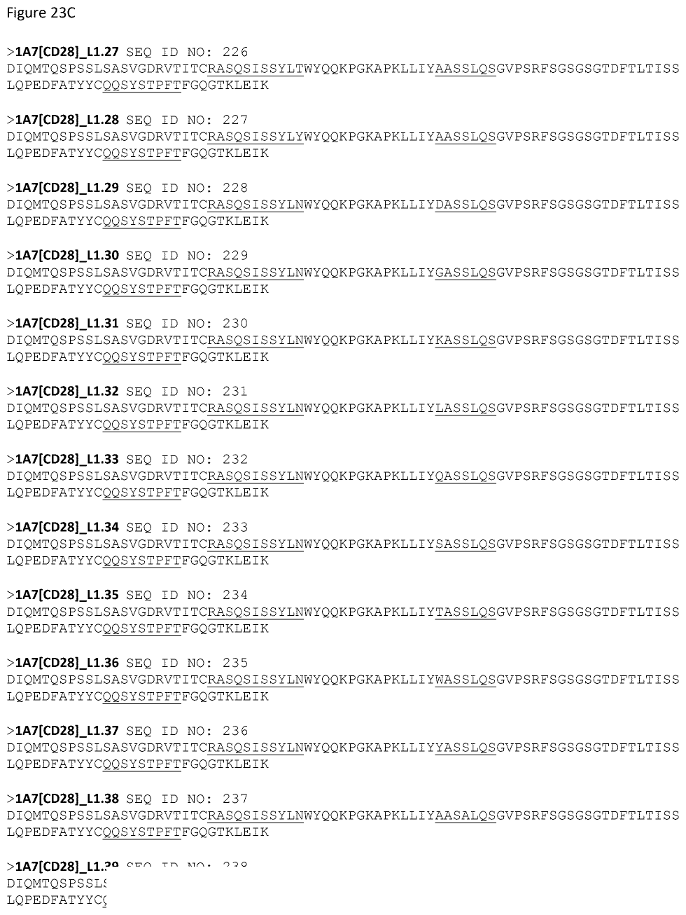

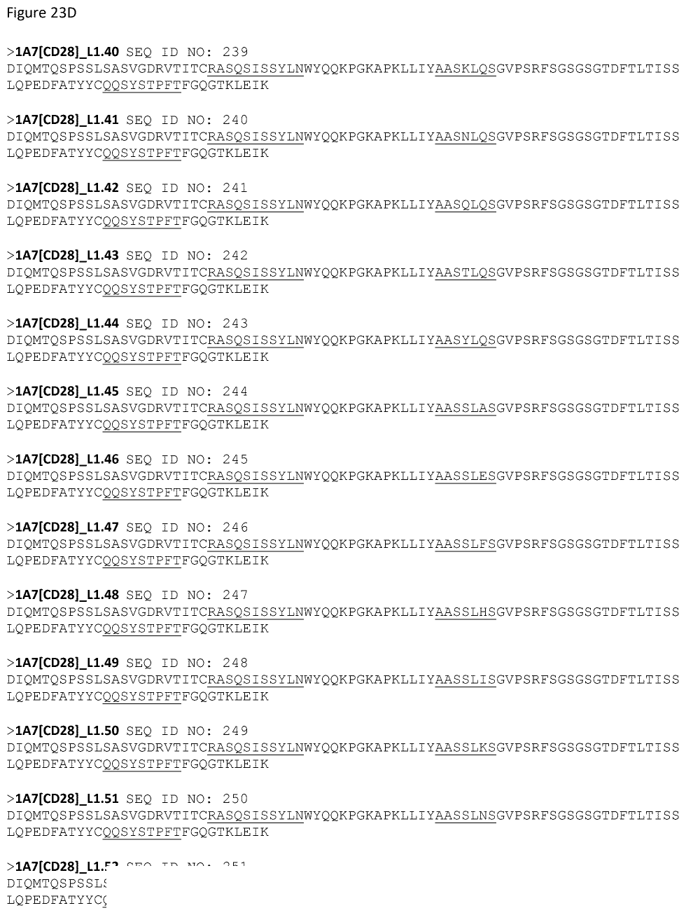

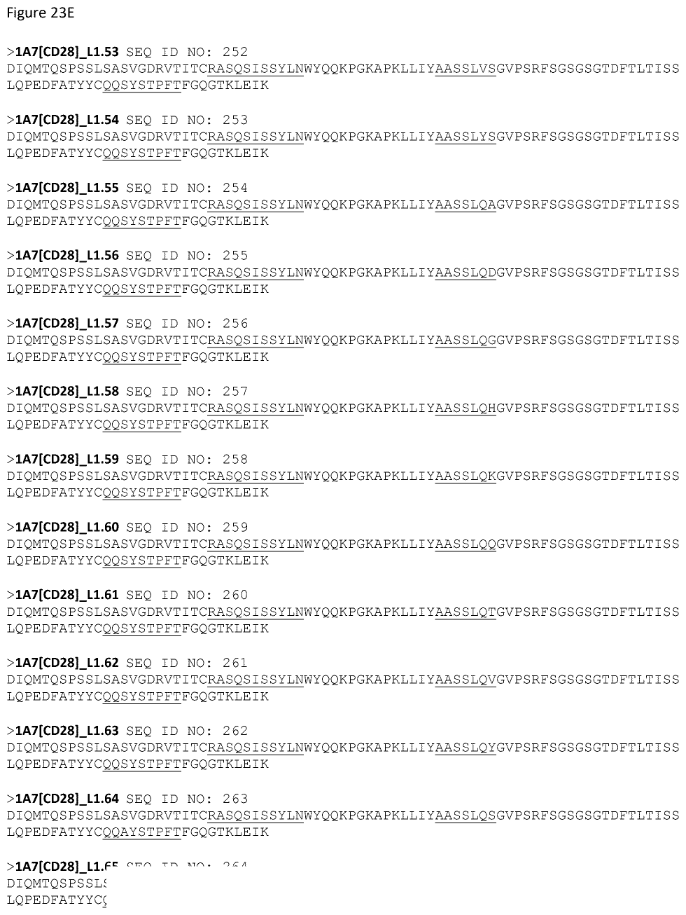

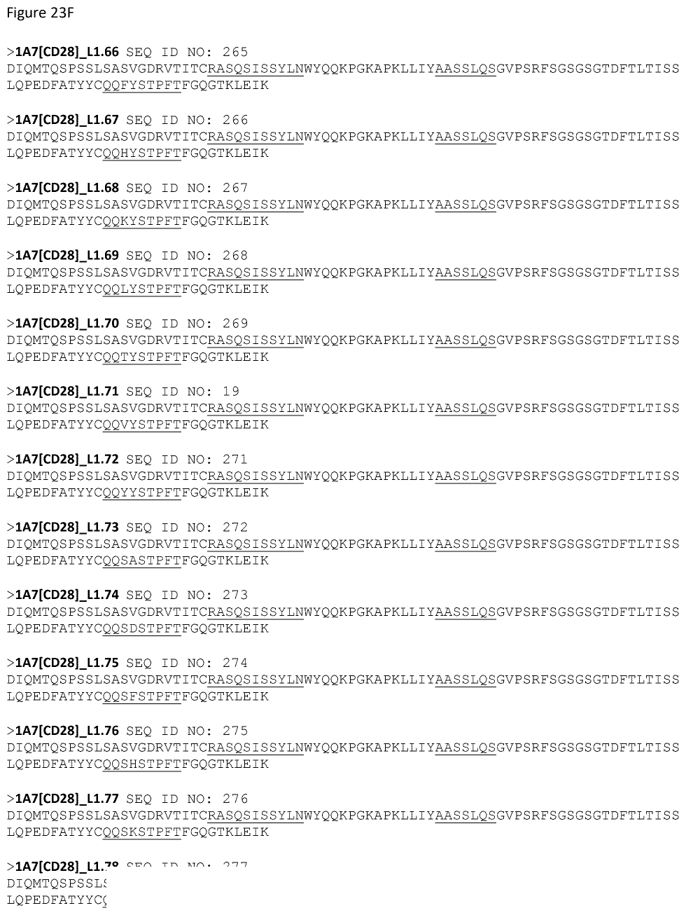

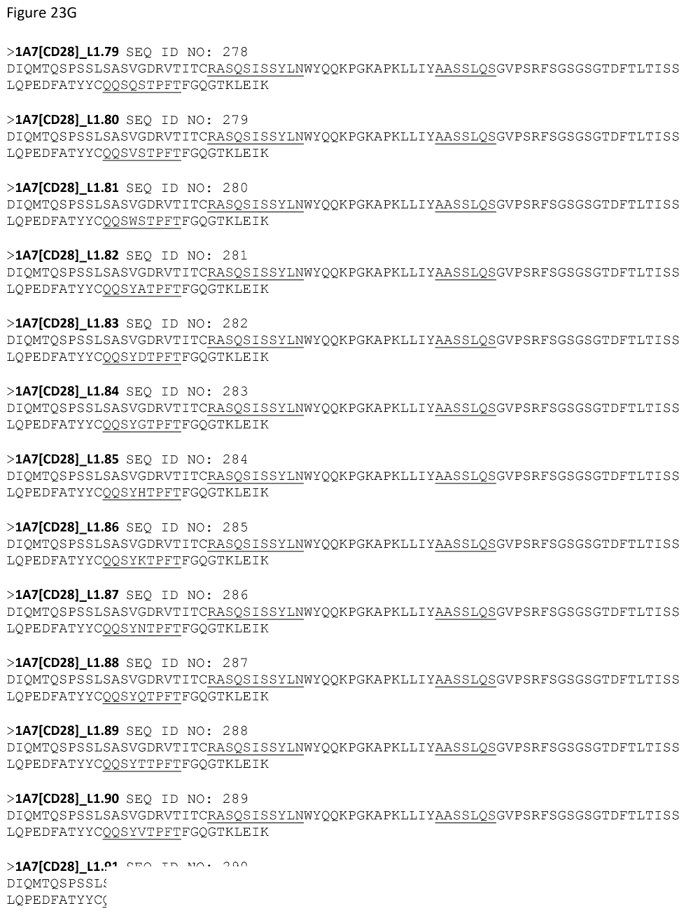

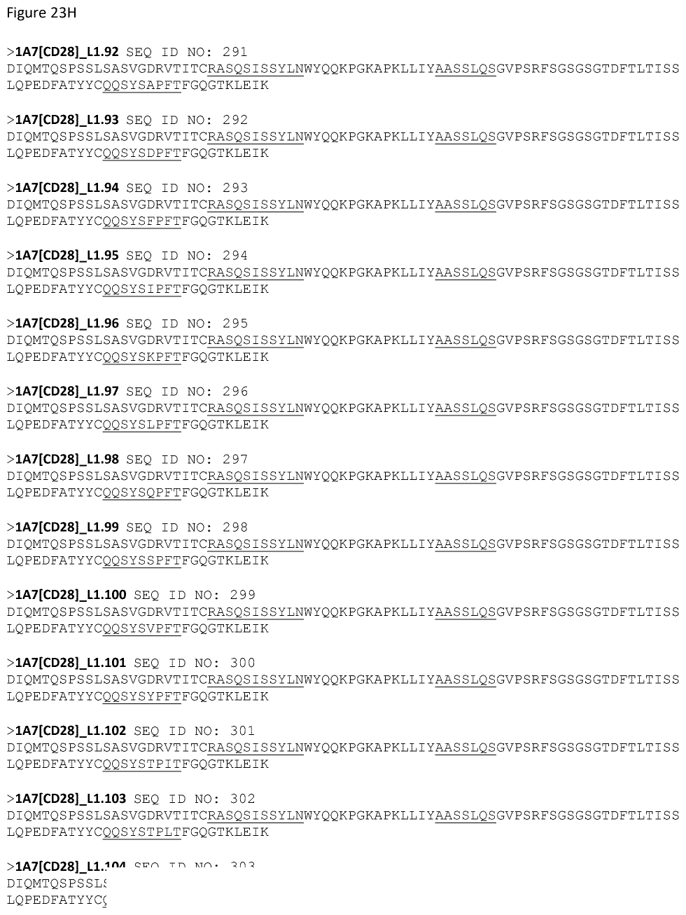

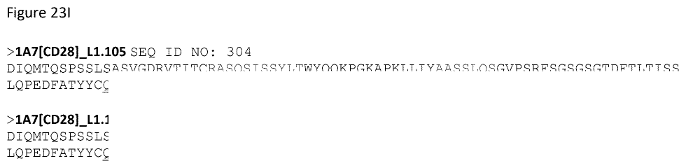

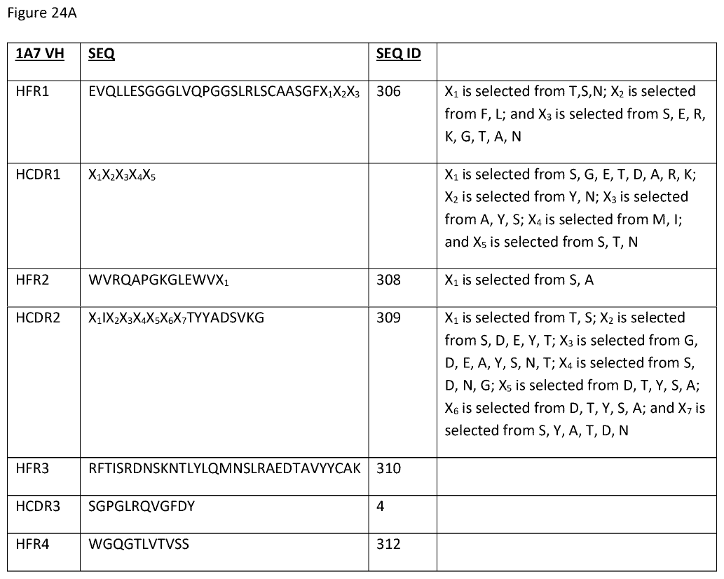

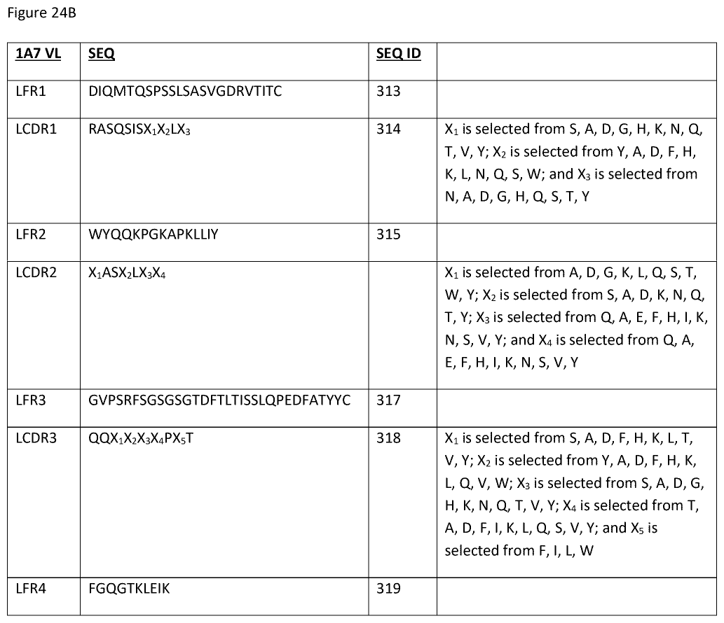

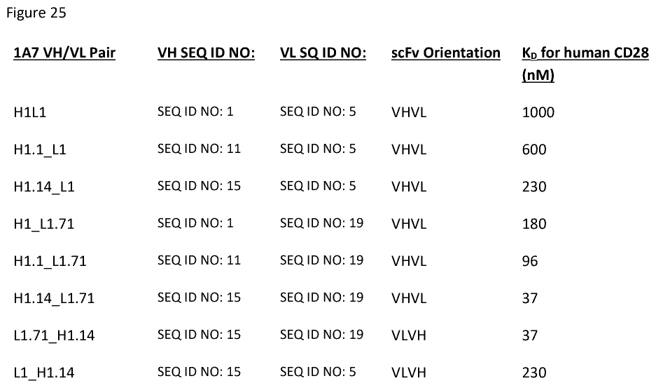

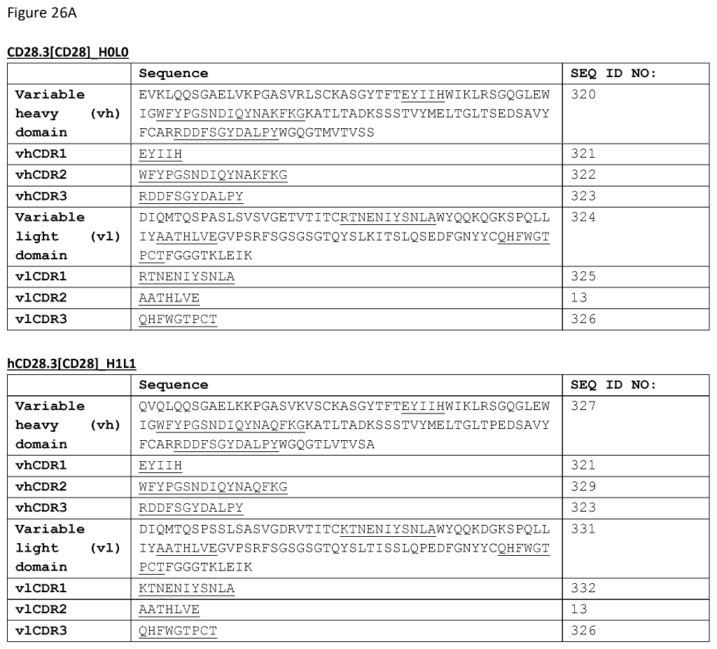

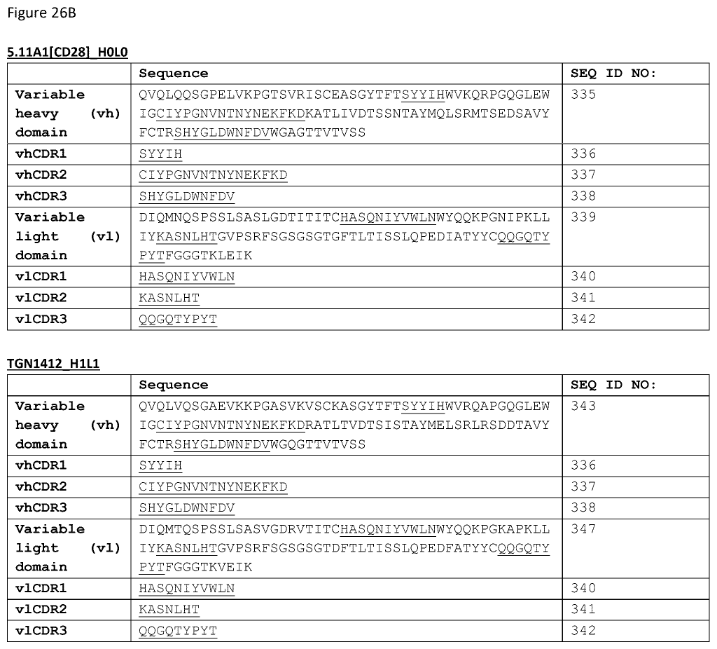

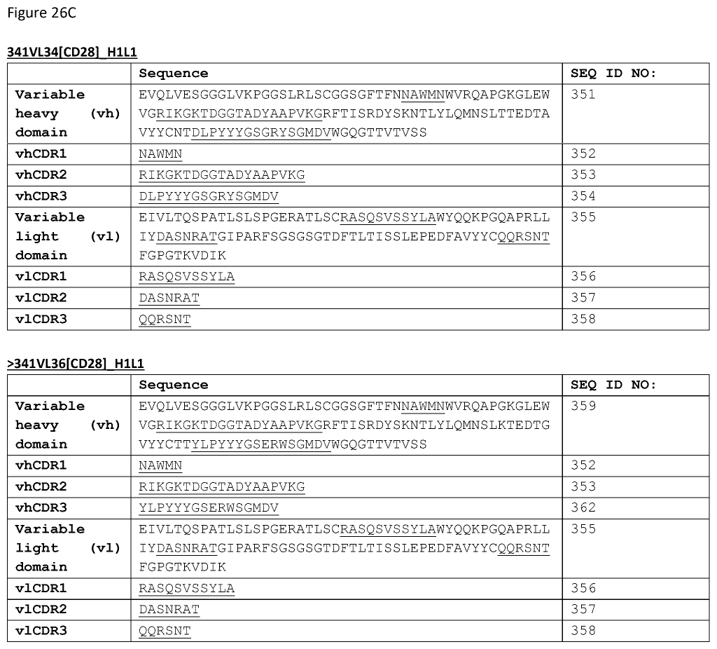

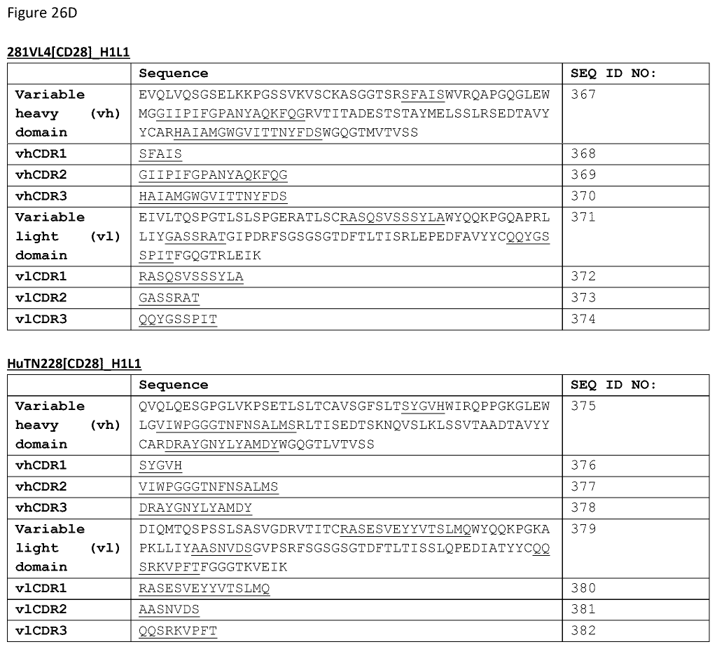

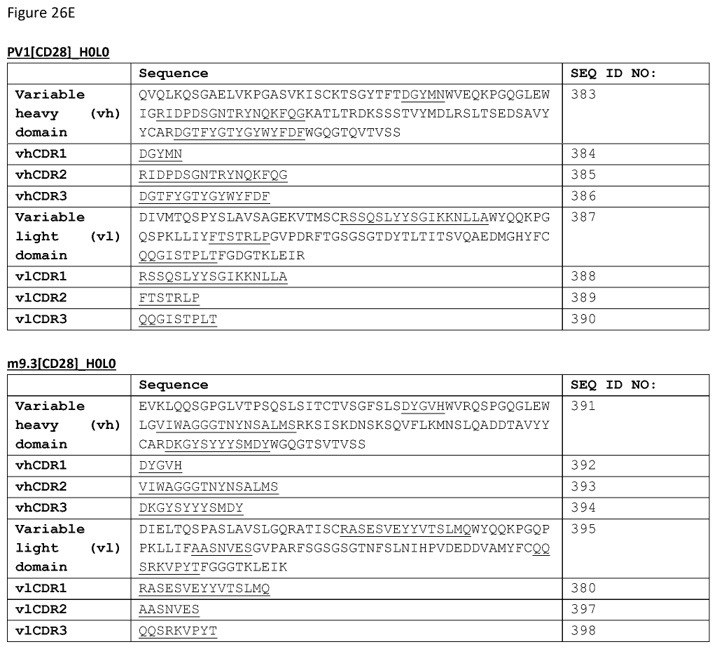

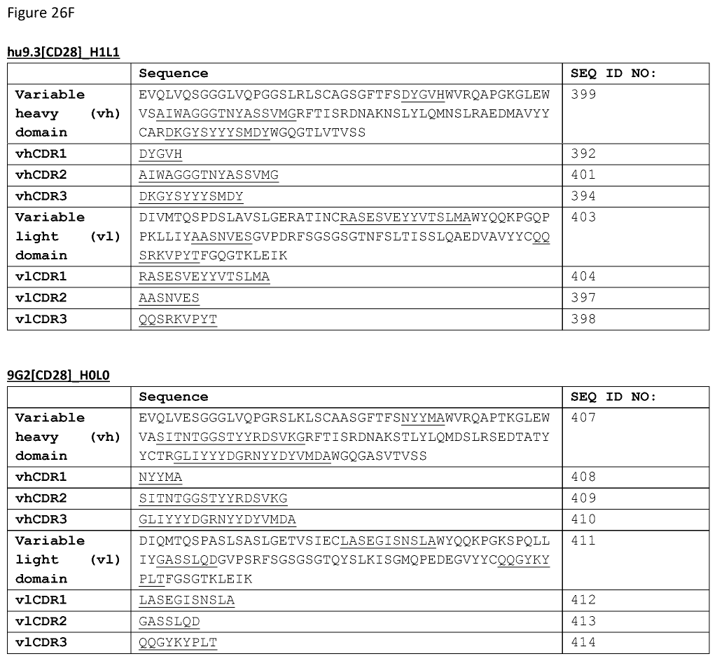

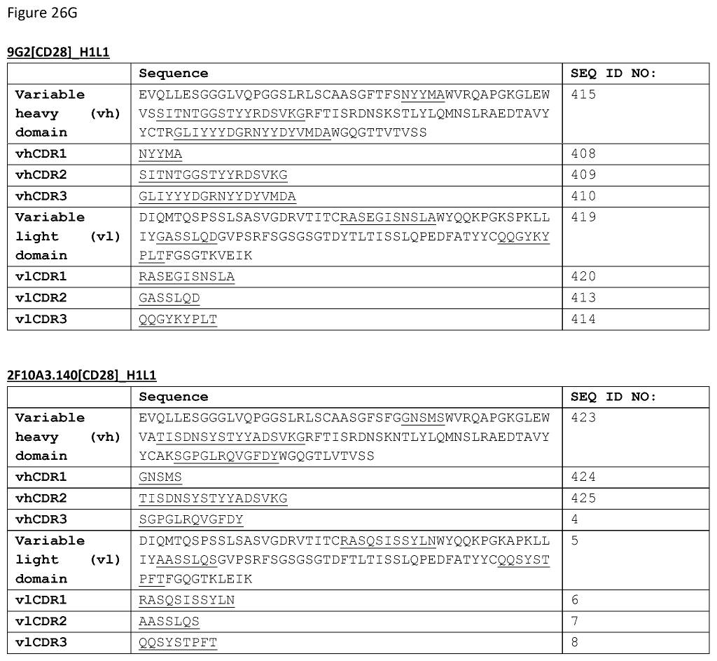

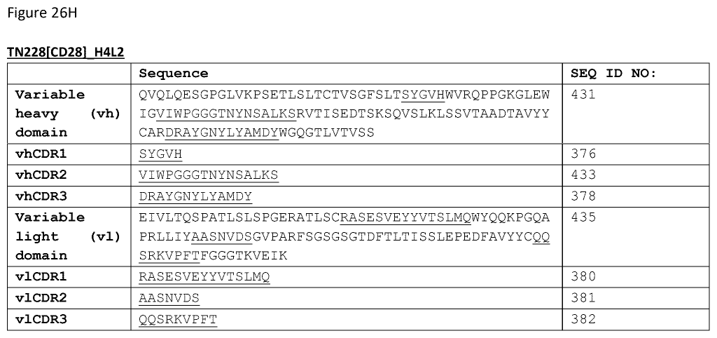

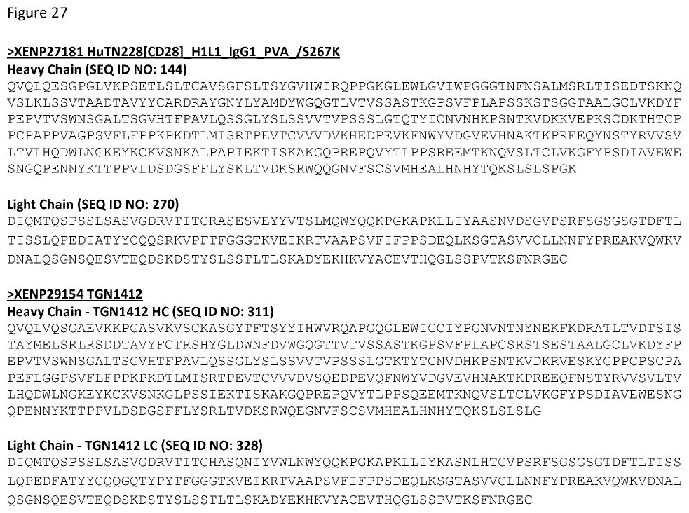

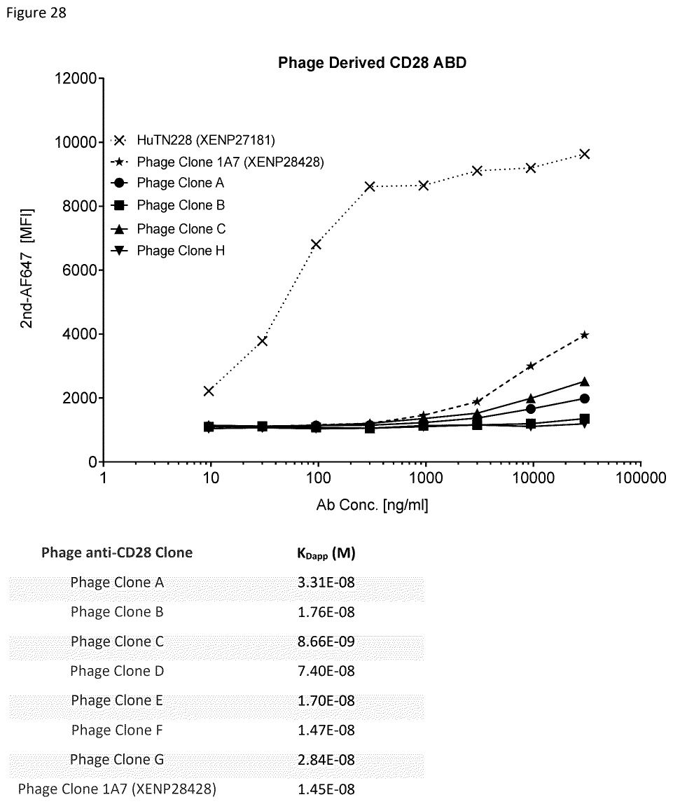

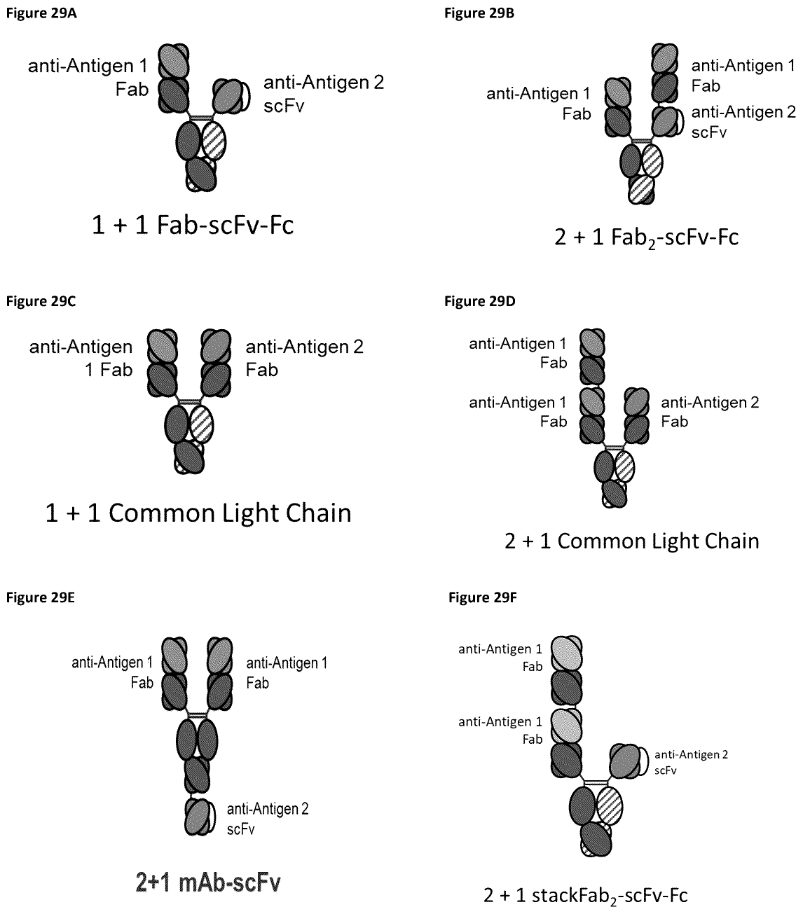

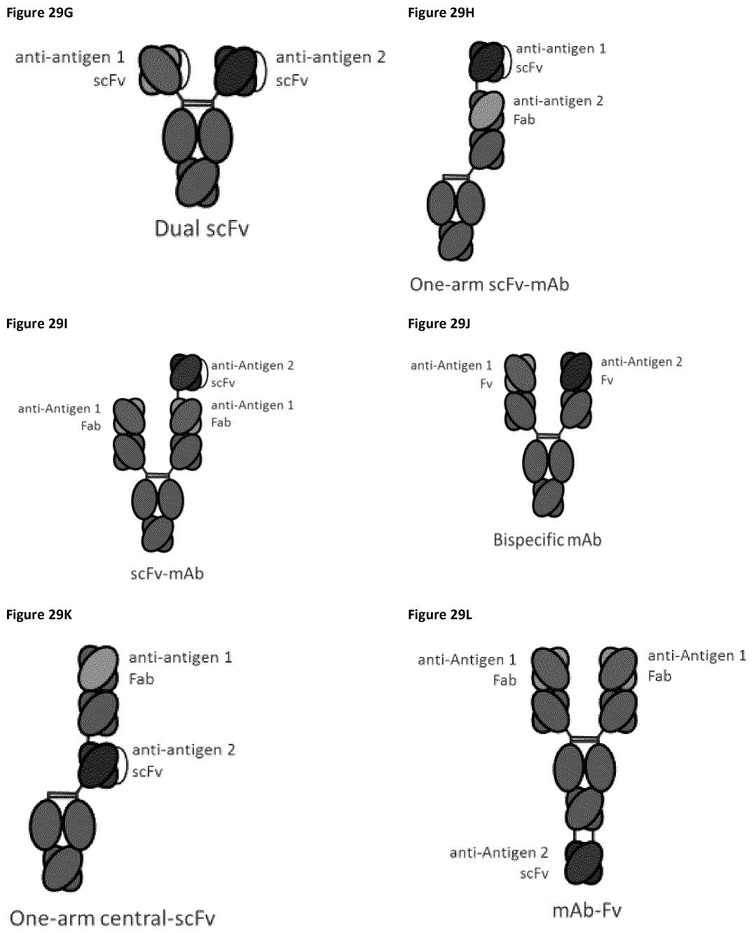

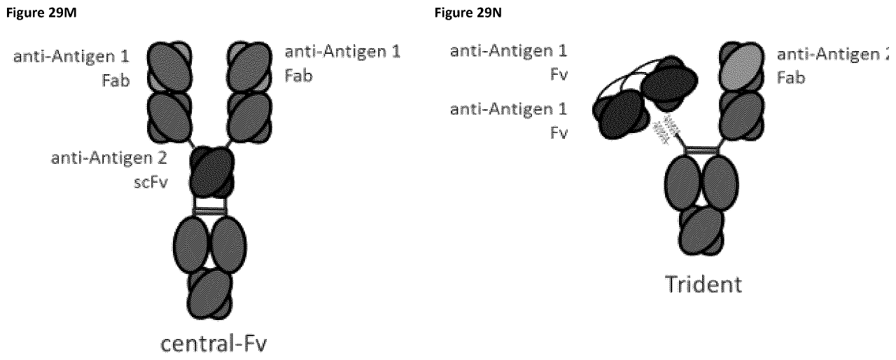

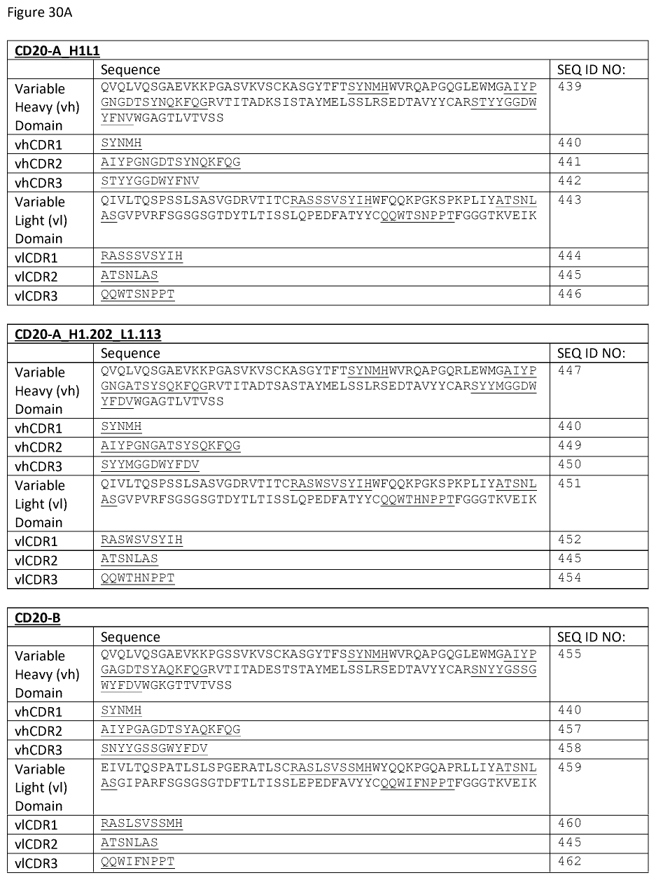

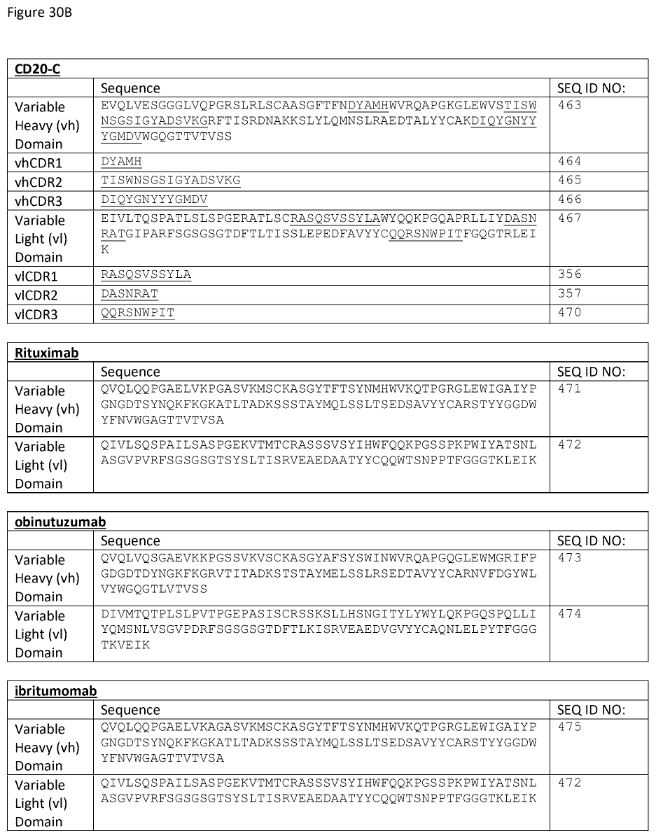

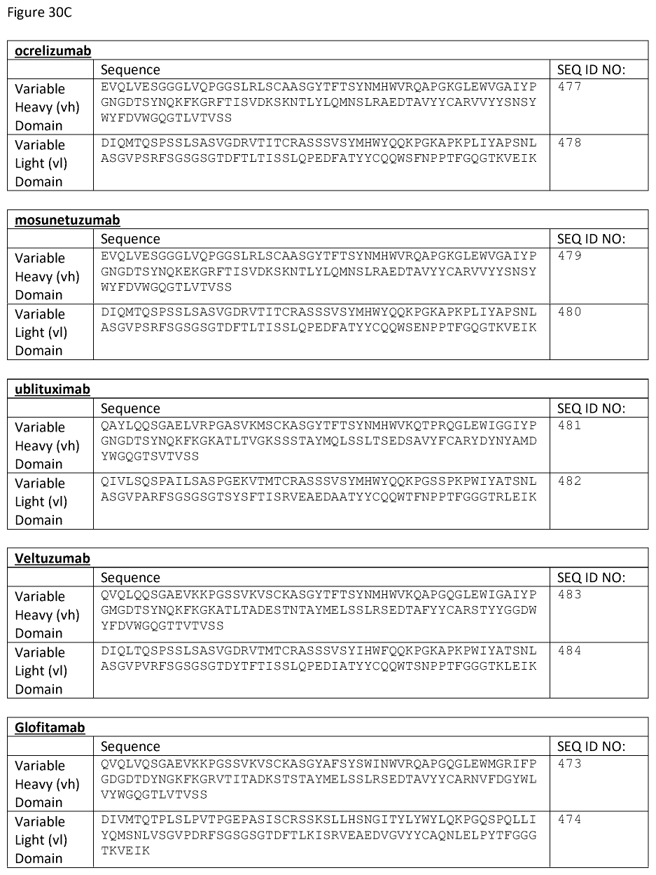

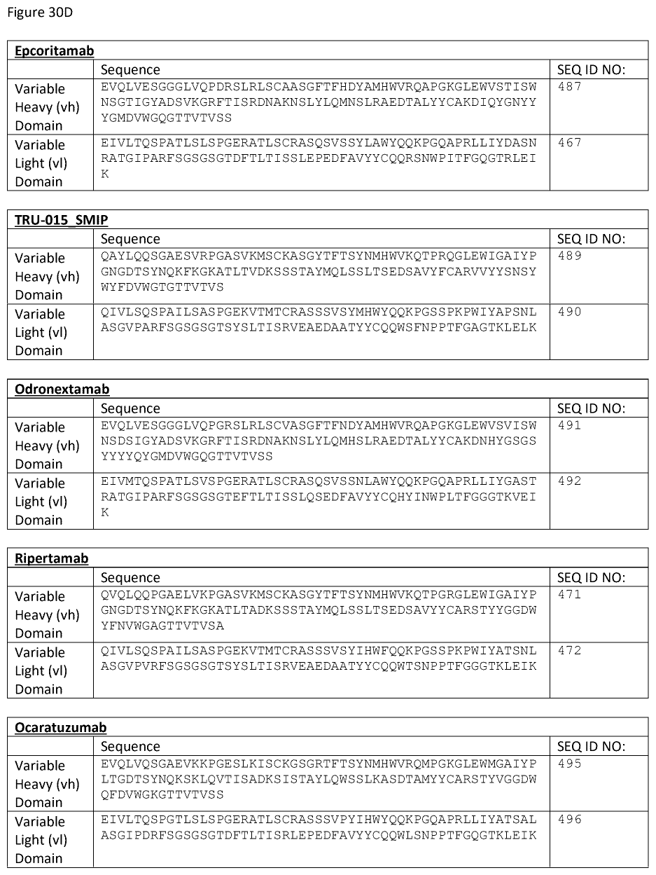

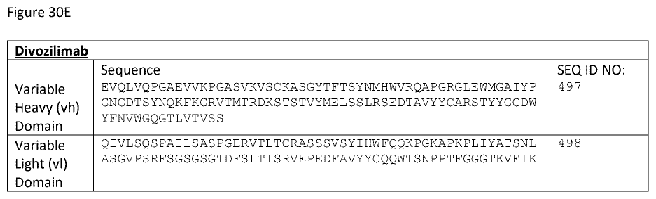

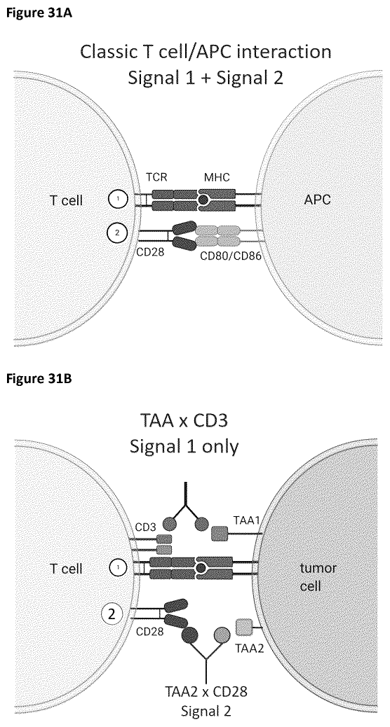

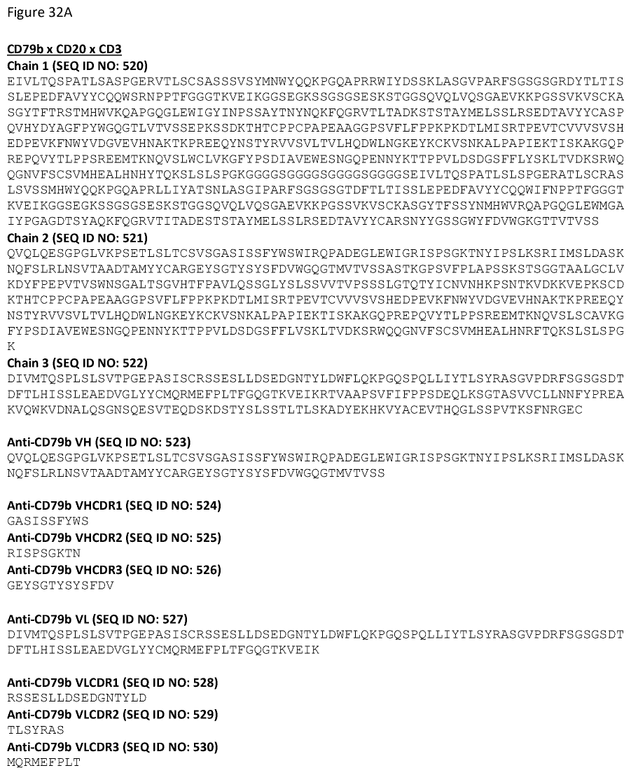

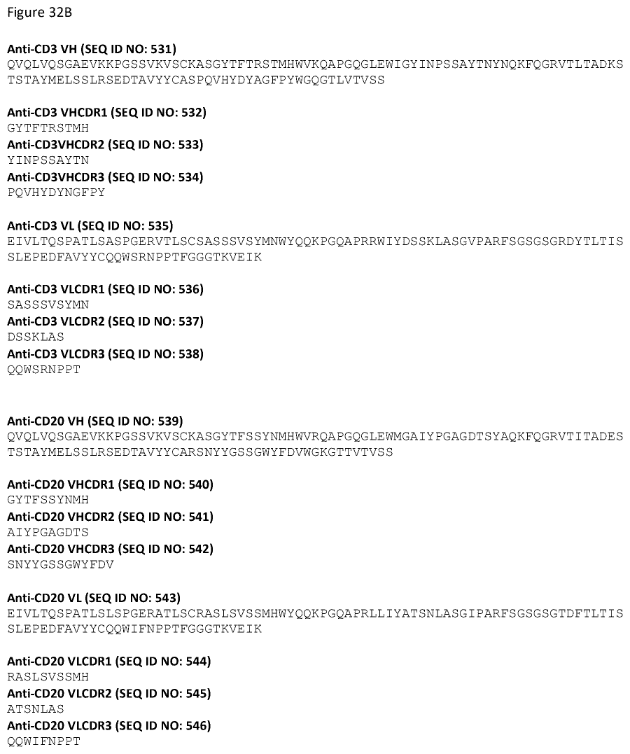

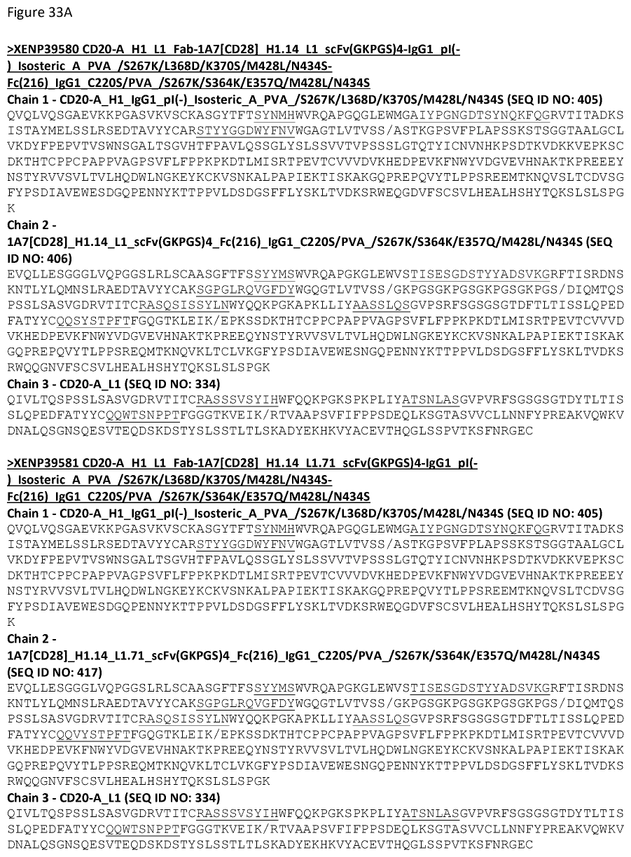

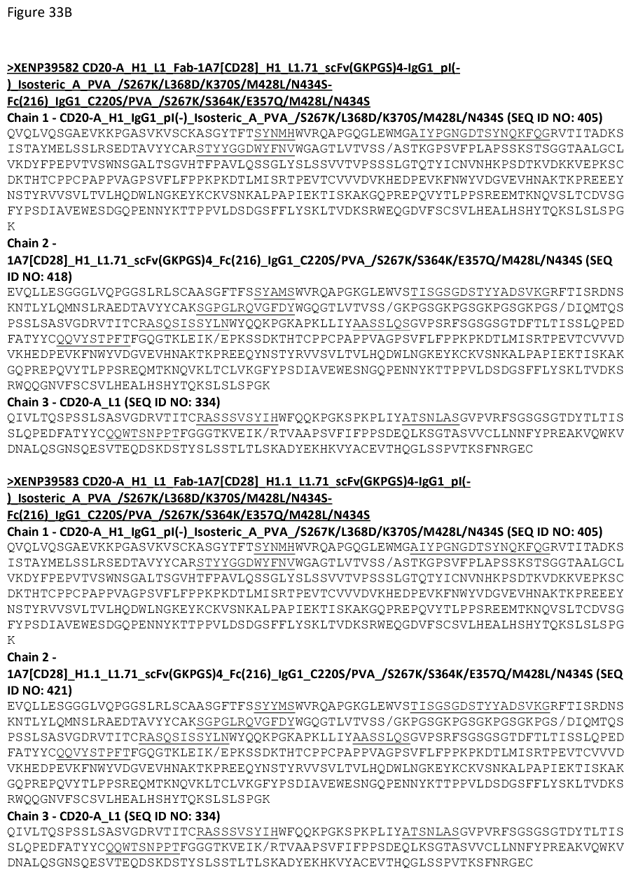

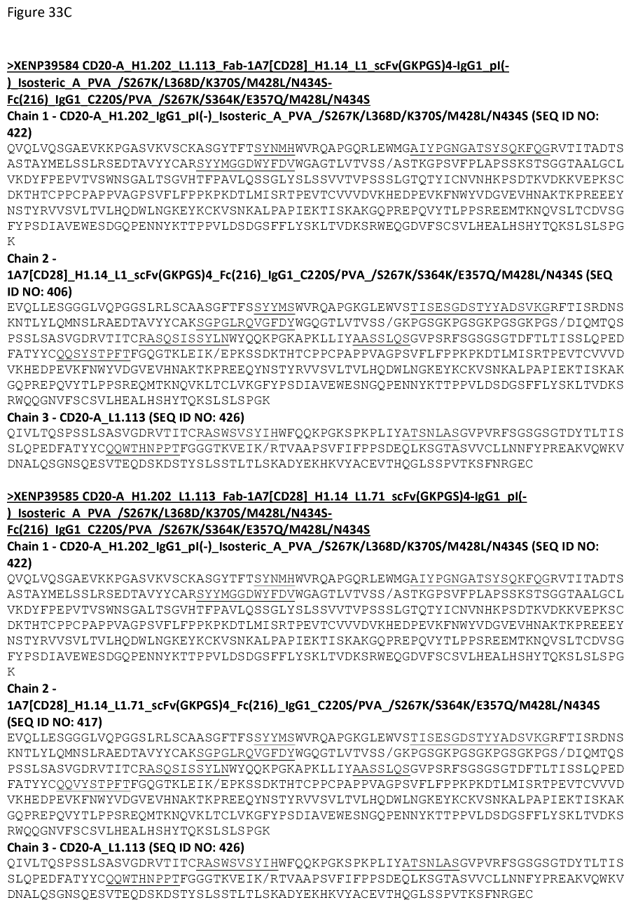

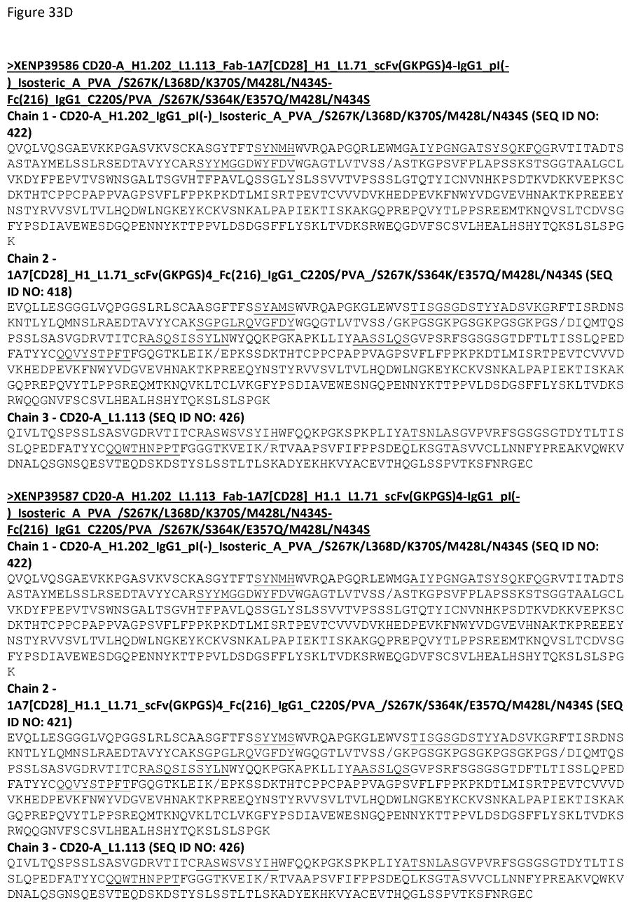

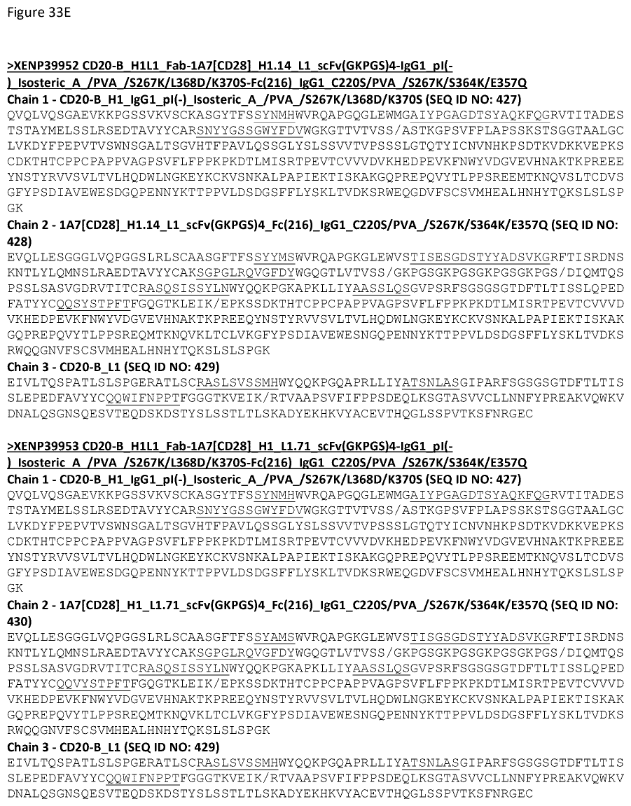

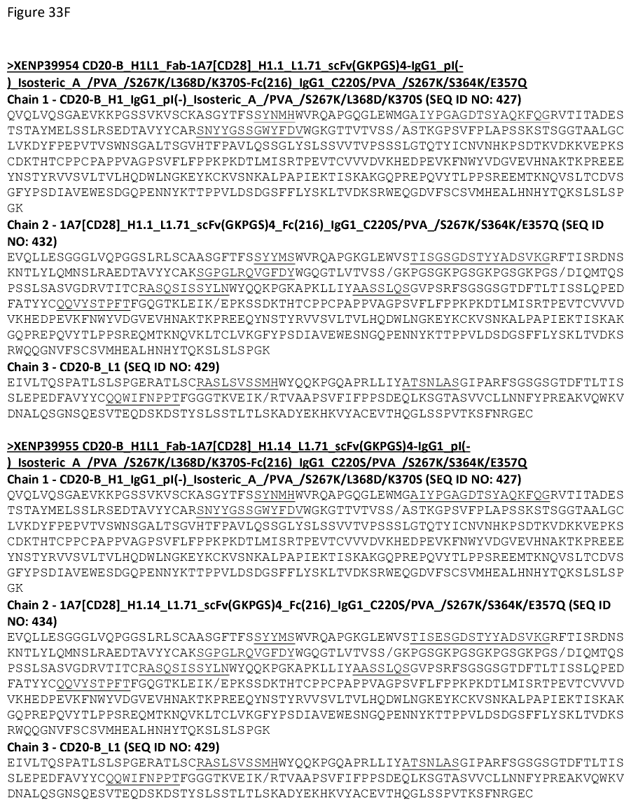

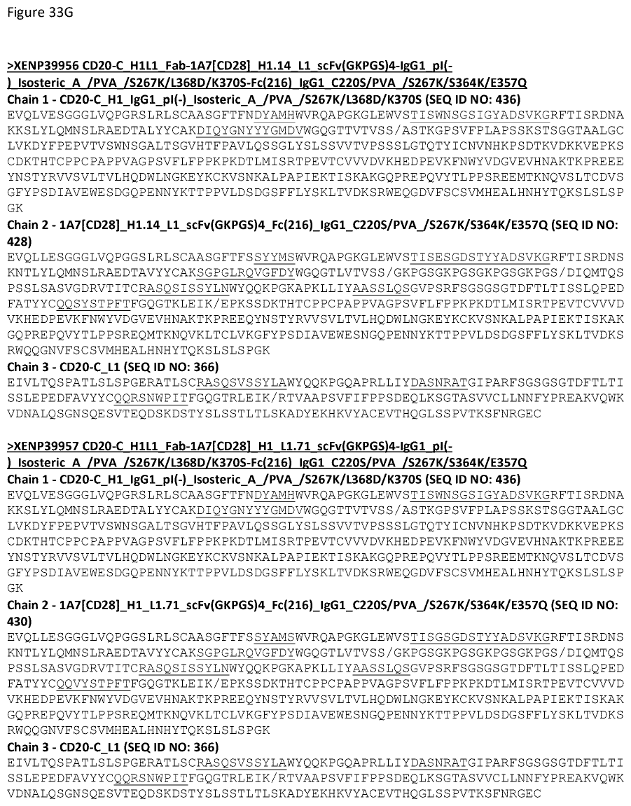

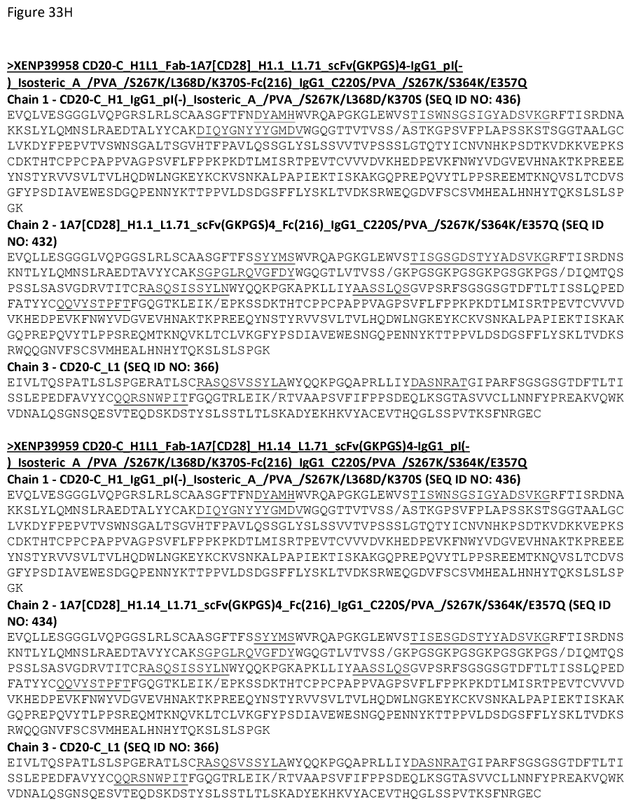

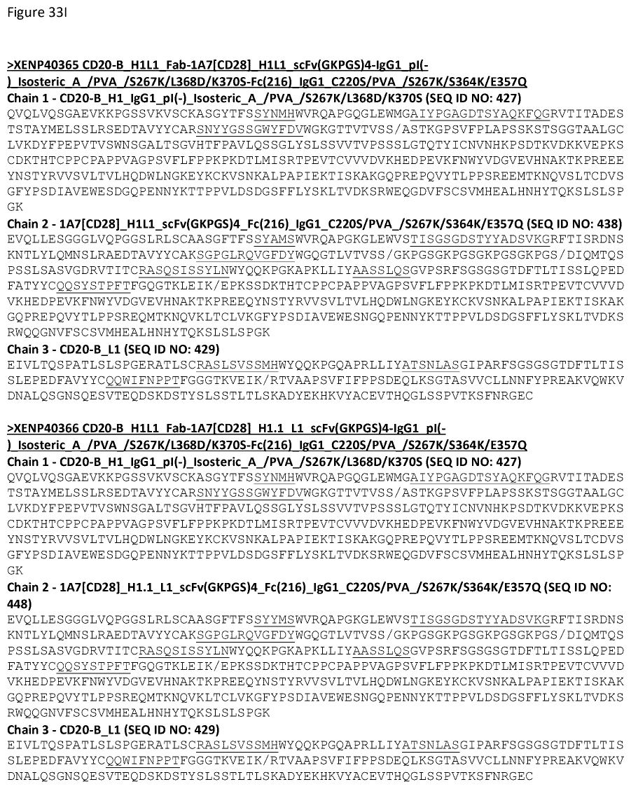

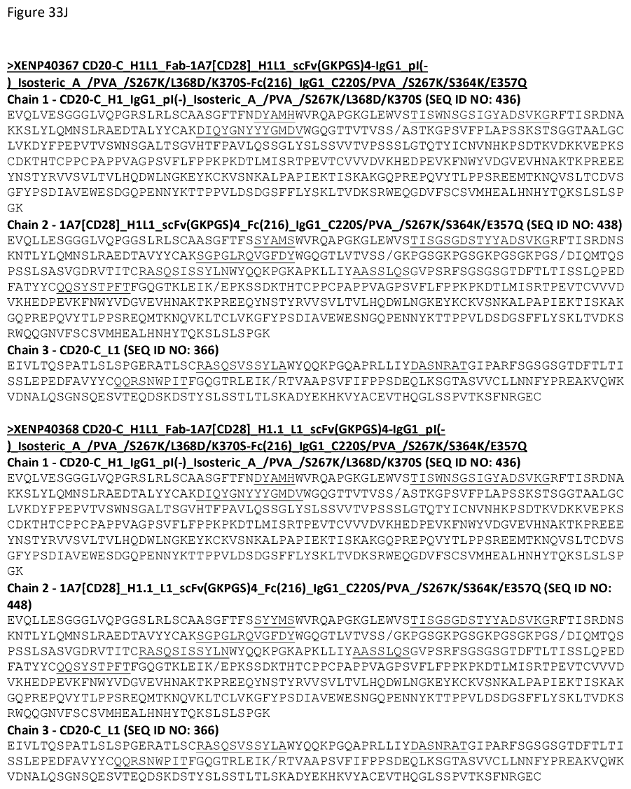

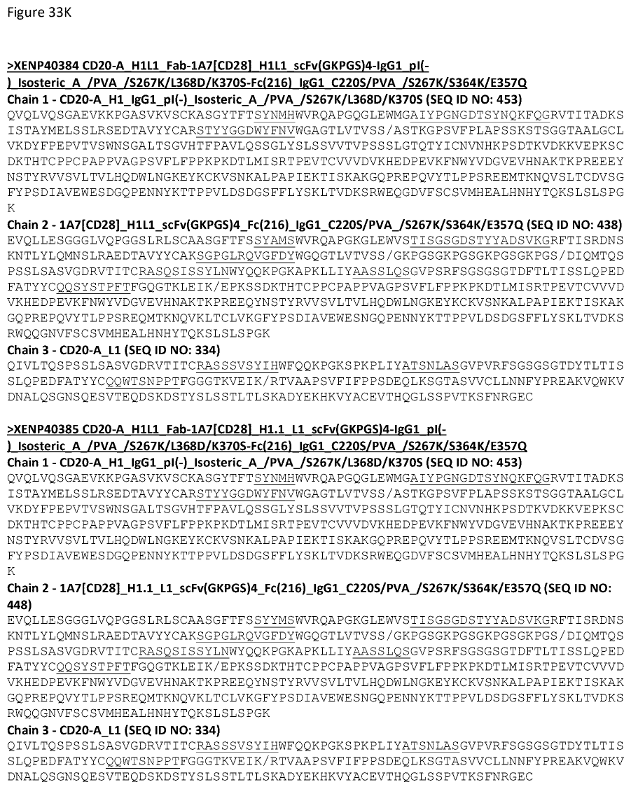

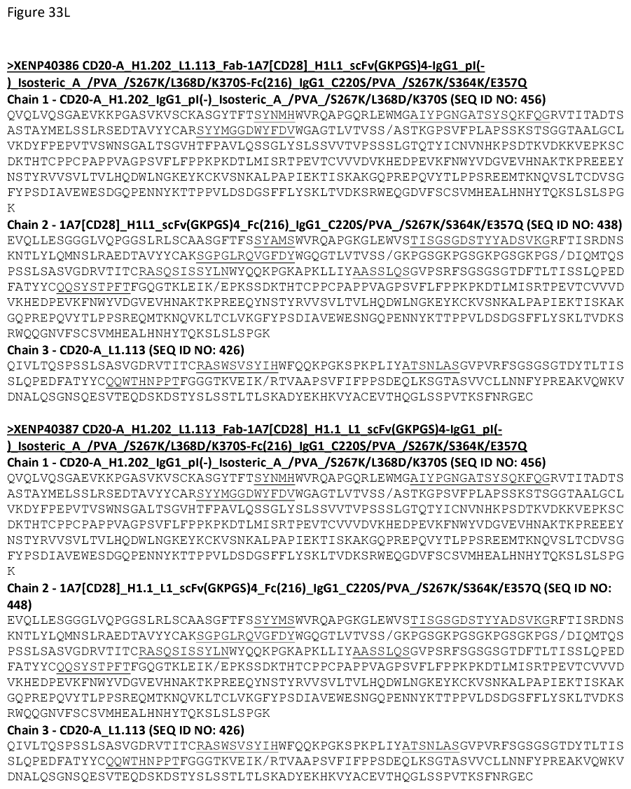

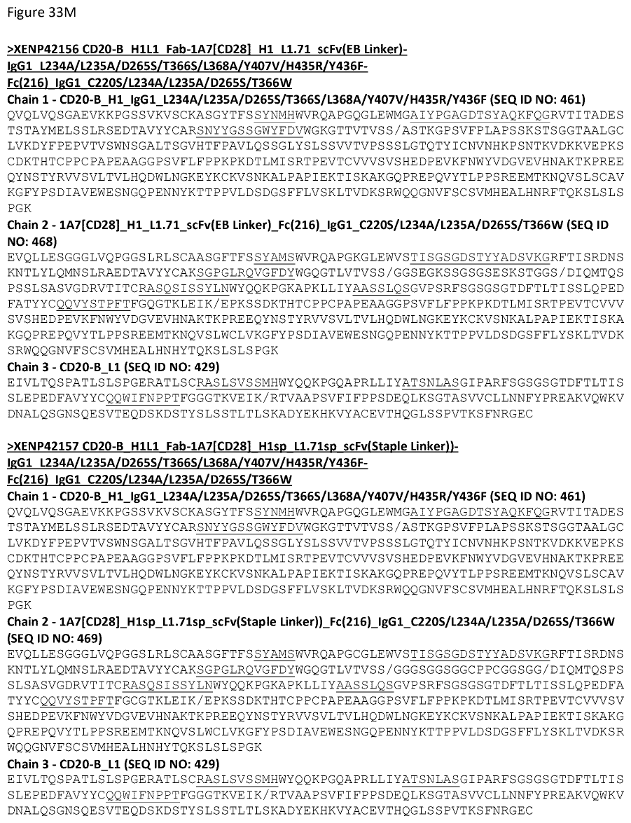

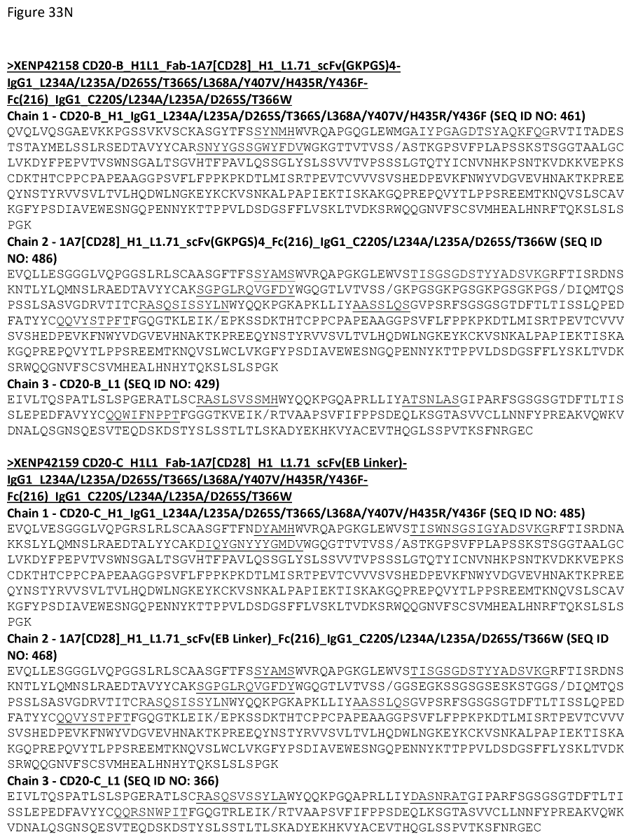

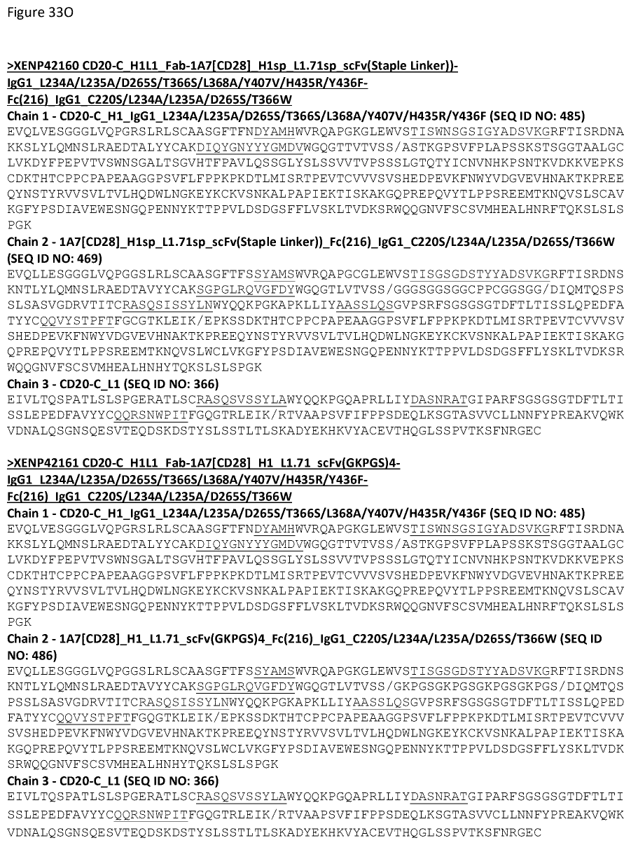

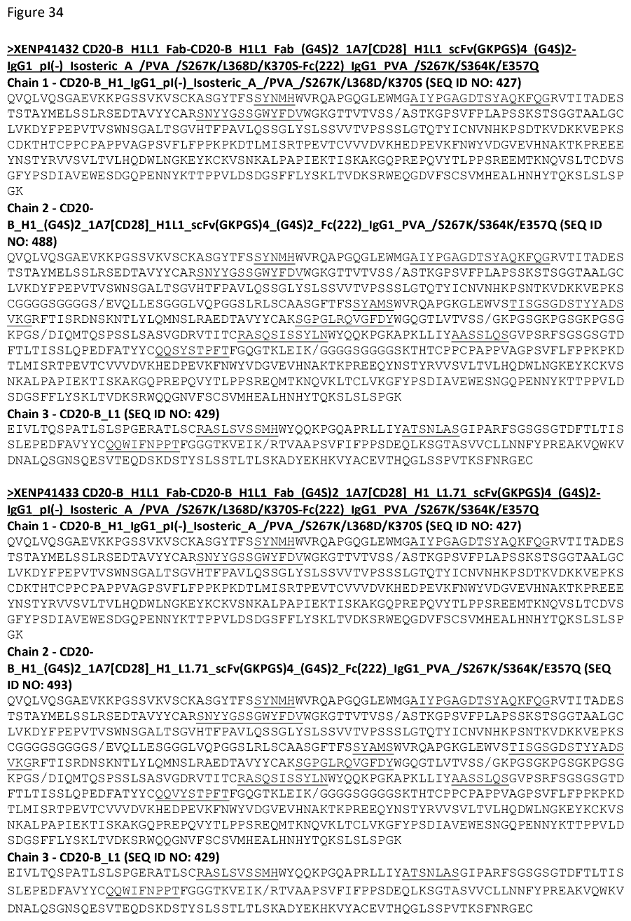

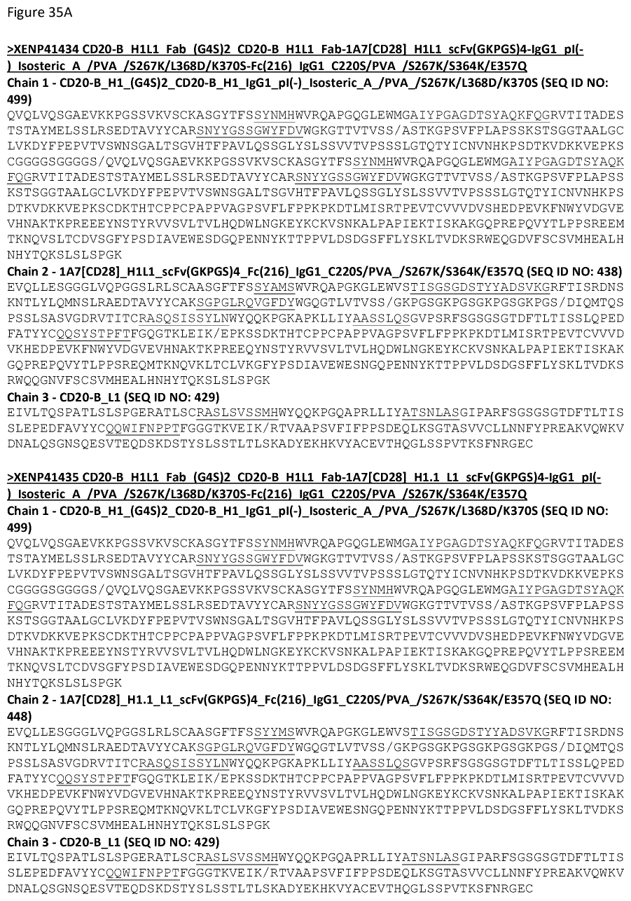

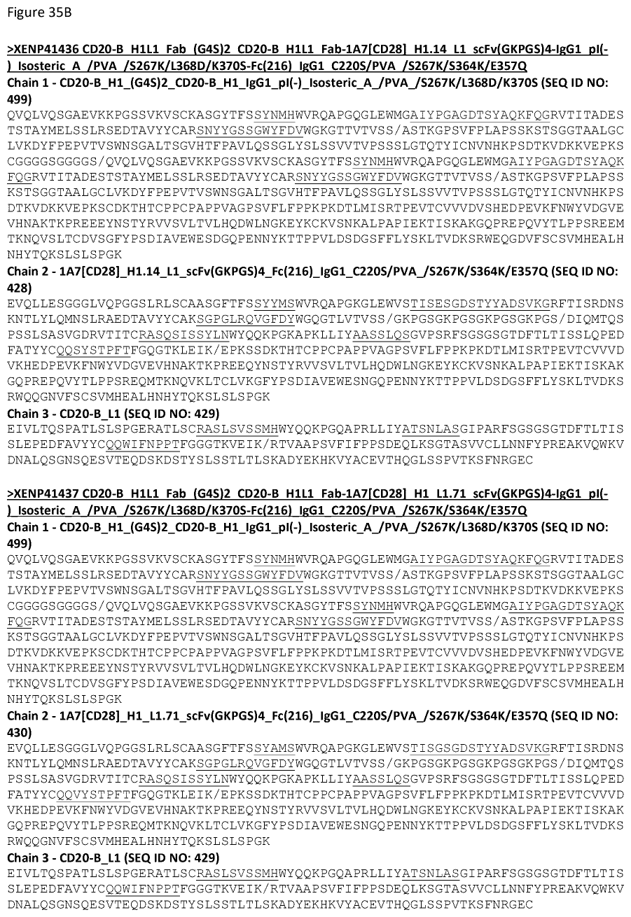

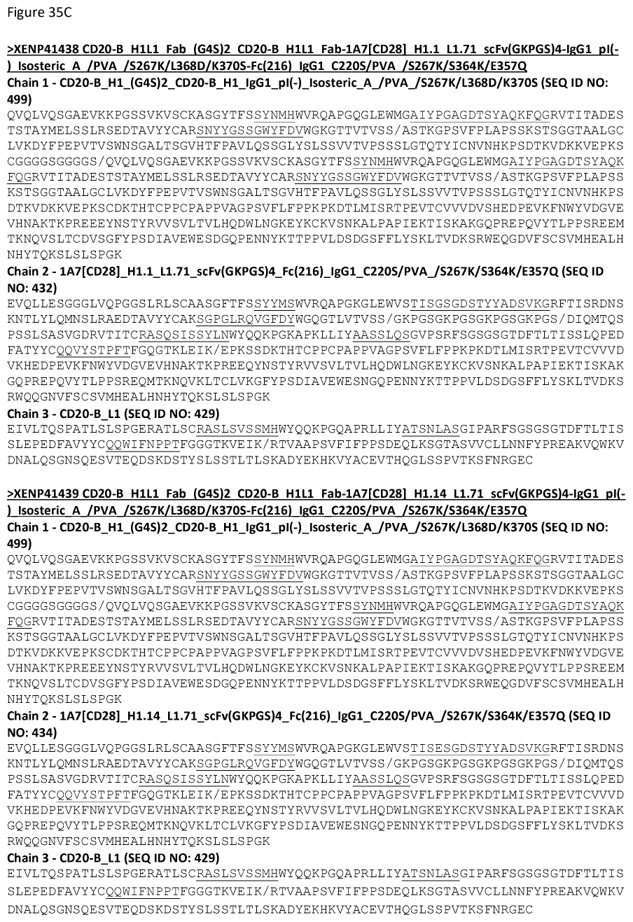

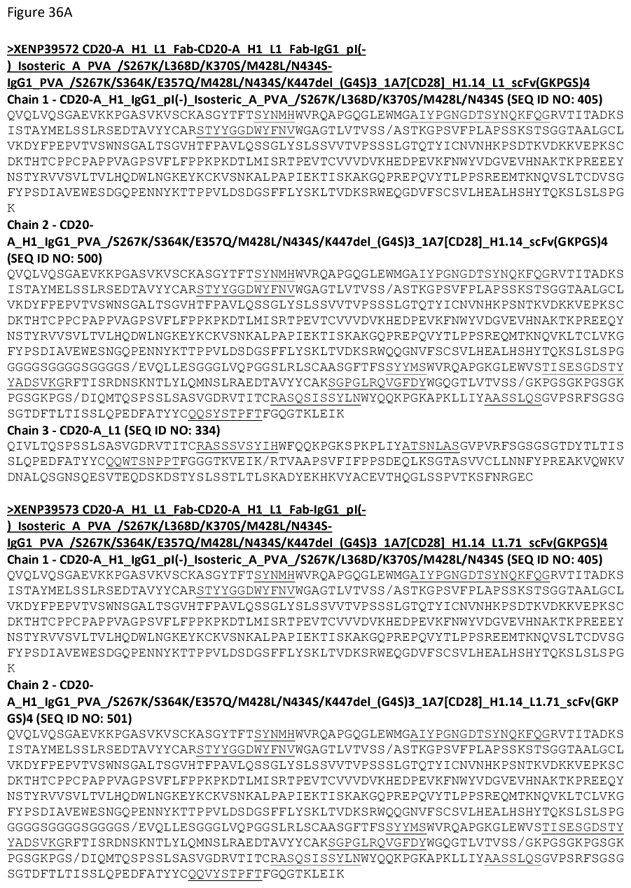

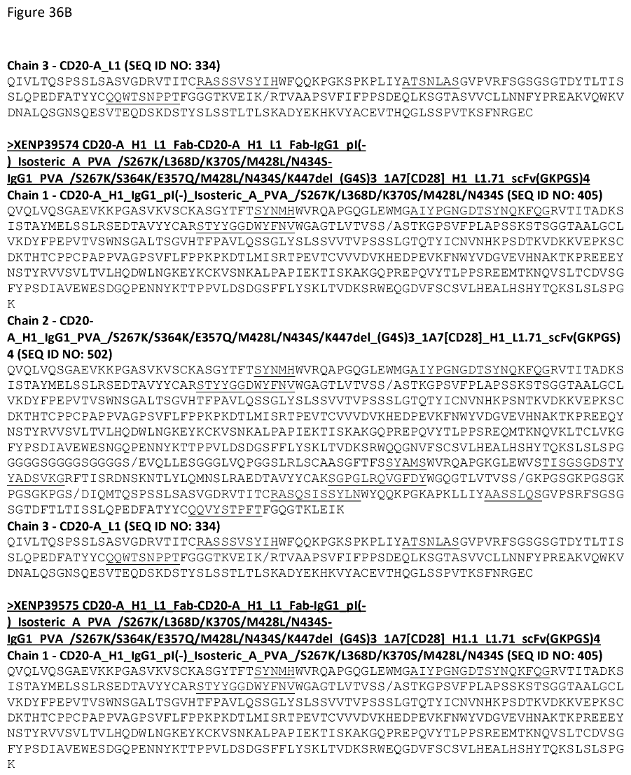

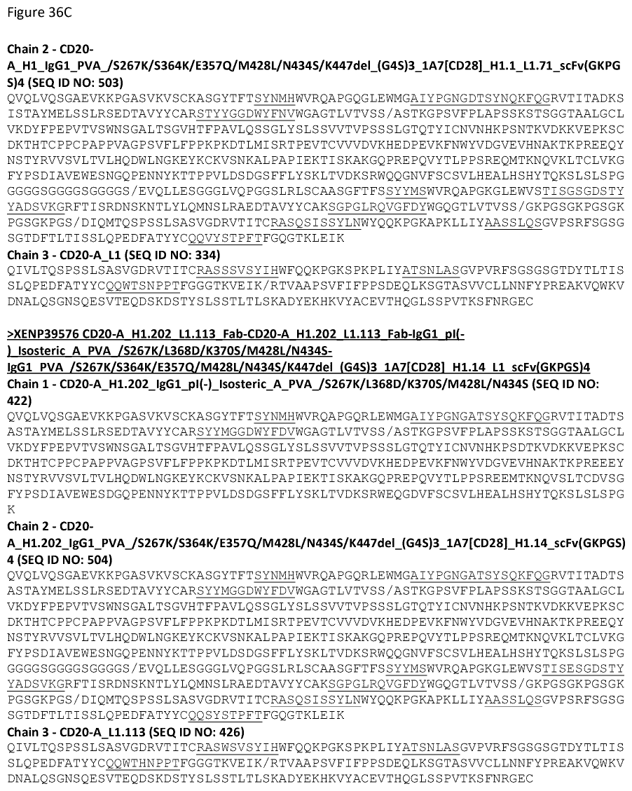

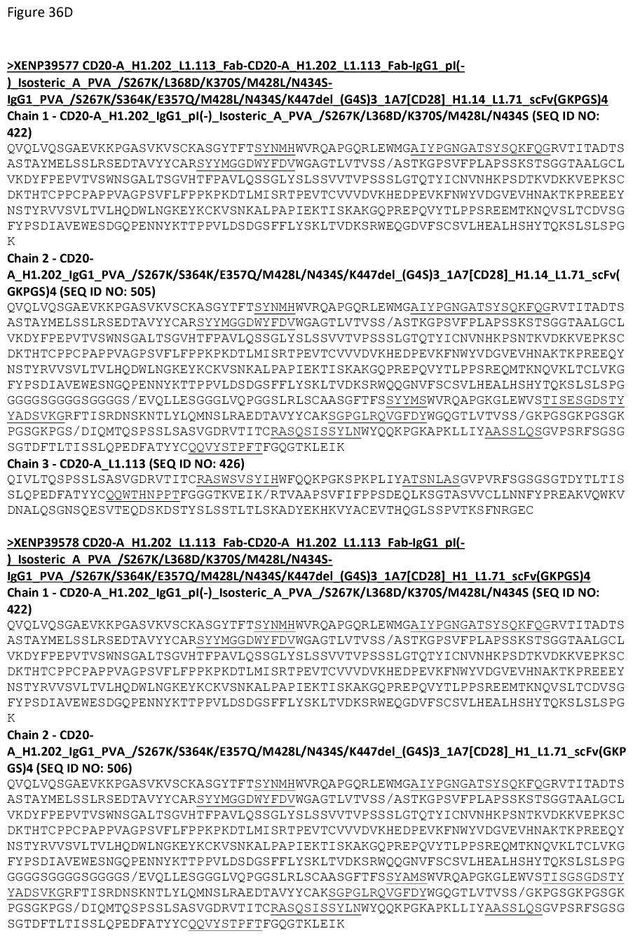

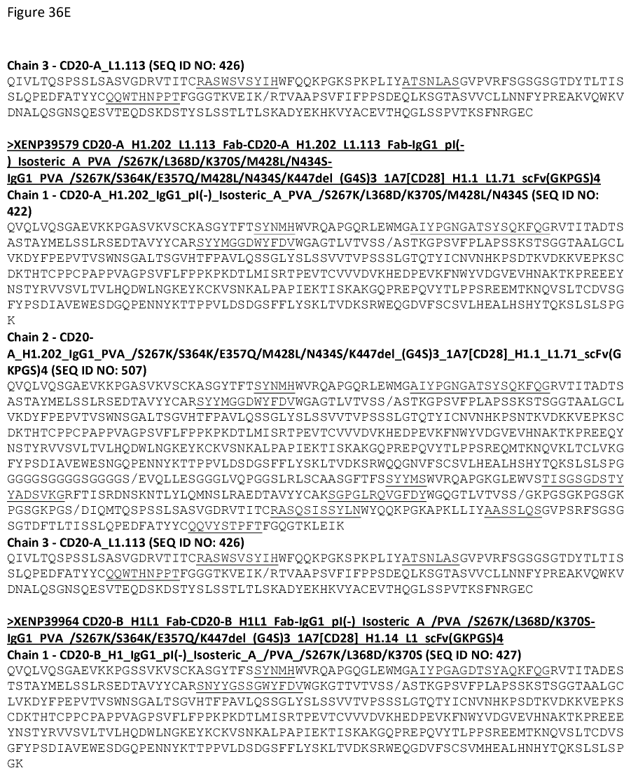

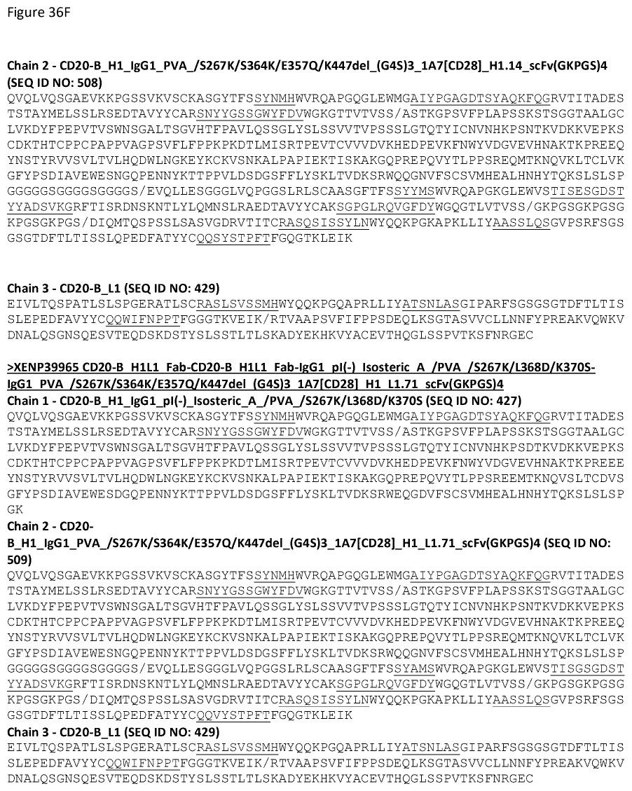

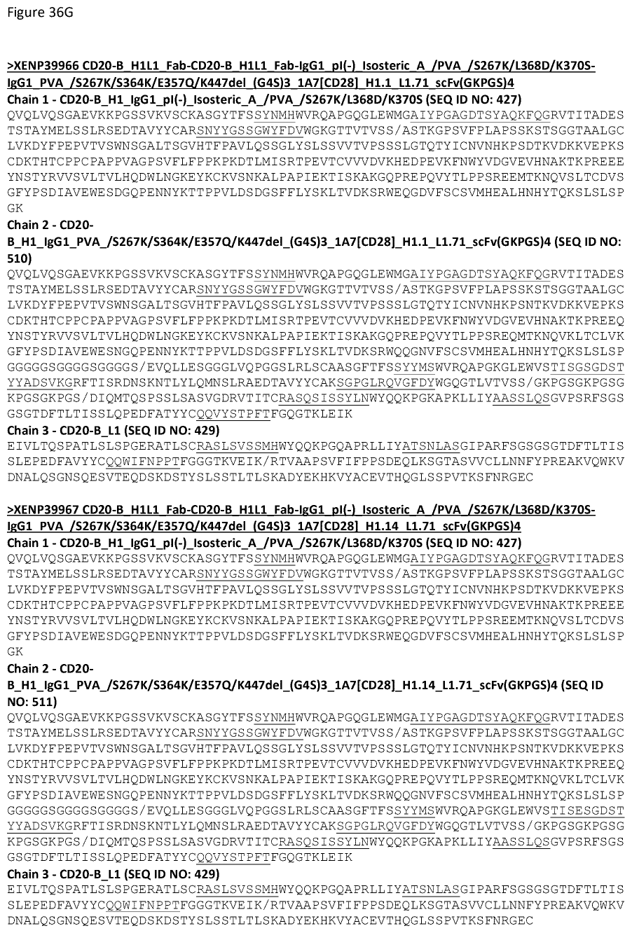

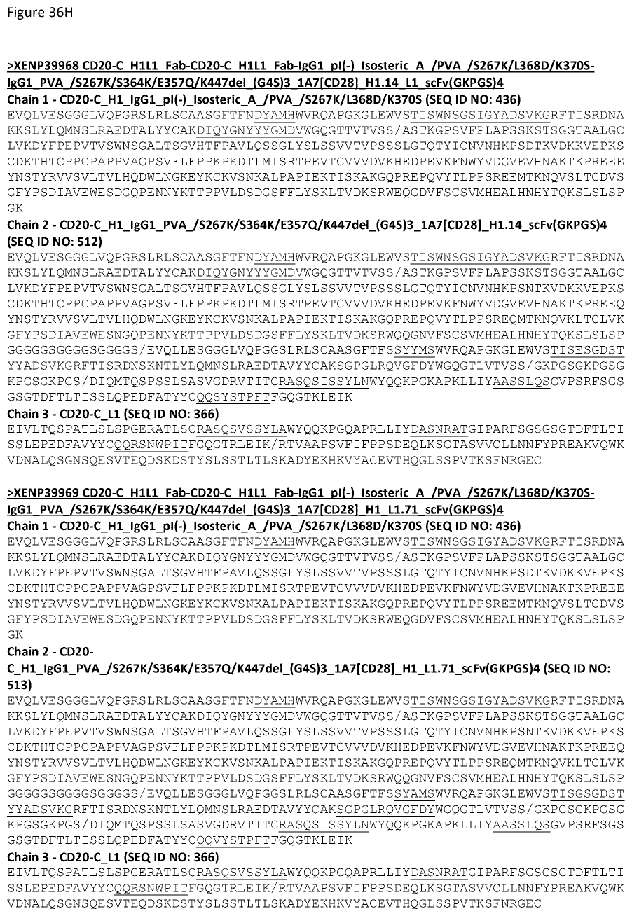

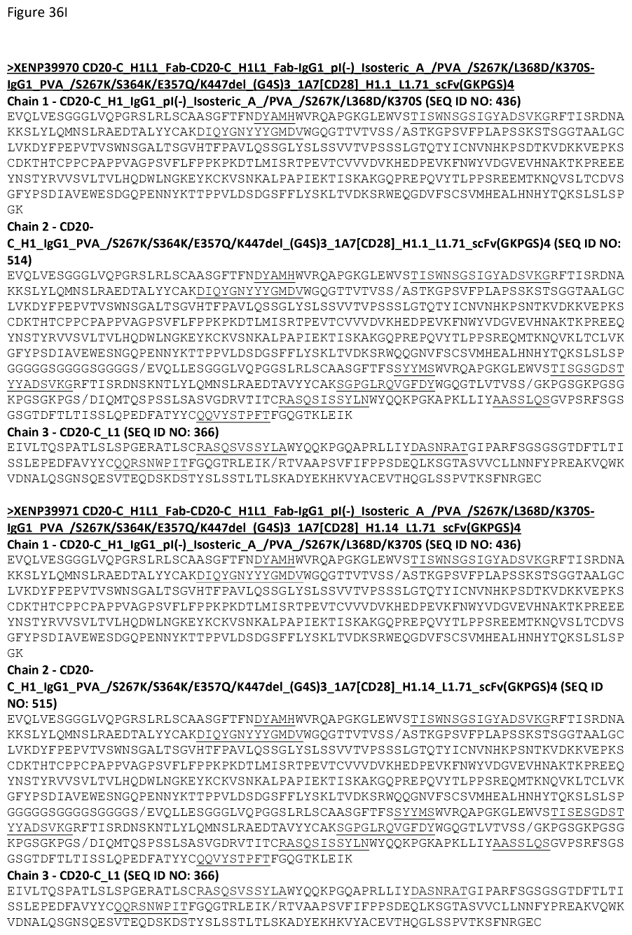

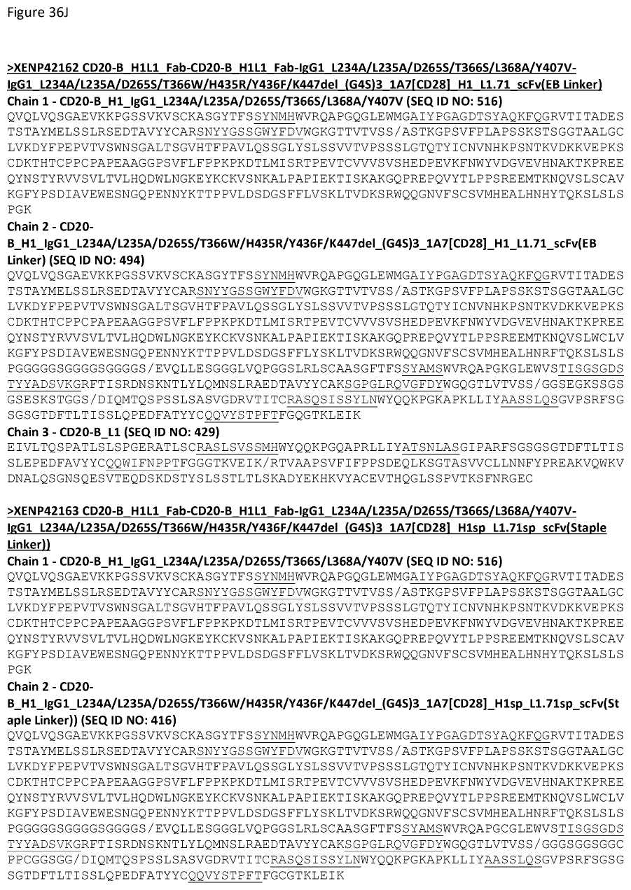

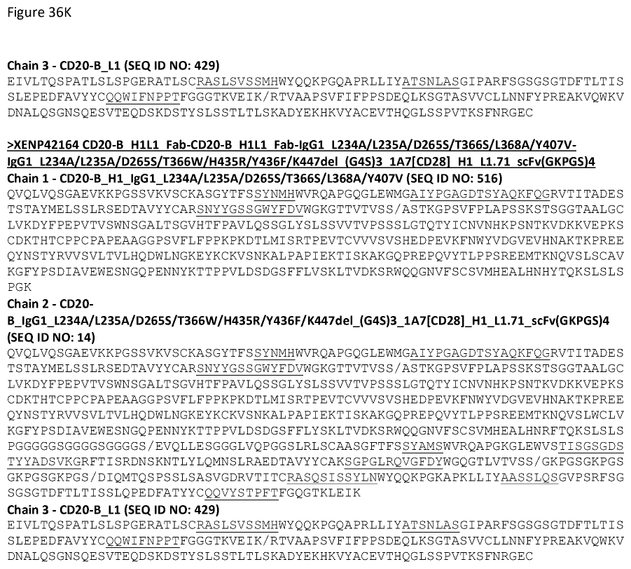

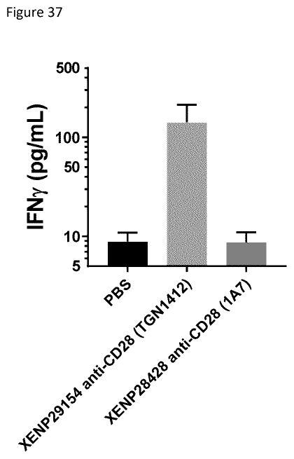

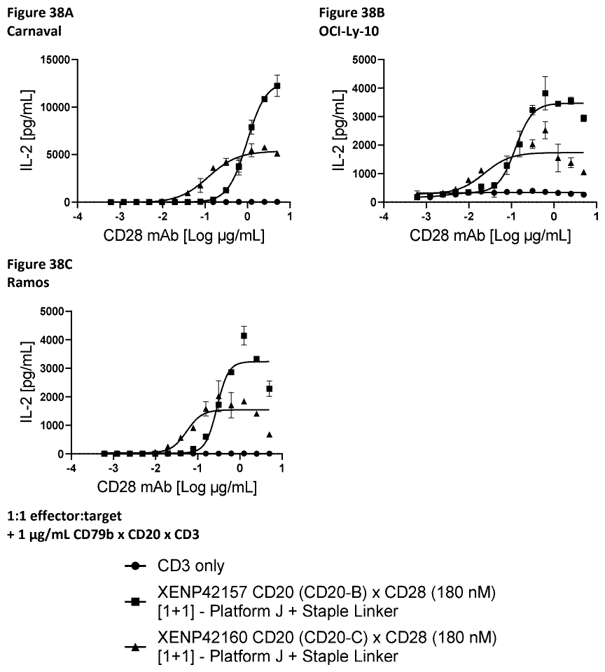

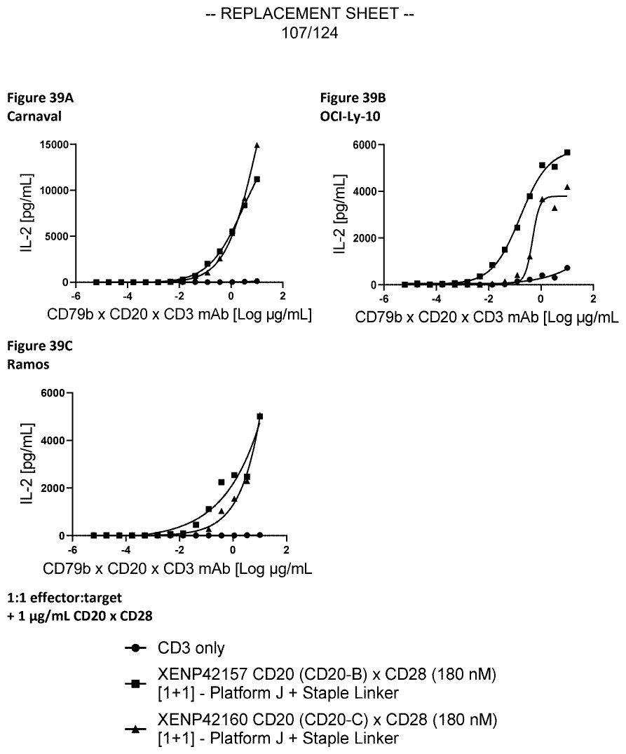

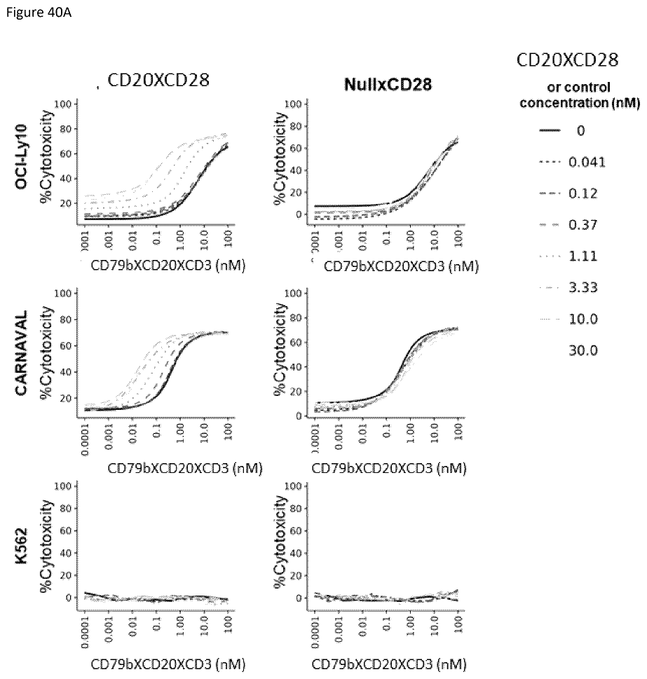

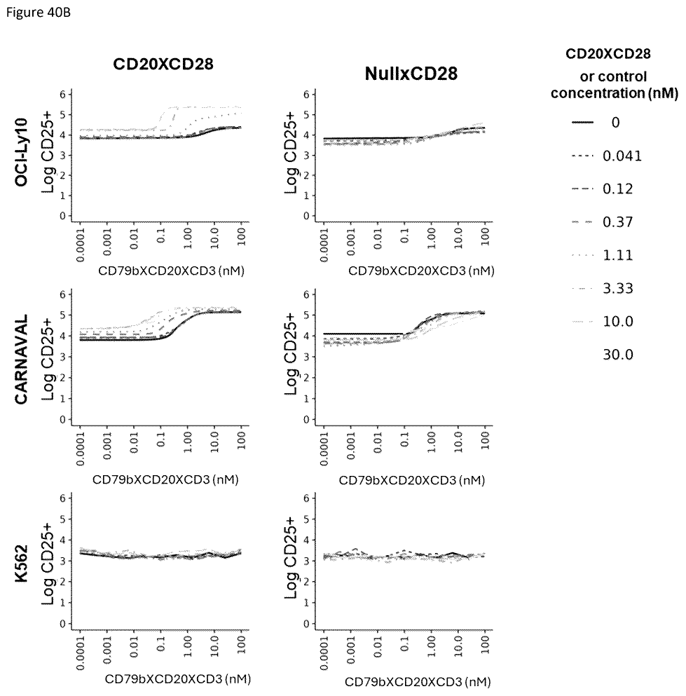

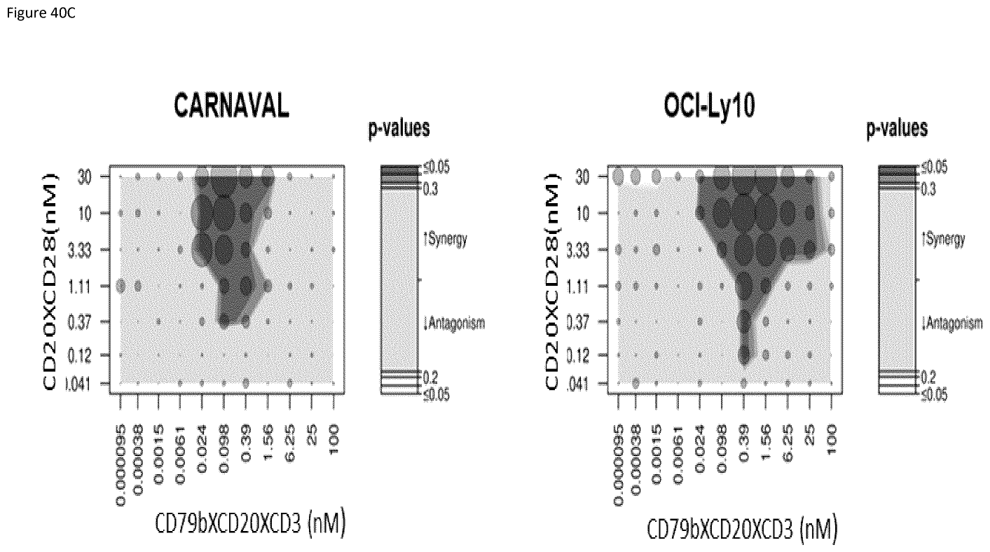

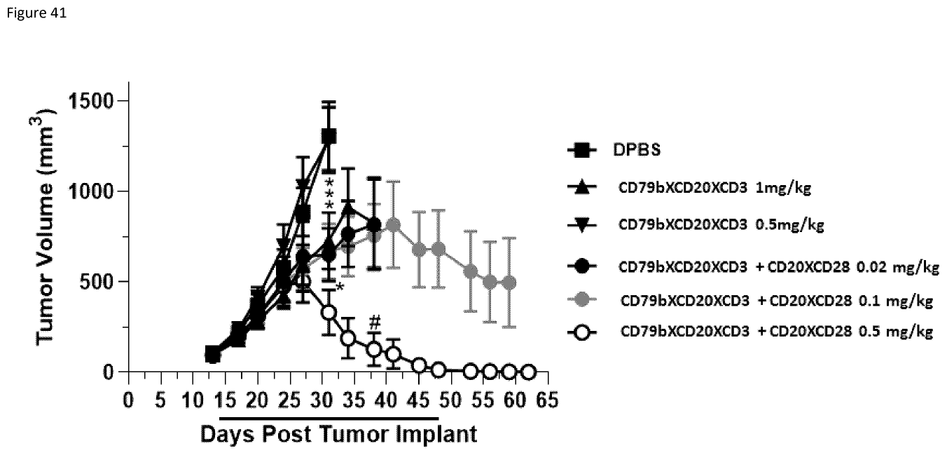

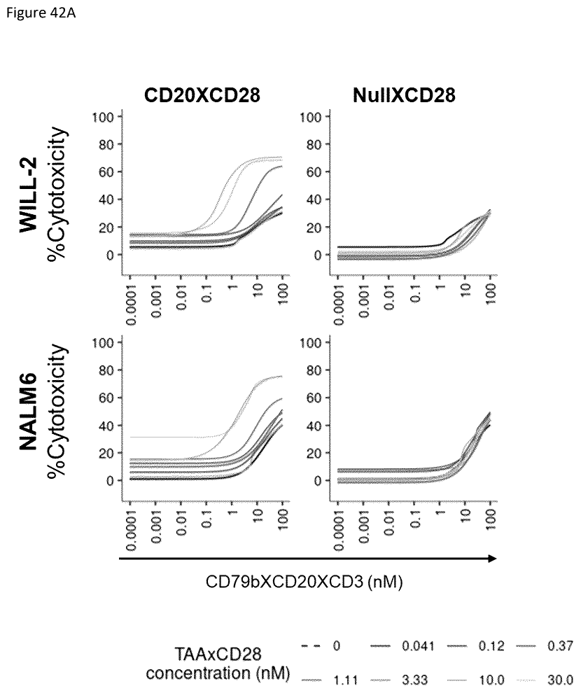

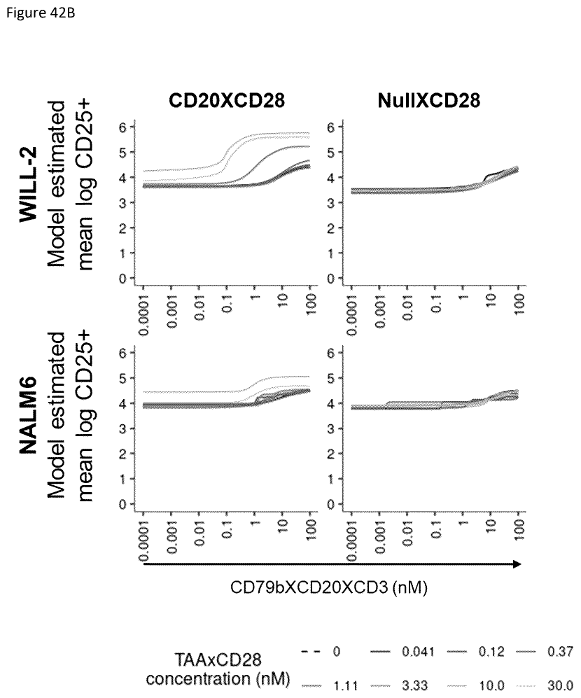

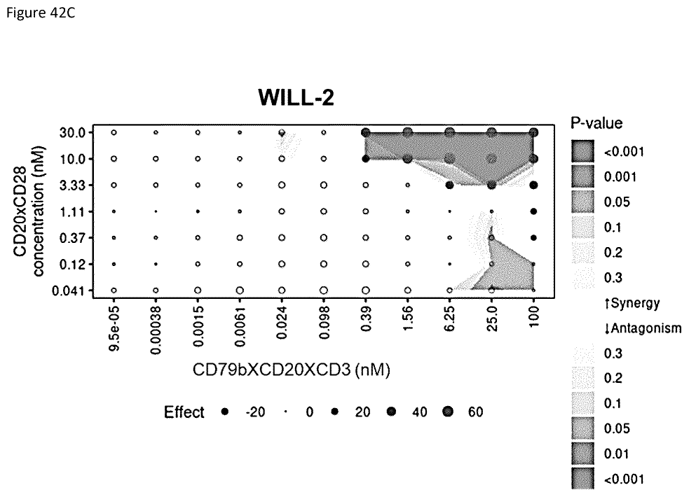

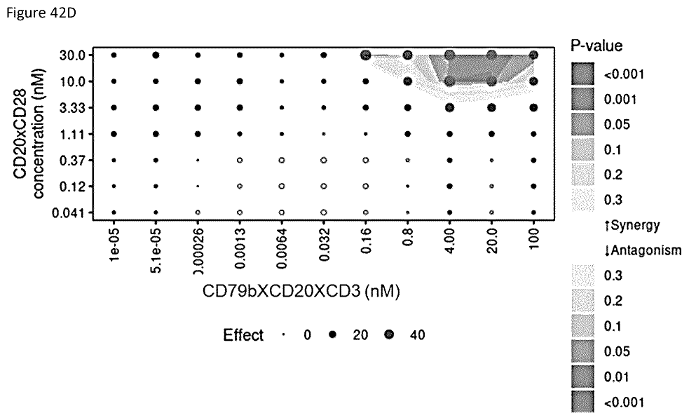

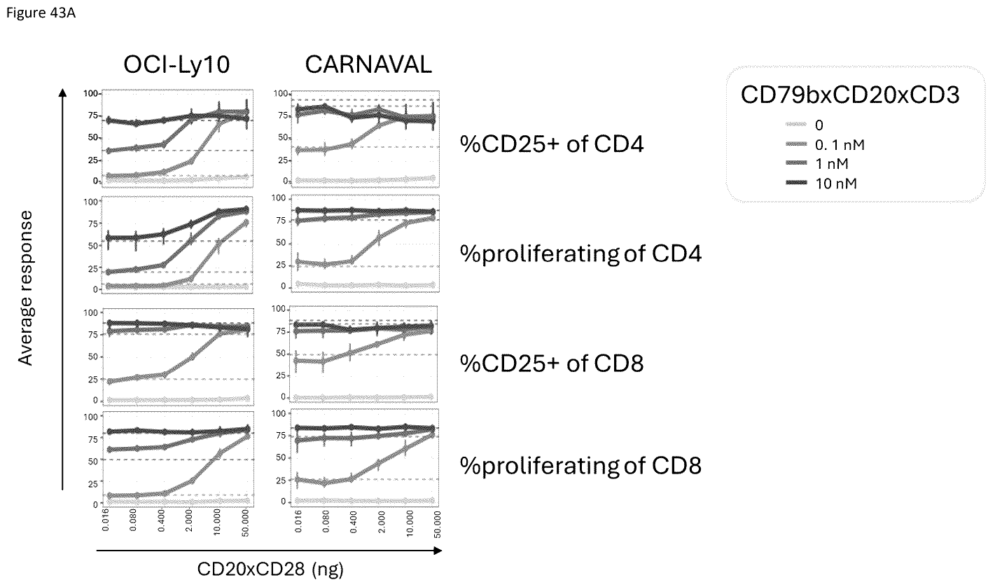

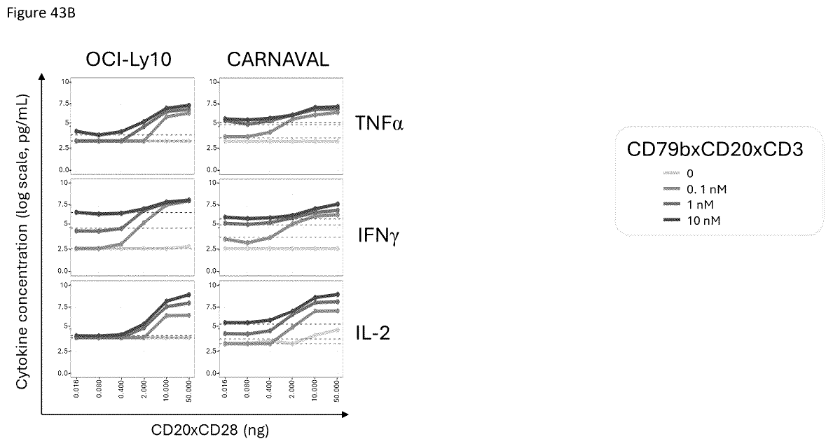

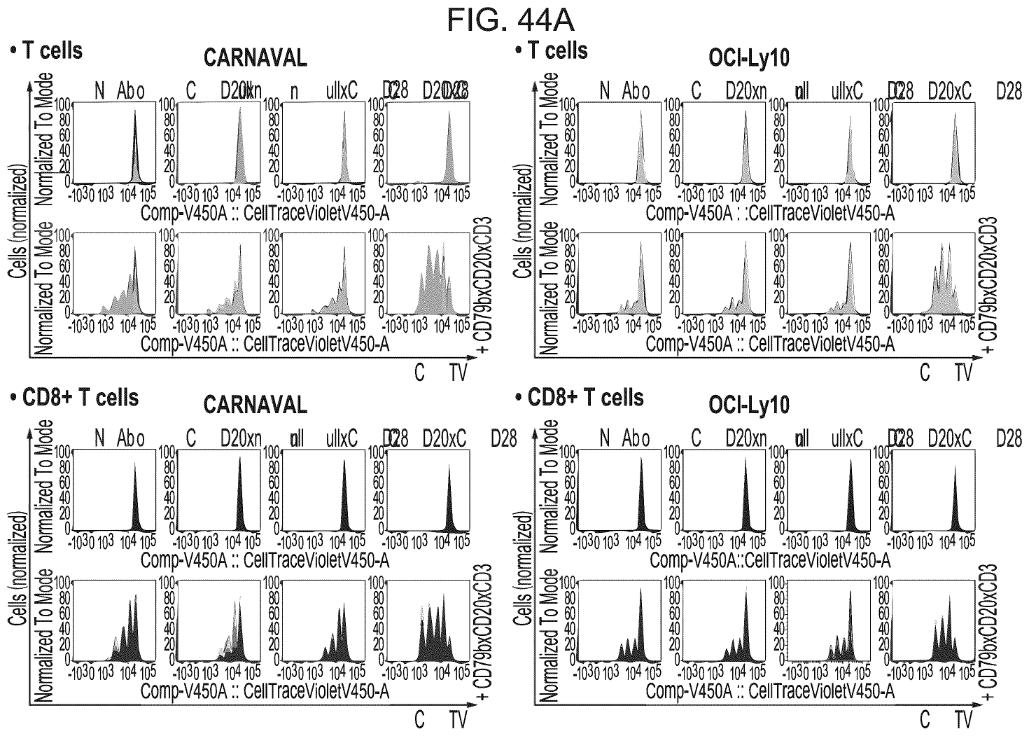

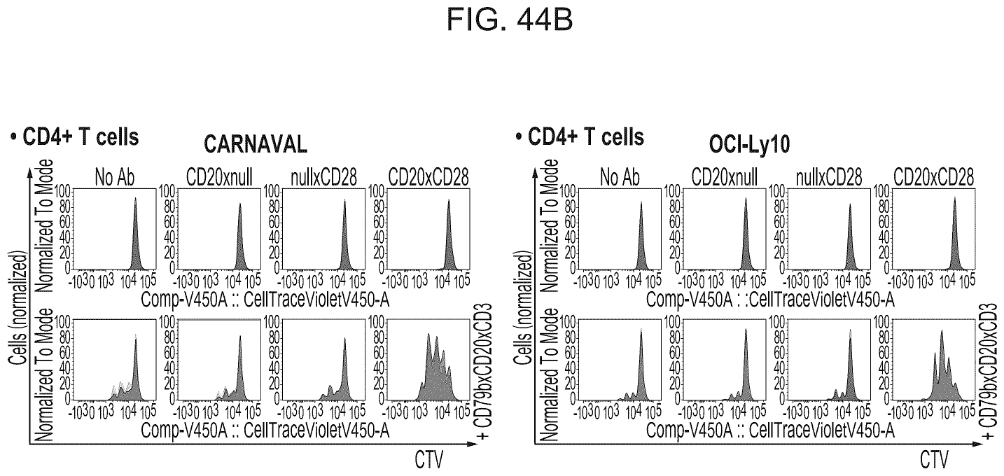

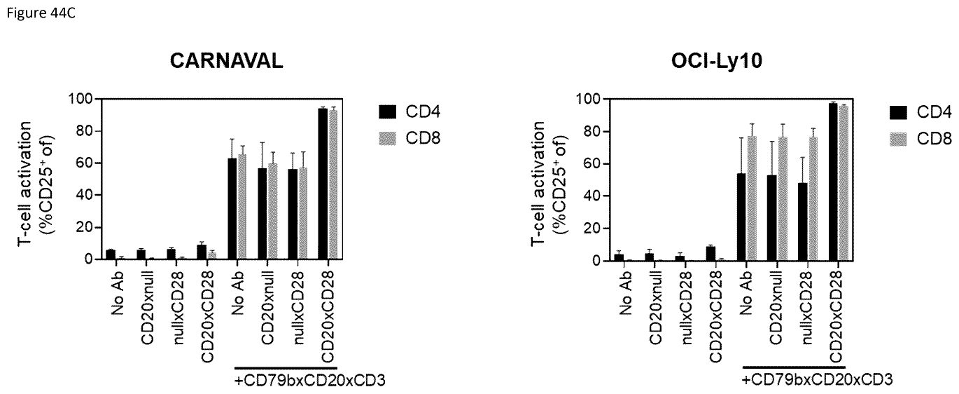

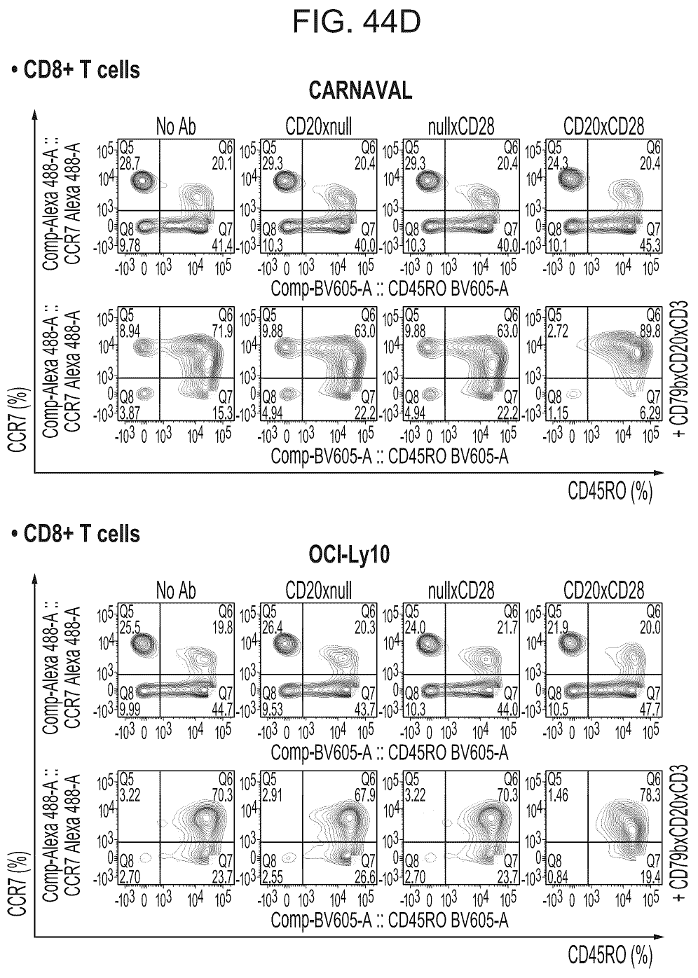

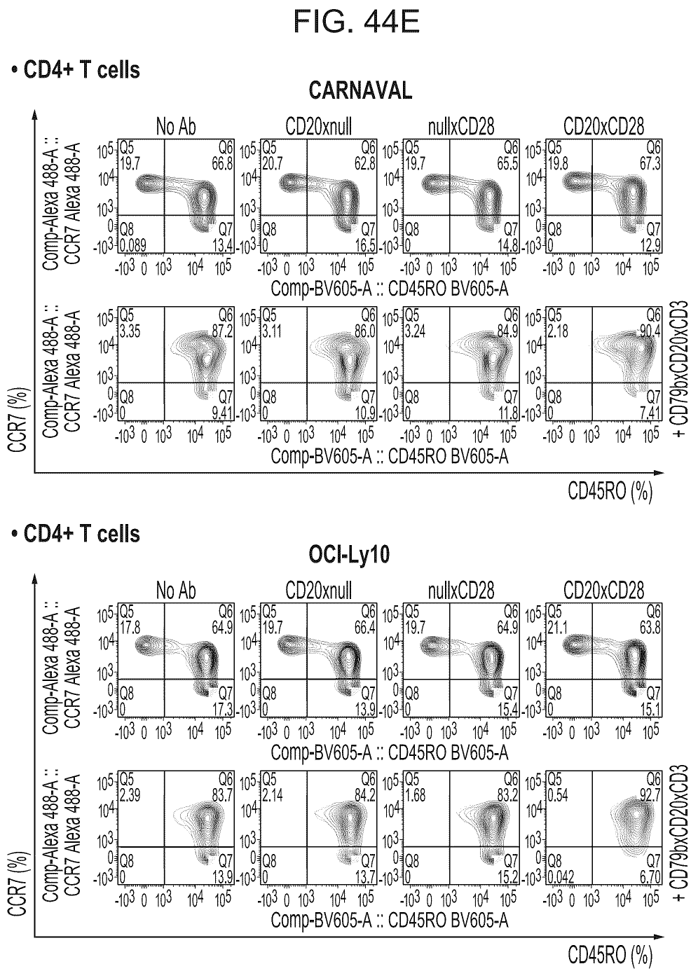

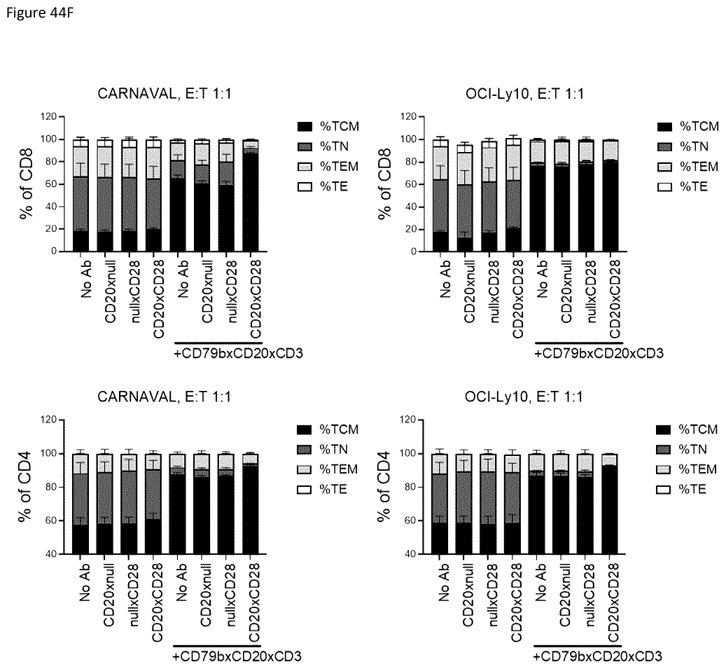

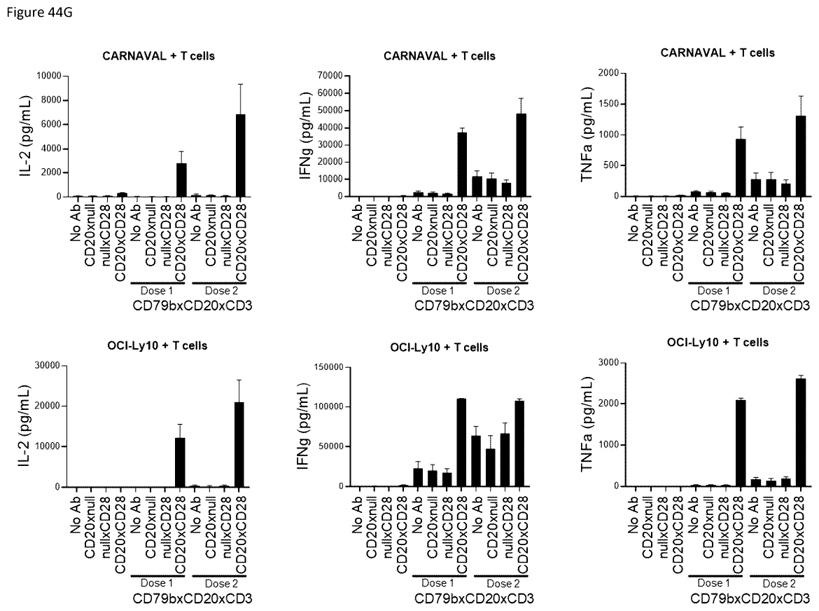

FIGS. 33A-330 depict the sequences for illustrative CD20 x CD28 bsAbs in the 1+1 Fab-scFv-Fc format. Although some of these sequences utilize Platform X and others use Platform J, any other suitable backbones including those depicted in FIG. 10 may be used. CDRs are underlined and slashes indicate the border(s) between the variable regions and other domains. It should be noted that the CD20 x CD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S, M428L/N434A, or M252Y/S254T/T256E variants in one or preferably both Fc domains, which including the variants results in longer half-life in serum.

FIG. 34 depicts the sequences for illustrative CD20 x CD28 bsAbs in the 2+1 Fab₂-scFv-Fc format. Although some of these sequences utilize Platform X and others use Platform J, any other suitable backbones including those depicted in FIG. 10 may be used. CDRs are underlined and slashes indicate the border(s) between the variable regions and other domains. It should be noted that the CD20 x CD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S, M428L/N434A, or M252Y/S254T/T256E variants in one or preferably both Fc domains, which including the variants results in longer half-life in serum.

FIGS. 35A-35C depict the sequences for illustrative CD20 x CD28 bsAbs in the 2+1 stackFab₂-scFv-Fc format. Although some of these sequences utilize Platform X and others use Platform J, any other suitable backbones including those depicted in FIG. 10 may be used. CDRs are underlined and slashes indicate the border(s) between the variable regions and other domains. It should be noted that the CD20 x CD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S, M428L/N434A, or M252Y/S254T/T256E variants in one or preferably both Fc domains, which including the variants results in longer half-life in serum.

FIGS. 36A-36K depict the sequences for illustrative CD20 x CD28 bsAbs in the 2+1 mAb-scFv format. Although some of these sequences utilize Platform X and others use Platform J, any other suitable backbones including those depicted in FIG. 11 may be used. CDRs are underlined and slashes indicate the border(s) between the variable regions and other domains. It should be noted that the CD20 x CD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S, M428L/N434A, or M252Y/S254T/T256E variants in one or preferably both Fc domains, which including the variants results in longer half-life in serum.

FIG. 37 depicts the release of IFNγ from human PBMCs treated with air-dried XENP28428 (anti-CD28 clone 1A7), TGN1412 (XENP29154), or negative control PBS.

FIGS. 38A-38C depicts induction of IL2 secretion by T cells incubated with A) Carnaval, B) OCI-Ly-10, and C) Ramos cancer cells and treated with CD79b x CD20 x CD3 in combination with XENP42157 or XENP42160 CD20 x CD28 bsAbs based on Platform J+Staple Linker (CD28 titration).

FIGS. 39A-39C depicts induction of IL2 secretion by T cells incubated with A) Carnaval, B) OCI-Ly-10, and C) Ramos cancer cells and treated with CD79b x CD20 x CD3 in combination with XENP42157 or XENP42160 CD20 x CD28 bsAbs based on Platform J+Staple Linker (CD3 titration).

FIGS. 40A-40C depict the effect of CD20XCD28 in combination with CD79bXCD20XCD3 on T-cell activation using healthy donor pan T cells and tumor cells expressing CD79b and CD20 (CARNAVAL and OCI-Ly10). (40A) depicts T-cell activation as measured by cytotoxicity assays. (40B) depicts T-cell activation as measured by CD25 expression. (40C) shows synergy plots for CD20XCD28 bsAb in combination with CD79bXCD20XCD3 on T-cell mediated cytotoxicity.

FIG. 41 shows effect of CD20XCD28 and CD79bXCD20XCD3 combination treatment on growth of established SC OCI-Ly10 DLBCL xenografts in T-cell-humanized mice.

FIGS. 42A-42D depict the effect of CD20XCD28 in combination with CD79bXCD20XCD3 on T cell activation using healthy donor pan T cells and tumor cells expressing low levels of CD79b and CD20 (WILL-2 and NALM6). WILL2 cell line used 6 donors and NALM6 cell line used 3 donors. (41A) depicts T-cell activation as measured by cytotoxicity assays. (41B) depicts T-cell activation as measured by CD25 expression. (41C and 41D) shows synergy plots for CD20XCD28 bsAb in combination with CD79bXCD20XCD3 on T-cell mediated cytotoxicity. Open circles represent negative effects and closed circles represent positive effects.

FIGS. 43A and 43B depict the effect of CD20XCD28 in combination with CD79bXCD20XCD3 on T-cell activation using healthy donor pan T cells and tumor cells expressing CD79b and CD20 (CARNAVAL and OCI-Ly10), as measured at 72 h timepoint. (43A) depicts CD4 and CD8 T-cell activation (% CD25 positive) and proliferation, and (43B) depicts cytokine release.

FIGS. 44A-44G depict the effect of CD20XCD28 in combination with CD79bXCD20XCD3 on T cell activation, proliferation, differentiation and cytokine release. (43A and 43B) depict pan T-cells, CD8 T-cells, and CD4 T-cells proliferation. (44C) depict CD8 T-cell and CD4 T-cell activation (% of CD25⁺ cells). (44D to 44F) depict CD4 and CD8 T-cells differentiation. (44G) depicts cytokine release.

### DETAILED DESCRIPTION

### I. Overview

The CD20 antigen is selectively expressed on mature B cells and provides a therapeutic target in the treatment of B-cell malignancies.

The activation of T cells in the treatment of cancer is being widely investigated. T cells require multiple signals for complete activation and differentiation. As shown in FIG. 31A, Signal 1, promoted by recognition of a peptide-MHC (pMHC) complex by the T cell receptor (TCR), is absolutely required for T cell activation. Signal 2, which synergizes with, and amplifies signal 1, is typically provided by the interaction of the CD28 ligands CD80 and CD86 with CD28 itself. Although CD28 engagement alone is inert, when combined with signal 1 activation, it enhances T-cell activation, survival, and proliferative signals, including IL-2 secretion. As CD80 and CD86 are naturally expressed by professional antigen-presenting cells (APC), the extent of CD28 costimulation in the tumor setting can be highly variable. Accordingly, the present invention is directed to a novel class of tumor-targeted anti-CD20 x anti-CD28 antibodies that mimic the CD80/CD86 engagement of CD28, thereby providing a source of signal 2. Notably, signal 1 can either be provided by the natural TCR:pMHC recognition of tumor cells, or it can be provided by combination of the CD28 bispecific with a CD3 T cell engager (e.g., anti-CD3 x anti-CD20 x anti-CD79b), which can mimic signal 1.

Accordingly, provided herein are novel anti-CD20 x anti-CD28 (also referred to as “αCD20 x αCD28” and sometimes “CD20 x CD28”) antibodies and methods of using such antibodies for the treatment of B-cell malignancies. In many cases, these antibodies are heterodimeric. Subject αCD20 x αCD28 antibodies are capable of binding to CD28 costimulatory molecules on T cells and targeting to CD20 on CD20-expressing malignant B-cells. Thus, such antibodies selectively enhance anti-tumor activity at CD20-expressing tumor sites while minimizing peripheral toxicity. The subject antibodies provided herein are particularly useful for enhancing anti-tumor activity either alone, as a monotherapy, or when used in combination with other anti-cancer therapies as more fully described herein.

Accordingly, in one embodiment, provided herein are heterodimeric antibodies or fragments thereof that bind to two different antigens, e.g., the antibodies are “bispecific,” in that they bind two different target antigens, generally CD20 and CD28 as described below. These heterodimeric antibodies can bind each of the target antigens either monovalently (e.g., there is a single antigen binding domain) or bivalently (there are two antigen binding domains that each independently bind the antigen). In some embodiments, the heterodimeric antibody provided herein includes one CD28 binding domain and one CD20 binding domain (e.g., heterodimeric antibodies in the “1+1 Fab-scFv-Fc” format described herein, which are thus bispecific and bivalent). In other embodiments, the heterodimeric antibody provided herein includes one CD28 binding domain and two CD20 binding domains (e.g., heterodimeric antibodies in the “2+1 Fab₂-scFv-Fc” formats described herein, which are thus bispecific but trivalent, as they contain three antigen binding domains (ABDs)). The heterodimeric antibodies provided herein are based on the use of different monomers that contain amino acid substitutions (i.e., skew variants”) that “skew” formation of heterodimers over homodimers, as is more fully outlined below. In some embodiments, the heterodimer antibodies are also coupled with purification variants (e.g., “pI variants”) that allow simple purification of the heterodimers away from the homodimers, as is similarly outlined below. The heterodimeric antibodies provided generally rely on the use of engineered or variant Fc domains that can self-assemble in production cells to produce heterodimeric proteins, and methods to generate and purify such heterodimeric proteins.

### II. Nomenclature

The naming nomenclature of particular antigen binding domains (e.g., CD20 and CD28 binding domains) use a “Hx.xx_Ly.yy” type of format, with the numbers being unique identifiers to particular variable chain sequences. Thus, for example, the CD28 binding domain “1A7[CD28]_H1_L1” (FIG. 15) includes a variable heavy domain, H1, and a variable light domain L1. In the case that these sequences are used as scFvs, the designation “H1_L1”, indicates that the binding domain includes a variable heavy domain “H1” combined with a variable light domain “L1,” and is in VH-linker-VL orientation, from N- to C-terminus. This molecule with the identical sequences of the heavy and light variable domains but in the reverse order (VL-linker-VH orientation, from N- to C-terminus) would be designated “L1_H1”. Similarly, different constructs may “mix and match” the heavy and light chains as will be evident from the sequence listing and the Figures.

### III. Definitions

In order that the application may be more completely understood, several definitions are set forth below. Such definitions are meant to encompass grammatical equivalents.

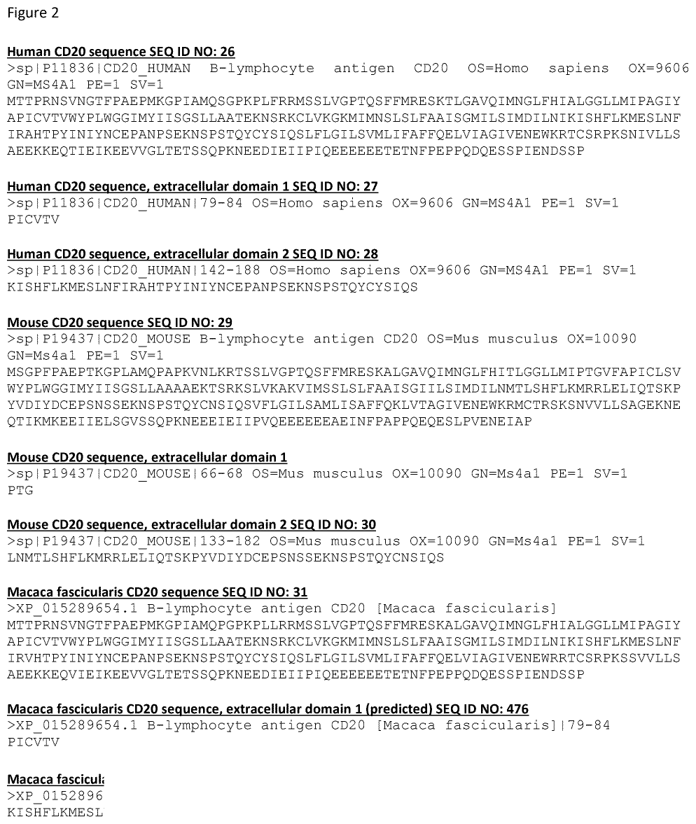

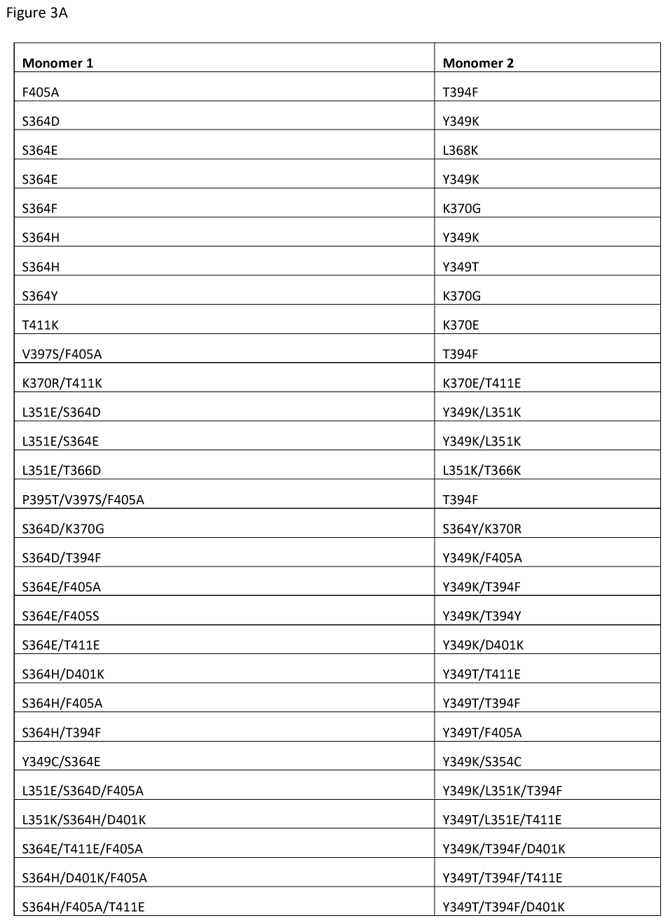

By “B-lymphocyte antigen CD20,” “CD20,” “MS4A1,” “B1,” “Bp35,” “CD20,” “CVID5,” “LEU-16,” “MS4A2,” “S7, membrane spanning 4-domains A1,” and “FMC7” (e.g., Genebank Accession Numbers NP_068769 (human), NP_690605 (human), NP_690606 (human)) herein is meant a B-lymphocyte surface molecule that plays a role in the development and differentiation of B-cells into plasma cells. In humans, CD20 is encoded by the MS4A1 gene. CD20 is present from late pro-B cells through memory cells in B cell development. It is found on B-cell lymphomas, hairy cell leukemia, B-cell chronic lymphocytic leukemia, and melanoma cancer stem cells. Exemplary CD20 sequences are depicted in FIG. 2. Unless otherwise noted, references to CD20 are to the human CD20 sequence.

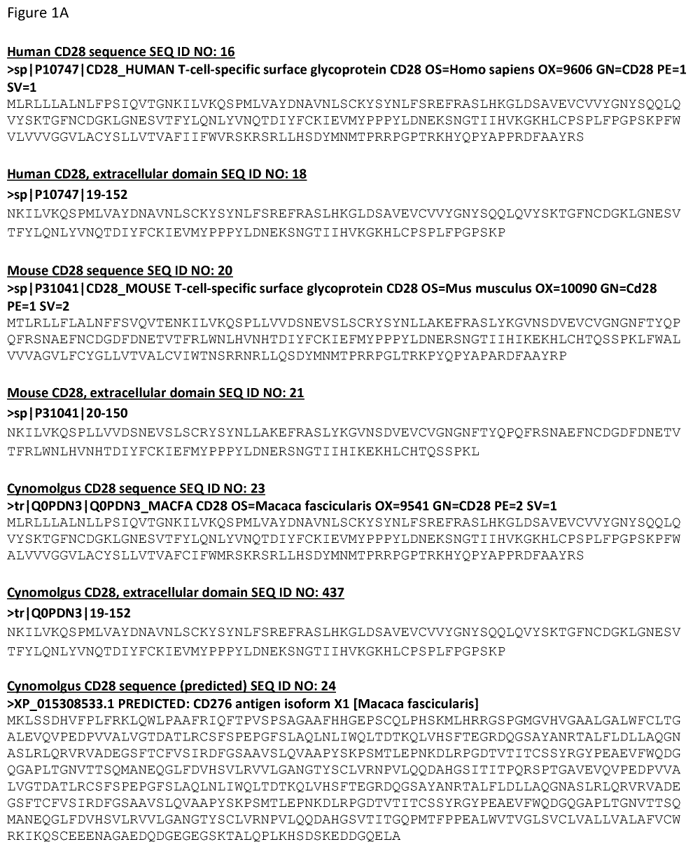

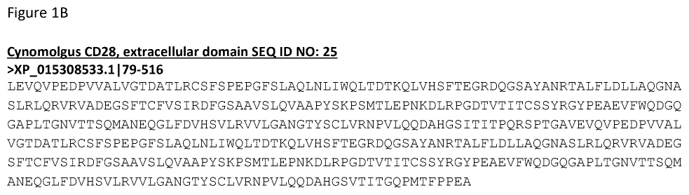

By “CD28,” “Cluster of Differentiation 28,” and “Tp44” (e.g., Genebank Accession Numbers NP_001230006 (human), NP_001230007 (human), NP_006130 (human), and NP_031668 (mouse)) herein is meant a B7 receptor expressed on T cells that provides co-stimulatory signals required for T cell activation and survival. T cell stimulation through CD28 in addition to the T cell receptor (TCR) provides a potent signal for the production of various interleukins. CD28 is the receptor for CD80 (B7.1) and CD86 (B7.2) proteins. CD28 includes an intercellular domain with a YMNM motif (SEQ ID NO:517) critical for the recruitment of SH2-domain containing proteins, particularly PI3K. CD28 also includes two proline-rich motifs that are able to bind SH3-containing proteins. Exemplary CD28 sequences are depicted in FIG. 1. Unless otherwise noted, references to CD28 are to the human CD28 sequence.

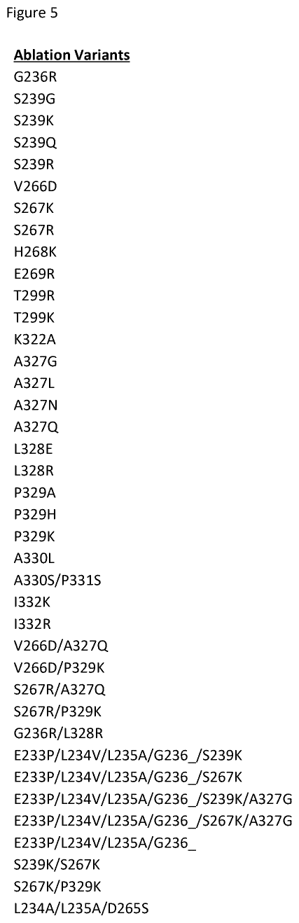

By “ablation” herein is meant a decrease or removal of activity. Thus, for example, “ablating FcγR binding” means the Fc region amino acid variant has less than 50% starting binding as compared to an Fc region not containing the specific variant. FcγR binding is assessed using a binding assay such as surface plasmon resonance (SPR) assays like Biacore or a biolayer interferometry (BLI), with ablation being defined as more than 50, 70, 80, 90, 95 or 98% loss of binding activity. Of particular use in the ablation of FcγR binding are those shown in FIG. 5, which generally are included in the bispecific antibodies disclosed herein.

By “ADCC” or “antibody dependent cell-mediated cytotoxicity” as used herein is meant the cell-mediated reaction, wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

By “ADCP” or antibody dependent cell-mediated phagocytosis as used herein is meant the cell-mediated reaction wherein nonspecific phagocytic cells that express FcγRs recognize bound antibody on a target cell and subsequently cause phagocytosis of the target cell.

As used herein, the term “antibody” is used generally. Antibodies provided herein can take on a number of formats as described herein, including traditional antibodies as well as antibody derivatives, fragments and mimetics, described herein.

Traditional immunoglobulin (Ig) antibodies are “Y” shaped tetramers. Each tetramer is typically composed of two identical pairs of polypeptide chains, each pair having one “light chain” monomer (typically having a molecular weight of about 25 kDa) and one “heavy chain” monomer (typically having a molecular weight of about 50-70 kDa).

Non-limiting examples of suitable antibody fragments include, without limitation diabodies and single-chain molecules as well as Fab, F(ab′)2, Fc, Fabc, and Fv molecules, single chain (Sc) antibodies, individual antibody light chains, individual antibody heavy chains, chimeric fusions between antibody chains or CDRs and other proteins, protein scaffolds, heavy chain monomers or dimers, light chain monomers or dimers, dimers consisting of one heavy and one light chain, a monovalent fragment consisting of the VL, VH, CL and CH1 domains, bivalent fragments comprising two Fab fragments linked by a disulfide bridge at the hinge region, a Fd fragment consisting essentially of the VH and CH1 domains; a Fv fragment consisting essentially of the VL and VH domains of a single arm of an antibody, a dAb fragment which consists essentially of a VH domain, camelid or nanobodies, an isolated complementarity determining region (CDR), and the like.

Other useful antibody formats include, but are not limited to, the “1+1 Fab-scFv-Fc,” “2+1 Fab₂-scFv-Fc,” “2+1 stack Fab₂-scFv-Fc,” and “2+1 mAb-scFv” formats described herein (FIG. 29). Additional useful antibody formats include, but are not limited to, “1+1 common light chain,” and “2+1 common light chain,” “mAb-Fv,” “mAb-scFv,” “central-Fv”, “one armed scFv-mAb,” “scFv-mAb,” “dual scFv,” and “trident” format antibodies (FIG. 29). See also, US20180127501A1, which is incorporated by reference herein, particularly in pertinent part relating to antibody formats (see, e.g., FIG. 2 of US20180127501A1).

Antibody heavy chains typically include a variable heavy (VH) domain, which includes vhCDR1-3, and an Fc domain, which includes a CH2-CH3 monomer. In some embodiments, antibody heavy chains include a hinge and CH1 domain. Traditional antibody heavy chains are monomers that are organized, from N- to C-terminus: VH-CH1-hinge-CH2-CH3. The CH1-hinge-CH2-CH3 is collectively referred to as the heavy chain “constant domain” or “constant region” of the antibody, of which there are five different categories or “isotypes”: IgA, IgD, IgG, IgE and IgM.

In some embodiments, the antibodies provided herein include IgG isotype constant domains, which has several subclasses, including, but not limited to IgG1, IgG2, IgG3, and IgG4. In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. As shown in Table 1, the exact numbering and placement of the heavy chain domains can be different among different numbering systems. As shown herein and described below, the pI variants can be in one or more of the CH regions, as well as the hinge region, discussed below.

It should be noted that IgG1 has different allotypes with polymorphisms at 356 (D or E) and 358 (L or M). The sequences depicted herein use the 356E/358M allotype, however the other allotype is included herein. That is, any sequence inclusive of an IgG1 Fc domain included herein can have 356D/358L replacing the 356E/358M allotype. It should be understood that therapeutic antibodies can also comprise hybrids of isotypes and/or subclasses. For example, as shown in US Publication 2009/0163699, incorporated by reference, the present antibodies, in some embodiments, include human IgG1/G2 hybrids.

By “Fc” or “Fc region” or “Fc domain” as used herein is meant the polypeptide comprising the constant region of an antibody, in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (Cγ2 and Cγ3), and optionally all or a portion of the hinge region between CH1 (Cγ1) and CH2 (Cγ2). Thus, in some cases, the Fc domain includes, from N- to C-terminal, CH2-CH3 and hinge-CH2-CH3. In some embodiments, the Fc domain is that from IgG1, IgG2, IgG3 or IgG4, with IgG1 hinge-CH2-CH3 and IgG4 hinge-CH2-CH3 finding particular use in many embodiments. Additionally, in the case of human IgG1 Fc domains, the hinge may include a C220S amino acid substitution. Furthermore, in the case of human IgG4 Fc domains, the hinge may include a S228P amino acid substitution. Although the boundaries of the Fc region may vary, the human IgG heavy chain Fc region is usually defined to include residues E216, C226, or A231 to its carboxyl-terminal, wherein the numbering is according to the EU index as in Kabat. In some embodiments, as is more fully described below, amino acid modifications are made to the Fc region, for example to alter binding to one or more FcγR or to the FcRn.

By “heavy chain constant region” herein is meant the CH1-hinge-CH2-CH3 portion of an antibody (or fragments thereof), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region.

Another type of domain of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (P230 in IgG1), wherein the numbering is according to the EU index as in Kabat. In some cases, a “hinge fragment” is used, which contains fewer amino acids at either or both of the N- and C-termini of the hinge domain. As noted herein, pI variants can be made in the hinge region as well. Many of the antibodies herein have at least one the cysteines at position 220 according to EU numbering (hinge region) replaced by a serine. Generally, this modification is on the “scFv monomer” side (when 1+1 or 2+1 formats are used) for most of the sequences depicted herein, although it can also be on the “Fab monomer” side, or both, to reduce disulfide formation. Specifically included within the sequences herein are one or both of these cysteines replaced (C220S).

As will be appreciated by those in the art, the exact numbering and placement of the heavy chain constant region domains (i.e., CH1, hinge, CH2 and CH3 domains) can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The antibody light chain generally comprises two domains: the variable light domain (VL), which includes light chain CDRs vICDR1-3, and a constant light chain region (often referred to as CL or Cκ). The antibody light chain is typically organized from N- to C-terminus: VL-CL.

By “antigen binding domain” or “ABD” herein is meant a set of six Complementary Determining Regions (CDRs) that, when present as part of a polypeptide sequence, specifically binds a target antigen (e.g., CD20 or CD28) as discussed herein. As is known in the art, these CDRs are generally present as a first set of variable heavy CDRs (vhCDRs or VHCDRs) and a second set of variable light CDRs (vICDRs or VLCDRs), each comprising three CDRs: vhCDR1, vhCDR2, vhCDR3 variable heavy CDRs and vICDR1, vICDR2 and vICDR3 vhCDR3 variable light CDRs. The CDRs are present in the variable heavy domain (vhCDR1-3) and variable light domain (vICDR1-3). The variable heavy domain and variable light domain from an Fv region.

The present invention provides a large number of different CDR sets. In this case, a “full CDR set” comprises the three variable light and three variable heavy CDRs, e.g., a vICDR1, vICDR2, vICDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vICDRs (e.g., vICDR1, vICDR2 and vICDR3). A useful comparison of CDR numbering is as below):

TABLE 2

| | Kabat + | | | | | |
|---|---|---|---|---|---|---|
| | Chothia | IMGT | Kabat | AbM | Chothia | Contact |
| vhCDR1 | 26-35 | 27-38 | 31-35 | 26-35 | 26-32 | 30-35 |
| vhCDR2 | 50-65 | 56-65 | 50-65 | 50-58 | 52-56 | 47-58 |
| vhCDR3 | 95-102 | 105-117 | 95-102 | 95-102 | 95-102 | 93-101 |
| vlCDR1 | 24-34 | 27-38 | 24-34 | 24-34 | 24-34 | 30-36 |
| vlCDR2 | 50-56 | 56-65 | 50-56 | 50-56 | 50-56 | 46-55 |
| vlCDR3 | 89-97 | 105-117 | 89-97 | 89-97 | 89-97 | 89-96 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions.

The CDRs contribute to the formation of the antigen-binding site, or more specifically, epitope binding site of the antigen binding domains and antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

In some embodiments, the six CDRs of the antigen binding domain are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vICDR1, vICDR2 and vICDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh. In general, the C-terminus of the scFv domain is attached to the N-terminus of all or part of the hinge in the second monomer.

By “variable region” or “variable domain” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vICDR1, vICDR2 and vICDR3 for the variable light domain) and four framework (FR) regions, arranged from amino-terminus to carboxy-terminus in the following order: FR1-CDR1-FR2-CDR2-FR3-CDR3-FR4.

By “Fab” or “Fab region” as used herein is meant the antibody region that comprises the VH, CH1, VL, and CL immunoglobulin domains, generally on two different polypeptide chains (e.g., VH-CH1 on one chain and VL-CL on the other). Fab may refer to this region in isolation, or this region in the context of a bispecific antibody of the invention. In the context of a Fab, the Fab comprises an Fv region in addition to the CH1 and CL domains.

By “Fv” or “Fv fragment” or “Fv region” as used herein is meant the antibody region that comprises the VL and VH domains. Fv regions can be formatted as both Fabs (as discussed above, generally two different polypeptides that also include the constant regions as outlined above) and single chain Fvs (scFvs), where the vl and vh domains are included in a single peptide, attached generally with a linker as discussed herein.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the figures, the order of the vh and vl domain is indicated in the name, e.g., H.X_L.Y means N- to C-terminal is vh-linker-vl, and L.Y_H.X is vl-Iinker-vh.

Some embodiments of the subject antibodies provided herein comprise at least one scFv domain, which, while not naturally occurring, generally includes a variable heavy domain and a variable light domain, linked together by a scFv linker. As outlined herein, while the scFv domain is generally from N- to C-terminus oriented as VH-scFv linker-VL, this can be reversed for any of the scFv domains (or those constructed using vh and vl sequences from Fabs), to VL-scFv linker-VH, with optional linkers at one or both ends depending on the format.

By “modification” or “variant” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution;” that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233-E233#, E233_, E233( ) and E233del all designate a deletion of glutamic acid at position 233. Additionally, EDA233- or EDA233# designates a deletion of the sequence GluAspAla that begins at position 233.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75, 80, 85, 90, 91, 92, 93, 94, 95, 96, 97, 98 or 99% identical to the parent protein, using the alignment programs described below, such as BLAST. See Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi.

“Fc variant” or “variant Fc” as used herein is meant a protein comprising an amino acid modification in an Fc domain. The modification can be an addition, deletion, or substitution. The Fc variants are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). The modification can be an addition, deletion, or substitution.

In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Alternatively, the variant Fc domains can have up to 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Additionally, as discussed herein, the variant Fc domains described herein still retain the ability to form a dimer with another Fc domain as measured using known techniques as described herein, such as non-denaturing gel electrophoresis.

By “protein” as used herein is meant at least two covalently attached amino acids, which includes proteins, polypeptides, oligopeptides and peptides. In addition, polypeptides that make up the antibodies of the invention may include synthetic derivatization of one or more side chains or termini, glycosylation, PEGylation, circular permutation, cyclization, linkers to other molecules, fusion to proteins or protein domains, and addition of peptide tags or labels.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs. Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants used to increase binding to the FcRn receptor, and in some cases, to increase serum half-life. An “FcRn variant” is an amino acid modification that contributes to increased binding to the FcRn receptor, and suitable FcRn variants are shown below.

By “parent polypeptide” as used herein is meant a starting polypeptide that is subsequently modified to generate a variant. The parent polypeptide may be a naturally occurring polypeptide, or a variant or engineered version of a naturally occurring polypeptide. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below. In this context, a “parent Fc domain” will be relative to the recited variant; thus, a “variant human IgG1 Fc domain” is compared to the parent Fc domain of human IgG1, a “variant human IgG4 Fc domain” is compared to the parent Fc domain human IgG4, etc.

By “strandedness” in the context of the monomers of the heterodimeric antibodies of the invention herein is meant that, similar to the two strands of DNA that “match”, heterodimerization variants are incorporated into each monomer so as to preserve the ability to “match” to form heterodimers. For example, if some pI variants are engineered into monomer A (e.g., making the pI higher) then steric variants that are “charge pairs” that can be utilized as well do not interfere with the pI variants, e.g., the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer one set of the pair will go, such that pI separation is maximized using the pI of the skews as well.

Provided herein are a number of antibody domains (e.g., Fc domains) that have sequence identity to human antibody domains. Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore, SPR or BLI assay.

### IV. Anti-CD28 x Anti-CD20 Antibodies

In one embodiment, provided herein are novel anti-CD20 x anti-CD28 antibodies In some embodiments, the anti-CD20 x anti-CD28 antibodies described herein are capable of binding to CD28 costimulatory molecules on T cells and CD20 on B cells in B cell malignancies. Such antibodies selectively enhance anti-tumor activity against B cells that exhibit CD20 expression in such B cell malignancies. In embodiments, the subject antibodies provided herein are particularly useful in combination with other anti-cancer therapies, including, for example, multivalent antibodies for the treatment of such B cell malignancies.

The anti-CD20 x anti-CD28 antibodies are multivalent and include at least two antigen binding domains (ABDs), wherein at least one antigen binding domain is a CD20 binding domain and at least one antigen binding domain is a CD28 binding domain. Any suitable CD20 binding domain and CD28 binding domain can be included in the subject anti-CD20 x anti-CD28 antibodies, including, for example, the CD20 binding domains and CD28 binding domains provided herein.

The antigen binding domains provided herein generally include a variable heavy domain (VH) having vhCDR1, vhCDR2, and vhCDR3; and a variable light domain (VL), and a variable light domain (VL) having vICDR1, vICDR2, and vICDR3.

In addition, as discussed above, the numbering used in the sequence listing and figures for the identification of the CDRs is Kabat, however, different numbering can be used, which will change the amino acid sequences of the CDRs as shown in Table 2.

For all of the variable heavy and light domains listed herein, further variants can be made. As outlined herein, in some embodiments the set of 6 CDRs can have from 0, 1, 2, 3, 4 or 5 amino acid modifications (with amino acid substitutions finding particular use), as well as changes in the framework regions of the variable heavy and light domains, as long as the frameworks (excluding the CDRs) retain at least about 80, 85 or 90% identity to a human germline sequence selected from those listed in FIG. 1 of U.S. Pat. No. 7,657,380, which Figure and Legend is incorporated by reference in its entirety herein. Thus, for example, the identical CDRs as described herein can be combined with different framework sequences from human germline sequences, as long as the framework regions retain at least 80, 85 90, 95 or 99% identity to a human germline sequence selected from those listed in FIG. 1 of U.S. Pat. No. 7,657,380. Alternatively, the CDRs can have amino acid modifications (e.g., from 1, 2, 3, 4 or 5 amino acid modifications in the set of CDRs (that is, the CDRs can be modified as long as the total number of changes in the set of 6 CDRs is less than 6 amino acid modifications, with any combination of CDRs being changed; e.g., there may be one change in vICDR1, two in vhCDR2, none in vhCDR3, etc.)), as well as having framework region changes, as long as the framework regions retain at least 80, 85 90, 95 or 99% identity to a human germline sequence selected from those listed in FIG. 1 of U.S. Pat. No. 7,657,380.

As will be appreciated by those in the art, any set of 6 CDRs or VH and VL domains can be in the scFv format or in the Fab format, which is then added to the heavy and light constant domains, where the heavy constant domains comprise variants (including within the CH1 domain as well as the Fc domain).

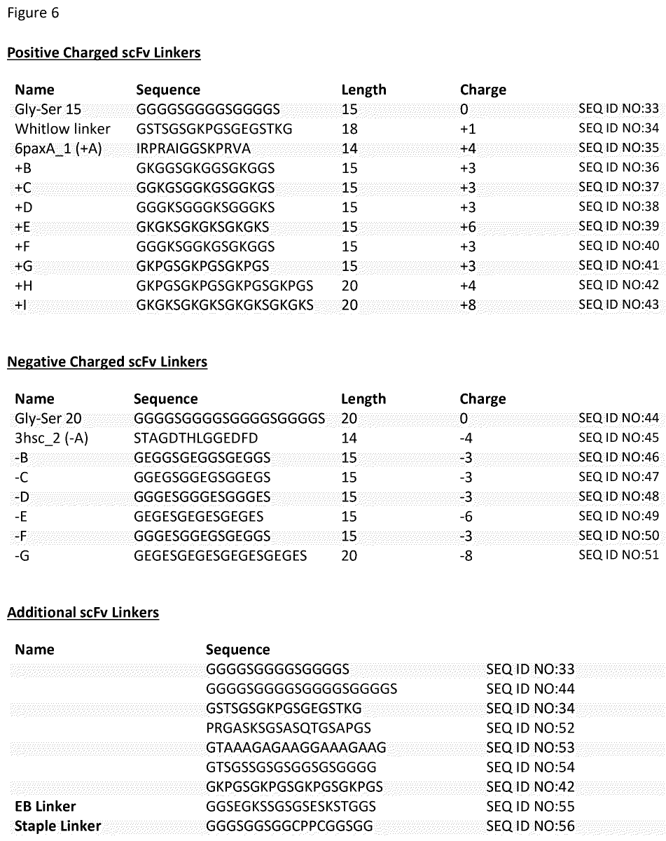

In addition, in embodiments wherein the subject antibody includes an scFv, the scFv can be in an orientation from N- to C-terminus of VH-scFv linker-VL or VL-scFv linker-VH. In some formats, one or more of the ABDs generally is a Fab that includes a VH domain on one protein chain (generally as a component of a heavy chain) and a VL on another protein chain (generally as a component of a light chain). Exemplary scFv linkers for use in the subject antibodies are depicted in FIG. 6.

Useful CD20 binding domains and CD28 binding domains that can be included in the subject anti-CD20 x anti-CD28 antibodies are further detailed herein.

In some embodiments, the anti-CD20 x anti-CD28 antibody is a bispecific antibody. In some embodiments, the anti-CD20 x anti-CD28 antibody is a bivalent antibody. In some embodiments, the anti-CD20 x anti-CD28 antibody is a trivalent antibody. In some embodiments, the anti-CD20 x anti-CD28 antibody is a bispecific, bivalent antibody. In some embodiments, the anti-CD20 x anti-CD28 antibodies include one CD28 binding domain and one CD20 binding domain. In exemplary embodiments, the anti-CD20 x anti-CD28 antibody is a bispecific, trivalent antibody. In some embodiments, the anti-CD20 x anti-CD28 antibodies include one CD28 binding domain and two CD20 binding domains.

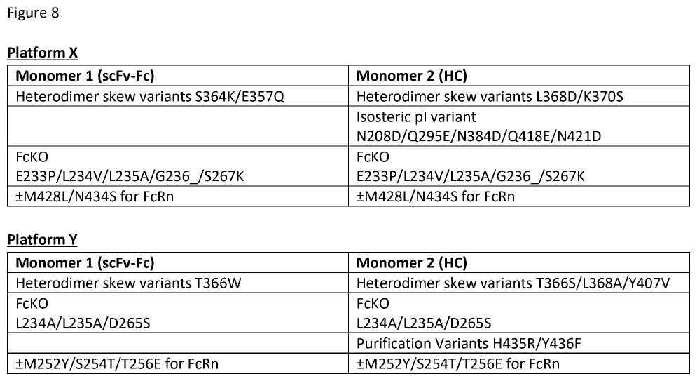

The anti-CD20 x anti-CD28 antibodies provided herein can be in any useful format, including, including, for example, canonical immunoglobulin, as well as the “1+1 Fab-scFv-Fc,” “2+1 Fab₂-scFv-Fc,” “2+1 stack Fab₂-scFv-Fc,” and “2+1 mAb-scFv” formats described herein (FIG. 29). Additional useful formats include, but are not limited to: “1+1 common light chain,” and “2+1 common light chain,” “mAb-Fv,” “mAb-scFv,” “central-Fv”, “one armed scFv-mAb,” “scFv-mAb,” “dual scFv,” and “trident” formats provided herein (see, e.g., FIG. 29). See also, US20180127501A1, which is incorporated by reference herein, particularly in pertinent part relating to antibody formats (see, e.g., FIG. 2). In some embodiments, the anti-CD20 x anti-CD28 antibodies are heterodimeric bispecific antibodies that include variant Fc domains having any of the heterodimerization skew variants, pI variants and/or ablation variants described herein. See, e.g. FIG. 8.

Note that unless specified herein, the order of the antigen list in the name does not confer structure; that is an anti-CD20 x anti-CD28 1+1 Fab-scFv-Fc antibody can have the scFv bind to CD20 or CD28, although in some cases, the order specifies structure as indicated.

The anti-CD20 x anti-CD28 antibodies provided herein further include different antibody domains. As described herein and known in the art, the antibodies described herein include different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

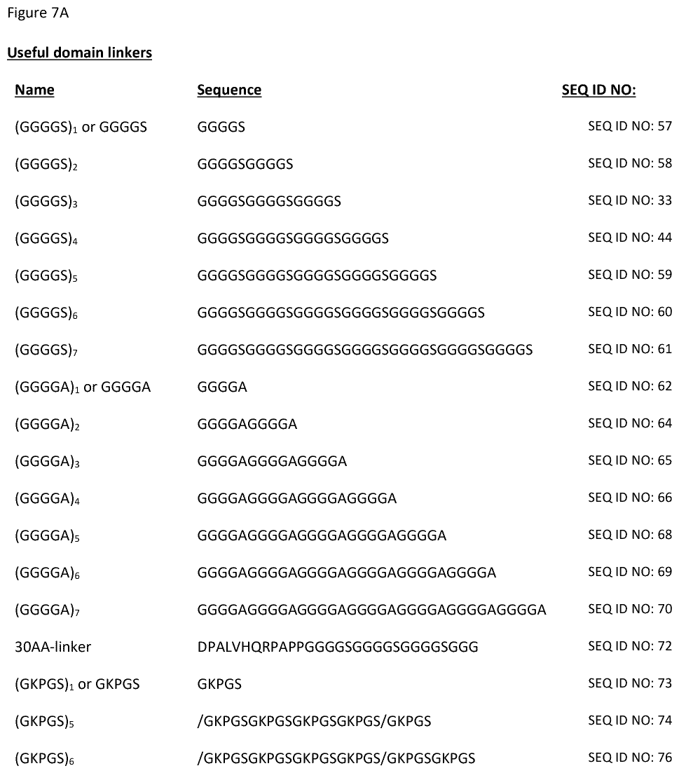

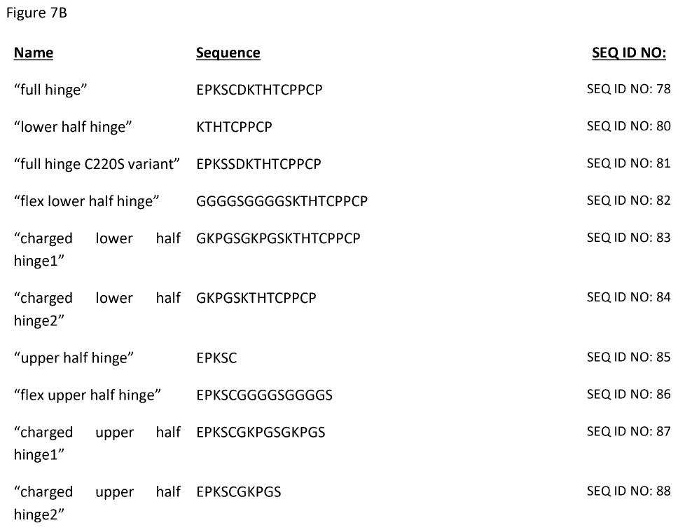

As shown herein, there are a number of suitable linkers (for use as either domain linkers or scFv linkers) that can be used to covalently attach the recited domains (e.g., scFvs, Fabs, Fc domains, VH domains, VL domains, etc.), including traditional peptide bonds, generated by recombinant techniques. Exemplary linkers to attach domains of the subject antibody to each other are depicted in FIG. 7. In some embodiments, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n (SEQ ID NO:518), (GGGGS)n (SEQ ID NO:57), and (GGGS)n (SEQ ID NO:519), where n is an integer of at least one (and generally from 3 to 4), glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

Other linker sequences may include any sequence of any length of CL/CH1 domain but not all residues of CL/CH1 domain; for example the first 5-12 amino acid residues of the CL/CH1 domains. Linkers can be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR), hinge region-derived sequences, and other natural sequences from other proteins.

In some embodiments, the linker is a “domain linker”, used to link any two domains as outlined herein together. For example, in the 2+1 Fab₂-scFv-Fc format, there may be a domain linker that attaches the C-terminus of the CH1 domain of the Fab to the N-terminus of the scFv, with another optional domain linker attaching the C-terminus of the scFv to the CH2 domain (although in many embodiments the hinge is used as this domain linker). While any suitable linker can be used, many embodiments utilize a glycine-serine polymer as the domain linker, including for example (GS)n, (GSGGS)n (SEQ ID NO:518), (GGGGS)n (SEQ ID NO:57), and (GGGS)n (SEQ ID NO:519), where n is an integer of at least one (and generally from 3 to 4 to 5) as well as any peptide sequence that allows for recombinant attachment of the two domains with sufficient length and flexibility to allow each domain to retain its biological function. In some cases, and with attention being paid to “strandedness”, as outlined below, charged domain linkers, as used in some embodiments of scFv linkers can be used. Exemplary useful domain linkers are depicted in FIG. 7.

In some embodiments, the linker is a scFv linker that is used to covalently attach the VH and VL domains as discussed herein. In many cases, the scFv linker is a charged scFv linker, a number of which are shown in FIG. 6. Accordingly, provided herein are charged scFv linkers, to facilitate the separation in pI between a first and a second monomer. That is, by incorporating a charged scFv linker, either positive or negative (or both, in the case of scaffolds that use scFvs on different monomers), this allows the monomer comprising the charged linker to alter the pI without making further changes in the Fc domains. These charged linkers can be substituted into any scFv containing standard linkers. Again, as will be appreciated by those in the art, charged scFv linkers are used on the correct “strand” or monomer, according to the desired changes in pI. For example, as discussed herein, to make 1+1 Fab-scFv-Fc format heterodimeric antibody, the original pI of the Fv region for each of the desired antigen binding domains are calculated, and one is chosen to make an scFv, and depending on the pI, either positive or negative linkers are chosen. Charged domain linkers can also be used to increase the pI separation of the monomers of the invention as well, and thus those included in FIG. 6 can be used in any embodiment herein where a linker is utilized. In some embodiments, the scFv is a “stapled” scFv that includes a “staple linker.” “Stapled” scFvs that exhibit improved stability and/or reduced aggregation are further described in detail herein. Exemplary staple linkers that are useful for inclusion in such “stapled” scFvs are provided in FIG. 6.

In some embodiments, wherein an scFv is included in the anti-CD20 x anti-CD28 antibody (e.g., the 1+1 Fab-scFv-Fc format or 2+1 Fab₂-scFv-Fc format antibody), the scFv includes “staple” modifications that improve scFv stability and/or reduces aggregation. In exemplary embodiments, such “stapled” scFvs include: a) a first disulfide bond between a structurally conserved surface exposed VH cysteine and a first scFv linker cysteine; b) a second disulfide bond between a structurally conserved surface exposed VL cysteine and a second scFv linker cysteine; or c) the first disulfide bond between the structurally conserved surface exposed VH cysteine and the first scFv linker cysteine and the second disulfide bond between the structurally considered surface exposed VL cysteine and the second scFv linker cysteine. Exemplary scFv “staple linkers” for inclusion in “stapled” scFvs are provided in FIG. 6. Methods for making “stapled” scFvs are described in WO 2021/030657, which is incorporated by reference in its entirety, including pertinent parts relating to methods for making “stapled” scFvs, and “stapled” scFv compositions.

Exemplary subject anti-CD20 x anti-CD28 antibodies are depicted, for example, in FIGS. 33-36. During the cell culture production of the anti-CD20 x anti-CD28 antibodies provided herein, the C-terminal lysine residue or C-terminal lysine and glycine residues may be cleaved from the heavy chain monomers, thereby leading to variants with C-terminal “clipping.” See, e.g., Jiang et al., Journal of Pharmaceutical Sciences 105:2066-2072 (2016). Thus, in some embodiments provided herein, the anti-CD20 x anti-CD28 antibody is a variant of one of the anti-CD20 x anti-CD28 antibodies depicted in FIGS. 33-36 that includes a deletion of a C-terminal lysine (−K) residues or lysine and glycine (−GK) residues in “chain 1” and/or “chain 2.” In some embodiments, the deletion is G446del and/or K447del. In some embodiments, the anti-CD20 x anti-CD28 antibody is engineered to include a G446del and/or K447del modification in one or both Fc domains of an anti-CD20 x anti-CD28 antibody described herein. In some embodiments, the anti-CD20 x anti-CD28 antibody includes a naturally occurring G446del and/or K447del modification in one or both Fc domains as compared to an anti-CD20 x anti-CD28 antibody provided herein.

Embodiments of the anti-CD20 x anti-CD28 antibodies are further described in detail below.

### A. CD28 Binding Domains

The anti-CD20 x anti-CD28 antibodies provided herein include at least one CD28 binding domain. Any suitable CD28 binding domain can be included in the anti-CD20 x anti-CD28 antibodies provided herein. In exemplary embodiments, the CD28 binding domain is an agonistic CD28 ABDs that advantageously provide T cell costimulatory activity.

As will be appreciated by those in the art, suitable CD28 binding domains can comprise a set of 6 CDRs as depicted in the figures, either as they are underlined or, in the case where a different numbering scheme is used, as described herein and as shown in Table 2, as the CDRs that are identified using other alignments within the variable heavy (VH) domain and variable light domain (VL) sequences of those depicted in FIGS. 15-24, and 26. Suitable CD28 ABDs can also include the entire VH and VL sequences as depicted in these sequences and figures, used as scFvs or as Fabs.

In some embodiments, the CD28 ABD comprises a variable heavy (VH) domain and a variable light (VL) domain, wherein the VH is selected from the amino acid sequences represented by SEQ ID NOs: 1, 11, 15, 63, 67, 71, 132-196, 320, 327, 335, 343, 351, 359, 367, 375, 383, 391, 399, 407, 415, 423, and 431, or a variant thereof; and wherein VL is selected from the amino acid sequences represented by SEQ ID NOs: 5, 19, 75, 79, 200-305, 324, 331, 339, 347, 355, 371, 379, 387, 395, 403, 411, 419, and 435, or a variant thereof.

In addition to the parental CDR sets disclosed in the figures and sequence listing that form an ABD to CD28, provided herein are variant CD28 ABDS having CDRs that include at least one modification of the CD28 ABD CDRs disclosed herein (e.g., (FIGS. 15-18, 21, and 26 and the sequence listing). In one embodiment, the CD28 ABD of the subject anti-CD20 x anti-CD28 antibody includes a set of 6 CDRs with 1, 2, 3, 4, 5, 6, 7, 8, 9, 10 amino acid modifications as compared to the 6 CDRs of a CD28 ABD as described herein, including the figures and sequence listing. In exemplary embodiments, the CD28 ABD of the subject anti-CD20 x anti-CD28 antibody includes a set of 6 CDRs with 1, 2, 3, 4, 5, 6, 7, 8, 9, 10 amino acid modifications as compared to the 6 CDRs of a CD28 ABD having one of the following VH and VL:
(i) a VH having an amino acid sequence of SEQ ID NO:1; and (ii) a VL having an amino acid sequence of SEQ ID NO:5; or
(i) a VH having an amino acid sequence of SEQ ID NO:11; and (ii) a VL having an amino acid sequence of SEQ ID NO:5; or
(i) a VH having an amino acid sequence of SEQ ID NO:1; and (ii) a VL having an amino acid sequence of SEQ ID NO:19; or
(i) a VH having an amino acid sequence of SEQ ID NO:11; and (ii) a VL having an amino acid sequence of SEQ ID NO:19; or
(i) a VH having an amino acid sequence of SEQ ID NO:15; and (ii) a VL having an amino acid sequence of SEQ ID NO:5; or
(i) a VH having an amino acid sequence of SEQ ID NO:15; and (ii) a VL having an amino acid sequence of SEQ ID NO:19; or
(i) a VH having an amino acid sequence of SEQ ID NO:63; and (ii) a VL having an amino acid sequence of SEQ ID NO:75; or
(i) a VH having an amino acid sequence of SEQ ID NO:67; and (ii) a VL having an amino acid sequence of SEQ ID NO:75; or
(i) a VH having an amino acid sequence of SEQ ID NO:63; and (ii) a VL having an amino acid sequence of SEQ ID NO:79; or
(i) a VH having an amino acid sequence of SEQ ID NO:67; and (ii) a VL having an amino acid sequence of SEQ ID NO:111; or
(i) a VH having an amino acid sequence of SEQ ID NO:71; and (ii) a VL having an amino acid sequence of SEQ ID NO:75; or
(i) a VH having an amino acid sequence of SEQ ID NO:71; and (ii) a VL having an amino acid sequence of SEQ ID NO:79; or
i) a VH having an amino acid sequence of SEQ ID NO:320; and (ii) a VL having an amino acid sequence of SEQ ID NO:324; or
(i) a VH having an amino acid sequence of SEQ ID NO:327; and (ii) a VL having an amino acid sequence of SEQ ID NO:331; or
(i) a VH having an amino acid sequence of SEQ ID NO:335; and (ii) a VL having an amino acid sequence of SEQ ID NO:339; or
(i) a VH having an amino acid sequence of SEQ ID NO:343; and (ii) a VL having an amino acid sequence of SEQ ID NO:347; or
(i) a VH having an amino acid sequence of SEQ ID NO:351; and (ii) a VL having an amino acid sequence of SEQ ID NO:355; or
(i) a VH having an amino acid sequence of SEQ ID NO:359; and (ii) a VL having an amino acid sequence of SEQ ID NO:355; or
(i) a VH having an amino acid sequence of SEQ ID NO:367; and (ii) a VL having an amino acid sequence of SEQ ID NO:371; or
(i) a VH having an amino acid sequence of SEQ ID NO:375; and (ii) a VL having an amino acid sequence of SEQ ID NO:380; or
(i) a VH having an amino acid sequence of SEQ ID NO:391; and (ii) a VL having an amino acid sequence of SEQ ID NO:395; or
(i) a VH having an amino acid sequence of SEQ ID NO:399; and (ii) a VL having an amino acid sequence of SEQ ID NO:403; or
(i) a VH having an amino acid sequence of SEQ ID NO:407; and (ii) a VL having an amino acid sequence of SEQ ID NO:411; or
(i) a VH having an amino acid sequence of SEQ ID NO:415; and (ii) a VL having an amino acid sequence of SEQ ID NO:419; or
(i) a VH having an amino acid sequence of SEQ ID NO:423; and (ii) a VL having an amino acid sequence of SEQ ID NO:5; or
(i) a VH having an amino acid sequence of SEQ ID NO:431; and (ii) a VL having an amino acid sequence of SEQ ID NO:435.

In certain embodiments, the CD28 ABD of the subject anti-CD20 x anti-CD28 antibody is capable of binding CD28 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In some embodiments, the CD28 ABD of the subject anti-CD20 x anti-CD28 antibody includes 6 CDRs that are at least 90, 95, 97, 98 or 99% identical to the 6 CDRs of a CD28 ABD as described herein, including the figures and sequence listing. In exemplary embodiments, the CD28 ABD of the subject anti-CD20 x anti-CD28 antibody includes 6 CDRs that are at least 90, 95, 97, 98 or 99% identical to the 6 CDRs of a CD28 ABD having one of the following VH and VL:
(i) a VH having an amino acid sequence of SEQ ID NO:1; and (ii) a VL having an amino acid sequence of SEQ ID NO:5; or
(i) a VH having an amino acid sequence of SEQ ID NO:11; and (ii) a VL having an amino acid sequence of SEQ ID NO:5; or
(i) a VH having an amino acid sequence of SEQ ID NO:1; and (ii) a VL having an amino acid sequence of SEQ ID NO:19; or
(i) a VH having an amino acid sequence of SEQ ID NO:11; and (ii) a VL having an amino acid sequence of SEQ ID NO:19; or
(i) a VH having an amino acid sequence of SEQ ID NO:15; and (ii) a VL having an amino acid sequence of SEQ ID NO:5; or
(i) a VH having an amino acid sequence of SEQ ID NO:15; and (ii) a VL having an amino acid sequence of SEQ ID NO:19; or
(i) a VH having an amino acid sequence of SEQ ID NO:63; and (ii) a VL having an amino acid sequence of SEQ ID NO:75; or
(i) a VH having an amino acid sequence of SEQ ID NO:67; and (ii) a VL having an amino acid sequence of SEQ ID NO:75; or
(i) a VH having an amino acid sequence of SEQ ID NO:63; and (ii) a VL having an amino acid sequence of SEQ ID NO:79; or
(i) a VH having an amino acid sequence of SEQ ID NO:67; and (ii) a VL having an amino acid sequence of SEQ ID NO:111; or
(i) a VH having an amino acid sequence of SEQ ID NO:71; and (ii) a VL having an amino acid sequence of SEQ ID NO:75; or
(i) a VH having an amino acid sequence of SEQ ID NO:71; and (ii) a VL having an amino acid sequence of SEQ ID NO:79; or
i) a VH having an amino acid sequence of SEQ ID NO:320; and (ii) a VL having an amino acid sequence of SEQ ID NO:324; or
(i) a VH having an amino acid sequence of SEQ ID NO:327; and (ii) a VL having an amino acid sequence of SEQ ID NO:331; or
(i) a VH having an amino acid sequence of SEQ ID NO:335; and (ii) a VL having an amino acid sequence of SEQ ID NO:339; or
(i) a VH having an amino acid sequence of SEQ ID NO:343; and (ii) a VL having an amino acid sequence of SEQ ID NO:347; or
(i) a VH having an amino acid sequence of SEQ ID NO:351; and (ii) a VL having an amino acid sequence of SEQ ID NO:355; or
(i) a VH having an amino acid sequence of SEQ ID NO:359; and (ii) a VL having an amino acid sequence of SEQ ID NO:355; or
(i) a VH having an amino acid sequence of SEQ ID NO:367; and (ii) a VL having an amino acid sequence of SEQ ID NO:371; or
(i) a VH having an amino acid sequence of SEQ ID NO:375; and (ii) a VL having an amino acid sequence of SEQ ID NO:380; or
(i) a VH having an amino acid sequence of SEQ ID NO:391; and (ii) a VL having an amino acid sequence of SEQ ID NO:395; or
(i) a VH having an amino acid sequence of SEQ ID NO:399; and (ii) a VL having an amino acid sequence of SEQ ID NO:403; or
(i) a VH having an amino acid sequence of SEQ ID NO:407; and (ii) a VL having an amino acid sequence of SEQ ID NO:411; or
(i) a VH having an amino acid sequence of SEQ ID NO:415; and (ii) a VL having an amino acid sequence of SEQ ID NO:419; or
(i) a VH having an amino acid sequence of SEQ ID NO:423; and (ii) a VL having an amino acid sequence of SEQ ID NO:5; or
(i) a VH having an amino acid sequence of SEQ ID NO:431; and (ii) a VL having an amino acid sequence of SEQ ID NO:435 (FIGS. 15, 18, 21, and 26).

In certain embodiments, the CD28 ABD is capable of binding to the CD28, as measured by at least one of a Biacore, surface plasmon resonance (SPR), flow cytometry, and/or BLI (biolayer interferometry, e.g., Octet assay) assay, with the latter finding particular use in many embodiments.

In particular embodiments, the CD28 ABD is capable of binding human CD28 antigen (see FIG. 1).

In some embodiments, the anti-CD20 x anti-CD28 antibody includes a CD28 ABD that includes a variable heavy domain and/or a variable light domain that are variants of a CD28 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a CD28 ABD described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of a CD28 ABD having one of the following VH and VL:
(i) a VH having an amino acid sequence of SEQ ID NO:1; and (ii) a VL having an amino acid sequence of SEQ ID NO:5; or
(i) a VH having an amino acid sequence of SEQ ID NO:11; and (ii) a VL having an amino acid sequence of SEQ ID NO:5; or
(i) a VH having an amino acid sequence of SEQ ID NO:1; and (ii) a VL having an amino acid sequence of SEQ ID NO:19; or
(i) a VH having an amino acid sequence of SEQ ID NO:11; and (ii) a VL having an amino acid sequence of SEQ ID NO:19; or
(i) a VH having an amino acid sequence of SEQ ID NO:15; and (ii) a VL having an amino acid sequence of SEQ ID NO:5; or
(i) a VH having an amino acid sequence of SEQ ID NO:15; and (ii) a VL having an amino acid sequence of SEQ ID NO:19; or
(i) a VH having an amino acid sequence of SEQ ID NO:63; and (ii) a VL having an amino acid sequence of SEQ ID NO:75; or
(i) a VH having an amino acid sequence of SEQ ID NO:67; and (ii) a VL having an amino acid sequence of SEQ ID NO:75; or
(i) a VH having an amino acid sequence of SEQ ID NO:63; and (ii) a VL having an amino acid sequence of SEQ ID NO:79; or
(i) a VH having an amino acid sequence of SEQ ID NO:67; and (ii) a VL having an amino acid sequence of SEQ ID NO:111; or
(i) a VH having an amino acid sequence of SEQ ID NO:71; and (ii) a VL having an amino acid sequence of SEQ ID NO:75; or
(i) a VH having an amino acid sequence of SEQ ID NO:71; and (ii) a VL having an amino acid sequence of SEQ ID NO:79; or
i) a VH having an amino acid sequence of SEQ ID NO:320; and (ii) a VL having an amino acid sequence of SEQ ID NO:324; or
(i) a VH having an amino acid sequence of SEQ ID NO:327; and (ii) a VL having an amino acid sequence of SEQ ID NO:331; or
(i) a VH having an amino acid sequence of SEQ ID NO:335; and (ii) a VL having an amino acid sequence of SEQ ID NO:339; or
(i) a VH having an amino acid sequence of SEQ ID NO:343; and (ii) a VL having an amino acid sequence of SEQ ID NO:347; or
(i) a VH having an amino acid sequence of SEQ ID NO:351; and (ii) a VL having an amino acid sequence of SEQ ID NO:355; or
(i) a VH having an amino acid sequence of SEQ ID NO:359; and (ii) a VL having an amino acid sequence of SEQ ID NO:355; or
(i) a VH having an amino acid sequence of SEQ ID NO:367; and (ii) a VL having an amino acid sequence of SEQ ID NO:371; or
(i) a VH having an amino acid sequence of SEQ ID NO:375; and (ii) a VL having an amino acid sequence of SEQ ID NO:380; or
(i) a VH having an amino acid sequence of SEQ ID NO:391; and (ii) a VL having an amino acid sequence of SEQ ID NO:395; or
(i) a VH having an amino acid sequence of SEQ ID NO:399; and (ii) a VL having an amino acid sequence of SEQ ID NO:403; or
(i) a VH having an amino acid sequence of SEQ ID NO:407; and (ii) a VL having an amino acid sequence of SEQ ID NO:411; or
(i) a VH having an amino acid sequence of SEQ ID NO:415; and (ii) a VL having an amino acid sequence of SEQ ID NO:419; or
(i) a VH having an amino acid sequence of SEQ ID NO:423; and (ii) a VL having an amino acid sequence of SEQ ID NO:5; or
(i) a VH having an amino acid sequence of SEQ ID NO:431; and (ii) a VL having an amino acid sequence of SEQ ID NO:435 (FIGS. 15, 18, 21, and 26).

In some embodiments, the changes are in a VH domain. In some embodiments, the changes are in a VL domain. In some embodiments, the changes are in a VH and VL domain. In exemplary embodiments, the CD28 ABD is an scFv and the amino acid changes introduce cysteine residues in the VH and/or VL framework regions (FR1-FR4) to allow for “stapling” of the scFv, as described herein. See also WO 2021/030657, which is incorporated by reference in its entirety, including pertinent parts relating to methods for making “stapled” scFvs, and “stapled” scFv compositions. Exemplary “stapled” CD28 binding domains and VH/VL domains that can be included in the subject anti-CD20 x anti-CD28 antibodies depicted herein are included in FIGS. 19-21. In certain embodiments, the CD28 ABD of the anti-CD20 x anti-CD28 antibody is capable of binding to CD28, as measured at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In one embodiment, the CD28 antigen binding domain of the anti-CD20 x anti-CD28 antibody includes a variable heavy domain (VH) having the vhCDR1-3 (i.e., vhCDR1-3) of 1A7_H1sp (SEQ ID NO:63, FIG. 19). In some embodiments, the CD28 antigen binding domain of the anti-CD20 x anti-CD28 antibody further includes any of the CD28 binding domain variable light domains provided herein. In exemplary embodiments, the variable light domain is 1A7_L1.71sp (SEQ ID NO:79, FIG. 20) or a variant thereof. In certain embodiments, the CD28 ABD of the anti-CD20 x anti-CD28 antibody is capable of binding CD28 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD of the anti-CD20 x anti-CD28 antibody is capable of binding human CD28 antigen (see FIG. 1). In exemplary embodiments, the anti-CD20 x anti-CD28 antibody is a “1+1 Fab-scFv-Fc,” “2+1 Fab₂-scFv-Fc,” “2+1 stack Fab₂-scFv-Fc,” or “2+1 mAb-scFv” format antibody.

In one embodiment, the CD28 ABD of the anti-CD20 x anti-CD28 antibody includes a variable heavy domain (VH) having vhCDR1-3s with 1, 2, 3, 4, 5, 6, 7, 8, 9, 10 amino acid modifications as compared to the vhCDR1-3 of 1A7_H1sp (SEQ ID NO:63, FIG. 19). In some embodiments, the CD28 antigen binding domain of the anti-CD20 x anti-CD28 antibody further includes any of the CD28 binding domain variable light domains provided herein. In exemplary embodiments, the variable light domain is 1A7_L1.71sp (SEQ ID NO:79, FIG. 20) or a variant thereof. In certain embodiments, the CD28 ABD of the anti-CD20 x anti-CD28 antibody is capable of binding CD28 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD of the anti-CD20 x anti-CD28 antibody is capable of binding human CD28 antigen (see FIG. 1). In particular embodiments, the CD28 ABD of the anti-CD20 x anti-CD28 antibody is capable of binding human CD28 antigen (see FIG. 1). In exemplary embodiments, the anti-CD20 x anti-CD28 antibody is a “1+1 Fab-scFv-Fc,” “2+1 Fab₂-scFv-Fc,” “2+1 stack Fab₂-scFv-Fc,” or “2+1 mAb-scFv” format antibody.

In some embodiments, the CD28 ABD of the anti-CD20 x anti-CD28 antibody includes a variable heavy domain (VH) having vhCDR1-3s that are at least 90, 95, 97, 98 or 99% identical to the vhCDR1-3 of 1A7_H1sp (SEQ ID NO:63, FIG. 19). In some embodiments, the CD28 antigen binding domain of the anti-CD20 x anti-CD28 antibody further includes any of the CD28 binding domain variable light domains provided herein. In exemplary embodiments, the variable light domain is 1A7_L1.71sp (SEQ ID NO:79, FIG. 20) or a variant thereof. In certain embodiments, the CD28 ABD of the anti-CD20 x anti-CD28 antibody is capable of binding to the CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD of the anti-CD20 x anti-CD28 antibody is capable of binding human CD28 antigen (see FIG. 1). In exemplary embodiments, the anti-CD20 x anti-CD28 antibody is a “1+1 Fab-scFv-Fc,” “2+1 Fab₂-scFv-Fc,” “2+1 stack Fab₂-scFv-Fc,” or “2+1 mAb-scFv” format antibody.

In exemplary embodiments, the CD28 binding domain of the anti-CD20 x anti-CD28 antibody is an scFv. In some embodiments, the anti-CD28 scFv includes one of the CD28 ABD VH and/or VLs described herein or a variant thereof. In some embodiments, the anti-CD28 scFv of the anti-CD20 x anti-CD28 antibody includes one or more “staple” modifications that improves the stability or reduce aggregation of the scFv. In some embodiments, the anti-CD20 x anti-CD28 antibody includes a “stapled” scFv, wherein the stapled scFv includes: a) a first disulfide bond between a structurally conserved surface exposed VH cysteine and a first scFv linker cysteine; b) a second disulfide bond between a structurally conserved surface exposed VL cysteine and a second scFv linker cysteine; or c) the first disulfide bond between the structurally conserved surface exposed VH cysteine and the first scFv linker cysteine and the second disulfide bond between the structurally considered surface exposed VL cysteine and the second scFv linker cysteine. In exemplary embodiments, the “stapled” scFv is a variant of one of the CD28 ABDs described herein, wherein the variant CD28 ABD includes: a) an amino acid substitution to introduce a surface exposed VH cysteine; b) an amino acid substitution to introduce a surface exposed VL cysteine; or c) amino acid substitutions to introduce both a surface exposed VH cysteine and a surface exposed VH cysteine. In some embodiments, the amino acid change(s) is in the VH and/or VL framework regions (FR1, FR2, FR3, and/or FR4). Exemplary scFv “staple linkers” for inclusion in “stapled” scFvs are provided in FIG. 6. Methods for making “stapled” scFvs are described, for example, in WO 2021/030657, which is incorporated by reference in its entirety, including pertinent parts relating to methods for making “stapled” scFvs, and “stapled” scFv compositions. In exemplary embodiments, the anti-CD20 x anti-CD28 antibody includes a “stapled” scFv that includes a modified VH and VL of a CD28 ABD having one of the following VH and VLs:
(i) a VH having an amino acid sequence of SEQ ID NO:1; and (ii) a VL having an amino acid sequence of SEQ ID NO:5; or
(i) a VH having an amino acid sequence of SEQ ID NO:11; and (ii) a VL having an amino acid sequence of SEQ ID NO:5; or
(i) a VH having an amino acid sequence of SEQ ID NO:1; and (ii) a VL having an amino acid sequence of SEQ ID NO:19; or
(i) a VH having an amino acid sequence of SEQ ID NO:11; and (ii) a VL having an amino acid sequence of SEQ ID NO:19; or
(i) a VH having an amino acid sequence of SEQ ID NO:15; and (ii) a VL having an amino acid sequence of SEQ ID NO:5; or
(i) a VH having an amino acid sequence of SEQ ID NO:15; and (ii) a VL having an amino acid sequence of SEQ ID NO:19; or
(i) a VH having an amino acid sequence of SEQ ID NO:63; and (ii) a VL having an amino acid sequence of SEQ ID NO:75; or
(i) a VH having an amino acid sequence of SEQ ID NO:67; and (ii) a VL having an amino acid sequence of SEQ ID NO:75; or
(i) a VH having an amino acid sequence of SEQ ID NO:63; and (ii) a VL having an amino acid sequence of SEQ ID NO:79; or
(i) a VH having an amino acid sequence of SEQ ID NO:67; and (ii) a VL having an amino acid sequence of SEQ ID NO:111; or
(i) a VH having an amino acid sequence of SEQ ID NO:71; and (ii) a VL having an amino acid sequence of SEQ ID NO:75; or
(i) a VH having an amino acid sequence of SEQ ID NO:71; and (ii) a VL having an amino acid sequence of SEQ ID NO:79; or
(i) a VH having an amino acid sequence of SEQ ID NO:320; and (ii) a VL having an amino acid sequence of SEQ ID NO:324; or
(i) a VH having an amino acid sequence of SEQ ID NO:327; and (ii) a VL having an amino acid sequence of SEQ ID NO:331; or
(i) a VH having an amino acid sequence of SEQ ID NO:335; and (ii) a VL having an amino acid sequence of SEQ ID NO:339; or
(i) a VH having an amino acid sequence of SEQ ID NO:343; and (ii) a VL having an amino acid sequence of SEQ ID NO:347; or
(i) a VH having an amino acid sequence of SEQ ID NO:351; and (ii) a VL having an amino acid sequence of SEQ ID NO:355; or
(i) a VH having an amino acid sequence of SEQ ID NO:359; and (ii) a VL having an amino acid sequence of SEQ ID NO:355; or
(i) a VH having an amino acid sequence of SEQ ID NO:367; and (ii) a VL having an amino acid sequence of SEQ ID NO:371; or
(i) a VH having an amino acid sequence of SEQ ID NO:375; and (ii) a VL having an amino acid sequence of SEQ ID NO:380; or
(i) a VH having an amino acid sequence of SEQ ID NO:391; and (ii) a VL having an amino acid sequence of SEQ ID NO:395; or
(i) a VH having an amino acid sequence of SEQ ID NO:399; and (ii) a VL having an amino acid sequence of SEQ ID NO:403; or
(i) a VH having an amino acid sequence of SEQ ID NO:407; and (ii) a VL having an amino acid sequence of SEQ ID NO:411; or
(i) a VH having an amino acid sequence of SEQ ID NO:415; and (ii) a VL having an amino acid sequence of SEQ ID NO:419; or
(i) a VH having an amino acid sequence of SEQ ID NO:423; and (ii) a VL having an amino acid sequence of SEQ ID NO:5; or
(i) a VH having an amino acid sequence of SEQ ID NO:431; and (ii) a VL having an amino acid sequence of SEQ ID NO:435 (FIGS. 15, 18, and 26).

Exemplary “stapled” CD28 binding domains include, but are not limited to those depicted in FIG. 21.

In some embodiments, the anti-CD20 x anti-CD28 antibody includes a CD28 binding domain that comprises the CDRs selected from the following:
(i) a vhCDR1, a vhCDR2 and a vhCDR3 of a variable heavy chain domain (VH) having an amino acid sequence of SEQ ID NO:1, and a light chain complimentarity determining region (vICDR1), a vICDR2 and a vICDR3 of a variable light chain domain (VL) having an amino acid sequence of SEQ ID NO:5;
(ii) a vhCDR1, a vhCDR2 and a vhCDR3 of a variable heavy chain domain (VH) having an amino acid sequence of SEQ ID NO:11, and a light chain complimentarity determining region (vICDR1), a vICDR2 and a vICDR3 of a variable light chain domain (VL) having an amino acid sequence of SEQ ID NO:5;
(iii) a vhCDR1, a vhCDR2 and a vhCDR3 of a variable heavy chain domain (VH) having an amino acid sequence of SEQ ID NO:1, and a light chain complimentarity determining region (vICDR1), a vICDR2 and a vICDR3 of a variable light chain domain (VL) having an amino acid sequence of SEQ ID NO:19;
(iv) a vhCDR1, a vhCDR2 and a vhCDR3 of a variable heavy chain domain (VH) having an amino acid sequence of SEQ ID NO:11, and a light chain complimentarity determining region (vICDR1), a vICDR2 and a vICDR3 of a variable light chain domain (VL) having an amino acid sequence of SEQ ID NO:19;
(v) a vhCDR1, a vhCDR2 and a vhCDR3 of a variable heavy chain domain (VH) having an amino acid sequence of SEQ ID NO:15, and a light chain complimentarity determining region (vICDR1), a vICDR2 and a vICDR3 of a variable light chain domain (VL) having an amino acid sequence of SEQ ID NO:5;
(vi) a vhCDR1, a vhCDR2 and a vhCDR3 of a variable heavy chain domain (VH) having an amino acid sequence of SEQ ID NO:15, and a light chain complimentarity determining region (vICDR1), a vICDR2 and a vICDR3 of a variable light chain domain (VL) having an amino acid sequence of SEQ ID NO:19;
(vii) a vhCDR1, a vhCDR2 and a vhCDR3 of a variable heavy chain domain (VH) having an amino acid sequence of SEQ ID NO:63, and a light chain complimentarity determining region (vICDR1), a vICDR2 and a vICDR3 of a variable light chain domain (VL) having an amino acid sequence of SEQ ID NO:75;
(viii) a vhCDR1, a vhCDR2 and a vhCDR3 of a variable heavy chain domain (VH) having an amino acid sequence of SEQ ID NO:67, and a light chain complimentarity determining region (vICDR1), a vlCDR2 and a vlCDR3 of a variable light chain domain (VL) having an amino acid sequence of SEQ ID NO:75;
(ix) a vhCDR1, a vhCDR2 and a vhCDR3 of a variable heavy chain domain (VH) having an amino acid sequence of SEQ ID NO:63, and a light chain complimentarity determining region (vICDR1), a vlCDR2 and a vlCDR3 of a variable light chain domain (VL) having an amino acid sequence of SEQ ID NO:79;
(x) a vhCDR1, a vhCDR2 and a vhCDR3 of a variable heavy chain domain (VH) having an amino acid sequence of SEQ ID NO:67, and a light chain complimentarity determining region (vICDR1), a vlCDR2 and a vlCDR3 of a variable light chain domain (VL) having an amino acid sequence of SEQ ID NO:111;
(xi) a vhCDR1, a vhCDR2 and a vhCDR3 of a variable heavy chain domain (VH) having an amino acid sequence of SEQ ID NO:71, and a light chain complimentarity determining region (vICDR1), a vlCDR2 and a vlCDR3 of a variable light chain domain (VL) having an amino acid sequence of SEQ ID NO:75;
(xii) a vhCDR1, a vhCDR2 and a vhCDR3 of a variable heavy chain domain (VH) having an amino acid sequence of SEQ ID NO:71, and a light chain complimentarity determining region (vICDR1), a vlCDR2 and a vlCDR3 of a variable light chain domain (VL) having an amino acid sequence of SEQ ID NO:79;
(xiii) a vhCDR1, a vhCDR2 and a vhCDR3 of a variable heavy chain domain (VH) having an amino acid sequence of SEQ ID NO:320, and a light chain complimentarity determining region (vICDR1), a vlCDR2 and a vlCDR3 of a variable light chain domain (VL) having an amino acid sequence of SEQ ID NO:324;
(xiv) a vhCDR1, a vhCDR2 and a vhCDR3 of a variable heavy chain domain (VH) having an amino acid sequence of SEQ ID NO:327, and a light chain complimentarity determining region (vICDR1), a vlCDR2 and a vlCDR3 of a variable light chain domain (VL) having an amino acid sequence of SEQ ID NO:331;
(xv) a vhCDR1, a vhCDR2 and a vhCDR3 of a variable heavy chain domain (VH) having an amino acid sequence of SEQ ID NO:335, and a light chain complimentarity determining region (vICDR1), a vlCDR2 and a vlCDR3 of a variable light chain domain (VL) having an amino acid sequence of SEQ ID NO:339;
(xvi) a vhCDR1, a vhCDR2 and a vhCDR3 of a variable heavy chain domain (VH) having an amino acid sequence of SEQ ID NO:343, and a light chain complimentarity determining region (vICDR1), a vlCDR2 and a vlCDR3 of a variable light chain domain (VL) having an amino acid sequence of SEQ ID NO:347;
(xvii) a vhCDR1, a vhCDR2 and a vhCDR3 of a variable heavy chain domain (VH) having an amino acid sequence of SEQ ID NO:351, and a light chain complimentarity determining region (vICDR1), a vlCDR2 and a vlCDR3 of a variable light chain domain (VL) having an amino acid sequence of SEQ ID NO:355;
(xviii) a vhCDR1, a vhCDR2 and a vhCDR3 of a variable heavy chain domain (VH) having an amino acid sequence of SEQ ID NO:359, and a light chain complimentarity determining region (vICDR1), a vlCDR2 and a vlCDR3 of a variable light chain domain (VL) having an amino acid sequence of SEQ ID NO:355;
(xix) a vhCDR1, a vhCDR2 and a vhCDR3 of a variable heavy chain domain (VH) having an amino acid sequence of SEQ ID NO:367, and a light chain complimentarity determining region (vICDR1), a vlCDR2 and a vlCDR3 of a variable light chain domain (VL) having an amino acid sequence of SEQ ID NO:371;
(xx) a vhCDR1, a vhCDR2 and a vhCDR3 of a variable heavy chain domain (VH) having an amino acid sequence of SEQ ID NO:375, and a light chain complimentarity determining region (vICDR1), a vlCDR2 and a vlCDR3 of a variable light chain domain (VL) having an amino acid sequence of SEQ ID NO:380;
(xxi) a vhCDR1, a vhCDR2 and a vhCDR3 of a variable heavy chain domain (VH) having an amino acid sequence of SEQ ID NO:391, and a light chain complimentarity determining region (vICDR1), a vlCDR2 and a vlCDR3 of a variable light chain domain (VL) having an amino acid sequence of SEQ ID NO:395;
(xxii) a vhCDR1, a vhCDR2 and a vhCDR3 of a variable heavy chain domain (VH) having an amino acid sequence of SEQ ID NO:399, and a light chain complimentarity determining region (vICDR1), a vlCDR2 and a vlCDR3 of a variable light chain domain (VL) having an amino acid sequence of SEQ ID NO:403;
(xxiii) a vhCDR1, a vhCDR2 and a vhCDR3 of a variable heavy chain domain (VH) having an amino acid sequence of SEQ ID NO:407, and a light chain complimentarity determining region (vICDR1), a vlCDR2 and a vlCDR3 of a variable light chain domain (VL) having an amino acid sequence of SEQ ID NO:411;
(xxiv) a vhCDR1, a vhCDR2 and a vhCDR3 of a variable heavy chain domain (VH) having an amino acid sequence of SEQ ID NO:415, and a light chain complimentarity determining region (vICDR1), a vlCDR2 and a vlCDR3 of a variable light chain domain (VL) having an amino acid sequence of SEQ ID NO:419;
(xxv) a vhCDR1, a vhCDR2 and a vhCDR3 of a variable heavy chain domain (VH) having an amino acid sequence of SEQ ID NO:423, and a light chain complimentarity determining region (vICDR1), a vICDR2 and a vICDR3 of a variable light chain domain (VL) having an amino acid sequence of SEQ ID NO:5; or
(xxvi) a vhCDR1, a vhCDR2 and a vhCDR3 of a variable heavy chain domain (VH) having an amino acid sequence of SEQ ID NO:431, and a light chain complimentarity determining region (vICDR1), a vICDR2 and a vICDR3 of a variable light chain domain (VL) having an amino acid sequence of SEQ ID NO:435.

In some embodiments, the anti-CD20 x anti-CD28 antibody includes a CD28 binding domain that comprises the CDRs selected from the following:
(i) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and (ii) a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and (ii) a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and
(ii) a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and
(ii) a vICDR1 having an amino acid sequence of SEQ ID NO:6, a vICDR2 having an amino acid sequence of SEQ ID NO:7, and a vICDR3 having an amino acid sequence of SEQ ID NO:22.

In some embodiments, the anti-CD20 x anti-CD28 antibody includes a CD28 binding domain that includes a VH and VL selected from the following:
(i) a VH having an amino acid sequence that is at least about 90, 95, 97, 98 or 99% identical to SEQ ID NO:1; and (ii) a VL having an amino acid sequence that is at least about 95% identical to SEQ ID NO:5; or
(i) a VH having an amino acid sequence that is at least about 90, 95, 97, 98 or 99% identical to SEQ ID NO:11; and (ii) a VL having an amino acid sequence that is at least about 90, 95, 97, 98 or 99% identical to SEQ ID NO:5; or
(i) a VH having an amino acid sequence that is at least about 90, 95, 97, 98 or 99% identical to SEQ ID NO:1; and (ii) a VL having an amino acid sequence that is at least about 90, 95, 97, 98 or 99% identical to SEQ ID NO:19; or
(i) a VH having an amino acid sequence that is at least about 90, 95, 97, 98 or 99% identical to SEQ ID NO:11; and (ii) a VL having an amino acid sequence that is at least about 90, 95, 97, 98 or 99% identical to SEQ ID NO:19; or
(i) a VH having an amino acid sequence that is at least about 90, 95, 97, 98 or 99% identical to SEQ ID NO:15; and (ii) a VL having an amino acid sequence that is at least about 90, 95, 97, 98 or 99% identical to SEQ ID NO:5; or
(i) a VH having an amino acid sequence that is at least about 90, 95, 97, 98 or 99% identical to SEQ ID NO:15; and (ii) a VL having an amino acid sequence that is at least about 90, 95, 97, 98 or 99% identical to SEQ ID NO:19; or
(i) a VH having an amino acid sequence that is at least about 90, 95, 97, 98 or 99% identical to SEQ ID NO:63; and (ii) a VL having an amino acid sequence that is at least about 90, 95, 97, 98 or 99% identical to SEQ ID NO:75; or
(i) a VH having an amino acid sequence that is at least about 90, 95, 97, 98 or 99% identical to SEQ ID NO:67; and (ii) a VL having an amino acid sequence that is at least about 90, 95, 97, 98 or 99% identical to SEQ ID NO:75; or
(i) a VH having an amino acid sequence that is at least about 90, 95, 97, 98 or 99% identical to SEQ ID NO:63; and (ii) a VL having an amino acid sequence that is at least about 90, 95, 97, 98 or 99% identical to SEQ ID NO:79; or
(i) a VH having an amino acid sequence that is at least about 90, 95, 97, 98 or 99% identical to SEQ ID NO:67; and (ii) a VL having an amino acid sequence that is at least about 90, 95, 97, 98 or 99% identical to SEQ ID NO:111; or
(i) a VH having an amino acid sequence that is at least about 90, 95, 97, 98 or 99% identical to SEQ ID NO:71; and (ii) a VL having an amino acid sequence that is at least about 90, 95, 97, 98 or 99% identical to SEQ ID NO:75; or
(i) a VH having an amino acid sequence that is at least about 90, 95, 97, 98 or 99% identical to SEQ ID NO:71; and (ii) a VL having an amino acid sequence that is at least about 90, 95, 97, 98 or 99% identical to SEQ ID NO:79.

In some embodiments, the CD28 ABD includes a VH that is at least 90, 95, 97, 98 or 99% identical to one of the following: SEQ ID NOs: 1, 11, 15, 63, 67, 71, 132-196, 320, 327, 335, 343, 351, 359, 367, 375, 383, 391, 399, 407, 415, 423, and 431. In some embodiments, the CD28 ABD includes a VL that is at least 90, 95, 97, 98 or 99% identical to one of the following: 5, 19, 75, 79, 200-305, 324, 331, 339, 347, 355, 371, 379, 387, 395, 403, 411, 419, and 435. In certain embodiments, the CD28 ABD is capable of binding to CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In some embodiments, the anti-CD20 x anti-CD28 antibody includes a CD28 binding domain that includes a VH and VL selected from the following:
(i) a VH having an amino acid sequence of SEQ ID NO:1; and (ii) a VL having an amino acid sequence of SEQ ID NO:5; or
(i) a VH having an amino acid sequence of SEQ ID NO:11; and (ii) a VL having an amino acid sequence of SEQ ID NO:5; or
(i) a VH having an amino acid sequence of SEQ ID NO:1; and (ii) a VL having an amino acid sequence of SEQ ID NO:19; or
(i) a VH having an amino acid sequence of SEQ ID NO:11; and (ii) a VL having an amino acid sequence of SEQ ID NO:19; or
(i) a VH having an amino acid sequence of SEQ ID NO:15; and (ii) a VL having an amino acid sequence of SEQ ID NO:5; or
(i) a VH having an amino acid sequence of SEQ ID NO:15; and (ii) a VL having an amino acid sequence of SEQ ID NO:19; or
(i) a VH having an amino acid sequence of SEQ ID NO:63; and (ii) a VL having an amino acid sequence of SEQ ID NO:75; or
(i) a VH having an amino acid sequence of SEQ ID NO:67; and (ii) a VL having an amino acid sequence of SEQ ID NO:75; or
(i) a VH having an amino acid sequence of SEQ ID NO:63; and (ii) a VL having an amino acid sequence of SEQ ID NO:79; or
(i) a VH having an amino acid sequence of SEQ ID NO:67; and (ii) a VL having an amino acid sequence of SEQ ID NO:111; or
(i) a VH having an amino acid sequence of SEQ ID NO:71; and (ii) a VL having an amino acid sequence of SEQ ID NO:75; or
(i) a VH having an amino acid sequence of SEQ ID NO:71; and (ii) a VL having an amino acid sequence of SEQ ID NO:79.

In some embodiments, the CD28 binding domain included in the anti-CD20 x anti-CD28 antibody includes a vhCDR1 having an amino acid of SEQ ID NO:307, a vhCDR2 having an amino acid sequence of SEQ ID NO:309, and/or a vhCDR3 having an amino acid sequence of SEQ ID NO: 311. In some embodiments, the CD28 binding domain included in the anti-CD20 x anti-CD28 antibody includes a vICDR1 having an amino acid of SEQ ID NO:314, a vICDR2 having an amino acid sequence of SEQ ID NO:316, and/or a vICDR3 having an amino acid sequence of SEQ ID NO:318.

### B. CD20 Binding Domains

CD20 binding domain. In some embodiments, subject antibodies that include such CD20 antigen binding domains (e.g., anti-CD20 x anti-CD28 bispecific antibodies) advantageously target cells that express CD20.

As will be appreciated by those in the art, suitable CD20 binding domains can comprise a set of 6 CDRs as depicted in the sequence listing and FIG. 30, either as the CDRs are underlined or, in the case where a different numbering scheme is used as described herein and as shown in Table 2, as the CDRs that are identified using other alignments within the variable heavy (VH) domain and variable light domain (VL) sequences of those depicted in FIG. 30 and the sequence listing (see Table 2). Suitable CD20 ABDs can also include the entire VH and VL sequences as depicted in these sequences and figures, used as scFvs or as Fab domains.

In some embodiments, the CD20 antigen binding domain comprises a variable heavy (VH) domain and a variable light (VL) domain, wherein the VH is selected from the amino acid sequences represented by SEQ ID NOs: 439, 447, 455, 463, 471, 473, 475, 477, 479, 481, 483, 487, 489, 491, 495, and 497; and the VL is selected from the amino acid sequences represented by SEQ ID NOs: 443, 451, 459, 467, 472, 474, 476, 478, 480, 482, 484, 490, 492, 496, and 498.

In one embodiment, the CD20 antigen binding domain of the anti-CD20 x anti-CD28 antibody includes the 6 CDRs (i.e., vhCDR1-3 and vICDR1-3) of a CD20 ABD described herein, including the Figures and sequence listing. In exemplary embodiments, the CD20 ABD is one of the following CD20 ABDs: CD20-A_H1L1, CD20-A_H1.202_L1.113, CD20-B_H1L1, CD20-C, rituximab, Obinutuzumab, ibritumomab, ocrelizumab, mosunetuzumab, ublituximab, veltuzumab, glofitamab, epcoritamab, TRU-015_SMIP, odronextamab, ripertamab, and ocaratuzumab (FIG. 30).

In addition to the parental CDR sets disclosed in the figures and sequence listing that form an ABD to CD20, provided herein are variant CD20 ABDs having CDRs that include at least one modification of the CD20 ABD CDRs disclosed herein. In one embodiment, the CD20 ABD includes a set of 6 CDRs with 1, 2, 3, 4, 5, 6, 7, 8, 9, 10 amino acid modifications as compared to the 6 CDRs of a CD20 ABD described herein, including the figures and sequence listing. In exemplary embodiments, the CD20 ABD of the anti-CD20 x anti-CD28 antibody includes a set of 6 CDRs with 1, 2, 3, 4, 5, 6, 7, 8, 9, 10 amino acid modifications as compared to the 6 CDRs of a CD20 ABD having one of the following VH and VL:
(i) a VH having an amino acid sequence of SEQ ID NO:439; and (ii) a VL having an amino acid sequence of SEQ ID NO:443; or
(i) a VH having an amino acid sequence of SEQ ID NO:447; and (ii) a VL having an amino acid sequence of SEQ ID NO:451; or
(i) a VH having an amino acid sequence of SEQ ID NO:455; and (ii) a VL having an amino acid sequence of SEQ ID NO:459; or
(i) a VH having an amino acid sequence of SEQ ID NO:463; and (ii) a VL having an amino acid sequence of SEQ ID NO:467; or
(i) a VH having an amino acid sequence of SEQ ID NO:471; and (ii) a VL having an amino acid sequence of SEQ ID NO:472; or
(i) a VH having an amino acid sequence of SEQ ID NO:473; and (ii) a VL having an amino acid sequence of SEQ ID NO:474; or
(i) a VH having an amino acid sequence of SEQ ID NO:475; and (ii) a VL having an amino acid sequence of SEQ ID NO:472; or
(i) a VH having an amino acid sequence of SEQ ID NO:477; and (ii) a VL having an amino acid sequence of SEQ ID NO:478; or
(i) a VH having an amino acid sequence of SEQ ID NO:479; and (ii) a VL having an amino acid sequence of SEQ ID NO:480; or
(i) a VH having an amino acid sequence of SEQ ID NO:481; and (ii) a VL having an amino acid sequence of SEQ ID NO:482; or
(i) a VH having an amino acid sequence of SEQ ID NO:483; and (ii) a VL having an amino acid sequence of SEQ ID NO:484; or
(i) a VH having an amino acid sequence of SEQ ID NO:487; and (ii) a VL having an amino acid sequence of SEQ ID NO:467; or
(i) a VH having an amino acid sequence of SEQ ID NO:489; and (ii) a VL having an amino acid sequence of SEQ ID NO:490; or
(i) a VH having an amino acid sequence of SEQ ID NO:491; and (ii) a VL having an amino acid sequence of SEQ ID NO:492; or
(i) a VH having an amino acid sequence of SEQ ID NO:495; and (ii) a VL having an amino acid sequence of SEQ ID NO:496; or
(i) a VH having an amino acid sequence of SEQ ID NO:497; and (ii) a VL having an amino acid sequence of SEQ ID NO:498 (FIG. 30).

In certain embodiments, the variant CD20 ABD is capable of binding CD20 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD20 ABD is capable of binding human CD20 antigen (see FIG. 2).

In one embodiment, the CD20 ABD of the anti-CD20 x anti-CD28 antibody includes 6 CDRs that are at least 90, 95, 97, 98 or 99% identical to the 6 CDRs of a CD20 ABD as described herein, including the figures and sequence listing. In exemplary embodiments, the CD20 ABD includes 6 CDRs that are at least 90, 95, 97, 98 or 99% identical to the 6 CDRs of a CD20 ABD having one of the following VH and VL:
(i) a VH having an amino acid sequence of SEQ ID NO:439; and (ii) a VL having an amino acid sequence of SEQ ID NO:443; or
(i) a VH having an amino acid sequence of SEQ ID NO:447; and (ii) a VL having an amino acid sequence of SEQ ID NO:451; or
(i) a VH having an amino acid sequence of SEQ ID NO:455; and (ii) a VL having an amino acid sequence of SEQ ID NO:459; or
(i) a VH having an amino acid sequence of SEQ ID NO:463; and (ii) a VL having an amino acid sequence of SEQ ID NO:467; or
(i) a VH having an amino acid sequence of SEQ ID NO:471; and (ii) a VL having an amino acid sequence of SEQ ID NO:472; or
(i) a VH having an amino acid sequence of SEQ ID NO:473; and (ii) a VL having an amino acid sequence of SEQ ID NO:474; or
(i) a VH having an amino acid sequence of SEQ ID NO:475; and (ii) a VL having an amino acid sequence of SEQ ID NO:472; or
(i) a VH having an amino acid sequence of SEQ ID NO:477; and (ii) a VL having an amino acid sequence of SEQ ID NO:478; or
(i) a VH having an amino acid sequence of SEQ ID NO:479; and (ii) a VL having an amino acid sequence of SEQ ID NO:480; or
(i) a VH having an amino acid sequence of SEQ ID NO:481; and (ii) a VL having an amino acid sequence of SEQ ID NO:482; or
(i) a VH having an amino acid sequence of SEQ ID NO:483; and (ii) a VL having an amino acid sequence of SEQ ID NO:484; or
(i) a VH having an amino acid sequence of SEQ ID NO:487; and (ii) a VL having an amino acid sequence of SEQ ID NO:467; or
(i) a VH having an amino acid sequence of SEQ ID NO:489; and (ii) a VL having an amino acid sequence of SEQ ID NO:490; or
(i) a VH having an amino acid sequence of SEQ ID NO:491; and (ii) a VL having an amino acid sequence of SEQ ID NO:492; or
(i) a VH having an amino acid sequence of SEQ ID NO:495; and (ii) a VL having an amino acid sequence of SEQ ID NO:496; or
(i) a VH having an amino acid sequence of SEQ ID NO:497; and (ii) a VL having an amino acid sequence of SEQ ID NO:498 (FIG. 30).

In certain embodiments, the CD20 ABD is capable of binding to CD20 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD20 ABD is capable of binding human CD20 antigen (see FIG. 2).

In another exemplary embodiment, the CD20 ABD of the anti-CD20 x anti-CD28 antibody include the variable heavy (VH) domain and variable light (VL) domain of any one of the CD20 ABDs described herein, including the figures and sequence listing. In exemplary embodiments, the CD20 ABD includes one of the following VH and VL:
(i) a VH having an amino acid sequence of SEQ ID NO:439; and (ii) a VL having an amino acid sequence of SEQ ID NO:443; or
(i) a VH having an amino acid sequence of SEQ ID NO:447; and (ii) a VL having an amino acid sequence of SEQ ID NO:451; or
(i) a VH having an amino acid sequence of SEQ ID NO:455; and (ii) a VL having an amino acid sequence of SEQ ID NO:459; or
(i) a VH having an amino acid sequence of SEQ ID NO:463; and (ii) a VL having an amino acid sequence of SEQ ID NO:467; or
(i) a VH having an amino acid sequence of SEQ ID NO:471; and (ii) a VL having an amino acid sequence of SEQ ID NO:472; or
(i) a VH having an amino acid sequence of SEQ ID NO:473; and (ii) a VL having an amino acid sequence of SEQ ID NO:474; or
(i) a VH having an amino acid sequence of SEQ ID NO:475; and (ii) a VL having an amino acid sequence of SEQ ID NO:472; or
(i) a VH having an amino acid sequence of SEQ ID NO:477; and (ii) a VL having an amino acid sequence of SEQ ID NO:478; or
(i) a VH having an amino acid sequence of SEQ ID NO:479; and (ii) a VL having an amino acid sequence of SEQ ID NO:480; or
(i) a VH having an amino acid sequence of SEQ ID NO:481; and (ii) a VL having an amino acid sequence of SEQ ID NO:482; or
(i) a VH having an amino acid sequence of SEQ ID NO:483; and (ii) a VL having an amino acid sequence of SEQ ID NO:484; or
(i) a VH having an amino acid sequence of SEQ ID NO:487; and (ii) a VL having an amino acid sequence of SEQ ID NO:467; or
(i) a VH having an amino acid sequence of SEQ ID NO:489; and (ii) a VL having an amino acid sequence of SEQ ID NO:490; or
(i) a VH having an amino acid sequence of SEQ ID NO:491; and (ii) a VL having an amino acid sequence of SEQ ID NO:492; or
(i) a VH having an amino acid sequence of SEQ ID NO:495; and (ii) a VL having an amino acid sequence of SEQ ID NO:496; or
(i) a VH having an amino acid sequence of SEQ ID NO:497; and (ii) a VL having an amino acid sequence of SEQ ID NO:498 (FIG. 30).

In addition to the parental CD20 ABD variable heavy and variable light domains disclosed herein, provided herein are CD20 ABDs that include a variable heavy domain and/or a variable light domain that are variants of a CD20 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a CD20 ABD described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of a CD20 ABD having one of the following VH and VL:
(i) a VH having an amino acid sequence of SEQ ID NO:439; and (ii) a VL having an amino acid sequence of SEQ ID NO:443; or
(i) a VH having an amino acid sequence of SEQ ID NO:447; and (ii) a VL having an amino acid sequence of SEQ ID NO:451; or
(i) a VH having an amino acid sequence of SEQ ID NO:455; and (ii) a VL having an amino acid sequence of SEQ ID NO:459; or
(i) a VH having an amino acid sequence of SEQ ID NO:463; and (ii) a VL having an amino acid sequence of SEQ ID NO:467; or
(i) a VH having an amino acid sequence of SEQ ID NO:471; and (ii) a VL having an amino acid sequence of SEQ ID NO:472; or
(i) a VH having an amino acid sequence of SEQ ID NO:473; and (ii) a VL having an amino acid sequence of SEQ ID NO:474; or
(i) a VH having an amino acid sequence of SEQ ID NO:475; and (ii) a VL having an amino acid sequence of SEQ ID NO:472; or
(i) a VH having an amino acid sequence of SEQ ID NO:477; and (ii) a VL having an amino acid sequence of SEQ ID NO:478; or
(i) a VH having an amino acid sequence of SEQ ID NO:479; and (ii) a VL having an amino acid sequence of SEQ ID NO:480; or
(i) a VH having an amino acid sequence of SEQ ID NO:481; and (ii) a VL having an amino acid sequence of SEQ ID NO:482; or
(i) a VH having an amino acid sequence of SEQ ID NO:483; and (ii) a VL having an amino acid sequence of SEQ ID NO:484; or
(i) a VH having an amino acid sequence of SEQ ID NO:487; and (ii) a VL having an amino acid sequence of SEQ ID NO:467; or
(i) a VH having an amino acid sequence of SEQ ID NO:489; and (ii) a VL having an amino acid sequence of SEQ ID NO:490; or
(i) a VH having an amino acid sequence of SEQ ID NO:491; and (ii) a VL having an amino acid sequence of SEQ ID NO:492; or
(i) a VH having an amino acid sequence of SEQ ID NO:495; and (ii) a VL having an amino acid sequence of SEQ ID NO:496; or
(i) a VH having an amino acid sequence of SEQ ID NO:497; and (ii) a VL having an amino acid sequence of SEQ ID NO:498 (FIG. 30).

In some embodiments, the changes are in a VH domain depicted in FIG. 30. In some embodiments, the changes are in a VL domain are depicted in FIG. 30. In some embodiments, the changes are in a VH and VL domain are depicted in FIG. 30. In some embodiments, the amino acid change(s) is in the VH and/or VL framework regions (FR1, FR2, FR3, and/or FR4). In certain embodiments, the CD20 ABD of the anti-CD20 x anti-CD28 antibody is capable of binding to CD20, as measured at least one of a Biacore, surface plasmon resonance (SPR), flow cytometry, and/or BLI (biolayer interferometry, e.g., Octet assay) assay, with the latter finding particular use in many embodiments. In particular embodiments, the CD20 ABD is capable of binding human CD20 antigen.

In one embodiment, the variant VH and/or VL domain is at least 90, 95, 97, 98 or 99% identical to the VH and/or VL of a CD20 ABD as described herein, including the figures and sequence listing. In exemplary embodiments, the variant VH and/or VL domain is at least 90, 95, 97, 98 or 99% identical to the VH and/or VL of a CD20 ABD having one of the following VH and VL:
(i) a VH having an amino acid sequence of SEQ ID NO:439; and (ii) a VL having an amino acid sequence of SEQ ID NO:443; or
(i) a VH having an amino acid sequence of SEQ ID NO:447; and (ii) a VL having an amino acid sequence of SEQ ID NO:451; or
(i) a VH having an amino acid sequence of SEQ ID NO:455; and (ii) a VL having an amino acid sequence of SEQ ID NO:459; or
(i) a VH having an amino acid sequence of SEQ ID NO:463; and (ii) a VL having an amino acid sequence of SEQ ID NO:467; or
(i) a VH having an amino acid sequence of SEQ ID NO:471; and (ii) a VL having an amino acid sequence of SEQ ID NO:472; or
(i) a VH having an amino acid sequence of SEQ ID NO:473; and (ii) a VL having an amino acid sequence of SEQ ID NO:474; or
(i) a VH having an amino acid sequence of SEQ ID NO:475; and (ii) a VL having an amino acid sequence of SEQ ID NO:472; or
(i) a VH having an amino acid sequence of SEQ ID NO:477; and (ii) a VL having an amino acid sequence of SEQ ID NO:478; or
(i) a VH having an amino acid sequence of SEQ ID NO:479; and (ii) a VL having an amino acid sequence of SEQ ID NO:480; or
(i) a VH having an amino acid sequence of SEQ ID NO:481; and (ii) a VL having an amino acid sequence of SEQ ID NO:482; or
(i) a VH having an amino acid sequence of SEQ ID NO:483; and (ii) a VL having an amino acid sequence of SEQ ID NO:484; or
(i) a VH having an amino acid sequence of SEQ ID NO:487; and (ii) a VL having an amino acid sequence of SEQ ID NO:467; or
(i) a VH having an amino acid sequence of SEQ ID NO:489; and (ii) a VL having an amino acid sequence of SEQ ID NO:490; or
(i) a VH having an amino acid sequence of SEQ ID NO:491; and (ii) a VL having an amino acid sequence of SEQ ID NO:492; or
(i) a VH having an amino acid sequence of SEQ ID NO:495; and (ii) a VL having an amino acid sequence of SEQ ID NO:496; or
(i) a VH having an amino acid sequence of SEQ ID NO:497; and (ii) a VL having an amino acid sequence of SEQ ID NO:498 (FIG. 30).

In some embodiments, the CD20 ABD includes a VH that is at least 90, 95, 97, 98 or 99% identical to VH domain depicted in FIG. 30. In some embodiments, the CD20 ABD includes a VL that is at least 90, 95, 97, 98 or 99% identical to VL domain depicted in FIG. 30. In some embodiments, the CD20 ABD includes a VH and a VL that is at least 90, 95, 97, 98 or 99% identical to a VH and a VL domains depicted in FIG. 30. In certain embodiments, the CD20 ABD of the anti-CD20 x anti-CD28 antibody is capable of binding to the CD20, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD20 ABD is capable of binding human CD20 antigen.

In some embodiments, the anti-CD20 x anti-CD28 antibody is a bivalent antibody (e.g., 1+1 Fab-scFv-Fc format antibody) that includes one CD20 binding domain. In other embodiments, the anti-CD20 x anti-CD28 antibody is a trivalent antibody (e.g., 2+1 Fab₂scFv-Fc format antibody) that includes two CD20 binding domains.

In some embodiments, the CDRs of the CD20 binding domain(s) are selected from the following:
(i) a heavy chain complimentarity determining region 1 (vhCDR1), a vhCDR2 and a vhCDR3 of a variable heavy chain domain (VH) having an amino acid sequence of SEQ ID NO:439, and a light chain complimentarity determining region 1 (vICDR1), a vICDR2 and a vICDR3 of a variable light chain domain (VL) having an amino acid sequence of SEQ ID NO:443;
(ii) a vhCDR1, a vhCDR2 and a vhCDR3 of a variable heavy chain domain (VH) having an amino acid sequence of SEQ ID NO:447, and a light chain complimentarity determining region (vICDR1), a vICDR2 and a vICDR3 of a variable light chain domain (VL) having an amino acid sequence of SEQ ID NO:451;
(iii) a vhCDR1, a vhCDR2 and a vhCDR3 of a variable heavy chain domain (VH) having an amino acid sequence of SEQ ID NO:455, and a light chain complimentarity determining region (vICDR1), a vICDR2 and a vICDR3 of a variable light chain domain (VL) having an amino acid sequence of SEQ ID NO:459;
(iv) a vhCDR1, a vhCDR2 and a vhCDR3 of a variable heavy chain domain (VH) having an amino acid sequence of SEQ ID NO:463, and a light chain complimentarity determining region (vICDR1), a vlCDR2 and a vlCDR3 of a variable light chain domain (VL) having an amino acid sequence of SEQ ID NO:467;
(v) a vhCDR1, a vhCDR2 and a vhCDR3 of a variable heavy chain domain (VH) having an amino acid sequence of SEQ ID NO:471, and a light chain complimentarity determining region (vICDR1), a vlCDR2 and a vlCDR3 of a variable light chain domain (VL) having an amino acid sequence of SEQ ID NO:472;
(vi) a vhCDR1, a vhCDR2 and a vhCDR3 of a variable heavy chain domain (VH) having an amino acid sequence of SEQ ID NO:473, and a light chain complimentarity determining region (vICDR1), a vlCDR2 and a vlCDR3 of a variable light chain domain (VL) having an amino acid sequence of SEQ ID NO:474;
(vii) a vhCDR1, a vhCDR2 and a vhCDR3 of a variable heavy chain domain (VH) having an amino acid sequence of SEQ ID NO:475, and a light chain complimentarity determining region (vICDR1), a vlCDR2 and a vlCDR3 of a variable light chain domain (VL) having an amino acid sequence of SEQ ID NO:472;
(viii) a vhCDR1, a vhCDR2 and a vhCDR3 of a variable heavy chain domain (VH) having an amino acid sequence of SEQ ID NO:477, and a light chain complimentarity determining region (vICDR1), a vlCDR2 and a vlCDR3 of a variable light chain domain (VL) having an amino acid sequence of SEQ ID NO:478;
(ix) a vhCDR1, a vhCDR2 and a vhCDR3 of a variable heavy chain domain (VH) having an amino acid sequence of SEQ ID NO:479, and a light chain complimentarity determining region (vICDR1), a vlCDR2 and a vlCDR3 of a variable light chain domain (VL) having an amino acid sequence of SEQ ID NO:480;
(x) a vhCDR1, a vhCDR2 and a vhCDR3 of a variable heavy chain domain (VH) having an amino acid sequence of SEQ ID NO:481, and a light chain complimentarity determining region (vICDR1), a vlCDR2 and a vlCDR3 of a variable light chain domain (VL) having an amino acid sequence of SEQ ID NO:482;
(xi) a vhCDR1, a vhCDR2 and a vhCDR3 of a variable heavy chain domain (VH) having an amino acid sequence of SEQ ID NO:483, and a light chain complimentarity determining region (vICDR1), a vlCDR2 and a vlCDR3 of a variable light chain domain (VL) having an amino acid sequence of SEQ ID NO:484;
(xii) a vhCDR1, a vhCDR2 and a vhCDR3 of a variable heavy chain domain (VH) having an amino acid sequence of SEQ ID NO:487, and a light chain complimentarity determining region (vICDR1), a vICDR2 and a vICDR3 of a variable light chain domain (VL) having an amino acid sequence of SEQ ID NO:467;
(xiii) a vhCDR1, a vhCDR2 and a vhCDR3 of a variable heavy chain domain (VH) having an amino acid sequence of SEQ ID NO:489, and a light chain complimentarity determining region (vICDR1), a vICDR2 and a vICDR3 of a variable light chain domain (VL) having an amino acid sequence of SEQ ID NO:490;
(xiv) a vhCDR1, a vhCDR2 and a vhCDR3 of a variable heavy chain domain (VH) having an amino acid sequence of SEQ ID NO:491, and a light chain complimentarity determining region (vICDR1), a vICDR2 and a vICDR3 of a variable light chain domain (VL) having an amino acid sequence of SEQ ID NO:492;
(xv) a vhCDR1, a vhCDR2 and a vhCDR3 of a variable heavy chain domain (VH) having an amino acid sequence of SEQ ID NO:495, and a light chain complimentarity determining region (vICDR1), a vICDR2 and a vICDR3 of a variable light chain domain (VL) having an amino acid sequence of SEQ ID NO:496; or
(xvi) a vhCDR1, a vhCDR2 and a vhCDR3 of a variable heavy chain domain (VH) having an amino acid sequence of SEQ ID NO:497, and a light chain complimentarity determining region (vICDR1), a vICDR2 and a vICDR3 of a variable light chain domain (VL) having an amino acid sequence of SEQ ID NO:498.

In some embodiments, the CDRs of the CD20 binding domain(s) are selected from the following:
(i) a vhCDR1 having an amino acid sequence of SEQ ID NO:440; a vhCDR2 having an amino acid sequence of SEQ ID NO:441; and a vhCDR3 having an amino acid sequence of SEQ ID NO:442; and (ii) a vICDR1 having an amino acid sequence of SEQ ID NO:444, a vICDR2 having an amino acid sequence of SEQ ID NO: 445, and a vICDR3 having an amino acid sequence of SEQ ID NO:446; or
(i) a vhCDR1 having an amino acid sequence of SEQ ID NO:440; a vhCDR2 having an amino acid sequence of SEQ ID NO:449; and a vhCDR3 having an amino acid sequence of SEQ ID NO:450; and (ii) a vICDR1 having an amino acid sequence of SEQ ID NO:452, a vICDR2 having an amino acid sequence of SEQ ID NO:445, and a vICDR3 having an amino acid sequence of SEQ ID NO:454;
(i) a vhCDR1 having an amino acid sequence of SEQ ID NO:440; a vhCDR2 having an amino acid sequence of SEQ ID NO:457; and a vhCDR3 having an amino acid sequence of SEQ ID NO:458; and (ii) a vICDR1 having an amino acid sequence of SEQ ID NO:460, a vICDR2 having an amino acid sequence of SEQ ID NO: 461, and a vICDR3 having an amino acid sequence of SEQ ID NO:462; or
(i) a vhCDR1 having an amino acid sequence of SEQ ID NO:464; a vhCDR2 having an amino acid sequence of SEQ ID NO:465; and a vhCDR3 having an amino acid sequence of SEQ ID NO:466; and (ii) a vICDR1 having an amino acid sequence of SEQ ID NO:356, a vICDR2 having an amino acid sequence of SEQ ID NO:357, and a vICDR3 having an amino acid sequence of SEQ ID NO:470.

In some embodiments, the VH and VL of the CD20 binding domain(s) are selected from the following:
(i) a VH having an amino acid sequence that is at least about 95% identical to SEQ ID NO:439; and (ii) a VL having an amino acid sequence that is at least about 95% identical to SEQ ID NO:443; or
(i) a VH having an amino acid sequence that is at least about 95% identical to SEQ ID NO:447; and (ii) a VL having an amino acid sequence that is at least about 95% identical to SEQ ID NO:451; or
(i) a VH having an amino acid sequence that is at least about 95% identical to SEQ ID NO:455; and (ii) a VL having an amino acid sequence that is at least about 95% identical to SEQ ID NO:459; or
(i) a VH having an amino acid sequence that is at least about 95% identical to SEQ ID NO:463; and (ii) a VL having an amino acid sequence that is at least about 95% identical to SEQ ID NO:467.

In some embodiments, the VH and VL of the CD20 binding domain(s) are selected from the following:
(i) a VH having an amino acid sequence of SEQ ID NO:439; and (ii) a VL having an amino acid sequence of SEQ ID NO:443; or
(i) a VH having an amino acid sequence of SEQ ID NO:447; and (ii) a VL having an amino acid sequence of SEQ ID NO:451; or
(i) a VH having an amino acid sequence of SEQ ID NO:455; and (ii) a VL having an amino acid sequence of SEQ ID NO:459; or
(i) a VH having an amino acid sequence of SEQ ID NO:463; and (ii) a VL having an amino acid sequence of SEQ ID NO:467.

### C. Chimeric and Humanized Antibodies

In certain embodiments, the subject antibodies provided herein include a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene (prior to the introduction of any skew, pI and ablation variants herein; that is, the number of variants is generally low, prior to the introduction of the variants of the invention). In certain cases, the humanized antibody may display no more than 5, or even no more than 4, 3, 2, or 1 amino acid difference from the amino acid sequence encoded by the germline immunoglobulin gene (again, prior to the introduction of any skew, pI and ablation variants herein; that is, the number of variants is generally low, prior to the introduction of the variants of the invention).

In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

### D. Heterodimeric Antibodies

In exemplary embodiments, the anti-CD20 x anti-CD28 antibodies provided herein are heterodimeric bispecific antibodies that include two variant Fc domain sequences. Such variant Fc domains include amino acid modifications to facilitate the self-assembly and/or purification of the heterodimeric antibodies.

An ongoing problem in antibody technologies is the desire for “bispecific” antibodies that bind to two different antigens simultaneously, in general thus allowing the different antigens to be brought into proximity and resulting in new functionalities and new therapies. In general, these antibodies are made by including genes for each heavy and light chain into the host cells. This generally results in the formation of the desired heterodimer (A-B), as well as the two homodimers (A-A and B-B (not including the light chain heterodimeric issues)). However, a major obstacle in the formation of bispecific antibodies is the difficulty in biasing the formation of the desired heterodimeric antibody over the formation of the homodimers and/or purifying the heterodimeric antibody away from the homodimers.

There are a number of mechanisms that can be used to generate the subject heterodimeric antibodies. In addition, as will be appreciated by those in the art, these different mechanisms can be combined to ensure high heterodimerization. Amino acid modifications that facilitate the production and purification of heterodimers are collectively referred to generally as “heterodimerization variants.” As discussed below, heterodimerization variants include “skew” variants (e.g., the “knobs and holes” and the “charge pairs” variants described below) as well as “pI variants,” which allow purification of heterodimers from homodimers. As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

Heterodimerization variants that are useful for the formation and purification of the subject heterodimeric antibody (e.g., bispecific antibodies) are further discussed in detailed below.

### 1. Skew Variants

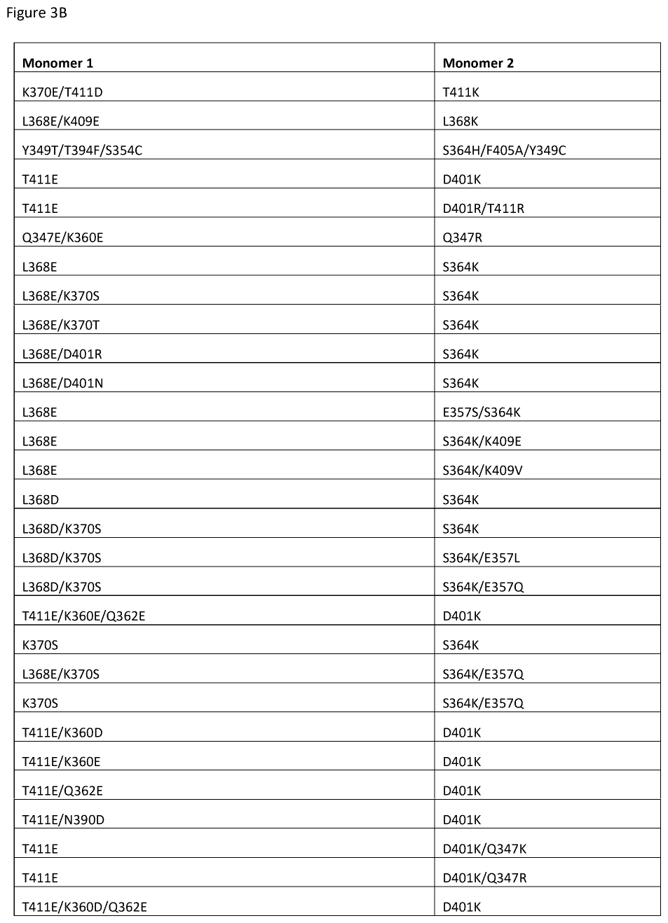

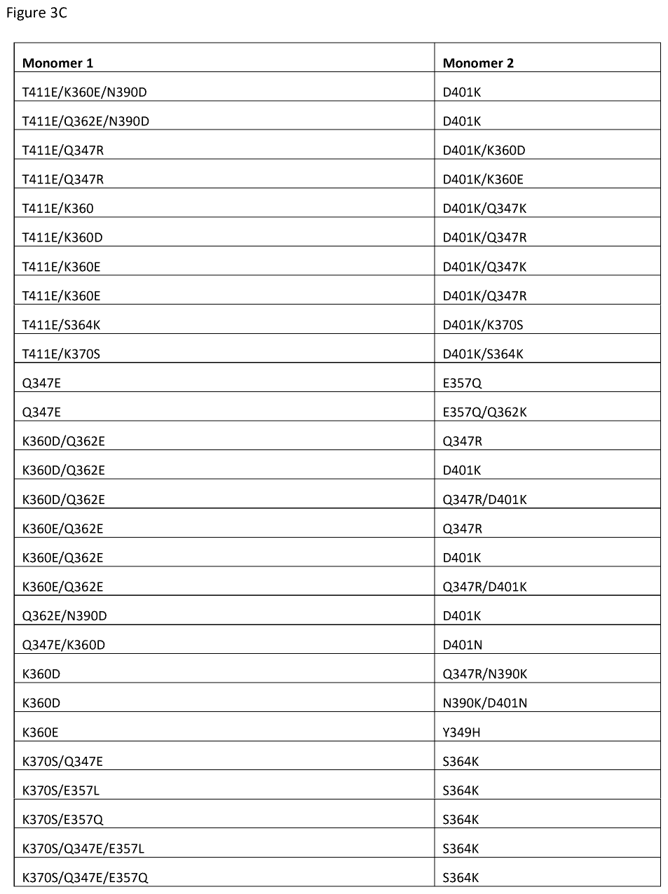

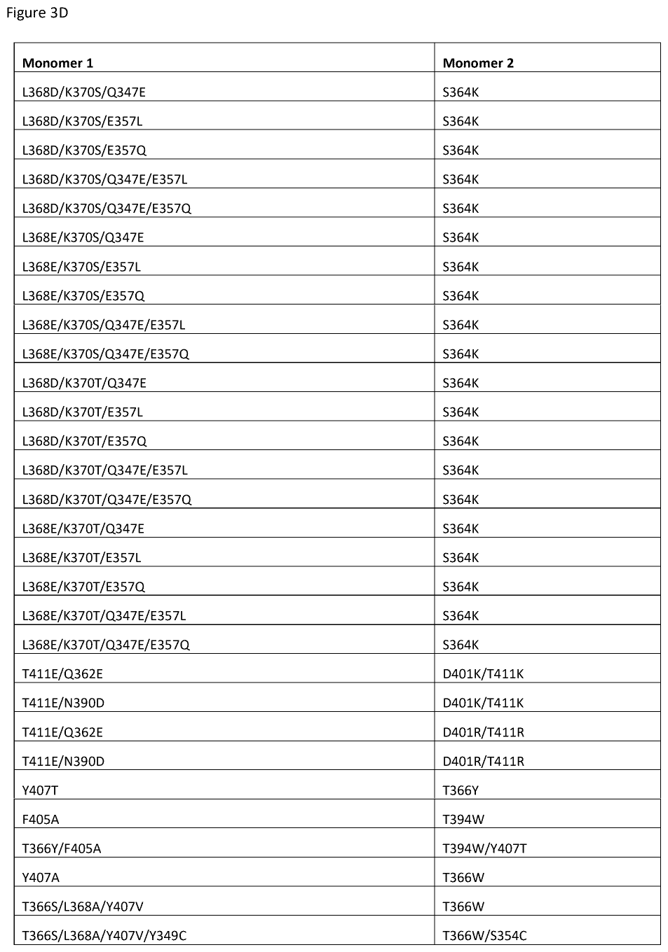

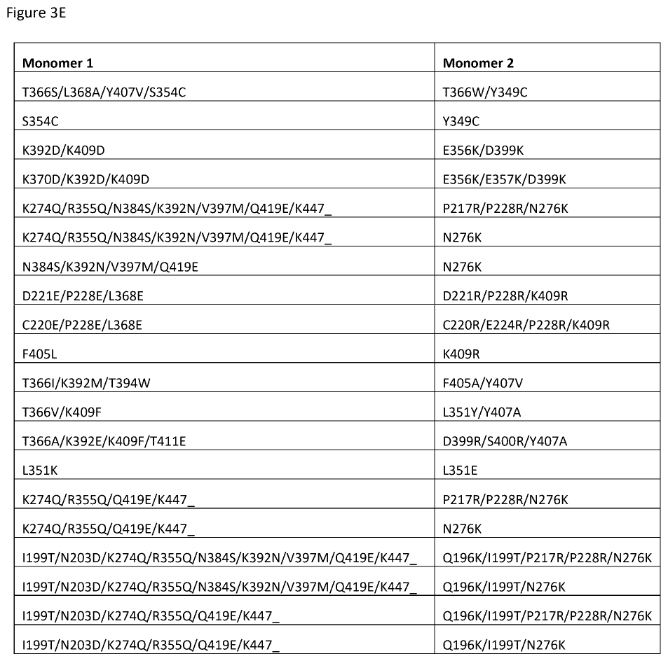

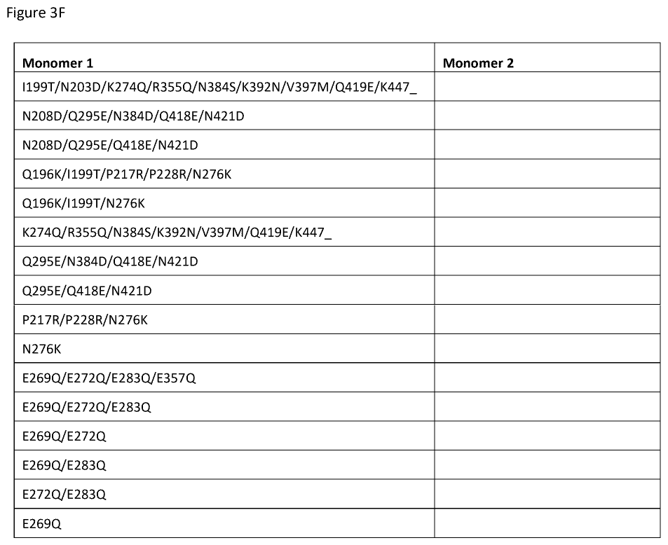

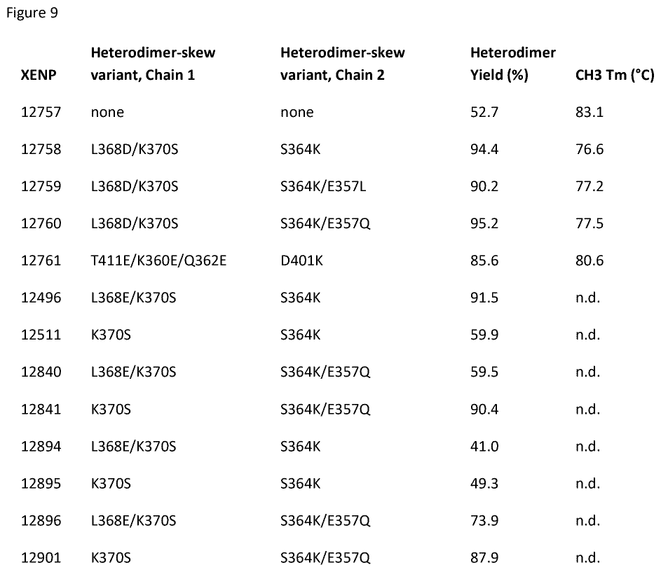

In some embodiments, the heterodimeric antibody includes skew variants which are one or more amino acid modifications in a first Fc domain (A) and/or a second Fc domain (B) that favor the formation of Fc heterodimers (Fc dimers that include the first and the second Fc domain; (A-B) over Fc homodimers (Fc dimers that include two of the first Fc domain or two of the second Fc domain; A-A or B-B). Suitable skew variants are included in the FIG. 29 of US Publ. App. No. 2016/0355608, hereby incorporated by reference in its entirety and specifically for its disclosure of skew variants, as well as in FIGS. 3 and 9.

One particular type of skew variants is generally referred to in the art as “knobs and holes,” referring to amino acid engineering that creates steric influences to favor heterodimeric formation and disfavor homodimeric formation, as described in U.S. Ser. No. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety and specifically for the disclosure of “knobs and holes” mutations. This is sometime referred to herein as “steric variants.” The figures identify a number of “monomer A-monomer B” pairs that rely on “knobs and holes”. In addition, as described in Merchant et al., Nature Biotech. 16:677 (1998), these “knobs and holes” mutations can be combined with disulfide bonds to further favor formation of Fc heterodimers.

Another method that finds use in the generation of heterodimers is sometimes referred to as “electrostatic steering” as described in Gunasekaran et al., J. Biol. Chem. 285(25):19637 (2010), hereby incorporated by reference in its entirety. This is sometimes referred to herein as “charge pairs”. In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “skew variants”. These include, but are not limited to, D221E/P228E/L368E paired with D221R/P228R/K409R (e.g., these are “monomer corresponding sets) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanism. In some embodiments, the heterodimeric antibody includes one or more sets of such heterodimerization skew variants. These variants come in “pairs” of “sets”. That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B). Exemplary heterodimerization “skew” variants are depicted in FIGS. 3 and 9. Such “skew” variants include, but are not limited to: S364K/E357Q:L368D/K370S; L368D/K370S:S364K; L368E/K370S:S364K; T411T/E360E/Q362E:D401K; L368D/K370S:S364K/E357L; K370S:S364K/E357Q (EU numbering).

In exemplary embodiments, the heterodimeric antibody includes a S364K/E357Q:L368D/K370S; L368D/K370S:S364K; L368E/K370S:S364K; T411T/E360E/Q362E:D401K; L368D/K370S:S364K/E357L; K370S:S364K/E357Q; or a T366S/L368A/Y407V:T366W (optionally including a bridging disulfide, T366S/L368A/Y407V/Y349C:T366W/S354C) “skew” variant amino acid substitution set (EU numbering). In an exemplary embodiment, the heterodimeric antibody includes a “S364K/E357Q:L368D/K370S” amino acid substitution set. In terms of nomenclature, the pair “S364K/E357Q:L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI.

In some embodiments, Fc heterodimerization domains that result in heterodimerization of two different Fc domains comprise amino acid variants in the Fc domain including, but not limited to, these pairs: S364K/E357Q:L368D/K370S; L368D/K370S:S364K; L368E/K370S:S364K; T411T/E360E/Q362E:D401K; L368D/K370S:S364K/E357L; K370S:S364K/E357Q; or a T366S/L368A/Y407V:T366W (optionally including a bridging disulfide, T366S/L368A/Y407V/Y349C:T366W/S354C or T366S/L368A/Y407V/Y354C:T366W/S349C) “skew” variant amino acid substitution set (EU numbering), as well as others in FIG. 1. In general, these heterodimerization Fc variants are made in human IgG1, IgG2 or IgG4 backbones.

In some embodiments, the skew variants provided herein can be optionally and independently incorporated with any other modifications, including, but not limited to, other skew variants (see, e.g., in FIG. 37 of US Publ. App. No. 2012/0149876, herein incorporated by reference, particularly for its disclosure of skew variants), pI variants, isotpypic variants, FcRn variants, ablation variants, etc. into one or both of the first and second Fc domains of the heterodimeric antibody. Further, individual modifications can also independently and optionally be included or excluded from the subject the heterodimeric antibody.

In some embodiments, the skew variants outlined herein can be optionally and independently incorporated with any pI variant (or other variants such as Fc variants, FcRn variants, etc.) into one or both heavy chain monomers, and can be independently and optionally included or excluded from the subject heterodimeric antibodies.

### 2. Purification Variants

In some embodiments, the heterodimeric antibody includes purification variants that advantageously allow for the separation of heterodimeric proteins (e.g., anti-CD20 x anti-CD28 bispecific antibody) from homodimeric proteins.

There are several basic mechanisms that can lead to ease of purifying heterodimeric antibodies. For example, modifications to one or both of the antibody heavy chain monomers A and B such that each monomer has a different pI allows for the isoelectric purification of heterodimeric A-B antibody from monomeric A-A and B-B proteins. Alternatively, some scaffold formats, such as the “1+1 Fab-scFv-Fc” format, and the “2+1 Fab₂-scFv-Fc” format, allows separation on the basis of size. As described above, it is also possible to “skew” the formation of heterodimers over homodimers using skew variants. Thus, a combination of heterodimerization skew variants and purification variants find particular use in the heterodimeric antibodies provided herein.

Additionally, as more fully outlined below, depending on the format of the heterodimeric antibody, purification variants either contained within the constant region and/or Fc domains of a monomer, and/or domain linkers can be used. In some embodiments, the heterodimeric antibody includes additional modifications for alternative functionalities that can also create pI changes, such as Fc, FcRn and KO variants.

In some embodiments, the subject heterodimeric antibodies provided herein include at least one monomer with one or more modifications that alter the pI of the monomer (i.e., a “pI variant”). In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

Depending on the format of the heterodimer antibody, pI variants can be either contained within the constant and/or Fc domains of a monomer, or charged linkers, either domain linkers or scFv linkers, can be used. That is, antibody formats that utilize scFv(s) such as “1+1 Fab-scFv-Fc”, format can include charged scFv linkers (either positive or negative), that give a further pI boost for purification purposes. As will be appreciated by those in the art, some 1+1 Fab-scFv-Fc and 2+1 Fab₂-scFv-Fc formats are useful with just charged scFv linkers and no additional pI adjustments, although the invention does provide pI variants that are on one or both of the monomers, and/or charged domain linkers as well. In addition, additional amino acid engineering for alternative functionalities may also confer pI changes, such as Fc, FcRn and KO variants.

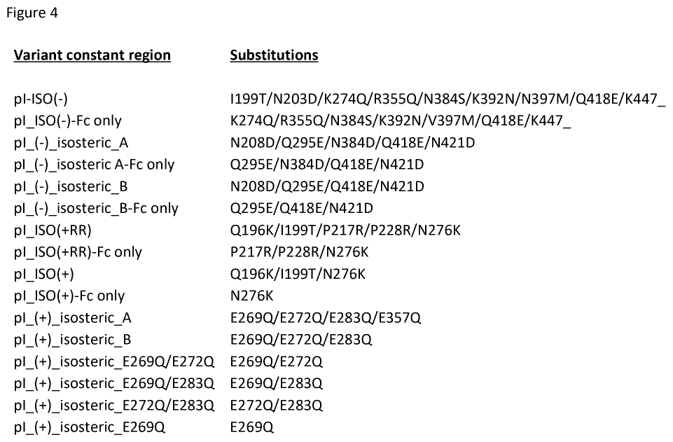

In subject heterodimeric antibodies that utilizes pI as a separation mechanism to allow the purification of heterodimeric proteins, amino acid variants are introduced into one or both of the monomer polypeptides. That is, the pI of one of the monomers (referred to herein for simplicity as “monomer A”) can be engineered away from monomer B, or both monomer A and B change be changed, with the pI of monomer A increasing and the pI of monomer B decreasing. As is outlined more fully below, the pI changes of either or both monomers can be done by removing or adding a charged residue (e.g., a neutral amino acid is replaced by a positively or negatively charged amino acid residue, e.g., glycine to glutamic acid), changing a charged residue from positive or negative to the opposite charge (aspartic acid to lysine) or changing a charged residue to a neutral residue (e.g., loss of a charge; lysine to serine). A number of these variants are shown in the FIGS. 3 and 4.

Thus, in some embodiments, the subject heterodimeric antibody includes amino acid modifications in the constant regions that alter the isoelectric point (pI) of at least one, if not both, of the monomers of a dimeric protein to form “pI antibodies”) by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the present invention.

As will be appreciated by those in the art, the number of pI variants to be included on each or both monomer(s) to get good separation will depend in part on the starting pI of the components, for example in the 1+1 Fab-scFv-Fc, 2+1 Fab₂-scFv-Fc, 1+1 CLC and 2+1 CLC formats, the starting pI of the scFv (1+1 Fab-scFv-Fc, 2+1 Fab₂-scFv-Fc) and Fab(s) of interest. That is, to determine which monomer to engineer or in which “direction” (e.g., more positive or more negative), the Fv sequences of the two target antigens are calculated and a decision is made from there. As is known in the art, different Fvs will have different starting pIs which are exploited in the present invention. In general, as outlined herein, the pIs are engineered to result in a total pI difference of each monomer of at least about 0.1 logs, with 0.2 to 0.5 being preferred as outlined herein.

In the case where pI variants are used to achieve heterodimerization, by using the constant region(s) of the heavy chain(s), a more modular approach to designing and purifying bispecific proteins, including antibodies, is provided. Thus, in some embodiments, heterodimerization variants (including skew and pI heterodimerization variants) are not included in the variable regions, such that each individual antibody must be engineered. In addition, in some embodiments, the possibility of immunogenicity resulting from the pI variants is significantly reduced by importing pI variants from different IgG isotypes such that pI is changed without introducing significant immunogenicity. Thus, an additional problem to be solved is the elucidation of low pI constant domains with high human sequence content, e.g., the minimization or avoidance of non-human residues at any particular position. Alternatively or in addition to isotypic substitutions, the possibility of immunogenicity resulting from the pI variants is significantly reduced by utilizing isosteric substitutions (e.g. Asn to Asp; and Gln to Glu).

As discussed below, a side benefit that can occur with this pI engineering is also the extension of serum half-life and increased FcRn binding. That is, as described in US Publ. App. No. US 2012/0028304 (incorporated by reference in its entirety), lowering the pI of antibody constant domains (including those found in antibodies and Fc fusions) can lead to longer serum retention in vivo. These pI variants for increased serum half-life also facilitate pI changes for purification.

In addition, it should be noted that the pI variants give an additional benefit for the analytics and quality control process of bispecific antibodies, as the ability to either eliminate, minimize and distinguish when homodimers are present is significant. Similarly, the ability to reliably test the reproducibility of the heterodimeric antibody production is important.

In general, embodiments of particular use rely on sets of variants that include skew variants, which encourage heterodimerization formation over homodimerization formation, coupled with pI variants, which increase the pI difference between the two monomers to facilitate purification of heterodimers away from homodimers.

Exemplary combinations of pI variants are shown in FIGS. 4 and 5, and FIG. 30 of US Publ. App. No. 2016/0355608, all of which are herein incorporated by reference in its entirety and specifically for the disclosure of pI variants. Preferred combinations of pI variants are shown in FIGS. 3 and 4. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)₄ (SEQ ID NO:42). However, as will be appreciated by those in the art, the first monomer includes a CH1 domain, including position 208. Accordingly, in constructs that do not include a CH1 domain (for example for antibodies that do not utilize a CH1 domain on one of the domains), a preferred negative pI variant Fc set includes 295E/384D/418E/421D variants (Q295E/N384D/Q418E/N421D when relative to human IgG1).

Accordingly, in some embodiments, one monomer has a set of substitutions from FIG. 8 and the other monomer has a charged linker (either in the form of a charged scFv linker because that monomer comprises an scFv or a charged domain linker, as the format dictates, which can be selected from those depicted in FIG. 6).

In some embodiments, modifications are made in the hinge of the Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., an antibody provided herein may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389,392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

In some embodiments, the anti-CD20 x anti-CD28 antibody includes amino acid substitutions in one of its Fc domains that reduces binding to Protein A. Such purification variants produces heterodimers with asymmetric binding to Protein A, which can in turn be used for separation of heterodimeric from homodimeric populations by a pH gradient. Exemplary purification amino acid substitutions that reduce binding to Protein A include, but are not limited to H435R and Y436F (IgG1 CH3 domain, EU numbering). See, e.g., US2010331527, which is incorporated by reference in its entirety, and specifically for pertinent disclosures relating to Fc domain modifications to reduce Protein A binding.

### 3. Isotypic Variants

In addition, many embodiments of the subject heterodimeric antibodies rely on the “importation” of pI amino acids at particular positions from one IgG isotype into another, thus reducing or eliminating the possibility of unwanted immunogenicity being introduced into the variants. A number of these are shown in FIG. 21 of US Publ. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significantly affect the pI of the variant antibody. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pI amino acid to a lower pI amino acid), or to allow accommodations in structure for stability, etc. as is further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point.

### 4. Calculating pI

The pI of each monomer of the antibodies provided herein can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of US Pub. 2014/0370013. As discussed herein, which monomer to engineer is generally decided by the inherent pI of the Fv and scaffold regions. Alternatively, the pI of each monomer can be compared.

5. pI Variants that Also Confer Better FcRn In Vivo Binding

In the case where the pI variant decreases the pI of the monomer, the pI variant can have the added benefit of improving serum retention in vivo.

Although still under examination, Fc regions are believed to have longer half-lives in vivo, because binding to FcRn at pH 6 in an endosome sequesters the Fc (Ghetie and Ward, 1997 Immunol Today. 18(12): 592-598, entirely incorporated by reference). The endosomal compartment then recycles the Fc to the cell surface. Once the compartment opens to the extracellular space, the higher pH, ˜7.4, induces the release of Fc back into the blood. In mice, Dall' Acqua et al. showed that Fc mutants with increased FcRn binding at pH 6 and pH 7.4 actually had reduced serum concentrations and the same half-life as wild-type Fc (Dall' Acqua et al. 2002, J. Immunol. 169:5171-5180, entirely incorporated by reference). The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

Recently it has been suggested that antibodies with variable regions that have lower isoelectric points may also have longer serum half-lives (Igawa et al., 2010 PEDS. 23(5): 385-392, entirely incorporated by reference). However, the mechanism of this is still poorly understood. Moreover, variable regions differ from antibody to antibody. Constant region variants with reduced pI and extended half-life would provide a more modular approach to improving the pharmacokinetic properties of antibodies, as described herein.

### E. Additional Fc Variants for Additional Functionality

In addition to the heterodimerization variants discussed above, there are a number of useful Fc amino acid modification that can be made for a variety of reasons, including, but not limited to, altering binding to one or more FcγR receptors, altered binding to FcRn receptors, etc., as discussed below.

Accordingly, the antibodies provided herein (heterodimeric, as well as homodimeric) can include such amino acid modifications with or without the heterodimerization variants outlined herein (e.g., the pI variants and steric variants). Each set of variants can be independently and optionally included or excluded from any particular heterodimeric protein.

### 1. FcγR and FcRn Variants

Accordingly, there are a number of useful Fc substitutions that can be made to alter binding to one or more of the FcγR receptors. In certain embodiments, the subject antibody includes modifications that alter the binding to one or more FcγR receptors (i.e., “FcγR variants”). Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the subject antibodies include those listed in U.S. Pat. No. 8,188,321 (particularly FIG. 41) and U.S. Pat. No. 8,084,582, and US Publ. App. Nos. 20060235208 and 20070148170, all of which are expressly incorporated herein by reference in their entirety and specifically for the variants disclosed therein that affect Fcγ receptor binding. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T. Such modification may be included in one or both Fc domains of the subject antibody.

In some embodiments, the subject antibody includes one or more Fc modifications that increase serum half-life. Fc substitutions that find use in increased binding to the FcRn receptor and increased serum half-life, as specifically disclosed in U.S. Ser. No. 12/341,769, hereby incorporated by reference in its entirety, including, but not limited to, 434S, 434A, 428L, 308F, 259I, 428L/434S, 259I/308F, 436I/428L, 436I or V/434S, 436V/428L, 259I/308F/428L, and M252Y/S254T/T256E. Such modification may be included in one or both Fc domains of the subject antibody.

### 2. Ablation Variants

In some embodiments, the heterodimeric antibody includes one or more modifications that reduce or remove the normal binding of the Fc domain to one or more or all of the Fcγ receptors (e.g., FcγR1, FcγRIIa, FcγRIIb, FcγRIIIa, etc.) to avoid additional mechanisms of action. Such modifications are referred to as “FcγR ablation variants” or “Fc knock out (FcKO or KO)” variants. In these embodiments, for some therapeutic applications, it is desirable to reduce or remove the normal binding of the Fc domain to one or more or all of the Fcγ receptors (e.g., FcγR1, FcγRIIa, FcγRIIb, FcγRIIIa, etc.) to avoid additional mechanisms of action. That is, for example, in many embodiments, particularly in the use of bispecific antibodies that bind CD28 monovalently, it is generally desirable to ablate FcγRIIIa binding to eliminate or significantly reduce ADCC activity. In some embodiments, of the subject antibodies described herein, at least one of the Fc domains comprises one or more Fcγ receptor ablation variants. In some embodiments, of the subject antibodies described herein, both of the Fc domains comprises one or more Fcγ receptor ablation variants. These ablation variants are depicted in FIG. 5, and each can be independently and optionally included or excluded, with preferred embodiments utilizing ablation variants selected from the group consisting of: L234A/L235A/D265S, G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del. It should be noted that the ablation variants referenced herein ablate FcγR binding but generally not FcRn binding.

As is known in the art, the Fc domain of human IgG1 has the highest binding to the Fcγ receptors, and thus ablation variants can be used when the constant domain (or Fc domain) in the backbone of the heterodimeric antibody is IgG1. Alternatively, or in addition to ablation variants in an IgG1 background, mutations at the glycosylation position 297 (generally to A or S) can significantly ablate binding to FcγRIIIa, for example. Human IgG2 and IgG4 have naturally reduced binding to the Fcγ receptors, and thus those backbones can be used with or without the ablation variants.

### F. Combination of Heterodimeric and Fc Variants

As will be appreciated by those in the art, all of the recited heterodimerization variants (including skew and/or purification variants) can be optionally and independently combined in any way, as long as they retain their “strandedness” or “monomer partition”. In addition, all of these variants can be combined into any of the heterodimerization formats.

In the case of pI variants, while embodiments finding particular use are shown in the figures, other combinations can be generated, following the basic rule of altering the pI difference between two monomers to facilitate purification.

In addition, any of the heterodimerization variants (skew and purification variants), are also independently and optionally combined with Fc ablation variants, Fc variants, FcRn variants, as generally outlined herein.

Exemplary combination of variants that are included in some embodiments of the heterodimeric 1+1 Fab-scFv-Fc, 2+1 Fab₂-scFv-Fc, 2+1 stack Fab₂-scFv-Fc, and 2+1 mAb-scFv format antibodies are included in FIG. 8. In some embodiments, the heterodimeric antibody includes a combination of variants as depicted in FIG. 8. In certain embodiments, the antibody is a heterodimeric 1+1 Fab-scFv-Fc, 2+1 Fab₂-scFv-Fc, 2+1 stack Fab₂-scFv-Fc, and 2+1 mAb-scFv format antibody that includes the “platform X” combination of variants depicted in FIG. 8. In certain embodiments, the antibody is a heterodimeric 1+1 Fab-scFv-Fc, 2+1 Fab₂-scFv-Fc, 2+1 stack Fab₂-scFv-Fc, and 2+1 mAb-scFv format antibody that includes the “platform Y” combination of variants depicted in FIG. 8.

### G. Useful Antibody Formats

As will be appreciated by those in the art and discussed more fully below, the heterodimeric bispecific antibodies provided herein can take on several different configurations as generally depicted in FIG. 29.

As will be appreciated by those in the art, the heterodimeric formats of the invention can have different valencies as well as be bispecific. That is, heterodimeric antibodies of the invention can be bivalent and bispecific, or trivalent and bispecific, wherein the first antigen is bound by two binding domains and the second antigen by a second binding domain. As is outlined herein, when CD28 is one of the target antigens, it is preferable that the CD28 is bound only monovalently.

The present invention utilizes CD28 binding domains in combination with CD20 binding domains. As will be appreciated by those in the art, any collection of anti-CD28 CDRs, anti-CD28 variable light and variable heavy domains, Fabs and scFvs as depicted in any of the Figures (see particularly FIGS. 15-24, and 26) can be used. Similarly, any of the CD20 antigen binding domains can be used, whether CDRs, variable light and variable heavy domains, Fabs and scFvs as depicted in any of the Figures (e.g., FIG. 30) can be used, optionally and independently combined in any combination.

### 1. 1+1 Fab-scFv-Fc Format

One heterodimeric antibody format that finds particular use in subject anti-CD20 x anti-CD28 antibodies provided herein is the “1+1 Fab-scFv-Fc” or “bottle opener” format as shown in FIG. 29A. The 1+1 Fab-scFv-Fc format antibody includes a first monomer that is a “regular” heavy chain (VH1-CH1-hinge-CH2-CH3), wherein VH1 is a first variable heavy domain and CH2-CH3 is a first Fc domain. The 1+1 Fab-scFv-Fc also includes a light chain that includes a first variable light domain VL1 and a constant light domain CL. The light chain interacts with the VH1-CH1 of the first monomer to form a first antigen binding domain that is a Fab. The second monomer of the antibody includes a second binding domain that is a single chain Fv (“scFv”, as defined below) and a second Fc domain. The scFv includes a second variable heavy domain (VH2) and a second variable light domain (VL2), wherein the VH2 is attached to the VL2 using an scFv linker that can be charged (see, e.g., FIG. 6). The scFv is attached to the heavy chain using a domain linker (see, e.g., FIG. 7). The two monomers are brought together by the use of amino acid variants (e.g., heterodimerization variants, discussed above) in the constant regions (e.g., the Fc domain, the CH1 domain and/or the hinge region) that promote the formation of heterodimeric antibodies as is described more fully below. This structure is sometimes referred to herein as the “bottle-opener” format, due to a rough visual similarity to a bottle-opener. In some embodiments, the 1+1 Fab-scFv-Fc format antibody is a bivalent antibody.

There are several distinct advantages to the present “1+1 Fab-scFv-Fc” format. As is known in the art, antibody analogs relying on two scFv constructs often have stability and aggregation problems, which can be alleviated in the present invention by the addition of a “regular” heavy and light chain pairing. In addition, as opposed to formats that rely on two heavy chains and two light chains, there is no issue with the incorrect pairing of heavy and light chains (e.g., heavy 1 pairing with light 2, etc.).

In some embodiments of the 1+1 Fab-scFv-Fc format antibody, one of the first or second antigen binding domain is a CD28 binding domain and the other binding domain is a CD20 binding domain. In some embodiments where the 1+1 Fab-scFv-Fc, it is the scFv that binds to the CD28, and the Fab that binds CD20. Exemplary anti-CD20 x anti-CD28 bispecific antibodies in the 1+1 Fab-scFv-Fc format are depicted in FIG. 33.

In some embodiments, the first and second Fc domains of the 1+1 Fab-scFv-Fc format antibody are variant Fc domains that include heterodimerization skew variants (e.g., a set of amino acid substitutions as shown in FIGS. 3 and 9). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 4). In exemplary embodiments, the constant domain (CH1-hinge-CH2-CH3) of the first monomer includes pI variants N208D/Q295E/N384D/Q418E/N421D, wherein numbering is according to EU numbering.

In exemplary embodiments, the 1+1 Fab-scFv-Fc format antibody include “Platform X” amino acid modifications as depicted in FIG. 8. In such embodiments, the CH1-hinge-CH2-CH3 of the first monomer comprises amino acid variants L368D/K370S/N208D/Q295E/N384D/Q418E/N421D/E233P/L234V/L235A/G236del/S267K, the second Fc domain comprises amino acid variants S364K/E357Q/E233P/L234V/L235A/G236del/S267K, wherein numbering is according to EU numbering.

In some embodiments, the scFv of the 1+1 Fab-scFv-Fc format antibody provided herein includes a charged scFv linker (including those shown in FIG. 6). In some embodiments, the 1+1 Fab-scFv-Fc format antibody provided herein includes FcRn variants M428L/N434S, wherein numbering is according to EU numbering.

In exemplary embodiments 1+1 Fab-scFv-Fc format antibody with “Platform X” variants, the first Fc domain includes heterodimerization skew variants L368D/K370S and the second Fc domain includes heterodimerization skew variants S364K/E357Q; each of the first and second Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 1+1 Fab-scFv-Fc format antibody provided herein includes a (GKPGS)₄ (SEQ ID NO:42) charged scFv linker. In some embodiments, the 1+1 Fab-scFv-Fc format antibody provided herein includes FcRn variants M428L/N434S, wherein numbering is according to EU numbering. In some embodiments, the scFv of the 1+1 Fab-scFv-Fc format antibody provided herein includes a charged scFv linker (including those shown in FIG. 6).

In exemplary embodiments, the 1+1 Fab-scFv-Fc format antibody include “Platform Y” amino acid modifications as depicted in FIG. 8. In such embodiments, the first Fc domain comprises amino acid substitutions L234A/L235A/D265S/T366S/L368A/Y407V/H435R/Y436F, and the first Fc domain comprises amino acid substitutions L234A/L235A/D265S/LT366W, wherein numbering is according to EU numbering.

In exemplary embodiments 1+1 Fab-scFv-Fc format antibody with Platform Y variants, the first Fc domain includes heterodimerization skew variants T366S/L368A/Y407V and the second Fc domain includes heterodimerization skew variants T366W; each of the first and second variant Fc domains include ablation variants L234A/L235A/D265S; and the second Fc domain includes purification variants H435R/Y436F, wherein numbering is according to EU numbering. In some embodiments, the scFv of the 1+1 Fab-scFv-Fc format antibody provided herein includes a (GKPGS)₄ (SEQ ID NO:42) charged scFv linker. In some embodiments, the 1+1 Fab-scFv-Fc format antibody provided herein includes FcRn variants M252Y/S254T/T256E, wherein numbering is according to EU numbering. In some embodiments, the scFv of the 1+1 Fab-scFv-Fc format antibody provided herein includes a charged scFv linker (including those shown in FIG. 6).

In exemplary embodiments, the 1+1 Fab-scFv-Fc format antibody with Platform J variants further includes a “stapled” scFv (e.g., a “stapled” anti-CD28 scFv). Stapled” scFvs that exhibit improved stability and/or reduced aggregation are further described in detail herein. Exemplary staple linkers that are useful for inclusion in such “stapled” scFvs are provided in FIG. 6. In exemplary embodiments, the stapled linker is GGGSGGSGGCPPCGGSGG (SEQ ID NO:56).

In some embodiments, one of the first binding domain or the second binding domain binds CD20 and the other binding domain binds a CD28. Any suitable CD28 binding domain can be included in subject 1+1 Fab-scFv-Fc format antibody, including any of the CD28 binding domains provided herein.

In some embodiments of the 1+1 Fab-scFv-Fc format, the anti-CD28 ABD has the CDRs selected from the following:
(i) a vhCDR1, a vhCDR2 and a vhCDR3 of a variable heavy chain domain (VH) having an amino acid sequence of SEQ ID NO:1, and a light chain complimentarity determining region (vICDR1), a vICDR2 and a vICDR3 of a variable light chain domain (VL) having an amino acid sequence of SEQ ID NO:5;
(ii) a vhCDR1, a vhCDR2 and a vhCDR3 of a variable heavy chain domain (VH) having an amino acid sequence of SEQ ID NO:11, and a light chain complimentarity determining region (vICDR1), a vICDR2 and a vICDR3 of a variable light chain domain (VL) having an amino acid sequence of SEQ ID NO:5;
(iii) a vhCDR1, a vhCDR2 and a vhCDR3 of a variable heavy chain domain (VH) having an amino acid sequence of SEQ ID NO:1, and a light chain complimentarity determining region (vICDR1), a vICDR2 and a vICDR3 of a variable light chain domain (VL) having an amino acid sequence of SEQ ID NO:19;
(iv) a vhCDR1, a vhCDR2 and a vhCDR3 of a variable heavy chain domain (VH) having an amino acid sequence of SEQ ID NO:11, and a light chain complimentarity determining region (vICDR1), a vICDR2 and a vICDR3 of a variable light chain domain (VL) having an amino acid sequence of SEQ ID NO:19;
(v) a vhCDR1, a vhCDR2 and a vhCDR3 of a variable heavy chain domain (VH) having an amino acid sequence of SEQ ID NO:15, and a light chain complimentarity determining region (vICDR1), a vlCDR2 and a vlCDR3 of a variable light chain domain (VL) having an amino acid sequence of SEQ ID NO:5;
(vi) a vhCDR1, a vhCDR2 and a vhCDR3 of a variable heavy chain domain (VH) having an amino acid sequence of SEQ ID NO:15, and a light chain complimentarity determining region (vICDR1), a vlCDR2 and a vlCDR3 of a variable light chain domain (VL) having an amino acid sequence of SEQ ID NO:19;
(vii) a vhCDR1, a vhCDR2 and a vhCDR3 of a variable heavy chain domain (VH) having an amino acid sequence of SEQ ID NO:63, and a light chain complimentarity determining region (vICDR1), a vlCDR2 and a vlCDR3 of a variable light chain domain (VL) having an amino acid sequence of SEQ ID NO:75;
(viii) a vhCDR1, a vhCDR2 and a vhCDR3 of a variable heavy chain domain (VH) having an amino acid sequence of SEQ ID NO:67, and a light chain complimentarity determining region (vICDR1), a vlCDR2 and a vlCDR3 of a variable light chain domain (VL) having an amino acid sequence of SEQ ID NO:75;
(ix) a vhCDR1, a vhCDR2 and a vhCDR3 of a variable heavy chain domain (VH) having an amino acid sequence of SEQ ID NO:63, and a light chain complimentarity determining region (vICDR1), a vlCDR2 and a vlCDR3 of a variable light chain domain (VL) having an amino acid sequence of SEQ ID NO:79;
(x) a vhCDR1, a vhCDR2 and a vhCDR3 of a variable heavy chain domain (VH) having an amino acid sequence of SEQ ID NO:67, and a light chain complimentarity determining region (vICDR1), a vlCDR2 and a vlCDR3 of a variable light chain domain (VL) having an amino acid sequence of SEQ ID NO:111;
(xi) a vhCDR1, a vhCDR2 and a vhCDR3 of a variable heavy chain domain (VH) having an amino acid sequence of SEQ ID NO:71, and a light chain complimentarity determining region (vICDR1), a vlCDR2 and a vlCDR3 of a variable light chain domain (VL) having an amino acid sequence of SEQ ID NO:75;
(xii) a vhCDR1, a vhCDR2 and a vhCDR3 of a variable heavy chain domain (VH) having an amino acid sequence of SEQ ID NO:71, and a light chain complimentarity determining region (vICDR1), a vlCDR2 and a vlCDR3 of a variable light chain domain (VL) having an amino acid sequence of SEQ ID NO:79;
(xiii) a vhCDR1, a vhCDR2 and a vhCDR3 of a variable heavy chain domain (VH) having an amino acid sequence of SEQ ID NO:320, and a light chain complimentarity determining region (vICDR1), a vlCDR2 and a vlCDR3 of a variable light chain domain (VL) having an amino acid sequence of SEQ ID NO:324;
(xiv) a vhCDR1, a vhCDR2 and a vhCDR3 of a variable heavy chain domain (VH) having an amino acid sequence of SEQ ID NO:327, and a light chain complimentarity determining region (vICDR1), a vlCDR2 and a vlCDR3 of a variable light chain domain (VL) having an amino acid sequence of SEQ ID NO:331;
(xv) a vhCDR1, a vhCDR2 and a vhCDR3 of a variable heavy chain domain (VH) having an amino acid sequence of SEQ ID NO:335, and a light chain complimentarity determining region (vICDR1), a vlCDR2 and a vlCDR3 of a variable light chain domain (VL) having an amino acid sequence of SEQ ID NO:339;
(xvi) a vhCDR1, a vhCDR2 and a vhCDR3 of a variable heavy chain domain (VH) having an amino acid sequence of SEQ ID NO:343, and a light chain complimentarity determining region (vICDR1), a vlCDR2 and a vlCDR3 of a variable light chain domain (VL) having an amino acid sequence of SEQ ID NO:347;
(xvii) a vhCDR1, a vhCDR2 and a vhCDR3 of a variable heavy chain domain (VH) having an amino acid sequence of SEQ ID NO:351, and a light chain complimentarity determining region (vICDR1), a vlCDR2 and a vlCDR3 of a variable light chain domain (VL) having an amino acid sequence of SEQ ID NO:355;
(xviii) a vhCDR1, a vhCDR2 and a vhCDR3 of a variable heavy chain domain (VH) having an amino acid sequence of SEQ ID NO:359, and a light chain complimentarity determining region (vICDR1), a vlCDR2 and a vlCDR3 of a variable light chain domain (VL) having an amino acid sequence of SEQ ID NO:355;
(xix) a vhCDR1, a vhCDR2 and a vhCDR3 of a variable heavy chain domain (VH) having an amino acid sequence of SEQ ID NO:367, and a light chain complimentarity determining region (vICDR1), a vlCDR2 and a vlCDR3 of a variable light chain domain (VL) having an amino acid sequence of SEQ ID NO:371;
(xx) a vhCDR1, a vhCDR2 and a vhCDR3 of a variable heavy chain domain (VH) having an amino acid sequence of SEQ ID NO:375, and a light chain complimentarity determining region (vICDR1), a vlCDR2 and a vlCDR3 of a variable light chain domain (VL) having an amino acid sequence of SEQ ID NO:380;
(xxi) a vhCDR1, a vhCDR2 and a vhCDR3 of a variable heavy chain domain (VH) having an amino acid sequence of SEQ ID NO:391, and a light chain complimentarity determining region (vICDR1), a vlCDR2 and a vlCDR3 of a variable light chain domain (VL) having an amino acid sequence of SEQ ID NO:395;
(xxii) a vhCDR1, a vhCDR2 and a vhCDR3 of a variable heavy chain domain (VH) having an amino acid sequence of SEQ ID NO:399, and a light chain complimentarity determining region (vICDR1), a vICDR2 and a vICDR3 of a variable light chain domain (VL) having an amino acid sequence of SEQ ID NO:403;
(xxiii) a vhCDR1, a vhCDR2 and a vhCDR3 of a variable heavy chain domain (VH) having an amino acid sequence of SEQ ID NO:407, and a light chain complimentarity determining region (vICDR1), a vICDR2 and a vICDR3 of a variable light chain domain (VL) having an amino acid sequence of SEQ ID NO:411;
(xxiv) a vhCDR1, a vhCDR2 and a vhCDR3 of a variable heavy chain domain (VH) having an amino acid sequence of SEQ ID NO:415, and a light chain complimentarity determining region (vICDR1), a vICDR2 and a vICDR3 of a variable light chain domain (VL) having an amino acid sequence of SEQ ID NO:419;
(xxv) a vhCDR1, a vhCDR2 and a vhCDR3 of a variable heavy chain domain (VH) having an amino acid sequence of SEQ ID NO:423, and a light chain complimentarity determining region (vICDR1), a vICDR2 and a vICDR3 of a variable light chain domain (VL) having an amino acid sequence of SEQ ID NO:5; or
(xxvi) a vhCDR1, a vhCDR2 and a vhCDR3 of a variable heavy chain domain (VH) having an amino acid sequence of SEQ ID NO:431, and a light chain complimentarity determining region (vICDR1), a vICDR2 and a vICDR3 of a variable light chain domain (VL) having an amino acid sequence of SEQ ID NO:435.

In some embodiments of the 1+1 Fab-scFv-Fc format, the anti-CD28 ABD has the CDRs selected from the following:
(i) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and (ii) a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and (ii) a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and
(ii) a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and
(ii) a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and (ii) a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and (ii) a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and
(ii) a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and
(ii) a vICDR1 having an amino acid sequence of SEQ ID NO:6, a vICDR2 having an amino acid sequence of SEQ ID NO:7, and a vICDR3 having an amino acid sequence of SEQ ID NO:22.

In some embodiments of the 1+1 Fab-scFv-Fc format, the anti-CD28 ABD has a VH and VL domain selected from the following or a variant thereof:
(i) a VH having an amino acid sequence of SEQ ID NO:1; and (ii) a VL having an amino acid sequence of SEQ ID NO:5; or
(i) a VH having an amino acid sequence of SEQ ID NO:11; and (ii) a VL having an amino acid sequence of SEQ ID NO:5; or
(i) a VH having an amino acid sequence of SEQ ID NO:1; and (ii) a VL having an amino acid sequence of SEQ ID NO:19; or
(i) a VH having an amino acid sequence of SEQ ID NO:11; and (ii) a VL having an amino acid sequence of SEQ ID NO:19; or
(i) a VH having an amino acid sequence of SEQ ID NO:15; and (ii) a VL having an amino acid sequence of SEQ ID NO:5; or
(i) a VH having an amino acid sequence of SEQ ID NO:15; and (ii) a VL having an amino acid sequence of SEQ ID NO:19; or
(i) a VH having an amino acid sequence of SEQ ID NO:63; and (ii) a VL having an amino acid sequence of SEQ ID NO:75; or
(i) a VH having an amino acid sequence of SEQ ID NO:67; and (ii) a VL having an amino acid sequence of SEQ ID NO:75; or
(i) a VH having an amino acid sequence of SEQ ID NO:63; and (ii) a VL having an amino acid sequence of SEQ ID NO:79; or
(i) a VH having an amino acid sequence of SEQ ID NO:67; and (ii) a VL having an amino acid sequence of SEQ ID NO:111; or
(i) a VH having an amino acid sequence of SEQ ID NO:71; and (ii) a VL having an amino acid sequence of SEQ ID NO:75; or
(i) a VH having an amino acid sequence of SEQ ID NO:71; and (ii) a VL having an amino acid sequence of SEQ ID NO:79; or
i) a VH having an amino acid sequence of SEQ ID NO:320; and (ii) a VL having an amino acid sequence of SEQ ID NO:324; or
(i) a VH having an amino acid sequence of SEQ ID NO:327; and (ii) a VL having an amino acid sequence of SEQ ID NO:331; or
(i) a VH having an amino acid sequence of SEQ ID NO:335; and (ii) a VL having an amino acid sequence of SEQ ID NO:339; or
(i) a VH having an amino acid sequence of SEQ ID NO:343; and (ii) a VL having an amino acid sequence of SEQ ID NO:347; or
(i) a VH having an amino acid sequence of SEQ ID NO:351; and (ii) a VL having an amino acid sequence of SEQ ID NO:355; or
(i) a VH having an amino acid sequence of SEQ ID NO:359; and (ii) a VL having an amino acid sequence of SEQ ID NO:355; or
(i) a VH having an amino acid sequence of SEQ ID NO:367; and (ii) a VL having an amino acid sequence of SEQ ID NO:371; or
(i) a VH having an amino acid sequence of SEQ ID NO:375; and (ii) a VL having an amino acid sequence of SEQ ID NO:380; or
(i) a VH having an amino acid sequence of SEQ ID NO:391; and (ii) a VL having an amino acid sequence of SEQ ID NO:395; or
(i) a VH having an amino acid sequence of SEQ ID NO:399; and (ii) a VL having an amino acid sequence of SEQ ID NO:403; or
(i) a VH having an amino acid sequence of SEQ ID NO:407; and (ii) a VL having an amino acid sequence of SEQ ID NO:411; or
(i) a VH having an amino acid sequence of SEQ ID NO:415; and (ii) a VL having an amino acid sequence of SEQ ID NO:419; or
(i) a VH having an amino acid sequence of SEQ ID NO:423; and (ii) a VL having an amino acid sequence of SEQ ID NO:5; or
(i) a VH having an amino acid sequence of SEQ ID NO:431; and (ii) a VL having an amino acid sequence of SEQ ID NO:435.

In some embodiments, one of the first binding domain or the second binding domain of the 1+1 Fab-scFv-Fc format antibody binds CD20. In some embodiments of the 1+1 Fab-scFv-Fc format, the anti-CD20 ABD has the CDRs selected from the following:
(i) a heavy chain complimentarity determining region 1 (vhCDR1), a vhCDR2 and a vhCDR3 of a variable heavy chain domain (VH) having an amino acid sequence of SEQ ID NO:439, and a light chain complimentarity determining region 1 (vICDR1), a vICDR2 and a vICDR3 of a variable light chain domain (VL) having an amino acid sequence of SEQ ID NO:443;
(ii) a vhCDR1, a vhCDR2 and a vhCDR3 of a variable heavy chain domain (VH) having an amino acid sequence of SEQ ID NO:447, and a light chain complimentarity determining region (vICDR1), a vICDR2 and a vICDR3 of a variable light chain domain (VL) having an amino acid sequence of SEQ ID NO:451;
(iii) a vhCDR1, a vhCDR2 and a vhCDR3 of a variable heavy chain domain (VH) having an amino acid sequence of SEQ ID NO:455, and a light chain complimentarity determining region (vICDR1), a vlCDR2 and a vlCDR3 of a variable light chain domain (VL) having an amino acid sequence of SEQ ID NO:459;
(iv) a vhCDR1, a vhCDR2 and a vhCDR3 of a variable heavy chain domain (VH) having an amino acid sequence of SEQ ID NO:463, and a light chain complimentarity determining region (vICDR1), a vlCDR2 and a vlCDR3 of a variable light chain domain (VL) having an amino acid sequence of SEQ ID NO:467;
(v) a vhCDR1, a vhCDR2 and a vhCDR3 of a variable heavy chain domain (VH) having an amino acid sequence of SEQ ID NO:471, and a light chain complimentarity determining region (vICDR1), a vlCDR2 and a vlCDR3 of a variable light chain domain (VL) having an amino acid sequence of SEQ ID NO:472;
(vi) a vhCDR1, a vhCDR2 and a vhCDR3 of a variable heavy chain domain (VH) having an amino acid sequence of SEQ ID NO:473, and a light chain complimentarity determining region (vICDR1), a vlCDR2 and a vlCDR3 of a variable light chain domain (VL) having an amino acid sequence of SEQ ID NO:474;
(vii) a vhCDR1, a vhCDR2 and a vhCDR3 of a variable heavy chain domain (VH) having an amino acid sequence of SEQ ID NO:475, and a light chain complimentarity determining region (vICDR1), a vlCDR2 and a vlCDR3 of a variable light chain domain (VL) having an amino acid sequence of SEQ ID NO:472;
(viii) a vhCDR1, a vhCDR2 and a vhCDR3 of a variable heavy chain domain (VH) having an amino acid sequence of SEQ ID NO:477, and a light chain complimentarity determining region (vICDR1), a vlCDR2 and a vlCDR3 of a variable light chain domain (VL) having an amino acid sequence of SEQ ID NO:478;
(ix) a vhCDR1, a vhCDR2 and a vhCDR3 of a variable heavy chain domain (VH) having an amino acid sequence of SEQ ID NO:479, and a light chain complimentarity determining region (vICDR1), a vlCDR2 and a vlCDR3 of a variable light chain domain (VL) having an amino acid sequence of SEQ ID NO:480;
(x) a vhCDR1, a vhCDR2 and a vhCDR3 of a variable heavy chain domain (VH) having an amino acid sequence of SEQ ID NO:481, and a light chain complimentarity determining region (vICDR1), a vlCDR2 and a vlCDR3 of a variable light chain domain (VL) having an amino acid sequence of SEQ ID NO:482;
(xi) a vhCDR1, a vhCDR2 and a vhCDR3 of a variable heavy chain domain (VH) having an amino acid sequence of SEQ ID NO:483, and a light chain complimentarity determining region (vICDR1), a vICDR2 and a vICDR3 of a variable light chain domain (VL) having an amino acid sequence of SEQ ID NO:484;
(xii) a vhCDR1, a vhCDR2 and a vhCDR3 of a variable heavy chain domain (VH) having an amino acid sequence of SEQ ID NO:487, and a light chain complimentarity determining region (vICDR1), a vICDR2 and a vICDR3 of a variable light chain domain (VL) having an amino acid sequence of SEQ ID NO:467;
(xiii) a vhCDR1, a vhCDR2 and a vhCDR3 of a variable heavy chain domain (VH) having an amino acid sequence of SEQ ID NO:489, and a light chain complimentarity determining region (vICDR1), a vICDR2 and a vICDR3 of a variable light chain domain (VL) having an amino acid sequence of SEQ ID NO:490;
(xiv) a vhCDR1, a vhCDR2 and a vhCDR3 of a variable heavy chain domain (VH) having an amino acid sequence of SEQ ID NO:491, and a light chain complimentarity determining region (vICDR1), a vICDR2 and a vICDR3 of a variable light chain domain (VL) having an amino acid sequence of SEQ ID NO:492;
(xv) a vhCDR1, a vhCDR2 and a vhCDR3 of a variable heavy chain domain (VH) having an amino acid sequence of SEQ ID NO:495, and a light chain complimentarity determining region (vICDR1), a vICDR2 and a vICDR3 of a variable light chain domain (VL) having an amino acid sequence of SEQ ID NO:496; or
(xvi) a vhCDR1, a vhCDR2 and a vhCDR3 of a variable heavy chain domain (VH) having an amino acid sequence of SEQ ID NO:497, and a light chain complimentarity determining region (vICDR1), a vICDR2 and a vICDR3 of a variable light chain domain (VL) having an amino acid sequence of SEQ ID NO:498.

In some embodiments of the 1+1 Fab-scFv-Fc format, the anti-CD20 ABD has the CDRs selected from the following:
(i) a vhCDR1 having an amino acid sequence of SEQ ID NO:440; a vhCDR2 having an amino acid sequence of SEQ ID NO:441; and a vhCDR3 having an amino acid sequence of SEQ ID NO:442; and (ii) a vICDR1 having an amino acid sequence of SEQ ID NO:444, a vICDR2 having an amino acid sequence of SEQ ID NO: 445, and a vICDR3 having an amino acid sequence of SEQ ID NO:446; or
(i) a vhCDR1 having an amino acid sequence of SEQ ID NO:440; a vhCDR2 having an amino acid sequence of SEQ ID NO:449; and a vhCDR3 having an amino acid sequence of SEQ ID NO:450; and (ii) a vICDR1 having an amino acid sequence of SEQ ID NO:452, a vICDR2 having an amino acid sequence of SEQ ID NO:445, and a vICDR3 having an amino acid sequence of SEQ ID NO:454;
(i) a vhCDR1 having an amino acid sequence of SEQ ID NO:440; a vhCDR2 having an amino acid sequence of SEQ ID NO:457; and a vhCDR3 having an amino acid sequence of SEQ ID NO:458; and (ii) a vICDR1 having an amino acid sequence of SEQ ID NO:460, a vICDR2 having an amino acid sequence of SEQ ID NO: 461, and a vICDR3 having an amino acid sequence of SEQ ID NO:462; or
(i) a vhCDR1 having an amino acid sequence of SEQ ID NO:464; a vhCDR2 having an amino acid sequence of SEQ ID NO:465; and a vhCDR3 having an amino acid sequence of SEQ ID NO:466; and (ii) a vICDR1 having an amino acid sequence of SEQ ID NO:356, a vICDR2 having an amino acid sequence of SEQ ID NO:357, and a vICDR3 having an amino acid sequence of SEQ ID NO:470.

In some embodiments of the 1+1 Fab-scFv-Fc format, the anti-CD20 ABD has a VH and VL domain selected from the following:
(i) a VH having an amino acid sequence of SEQ ID NO:439; and (ii) a VL having an amino acid sequence of SEQ ID NO:443; or
(i) a VH having an amino acid sequence of SEQ ID NO:447; and (ii) a VL having an amino acid sequence of SEQ ID NO:451; or
(i) a VH having an amino acid sequence of SEQ ID NO:455; and (ii) a VL having an amino acid sequence of SEQ ID NO:459; or
(i) a VH having an amino acid sequence of SEQ ID NO:463; and (ii) a VL having an amino acid sequence of SEQ ID NO:467 or
(i) a VH having an amino acid sequence of SEQ ID NO:471; and (ii) a VL having an amino acid sequence of SEQ ID NO:472; or
(i) a VH having an amino acid sequence of SEQ ID NO:473; and (ii) a VL having an amino acid sequence of SEQ ID NO:474; or
(i) a VH having an amino acid sequence of SEQ ID NO:475; and (ii) a VL having an amino acid sequence of SEQ ID NO:472; or
(i) a VH having an amino acid sequence of SEQ ID NO:477; and (ii) a VL having an amino acid sequence of SEQ ID NO:478; or
(i) a VH having an amino acid sequence of SEQ ID NO:479; and (ii) a VL having an amino acid sequence of SEQ ID NO:480; or
(i) a VH having an amino acid sequence of SEQ ID NO:481; and (ii) a VL having an amino acid sequence of SEQ ID NO:482; or
(i) a VH having an amino acid sequence of SEQ ID NO:483; and (ii) a VL having an amino acid sequence of SEQ ID NO:484; or
(i) a VH having an amino acid sequence of SEQ ID NO:487; and (ii) a VL having an amino acid sequence of SEQ ID NO:467; or
(i) a VH having an amino acid sequence of SEQ ID NO:489; and (ii) a VL having an amino acid sequence of SEQ ID NO:490; or
(i) a VH having an amino acid sequence of SEQ ID NO:491; and (ii) a VL having an amino acid sequence of SEQ ID NO:492; or
(i) a VH having an amino acid sequence of SEQ ID NO:495; and (ii) a VL having an amino acid sequence of SEQ ID NO:496; or
(i) a VH having an amino acid sequence of SEQ ID NO:497; and (ii) a VL having an amino acid sequence of SEQ ID NO:498 (FIG. 30).

In some embodiments, the anti-CD20 x anti-CD28 antibody comprises:
(a) a CD28 binding domain comprising:
(i) a VH comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO: 7, and a vICDR3 having an amino acid sequence of SEQ ID NO:22; and
(b) a CD20 binding domain comprising:
(i) a VH comprising a vhCDR1 having an amino acid sequence of SEQ ID NO:440; a vhCDR2 having an amino acid sequence of SEQ ID NO:457; and a vhCDR3 having an amino acid sequence of SEQ ID NO:458; and (ii) a VL comprising a vICDR1 having an amino acid sequence of SEQ ID NO:460, a vICDR2 having an amino acid sequence of SEQ ID NO:445, and a vICDR3 having an amino acid sequence of SEQ ID NO:462.

FIG. 10 shows some exemplary Fc domain sequences that are useful in the 1+1 Fab-scFv-Fc format antibodies. The “monomer 1” sequences depicted in FIG. 10 typically refer to the Fc domain of the “Fab-Fc heavy chain” and the “monomer 2” sequences refer to the Fc domain of the “scFv-Fc heavy chain.” In addition, FIGS. 12 and 13 provide exemplary CH1-hinge domains, CH1 domains, and hinge domains that can be included in the first or second monomer of the 1+1 Fab-scFv-Fc format. Further, FIG. 14 provides useful CL sequences that can be used with this format.

Exemplary subject anti-CD20 x anti-CD28 antibodies in the 1+1 Fab-scFv-Fc format are provided in FIG. 33.

In some embodiments, the anti-CD20 x anti-CD28 antibodies comprises:
a) a first monomer selected from the amino acid sequences represented by SEQ ID NOs: 405, 422, 427, 436, 453, 456. 461, and 485;
b) a second monomer selected from the amino acid sequences represented by SEQ ID NOs: 406, 417, 418, 421, 428, 430, 432, 434, 438, 448, 468, 469, and 486; and
c) a light chain selected from the amino acid sequences represented by SEQ ID NOs: 334, 366, 426, and 429.

In some embodiments, the anti-CD20 x anti-CD28 antibody in the 1+1 Fab-scFv-Fc format is XENP42157 or a variant thereof (see FIG. 33M). In some embodiments, the anti-CD20 x anti-CD28 antibody in the 1+1 Fab-scFv-Fc format includes: a) a first monomer having an amino acid sequence of SEQ ID NO:461; b) a second monomer having an amino acid sequence of SEQ ID NO:469; and c) a light chain having an amino acid sequence of SEQ ID NO:429. In some embodiments, the XENP42157 variant includes a deletion of a C-terminal lysine (−K) or lysine and glycine (−GK) in the first monomer or second monomer

### 2. 2+1 Fab₂-scFv-Fc Format

One heterodimeric antibody format that finds particular use in subject anti-CD20 x anti-CD28 antibodies provided herein is the 2+1 Fab₂-scFv-Fc format (also referred to as “central-scFv format”) shown in FIG. 29B. This antibody format includes three antigen binding domains: two Fab portions and an scFv that is inserted between the VH-CH1 and CH2-CH3 regions of one of the monomers. In some embodiments of this format, the Fab portions each bind CD20 and the “extra” scFv domain binds CD28. In some embodiments, the 2+1 Fab₂-scFv-Fc format antibody is a trivalent antibody.

In some embodiments of the 2+1 Fab₂-scFv-Fc format, a first monomer includes a standard heavy chain (i.e., VH1-CH1-hinge-CH2-CH3), wherein VH1 is a first variable heavy domain and CH2-CH3 is a first Fc domain. A second monomer includes another first variable heavy domain (VH1), a CH1 domain (and optional hinge), a second Fc domain, and an scFv that includes an scFv variable light domain (VL2), an scFv linker and a scFv variable heavy domain (VH2). The scFv is covalently attached between the C-terminus of the CH1 domain of the second monomer and the N-terminus of the second Fc domain using optional domain linkers (VH1-CH1-[optional linker]-VH2-scFv linker-VH2-[optional linker]-CH2-CH3, or the opposite orientation for the scFv, VH1-CH1-[optional linker]-VL2-scFv linker-VH2-[optional linker]-CH2-CH3). The optional linkers can be any suitable peptide linkers, including, for example, the domain linkers included in FIG. 7. This embodiment further utilizes a first and second light chain that each include a variable light domain (VL1) and a constant light domain (CL). The first light chain associates with the VH1-CH1 of the first monomer and the second light chain associates with the VH1-CH1 of the second monomers to form two identical Fabs. In some embodiments, the identical Fabs each bind CD20. As for many of the embodiments herein, these constructs can include skew variants, pI variants, ablation variants, additional Fc variants, etc. as desired and described herein.

In some embodiments, the first and second Fc domains of the 2+1 Fab₂-scFv-Fc format antibody are variant Fc domains that include heterodimerization skew variants (e.g., a set of amino acid substitutions as shown in FIGS. 3 and 9). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 4). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 Fab₂-scFv-Fc format antibody provided herein includes a charged scFv linker (including those shown in FIG. 6). In some embodiments, the 2+1 Fab₂-scFv-Fc format antibody provided herein includes FcRn variants M428L/N434S, wherein numbering is according to EU numbering.

In exemplary embodiments 2+1 Fab₂-scFv-Fc format antibody with “Platform X” variants, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 Fab₂-scFv-Fc format antibody provided herein includes a (GKPGS)₄ (SEQ ID NO:42) charged scFv linker. In some embodiments, the 2+1 Fab₂-scFv-Fc format antibody provided herein includes FcRn variants M428L/N434S, wherein numbering is according to EU numbering.

In some embodiments, the CH1-hinge-CH2-CH3 of the first monomer comprises amino acid variants L368D/K370S/N208D/Q295E/N384D/Q418E/N421D/E233P/L234V/L235A/G236del/S267K, and the second Fc domain comprises amino acid variants S364K/E357Q/E233P/L234V/L235A/G236del/S267K, wherein numbering is according to EU numbering.

In exemplary embodiments, the 2+1 Fab₂-scFv-Fc format antibody include “Platform Y” amino acid modifications as depicted in FIG. 8. In such embodiments, the first Fc domain comprises amino acid substitutions L234A/L235A/D265S/T366S/L368A/Y407V/H435R/Y436F, and the first Fc domain comprises amino acid substitutions L234A/L235A/D265S/LT366W, wherein numbering is according to EU numbering.

In exemplary embodiments of the 2+1 Fab₂-scFv-Fc format antibody with Platform Y variants, the first Fc domain includes heterodimerization skew variants T366S/L368A/Y407V and the second Fc domain includes heterodimerization skew variants T366W; each of the first and second variant Fc domains include ablation variants L234A/L235A/D265S; and the second Fc domain includes purification variants H435R/Y436F, wherein numbering is according to EU numbering. In some embodiments, the scFv of the 2+1 Fab₂-scFv-Fc format antibody provided herein includes a (GKPGS)₄ (SEQ ID NO:42) charged scFv linker. In some embodiments, the 2+1 Fab₂-scFv-Fc format antibody provided herein includes FcRn variants M252Y/S254T/T256E, wherein numbering is according to EU numbering. In some embodiments, the scFv of the 2+1 Fab₂-scFv-Fc Fc format antibody provided herein includes a charged scFv linker (including those shown in FIG. 6).

In exemplary embodiments, the 2+1 Fab₂-scFv-Fc format antibody with Platform Y variants further includes a “stapled” scFv (e.g., a “stapled” anti-CD28 scFv). Stapled” scFvs that exhibit improved stability and/or reduced aggregation are further described in detail herein. Exemplary staple linkers that are useful for inclusion in such “stapled” scFvs are provided in FIG. 6. In exemplary embodiments, the stapled linker is GGGSGGSGGCPPCGGSGG (SEQ ID NO:56).

In some embodiments, the scFv of the second monomer of the 2+1 Fab₂-scFv-Fc format antibody is a CD28 binding domain and the VH1 of the first monomer and the VL of the first light chain, and the VH1 of the second monomer and the VL1 of the second light chain each form a binding domain that binds CD20. Any suitable CD28 binding domain can be included in subject 2+1 Fab₂-scFv-Fc format antibody, including any of the CD28 binding domains provided herein.

In some embodiments of the 2+1 Fab₂-scFv-Fc format, the anti-CD28 ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:1; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:5; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:11; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:5; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:1; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:19; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:11; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:19; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:15; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:5; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:15; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:19; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:63; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:75; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:67; and (ii) a VL comprising a vlCDR1, a vlCDR2, and a vlCDR3 having an amino acid sequence of a vICDR1, a vICDR2, and a vICDR3, respectively, of a VL having an amino acid sequence of SEQ ID NO:75; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:63; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:79; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:67; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:111; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:71; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:75; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:71; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:79.

In some embodiments of the 2+1 Fab₂-scFv-Fc format, the anti-CD28 ABD has a VH and VL domain selected from the following:
(i) a VH comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a VH comprising a vhCDR1 having an amino acid sequence of SEQ ID NO:12; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a VH comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a VH comprising a vhCDR1 having an amino acid sequence of SEQ ID NO:12; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a VH comprising a vhCDR1 having an amino acid sequence of SEQ ID NO:12; a vhCDR2 having an amino acid sequence of SEQ ID NO:17; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a VH comprising a vhCDR1 having an amino acid sequence of SEQ ID NO:12; a vhCDR2 having an amino acid sequence of SEQ ID NO:17; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a VH comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a VH comprising a vhCDR1 having an amino acid sequence of SEQ ID NO:12; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a VH comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a VH comprising a vhCDR1 having an amino acid sequence of SEQ ID NO:12; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a VH comprising a vhCDR1 having an amino acid sequence of SEQ ID NO:12; a vhCDR2 having an amino acid sequence of SEQ ID NO:17; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a VH comprising a vhCDR1 having an amino acid sequence of SEQ ID NO:12; a vhCDR2 having an amino acid sequence of SEQ ID NO:17; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22.

In some embodiments of the 2+1 Fab₂-scFv-Fc format, the anti-CD28 ABD has a VH and VL domain selected from the following or a variant thereof:
(i) a VH having an amino acid sequence of SEQ ID NO:1; and (ii) a VL having an amino acid sequence of SEQ ID NO:5; or
(i) a VH having an amino acid sequence of SEQ ID NO:11; and (ii) a VL having an amino acid sequence of SEQ ID NO:5; or
(i) a VH having an amino acid sequence of SEQ ID NO:1; and (ii) a VL having an amino acid sequence of SEQ ID NO:19; or
(i) a VH having an amino acid sequence of SEQ ID NO:11; and (ii) a VL having an amino acid sequence of SEQ ID NO:19; or
(i) a VH having an amino acid sequence of SEQ ID NO:15; and (ii) a VL having an amino acid sequence of SEQ ID NO:5; or
(i) a VH having an amino acid sequence of SEQ ID NO:15; and (ii) a VL having an amino acid sequence of SEQ ID NO:19; or
(i) a VH having an amino acid sequence of SEQ ID NO:63; and (ii) a VL having an amino acid sequence of SEQ ID NO:75; or
(i) a VH having an amino acid sequence of SEQ ID NO:67; and (ii) a VL having an amino acid sequence of SEQ ID NO:75; or
(i) a VH having an amino acid sequence of SEQ ID NO:63; and (ii) a VL having an amino acid sequence of SEQ ID NO:79; or
(i) a VH having an amino acid sequence of SEQ ID NO:67; and (ii) a VL having an amino acid sequence of SEQ ID NO:79; or
(i) a VH having an amino acid sequence of SEQ ID NO:71; and (ii) a VL having an amino acid sequence of SEQ ID NO:75; or
(i) a VH having an amino acid sequence of SEQ ID NO:71; and (ii) a VL having an amino acid sequence of SEQ ID NO:79; or
i) a VH having an amino acid sequence of SEQ ID NO:320; and (ii) a VL having an amino acid sequence of SEQ ID NO:324; or
(i) a VH having an amino acid sequence of SEQ ID NO:327; and (ii) a VL having an amino acid sequence of SEQ ID NO:331; or
(i) a VH having an amino acid sequence of SEQ ID NO:335; and (ii) a VL having an amino acid sequence of SEQ ID NO:339; or
(i) a VH having an amino acid sequence of SEQ ID NO:343; and (ii) a VL having an amino acid sequence of SEQ ID NO:347; or
(i) a VH having an amino acid sequence of SEQ ID NO:351; and (ii) a VL having an amino acid sequence of SEQ ID NO:355; or
(i) a VH having an amino acid sequence of SEQ ID NO:359; and (ii) a VL having an amino acid sequence of SEQ ID NO:355; or
(i) a VH having an amino acid sequence of SEQ ID NO:367; and (ii) a VL having an amino acid sequence of SEQ ID NO:371; or
(i) a VH having an amino acid sequence of SEQ ID NO:375; and (ii) a VL having an amino acid sequence of SEQ ID NO:380; or
(i) a VH having an amino acid sequence of SEQ ID NO:391; and (ii) a VL having an amino acid sequence of SEQ ID NO:395; or
(i) a VH having an amino acid sequence of SEQ ID NO:399; and (ii) a VL having an amino acid sequence of SEQ ID NO:403; or
(i) a VH having an amino acid sequence of SEQ ID NO:407; and (ii) a VL having an amino acid sequence of SEQ ID NO:411; or
(i) a VH having an amino acid sequence of SEQ ID NO:415; and (ii) a VL having an amino acid sequence of SEQ ID NO:419; or
(i) a VH having an amino acid sequence of SEQ ID NO:423; and (ii) a VL having an amino acid sequence of SEQ ID NO:5; or
(i) a VH having an amino acid sequence of SEQ ID NO:431; and (ii) a VL having an amino acid sequence of SEQ ID NO:435.

In some embodiments, the VH1 of the first and second monomer and the VL1 of the common light chain of the 2+1 Fab₂-scFv-Fc format antibody each form a binding domain that binds CD20. In some embodiments of the 2+1 Fab₂-scFv-Fc format, the anti-CD20 ABDs each include a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:439; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:443; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:447; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:451; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:455; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:459; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:463; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:467.

In some embodiments of the 2+1 Fab₂-scFv-Fc format, the anti-CD20 ABDs each include a VH and VL domain selected from the following:
(i) a VH comprising a vhCDR1 having an amino acid sequence of SEQ ID NO:440; a vhCDR2 having an amino acid sequence of SEQ ID NO:441; and a vhCDR3 having an amino acid sequence of SEQ ID NO:442; and (ii) a VL comprising a vICDR1 having an amino acid sequence of SEQ ID NO:444, a vICDR2 having an amino acid sequence of SEQ ID NO: 445, and a vICDR3 having an amino acid sequence of SEQ ID NO:446; or
(i) a VH comprising a vhCDR1 having an amino acid sequence of SEQ ID NO:440; a vhCDR2 having an amino acid sequence of SEQ ID NO:449; and a vhCDR3 having an amino acid sequence of SEQ ID NO:450; and (ii) a VL comprising a vICDR1 having an amino acid sequence of SEQ ID NO:452, a vICDR2 having an amino acid sequence of SEQ ID NO:445, and a vICDR3 having an amino acid sequence of SEQ ID NO:454;
(i) a VH comprising a vhCDR1 having an amino acid sequence of SEQ ID NO:440; a vhCDR2 having an amino acid sequence of SEQ ID NO:457; and a vhCDR3 having an amino acid sequence of SEQ ID NO:458; and (ii) a VL comprising a vICDR1 having an amino acid sequence of SEQ ID NO:460, a vICDR2 having an amino acid sequence of SEQ ID NO:445, and a vICDR3 having an amino acid sequence of SEQ ID NO:462; or
(i) a VH comprising a vhCDR1 having an amino acid sequence of SEQ ID NO:464; a vhCDR2 having an amino acid sequence of SEQ ID NO:465; and a vhCDR3 having an amino acid sequence of SEQ ID NO:466; and (ii) a VL comprising a vICDR1 having an amino acid sequence of SEQ ID NO:356, a vICDR2 having an amino acid sequence of SEQ ID NO:357, and a vICDR3 having an amino acid sequence of SEQ ID NO:470.

In some embodiments of the 2+1 Fab₂-scFv-Fc format, the anti-CD20 ABDs each include a VH and VL domain selected from the following:
(i) a VH having an amino acid sequence of SEQ ID NO:439; and (ii) a VL having an amino acid sequence of SEQ ID NO:443; or
(i) a VH having an amino acid sequence of SEQ ID NO:447; and (ii) a VL having an amino acid sequence of SEQ ID NO:451; or
(i) a VH having an amino acid sequence of SEQ ID NO:455; and (ii) a VL having an amino acid sequence of SEQ ID NO:459; or
(i) a VH having an amino acid sequence of SEQ ID NO:463; and (ii) a VL having an amino acid sequence of SEQ ID NO:467; or
(i) a VH having an amino acid sequence of SEQ ID NO:471; and (ii) a VL having an amino acid sequence of SEQ ID NO:472; or
(i) a VH having an amino acid sequence of SEQ ID NO:473; and (ii) a VL having an amino acid sequence of SEQ ID NO:474; or
(i) a VH having an amino acid sequence of SEQ ID NO:475; and (ii) a VL having an amino acid sequence of SEQ ID NO:472; or
(i) a VH having an amino acid sequence of SEQ ID NO:477; and (ii) a VL having an amino acid sequence of SEQ ID NO:478; or
(i) a VH having an amino acid sequence of SEQ ID NO:479; and (ii) a VL having an amino acid sequence of SEQ ID NO:480; or
(i) a VH having an amino acid sequence of SEQ ID NO:481; and (ii) a VL having an amino acid sequence of SEQ ID NO:482; or
(i) a VH having an amino acid sequence of SEQ ID NO:483; and (ii) a VL having an amino acid sequence of SEQ ID NO:484; or
(i) a VH having an amino acid sequence of SEQ ID NO:487; and (ii) a VL having an amino acid sequence of SEQ ID NO:467; or
(i) a VH having an amino acid sequence of SEQ ID NO:489; and (ii) a VL having an amino acid sequence of SEQ ID NO:490; or
(i) a VH having an amino acid sequence of SEQ ID NO:491; and (ii) a VL having an amino acid sequence of SEQ ID NO:492; or
(i) a VH having an amino acid sequence of SEQ ID NO:471; and (ii) a VL having an amino acid sequence of SEQ ID NO:472; or
(i) a VH having an amino acid sequence of SEQ ID NO:495; and (ii) a VL having an amino acid sequence of SEQ ID NO:496; or
(i) a VH having an amino acid sequence of SEQ ID NO:497; and (ii) a VL having an amino acid sequence of SEQ ID NO:498 (FIG. 30).

In some embodiments, the anti-CD20 x anti-CD28 antibody comprises:
(a) a CD28 binding domain comprising:
(i) a VH comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22; and
(b) two CD20 binding domains, each comprising:
(i) a VH comprising a vhCDR1 having an amino acid sequence of SEQ ID NO:440; a vhCDR2 having an amino acid sequence of SEQ ID NO:457; and a vhCDR3 having an amino acid sequence of SEQ ID NO:458; and (ii) a VL comprising a vICDR1 having an amino acid sequence of SEQ ID NO:460, a vICDR2 having an amino acid sequence of SEQ ID NO:445, and a vICDR3 having an amino acid sequence of SEQ ID NO:462.

FIG. 11 shows some exemplary Fc domain sequences that are useful with the 2+1 Fab₂-scFv-Fc format. The “monomer 1” sequences depicted in FIG. 11 typically refer to the Fc domain of the “Fab-Fc heavy chain” and the “monomer 2” sequences refer to the Fc domain of the “Fab-scFv-Fc heavy chain.” In addition, FIGS. 12 and 13 provide exemplary CH1-hinge domains, CH1 domains, and hinge domains that can be included in the first or second monomer of the 2+1 Fab₂-scFv-Fc format. Further, FIG. 14 provides useful CL sequences that can be used with this format. Exemplary anti-CD20 x anti-CD28 antibodies in the 2+1 Fab₂-scFv-Fc format are depicted in FIG. 34.

In some embodiments, the anti-CD20 x anti-CD28 antibodies comprises:
a) a first monomer selected from the amino acid sequences represented by SEQ ID NOs: a) a first monomer having the amino acid sequence of SEQ ID NO: 427;
b) a second monomer selected from the amino acid sequences represented by SEQ ID NOs: 488 and 493;
c) a first light chain having the amino acid sequence of SEQ ID NO: 429; and
d) a second light chain having the amino acid sequence of SEQ ID NO: 429.

### 3. 1+1 CLC Format

One heterodimeric antibody format that finds particular use in subject anti-CD20 x anti-CD28 antibodies provided herein is the “1+1 Common Light Chain” or “1+1 CLC” format, which is depicted in FIG. 29C. The 1+1 CLC format antibody includes a first monomer that includes a VH1-CH1-hinge-CH2-CH3, wherein VH1 is a first variable heavy domain and CH2-CH3 is a first Fc domain; a second monomer that includes a VH2-CH1-hinge-CH2-CH3, wherein VH2 is a second variable heavy domain and CH2-C3 is a second Fc domain; and a third monomer “common light chain” comprising VL-CL, wherein VL is a common variable light domain and CL is a constant light domain. In such embodiments, the VL pairs with the VH1 to form a first binding domain with a first antigen binding specificity; and the VL pairs with the VH2 to form a second binding domain with a second antigen binding specificity. In some embodiments, the 1+1 CLC format antibody is a bivalent antibody.

In some embodiments, the first and second Fc domains of the 1+1 CLC format are variant Fc domains that include heterodimerization skew variants (e.g., a set of amino acid substitutions as shown in FIGS. 3 and 9). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first or second monomer includes pI variants (including those shown in FIG. 4). In exemplary embodiments, the constant domain (CH1-hinge-CH2-CH3) of the first or second monomer includes pI variants N208D/Q295E/N384D/Q418E/N421D, wherein numbering is according to EU numbering.

In some embodiments, the 1+1 CLC format antibody provided herein includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236/S267K; and the constant domain (CH1-hinge-CH2-CH3) of the first monomer includes pI variants N208D/Q295E/N384D/Q418E/N421D, wherein numbering is according to EU numbering.

In some embodiments, the CH1-hinge-CH2-CH3 of the first monomer comprises amino acid variants L368D/K370S/N208D/Q295E/N384D/Q418E/N421D/E233P/L234V/L235A/G236del/S267K, and the second Fc domain comprises amino acid variants S364K/E357Q/E233P/L234V/L235A/G236del/S267K, wherein numbering is according to EU numbering.

In some embodiments, the 1+1 CLC format antibody provided herein further includes FcRn variants M428L/N434S, wherein numbering is according to EU numbering.

In some embodiments, one of the first binding domain or the second binding domain binds CD20 and the other binding domain binds CD28. Any suitable CD20 binding domain and CD28 domain can be included in subject 1+1 CLC format antibody, including any of the CD20 binding domains and CD28 binding domains provided herein or a variant thereof (see, e.g., FIGS. 15-24, 26, and 30).

### 4. 2+1 CLC Format

Another heterodimeric antibody format that finds particular use in subject anti-CD20 x anti-CD28 antibodies provided herein is the “2+1 Common Light Chain” or “2+1 CLC” format, which is depicted in FIG. 29D. The 2+1 CLC format includes a first monomer that includes a VH1-CH1-linker-VH1-CH1-hinge-CH2-CH3, wherein the VH1s are each a first variable heavy domain and CH2-CH3 is a first Fc domain; a second monomer that includes a VH2-CH1-hinge-CH2-CH3, wherein VH2 is a second variable heavy domain and CH2-CH3 is a second Fc domain; and a third monomer that includes a “common light chain” VL-CL, wherein VL is a common variable light domain and CL is a constant light domain. The VL pairs with each of the VH1s of the first monomer to form two first binding domains, each with a first antigen binding specificity; and the VL pairs with the VH2 to form a second binding domain with a second antigen binding specificity. The linker of the first monomer can be any suitable linker, including any one of the domain linkers or combinations thereof described in FIG. 7. In some embodiments, the linker is EPKSCGKPGSGKPGS (SEQ ID NO:87). In some embodiments, the 2+1 CLC format antibody is a trivalent antibody.

In some embodiments, the first and second Fc domains of the 2+1 CLC format are variant Fc domains that include heterodimerization skew variants (e.g., a set of amino acid substitutions as shown in FIGS. 3 and 9). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first or second monomer includes pI variants (including those shown in FIG. 4). In exemplary embodiments, the constant domain (CH1-hinge-CH2-CH3) of the first or second monomer includes pI variants N208D/Q295E/N384D/Q418E/N421D, wherein numbering is according to EU numbering.

In some embodiments, the 2+1 CLC format antibody provided herein further includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the 2+1 CLC format antibody provided herein further includes FcRn variants M428L/N434S, wherein numbering is according to EU numbering.

In some embodiments, the CH1-hinge-CH2-CH3 of the second monomer comprises amino acid variants L368D/K370S/N208D/Q295E/N384D/Q418E/N421D/E233P/L234V/L235A/G236del/S267K, and the first Fc domain comprises amino acid variants S364K/E357Q/E233P/L234V/L235A/G236del/S267K, wherein numbering is according to EU numbering.

In some embodiments, each of the two first binding domains binds CD20 and the second binding domain binds CD28. Any suitable CD20 binding domain and CD28 binding domain can be included in the subject 2+1 CLC format antibody, including any of the CD20 binding domains and CD28 binding domains provided herein or a variant thereof (see, e.g., FIGS. 15-24, 26, and 30).

5. 2+1 mAb-scFv Format

One heterodimeric antibody format that finds particular use in the subject bispecific anti-CD20 x anti-CD28 antibodies is the 2+1 mAb-scFv format shown in FIG. 29E. This antibody format includes three antigen binding domains: two Fab portions and an scFv that is attached to the C-terminal of one of the heavy chains. In some embodiments of this format, the Fab portions each bind CD20, in this case, human CD20, and the “extra” scFv domain binds CD28. That is, this mAb-scFv format is a trivalent antibody.

In these embodiments, the first chain or monomer comprises, from N- to C-terminal, VH1-CH1-hinge-CH2-CH3, the second monomer comprises, from N- to C-terminal, VH1-CH1-hinge-CH2-CH3-domain linker-scFv domain, where the scFv domain comprises a second VH (VH2), a second VL (VL2) and a scFv linker. As for all the scFv domains herein, the scFv domain can be in either orientation, from N- to C-terminal, VH2-scFv linker-VL2 or VL2-scFv linker-VH2. Accordingly, the second monomer may comprise, from N- to C-terminal, VH1-CH1-hinge-CH2-CH3-domain linker-VH2-scFv linker-VL2 or VH1-CH1-hinge-CH2-CH3-domain linker-VL2-scFv linker-VH2. The composition also comprises a first and second light chain that are each, VL1-CL. In these embodiments, the VH1 of the first monomer and the VL1 of the first light chain, and the VH1 of the second monomer and the VL1 of the second light chain each form a first ABD, and the VH2-VL2 form a second ABD. In some embodiments, the first ABDs bind to CD20, and the second ABD binds human CD28.

In some embodiments, the first and second Fc domains of the 2+1 mAb-scFv format antibody are variant Fc domains that include heterodimerization skew variants (e.g., a set of amino acid substitutions as shown in FIGS. 3 and 9). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 4). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 mAb-scFv format antibody provided herein includes a charged scFv linker (including those shown in FIG. 6). In some embodiments, the 2+1 mAb-scFv format antibody provided herein includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 mAb-scFv format antibody provided herein includes a (GKPGS)₄ (SEQ ID NO:42) charged scFv linker. In some embodiments, 2+1 mAb-scFv format antibody provided herein includes FcRn variants M428L/N434S, wherein numbering is according to EU numbering.

In some embodiments, the scFv of the second monomer of the 2+1 mAb-scFv format antibody is a CD28 binding domain, and the VH1 of the first monomer and the VL1 of the first light chain, and the VL1 of the second monomer and the VL1 of the second light chain each form binding domains that bind CD20. Any suitable CD20 binding domain and CD28 binding domain can be included in the 2+1 mAb-scFv format antibody, including any of the CD20 binding domains and CD28 binding domains provided herein or a variant thereof (see, e.g., FIGS. 15-24, 26, and 30).

Any suitable CD28 binding domain can be included in subject 2+1 mAb-scFv-Fc format antibody, including any of the CD28 binding domains provided herein. In some embodiments of the 2+1 mAb-scFv-Fc format, the anti-CD28 ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:1; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:5; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:11; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:5; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:1; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:19; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:11; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:19; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:15; and (ii) a VL comprising a vICDR1, a vICDR2, and a vICDR3 having an amino acid sequence of a vlCDR1, a vlCDR2, and a vlCDR3, respectively, of a VL having an amino acid sequence of SEQ ID NO:5; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:15; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:19; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:63; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:75; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:67; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:75; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:63; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:79; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:67; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:79; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:71; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:75; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:71; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:79.

In some embodiments of the 2+1 mAb-scFv-Fc format, the anti-CD28 ABD has a VH and VL domain selected from the following:
(i) a VH comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a VH comprising a vhCDR1 having an amino acid sequence of SEQ ID NO:12; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a VH comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a VH comprising a vhCDR1 having an amino acid sequence of SEQ ID NO:12; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a VH comprising a vhCDR1 having an amino acid sequence of SEQ ID NO:12; a vhCDR2 having an amino acid sequence of SEQ ID NO:17; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a VH comprising a vhCDR1 having an amino acid sequence of SEQ ID NO:12; a vhCDR2 having an amino acid sequence of SEQ ID NO:17; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a VH comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a VH comprising a vhCDR1 having an amino acid sequence of SEQ ID NO:12; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a VH comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a VH comprising a vhCDR1 having an amino acid sequence of SEQ ID NO:12; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a VH comprising a vhCDR1 having an amino acid sequence of SEQ ID NO:12; a vhCDR2 having an amino acid sequence of SEQ ID NO:17; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a VH comprising a vhCDR1 having an amino acid sequence of SEQ ID NO:12; a vhCDR2 having an amino acid sequence of SEQ ID NO:17; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22.

In some embodiments of the 2+1 mAb-scFv-Fc format, the anti-CD28 ABD has a VH and VL domain selected from the following or a variant thereof:
(i) a VH having an amino acid sequence of SEQ ID NO:1; and (ii) a VL having an amino acid sequence of SEQ ID NO:5; or
(i) a VH having an amino acid sequence of SEQ ID NO:11; and (ii) a VL having an amino acid sequence of SEQ ID NO:5; or
(i) a VH having an amino acid sequence of SEQ ID NO:1; and (ii) a VL having an amino acid sequence of SEQ ID NO:19; or
(i) a VH having an amino acid sequence of SEQ ID NO:11; and (ii) a VL having an amino acid sequence of SEQ ID NO:19; or
(i) a VH having an amino acid sequence of SEQ ID NO:15; and (ii) a VL having an amino acid sequence of SEQ ID NO:5; or
(i) a VH having an amino acid sequence of SEQ ID NO:15; and (ii) a VL having an amino acid sequence of SEQ ID NO:19; or
(i) a VH having an amino acid sequence of SEQ ID NO:63; and (ii) a VL having an amino acid sequence of SEQ ID NO:75; or
(i) a VH having an amino acid sequence of SEQ ID NO:67; and (ii) a VL having an amino acid sequence of SEQ ID NO:75; or
(i) a VH having an amino acid sequence of SEQ ID NO:63; and (ii) a VL having an amino acid sequence of SEQ ID NO:79; or
(i) a VH having an amino acid sequence of SEQ ID NO:67; and (ii) a VL having an amino acid sequence of SEQ ID NO:79; or
(i) a VH having an amino acid sequence of SEQ ID NO:71; and (ii) a VL having an amino acid sequence of SEQ ID NO:75; or
(i) a VH having an amino acid sequence of SEQ ID NO:71; and (ii) a VL having an amino acid sequence of SEQ ID NO:79; or
i) a VH having an amino acid sequence of SEQ ID NO:320; and (ii) a VL having an amino acid sequence of SEQ ID NO:324; or
(i) a VH having an amino acid sequence of SEQ ID NO:327; and (ii) a VL having an amino acid sequence of SEQ ID NO:331; or
(i) a VH having an amino acid sequence of SEQ ID NO:335; and (ii) a VL having an amino acid sequence of SEQ ID NO:339; or
(i) a VH having an amino acid sequence of SEQ ID NO:343; and (ii) a VL having an amino acid sequence of SEQ ID NO:347; or
(i) a VH having an amino acid sequence of SEQ ID NO:351; and (ii) a VL having an amino acid sequence of SEQ ID NO:355; or
(i) a VH having an amino acid sequence of SEQ ID NO:359; and (ii) a VL having an amino acid sequence of SEQ ID NO:355; or
(i) a VH having an amino acid sequence of SEQ ID NO:367; and (ii) a VL having an amino acid sequence of SEQ ID NO:371; or
(i) a VH having an amino acid sequence of SEQ ID NO:375; and (ii) a VL having an amino acid sequence of SEQ ID NO:380; or
(i) a VH having an amino acid sequence of SEQ ID NO:391; and (ii) a VL having an amino acid sequence of SEQ ID NO:395; or
(i) a VH having an amino acid sequence of SEQ ID NO:399; and (ii) a VL having an amino acid sequence of SEQ ID NO:403; or
(i) a VH having an amino acid sequence of SEQ ID NO:407; and (ii) a VL having an amino acid sequence of SEQ ID NO:411; or
(i) a VH having an amino acid sequence of SEQ ID NO:415; and (ii) a VL having an amino acid sequence of SEQ ID NO:419; or
(i) a VH having an amino acid sequence of SEQ ID NO:423; and (ii) a VL having an amino acid sequence of SEQ ID NO:5; or
(i) a VH having an amino acid sequence of SEQ ID NO:431; and (ii) a VL having an amino acid sequence of SEQ ID NO:435.

In some embodiments, the VH1 of the first and second monomer and the VL1 of the common light chain of the 2+1 mAb-scFv-Fc format antibody each form a binding domain that binds CD20. In some embodiments of the 2+1 mAb-scFv-Fc format, the anti-CD20 ABDs each include a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:439; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:443; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:447; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:451; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:455; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:459; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:463; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:467.

In some embodiments of the 2+1 mAb-scFv-Fc format, the anti-CD20 ABDs each include a VH and VL domain selected from the following:
(i) a VH comprising a vhCDR1 having an amino acid sequence of SEQ ID NO:440; a vhCDR2 having an amino acid sequence of SEQ ID NO:441; and a vhCDR3 having an amino acid sequence of SEQ ID NO:442; and (ii) a VL comprising a vICDR1 having an amino acid sequence of SEQ ID NO:444, a vICDR2 having an amino acid sequence of SEQ ID NO: 445, and a vICDR3 having an amino acid sequence of SEQ ID NO:446; or
(i) a VH comprising a vhCDR1 having an amino acid sequence of SEQ ID NO:440; a vhCDR2 having an amino acid sequence of SEQ ID NO:449; and a vhCDR3 having an amino acid sequence of SEQ ID NO:450; and (ii) a VL comprising a vICDR1 having an amino acid sequence of SEQ ID NO:452, a vICDR2 having an amino acid sequence of SEQ ID NO:445, and a vICDR3 having an amino acid sequence of SEQ ID NO:454;
(i) a VH comprising a vhCDR1 having an amino acid sequence of SEQ ID NO:440; a vhCDR2 having an amino acid sequence of SEQ ID NO:457; and a vhCDR3 having an amino acid sequence of SEQ ID NO:458; and (ii) a VL comprising a vICDR1 having an amino acid sequence of SEQ ID NO:460, a vICDR2 having an amino acid sequence of SEQ ID NO:445, and a vICDR3 having an amino acid sequence of SEQ ID NO:462; or
(i) a VH comprising a vhCDR1 having an amino acid sequence of SEQ ID NO:464; a vhCDR2 having an amino acid sequence of SEQ ID NO:465; and a vhCDR3 having an amino acid sequence of SEQ ID NO:466; and (ii) a VL comprising a vICDR1 having an amino acid sequence of SEQ ID NO:356, a vICDR2 having an amino acid sequence of SEQ ID NO:357, and a vICDR3 having an amino acid sequence of SEQ ID NO:470.

In some embodiments of the 2+1 mAb-scFv-Fc format, the anti-CD20 ABDs each include a VH and VL domain selected from the following or a variant thereof:
(i) a VH having an amino acid sequence of SEQ ID NO:439; and (ii) a VL having an amino acid sequence of SEQ ID NO:443; or
(i) a VH having an amino acid sequence of SEQ ID NO:447; and (ii) a VL having an amino acid sequence of SEQ ID NO:451; or
(i) a VH having an amino acid sequence of SEQ ID NO:455; and (ii) a VL having an amino acid sequence of SEQ ID NO:459; or
(i) a VH having an amino acid sequence of SEQ ID NO:463; and (ii) a VL having an amino acid sequence of SEQ ID NO:467; or
(i) a VH having an amino acid sequence of SEQ ID NO:471; and (ii) a VL having an amino acid sequence of SEQ ID NO:472; or
(i) a VH having an amino acid sequence of SEQ ID NO:473; and (ii) a VL having an amino acid sequence of SEQ ID NO:474; or
(i) a VH having an amino acid sequence of SEQ ID NO:475; and (ii) a VL having an amino acid sequence of SEQ ID NO:472; or
(i) a VH having an amino acid sequence of SEQ ID NO:477; and (ii) a VL having an amino acid sequence of SEQ ID NO:478; or
(i) a VH having an amino acid sequence of SEQ ID NO:479; and (ii) a VL having an amino acid sequence of SEQ ID NO:480; or
(i) a VH having an amino acid sequence of SEQ ID NO:481; and (ii) a VL having an amino acid sequence of SEQ ID NO:482; or
(i) a VH having an amino acid sequence of SEQ ID NO:483; and (ii) a VL having an amino acid sequence of SEQ ID NO:484; or
(i) a VH having an amino acid sequence of SEQ ID NO:487; and (ii) a VL having an amino acid sequence of SEQ ID NO:467; or
(i) a VH having an amino acid sequence of SEQ ID NO:489; and (ii) a VL having an amino acid sequence of SEQ ID NO:490; or
(i) a VH having an amino acid sequence of SEQ ID NO:491; and (ii) a VL having an amino acid sequence of SEQ ID NO:492; or
(i) a VH having an amino acid sequence of SEQ ID NO:471; and (ii) a VL having an amino acid sequence of SEQ ID NO:472; or
(i) a VH having an amino acid sequence of SEQ ID NO:495; and (ii) a VL having an amino acid sequence of SEQ ID NO:496; or
(i) a VH having an amino acid sequence of SEQ ID NO:497; and (ii) a VL having an amino acid sequence of SEQ ID NO:498 (FIG. 30).

In some embodiments, the anti-CD20 x anti-CD28 mAb-scFv format antibody comprises:
(a) a CD28 binding domain comprising:
(i) a VH comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22; and
(b) two CD20 binding domains, each comprising:
(i) a VH comprising a vhCDR1 having an amino acid sequence of SEQ ID NO:440; a vhCDR2 having an amino acid sequence of SEQ ID NO:457; and a vhCDR3 having an amino acid sequence of SEQ ID NO:458; and (ii) a VL comprising a vICDR1 having an amino acid sequence of SEQ ID NO:460, a vICDR2 having an amino acid sequence of SEQ ID NO:445, and a vICDR3 having an amino acid sequence of SEQ ID NO:462.

Exemplary anti-CD20 x anti-CD28 antibodies in the 2+1 mAb-scFv format are depicted in FIG. 36.

In some embodiments, the anti-CD20 x anti-CD28 antibodies comprises:
a) a first monomer selected from the amino acid sequences represented by SEQ ID NOs: 405, 422, 427, 436, and 516;
b) a second monomer selected from the amino acid sequences represented by SEQ ID NOs: 14, 416, 494, 500, 501, 502, 503, 504, 505, 506, 507, 508, 509, 510, 511, 512, 513, 514, and 515;
c) a first light chain selected from the amino acid sequences represented by SEQ ID NOs: 334, 366, 426, and 429; and
d) a second light chain selected from the amino acid sequences represented by SEQ ID NOs: 334, 366, 426, and 429.

### 6. 2+1 StackFab₂-Scfv-Fc

One heterodimeric antibody format that finds particular use in the subject bispecific anti-CD20 x anti-CD28 antibodies is the 2+1 stackFab₂-scFv-Fc format antibodies (FIG. 29F). This antibody format includes three antigen binding domains: two Fab portions and an scFv binding domain. Each of the Fab portions include a first and second common variable light chain. This heterodimeric antibody format that finds particular use in the subject trispecific antibodies provided herein.

The 2+1 stackFab₂-scFv-Fc format includes a first monomer, second monomer, a first common light chain, and a second common light chain. The first monomer includes from N-terminal to C-terminal, scFv-linker-CH2-CH3 wherein CH2-CH3 is a first Fc domain. The second monomer includes from N-terminal to C-terminal, VH1-CH1-linker-VH1-CH1-hinge-CH2-CH3 wherein VH1s are each a first variable heavy domain, and CH2-CH3 is a second Fc domain. Each of the first and second common light chains include from N-terminal to C-terminal, VL1-CL, wherein VL1 is a first variable light domain and CL is a constant light domain. Further, the scFv comprises a second VH domain (VH2), a scFv linker, and a second variable light domain (VL2). In this format, one of the VH1 of the second monomer and the VL1 of the first common light chain, and the other of the VH1 of the second monomer and the VL1 of the second common light chain each form a first antigen binding domain, and the second variable heavy domain and second variable light domain form a second antigen binding domain. In some embodiments, the first antigen binding domains each bind CD20, and the second antigen binding domain binds CD28.

In exemplary embodiments, the first and second Fc domains are variant Fc domains. In some embodiments, the first and second Fc domains of the 2+1 stackFab₂-scFv-Fc format antibody are variant Fc domains that include heterodimerization skew variants (e.g., a set of amino acid substitutions as shown in FIGS. 3 and 9). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering. In exemplary embodiments, the first variant Fc domain includes heterodimerization skew variants S364K/E357Q and the second variant Fc domain includes heterodimerization skew variants L368D/K370S, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 4). In exemplary embodiments, the constant domain (CH1-hinge-CH2-CH3) of the second monomer includes pI variants N208D/Q295E/N384D/Q418E/N421D, wherein numbering is according to EU numbering.

In exemplary embodiments, the CH1-hinge-CH2-CH3 of the first monomer comprises amino acid variants S364K/E357Q/E233P/L234V/L235A/G236del/S267K, the second Fc domain comprises amino acid variants L368D/K370S/N208D/Q295E/N384D/Q418E/N421D/E233P/L234V/L235A/G236del/S267K, wherein numbering is according to EU numbering.

In some embodiments, the scFv of the 2+1 stackFab₂-scFv-Fc format antibody provided herein includes a charged scFv linker (including those shown in FIG. 6). In some embodiments, the 2+1 stackFab₂-scFv-Fc format antibody provided herein includes FcRn variants M428L/N434S, wherein numbering is according to EU numbering.

In some embodiments, the scFv of the first monomer of the 2+1 stackFab₂-scFv-Fc format antibody is a CD28 binding domain and one of the VH1 of the second monomer and the VL of the first common light chain, and the other VH1 of the second monomer and the VL1 of the second common light chain each form a binding domain that bind CD20. Any suitable CD20 binding domain and CD28 binding domain can be included in the 2+1 stackFab₂-scFv-Fc, including any of the CD20 binding domains and CD28 binding domains provided herein or a variant thereof (see, e.g., FIGS. 15-24, 26, and 30).

Any suitable CD28 binding domain can be included in subject 2+1 stackFab₂-scFv-Fc antibody, including any of the CD28 binding domains provided herein. In some embodiments of the 2+1 stackFab₂-scFv-Fc format, the anti-CD28 ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:1; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:5; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:11; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:5; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:1; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:19; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:11; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:19; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:15; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:5; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:15; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:19; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:63; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:75; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:67; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:75; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:63; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:79; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:67; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:79; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:71; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:75; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:71; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:79.

In some embodiments of the 2+1 stackFab₂-scFv-Fc format, the anti-CD28 ABD has a VH and VL domain selected from the following:
(i) a VH comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a VH comprising a vhCDR1 having an amino acid sequence of SEQ ID NO:12; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a VH comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a VH comprising a vhCDR1 having an amino acid sequence of SEQ ID NO:12; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a VH comprising a vhCDR1 having an amino acid sequence of SEQ ID NO:12; a vhCDR2 having an amino acid sequence of SEQ ID NO:17; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a VH comprising a vhCDR1 having an amino acid sequence of SEQ ID NO:12; a vhCDR2 having an amino acid sequence of SEQ ID NO:17; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a VH comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a VH comprising a vhCDR1 having an amino acid sequence of SEQ ID NO:12; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a VH comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a VH comprising a vhCDR1 having an amino acid sequence of SEQ ID NO:12; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22; or
(i) a VH comprising a vhCDR1 having an amino acid sequence of SEQ ID NO:12; a vhCDR2 having an amino acid sequence of SEQ ID NO:17; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:8; or
(i) a VH comprising a vhCDR1 having an amino acid sequence of SEQ ID NO:12; a vhCDR2 having an amino acid sequence of SEQ ID NO:17; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22.

In some embodiments of the 2+1 stackFab₂-scFv-Fc format, the anti-CD28 ABD has a VH and VL domain selected from the following or a variant thereof:
(i) a VH having an amino acid sequence of SEQ ID NO:1; and (ii) a VL having an amino acid sequence of SEQ ID NO:5; or
(i) a VH having an amino acid sequence of SEQ ID NO:11; and (ii) a VL having an amino acid sequence of SEQ ID NO:5; or
(i) a VH having an amino acid sequence of SEQ ID NO:1; and (ii) a VL having an amino acid sequence of SEQ ID NO:19; or
(i) a VH having an amino acid sequence of SEQ ID NO:11; and (ii) a VL having an amino acid sequence of SEQ ID NO:19; or
(i) a VH having an amino acid sequence of SEQ ID NO:15; and (ii) a VL having an amino acid sequence of SEQ ID NO:5; or
(i) a VH having an amino acid sequence of SEQ ID NO:15; and (ii) a VL having an amino acid sequence of SEQ ID NO:19; or
(i) a VH having an amino acid sequence of SEQ ID NO:63; and (ii) a VL having an amino acid sequence of SEQ ID NO:75; or
(i) a VH having an amino acid sequence of SEQ ID NO:67; and (ii) a VL having an amino acid sequence of SEQ ID NO:75; or
(i) a VH having an amino acid sequence of SEQ ID NO:63; and (ii) a VL having an amino acid sequence of SEQ ID NO:79; or
(i) a VH having an amino acid sequence of SEQ ID NO:67; and (ii) a VL having an amino acid sequence of SEQ ID NO:79; or
(i) a VH having an amino acid sequence of SEQ ID NO:71; and (ii) a VL having an amino acid sequence of SEQ ID NO:75; or
(i) a VH having an amino acid sequence of SEQ ID NO:71; and (ii) a VL having an amino acid sequence of SEQ ID NO:79; or
i) a VH having an amino acid sequence of SEQ ID NO:320; and (ii) a VL having an amino acid sequence of SEQ ID NO:324; or
(i) a VH having an amino acid sequence of SEQ ID NO:327; and (ii) a VL having an amino acid sequence of SEQ ID NO:331; or
(i) a VH having an amino acid sequence of SEQ ID NO:335; and (ii) a VL having an amino acid sequence of SEQ ID NO:339; or
(i) a VH having an amino acid sequence of SEQ ID NO:343; and (ii) a VL having an amino acid sequence of SEQ ID NO:347; or
(i) a VH having an amino acid sequence of SEQ ID NO:351; and (ii) a VL having an amino acid sequence of SEQ ID NO:355; or
(i) a VH having an amino acid sequence of SEQ ID NO:359; and (ii) a VL having an amino acid sequence of SEQ ID NO:355; or
(i) a VH having an amino acid sequence of SEQ ID NO:367; and (ii) a VL having an amino acid sequence of SEQ ID NO:371; or
(i) a VH having an amino acid sequence of SEQ ID NO:375; and (ii) a VL having an amino acid sequence of SEQ ID NO:380; or
(i) a VH having an amino acid sequence of SEQ ID NO:391; and (ii) a VL having an amino acid sequence of SEQ ID NO:395; or
(i) a VH having an amino acid sequence of SEQ ID NO:399; and (ii) a VL having an amino acid sequence of SEQ ID NO:403; or
(i) a VH having an amino acid sequence of SEQ ID NO:407; and (ii) a VL having an amino acid sequence of SEQ ID NO:411; or
(i) a VH having an amino acid sequence of SEQ ID NO:415; and (ii) a VL having an amino acid sequence of SEQ ID NO:419; or
(i) a VH having an amino acid sequence of SEQ ID NO:423; and (ii) a VL having an amino acid sequence of SEQ ID NO:5; or
(i) a VH having an amino acid sequence of SEQ ID NO:431; and (ii) a VL having an amino acid sequence of SEQ ID NO:435.

In some embodiments, the VH1 of the first and second monomer and the VL1 of the common light chain of the 2+1 stackFab₂-scFv-Fc format antibody each form a binding domain that binds CD20. In some embodiments of the 2+1 stackFab₂-scFv-Fc format, the anti-CD20 ABDs each include a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:439; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:443; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:447; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:451; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:455; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:459; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:463; and (ii) a VL comprising a vICDR1, a vICDR2, and a vICDR3 having an amino acid sequence of a vICDR1, a vICDR2, and a vICDR3, respectively, of a VL having an amino acid sequence of SEQ ID NO:467.

In some embodiments of the 2+1 stackFab₂-scFv-Fc format, the anti-CD20 ABDs each include a VH and VL domain selected from the following:
(i) a VH comprising a vhCDR1 having an amino acid sequence of SEQ ID NO:440; a vhCDR2 having an amino acid sequence of SEQ ID NO:441; and a vhCDR3 having an amino acid sequence of SEQ ID NO:442; and (ii) a VL comprising a vICDR1 having an amino acid sequence of SEQ ID NO:444, a vICDR2 having an amino acid sequence of SEQ ID NO: 445, and a vICDR3 having an amino acid sequence of SEQ ID NO:446; or
(i) a VH comprising a vhCDR1 having an amino acid sequence of SEQ ID NO:440; a vhCDR2 having an amino acid sequence of SEQ ID NO:449; and a vhCDR3 having an amino acid sequence of SEQ ID NO:450; and (ii) a VL comprising a vICDR1 having an amino acid sequence of SEQ ID NO:452, a vICDR2 having an amino acid sequence of SEQ ID NO:445, and a vICDR3 having an amino acid sequence of SEQ ID NO:454;
(i) a VH comprising a vhCDR1 having an amino acid sequence of SEQ ID NO:440; a vhCDR2 having an amino acid sequence of SEQ ID NO:457; and a vhCDR3 having an amino acid sequence of SEQ ID NO:458; and (ii) a VL comprising a vICDR1 having an amino acid sequence of SEQ ID NO:460, a vICDR2 having an amino acid sequence of SEQ ID NO:445, and a vICDR3 having an amino acid sequence of SEQ ID NO:462; or
(i) a VH comprising a vhCDR1 having an amino acid sequence of SEQ ID NO:464; a vhCDR2 having an amino acid sequence of SEQ ID NO:465; and a vhCDR3 having an amino acid sequence of SEQ ID NO:466; and (ii) a VL comprising a vICDR1 having an amino acid sequence of SEQ ID NO:356, a vICDR2 having an amino acid sequence of SEQ ID NO:357, and a vICDR3 having an amino acid sequence of SEQ ID NO:470.

In some embodiments of the 2+1 stackFab₂-scFv-Fc format, the anti-CD20 ABDs each include a VH and VL domain selected from the following or a variant thereof:
(i) a VH having an amino acid sequence of SEQ ID NO:439; and (ii) a VL having an amino acid sequence of SEQ ID NO:443; or
(i) a VH having an amino acid sequence of SEQ ID NO:447; and (ii) a VL having an amino acid sequence of SEQ ID NO:451; or
(i) a VH having an amino acid sequence of SEQ ID NO:455; and (ii) a VL having an amino acid sequence of SEQ ID NO:459; or
(i) a VH having an amino acid sequence of SEQ ID NO:463; and (ii) a VL having an amino acid sequence of SEQ ID NO:467; or
(i) a VH having an amino acid sequence of SEQ ID NO:471; and (ii) a VL having an amino acid sequence of SEQ ID NO:472; or
(i) a VH having an amino acid sequence of SEQ ID NO:473; and (ii) a VL having an amino acid sequence of SEQ ID NO:474; or
(i) a VH having an amino acid sequence of SEQ ID NO:475; and (ii) a VL having an amino acid sequence of SEQ ID NO:472; or
(i) a VH having an amino acid sequence of SEQ ID NO:477; and (ii) a VL having an amino acid sequence of SEQ ID NO:478; or
(i) a VH having an amino acid sequence of SEQ ID NO:479; and (ii) a VL having an amino acid sequence of SEQ ID NO:480; or
(i) a VH having an amino acid sequence of SEQ ID NO:481; and (ii) a VL having an amino acid sequence of SEQ ID NO:482; or
(i) a VH having an amino acid sequence of SEQ ID NO:483; and (ii) a VL having an amino acid sequence of SEQ ID NO:484; or
(i) a VH having an amino acid sequence of SEQ ID NO:487; and (ii) a VL having an amino acid sequence of SEQ ID NO:467; or
(i) a VH having an amino acid sequence of SEQ ID NO:489; and (ii) a VL having an amino acid sequence of SEQ ID NO:490; or
(i) a VH having an amino acid sequence of SEQ ID NO:491; and (ii) a VL having an amino acid sequence of SEQ ID NO:492; or
(i) a VH having an amino acid sequence of SEQ ID NO:471; and (ii) a VL having an amino acid sequence of SEQ ID NO:472; or
(i) a VH having an amino acid sequence of SEQ ID NO:495; and (ii) a VL having an amino acid sequence of SEQ ID NO:496; or
(i) a VH having an amino acid sequence of SEQ ID NO:497; and (ii) a VL having an amino acid sequence of SEQ ID NO:498 (FIG. 30).

In some embodiments, the anti-CD20 x anti-CD28 antibody comprises:
(a) a CD28 binding domain comprising:
(i) a VH comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (ii) a VL comprising a vICDR1 having an amino acid sequence of SEQ ID NO:6, a vICDR2 having an amino acid sequence of SEQ ID NO:7, and a vICDR3 having an amino acid sequence of SEQ ID NO:22; and
(b) two CD20 binding domains, each comprising:
(i) a VH comprising a vhCDR1 having an amino acid sequence of SEQ ID NO:440; a vhCDR2 having an amino acid sequence of SEQ ID NO:457; and a vhCDR3 having an amino acid sequence of SEQ ID NO:458; and (ii) a VL comprising a vICDR1 having an amino acid sequence of SEQ ID NO:460, a vICDR2 having an amino acid sequence of SEQ ID NO:445, and a vICDR3 having an amino acid sequence of SEQ ID NO:462.

Exemplary anti-CD20 x anti-CD28 antibodies in the 2+1 stackFab₂-scFv-Fc format are depicted in FIG. 35.

In some embodiments, the anti-CD20 x anti-CD28 antibodies comprises: a first monomer selected from the amino acid sequences represented by SEQ ID NO: 499; a second monomer selected from the amino acid sequences represented by SEQ ID NOs: 428,432,434, 438, 448; a first light chain selected from the amino acid sequences represented by SEQ ID NO:429; and a second light chain selected from the amino acid sequences represented by SEQ ID NO:429

7. Dual scFv Formats

One heterodimeric antibody format that finds particular use in the subject bispecific anti-CD20 x anti-CD28 antibodies is the dual scFv format, as are known in the art and shown in FIG. 29G. In this embodiment, the heterodimeric bispecific antibody is made up of two scFv-Fc monomers (both in either (vh-scFv linker-vl-[optional domain linker]-CH2-CH3) format or (vl-scFv linker-vh-[optional domain linker]-CH2-CH3) format, or with one monomer in one orientation and the other in the other orientation.

In this case, all ABDs are in the scFv format. Any suitable CD20 binding domain and CD28 binding domain can be included in anti-CD20 x anti-CD28 antibody in the dual scFv format, including any of the CD20 binding domains and CD28 binding domains provided herein.

In addition, the Fc domains of the dual scFv format comprise skew variants (e.g. a set of amino acid substitutions as shown in FIG. 4, with particularly useful skew variants being selected from the group consisting of S364K/E357Q:L368D/K370S; L368D/K370S:S364K; L368E/K370S:S364K; T411T/E360E/Q362E:D401K; L368D/K370S:S364K/E357L, K370S:S364K/E357Q, T366S/L368A/Y407V:T366W and T366S/L368A/Y407V/Y349C:T366W/S354C), optionally ablation variants (including those shown in FIG. 5), optionally charged scFv linkers (including those shown in FIG. 6) and the heavy chain comprises pI variants (including those shown in FIG. 4).

In some embodiments, the dual scFv format includes skew variants, pI variants, and ablation variants. Accordingly, some embodiments include formats that comprise: a) a first monomer that comprises the skew variants S364K/E357Q, the ablation variants E233P/L234V/L235A/G236del/S267K, and a scFv that binds a first antigen (VH1-scFv linker-VL1-[optional domain linker]-CH2-CH3 or VL1-scFv linker-VH1-[optional domain linker]-CH2-CH3) and b) a first monomer that comprises the skew variants L368D/K370S, the ablation variants E233P/L234V/L235A/G236del/S267K, and a scFv that binds a second antigen (VH1-scFv linker-VL1-[optional domain linker]-CH2-CH3 or VL1-scFv linker-VH1-[optional domain linker]-CH2-CH3). pI variants can be as outlined herein, but most common will be charged scFv linkers of opposite charge for each monomer. FcRn variants, particularly 428L/434S, can optionally be included.

Any suitable CD20 binding domain and CD28 binding domain can be included in the dual scFv format antibody, including any of the CD20 binding domains and CD28 binding domains provided herein or a variant thereof (see, e.g., FIGS. 15-24, 26, and 30).

8. One-Armed scFv-mAb Format

One heterodimeric antibody format that finds particular use in subject anti-CD20 x anti-CD28 antibodies provided herein is the one-armed mAb-scFv format shown in FIG. 29H. This format includes: 1) a first monomer that comprises an “empty” Fc domain; 2) a second monomer that includes a first variable heavy domain (VH), a scFv domain (a second antigen binding domain) and an Fc domain, where the scFv domain is attached to the N-terminus of the first variable heavy domain; and 3) a light chain that includes a first variable light domain and a constant light domain. The first variable heavy domain and the first variable light domain form a first antigen binding domain and the scFv is a second antigen binding domain. In this format, one of the first antigen binding domain and second binding domain binds CD28, and the other antigen binding domain binds CD20. As for many of the embodiments herein, these constructs include skew variants, pI variants, ablation variants, additional Fc variants, etc. as desired and described herein.

Any suitable CD20 binding domain and CD28 binding domain can be included in the one-armed scFv-mAb antibody format, including any of the CD20 binding domains and CD28 binding domains provided herein or a variant thereof (see, e.g., FIGS. 15-24, 26, and 30).

9. scFv-mAb Format

One heterodimeric antibody format that finds particular use in subject anti-CD20 x anti-CD28 antibodies provided herein is the mAb-scFv format shown in FIG. 291. In this embodiment, the format relies on the use of a N-terminal attachment of a scFv to one of the monomers, thus forming a third antigen binding domain, wherein the Fab portions of the two monomers each bind one target and the “extra” scFv domain binds a different target.

In this embodiment, the first monomer comprises a first heavy chain (comprising a variable heavy domain and a constant domain), with a N-terminally covalently attached scFv comprising a scFv variable light domain, an scFv linker and a scFv variable heavy domain in either orientation ((vh1-scFv linker-vl1-[optional domain linker]-vh2-CH1-hinge-CH2-CH3) or (with the scFv in the opposite orientation) ((vl1-scFv linker-vh1-[optional domain linker]-vh2-CH1-hinge-CH2-CH3)). The second monomer comprises a heavy chain VH2-CH1-hinge-CH2-CH3. This embodiment further utilizes a common light chain comprising a variable light domain and a constant light domain, that associates with the heavy chains to form two identical Fabs. As for many of the embodiments herein, these constructs include skew variants, pI variants, ablation variants, additional Fc variants, etc. as desired and described herein.

Any suitable CD20 binding domain and CD28 binding domain can be included in the scFv-mAb antibody format, including any of the CD20 binding domains and CD28 binding domains provided herein or a variant thereof (see, e.g., FIGS. 15-24, 26, and 30).

### 10. Non-Heterodimeric Bispecific Antibodies

As will be appreciated by those in the art, the anti-CD20 x anti-CD28 antibodies provided herein can also be included in non-heterodimeric bispecific formats (see FIG. 29J). In this format, the anti-CD20 x anti-CD28 includes: 1) a first monomer comprising a VH1-CH1-hinge-CH2-CH3; 2) a second monomer comprising a VH2-CH1-hinge-CH2-CH3; 3) a first light chain comprising a VL1-CL; and 4) a second light chain comprising a VL2-CL. In such embodiments, the VH1 and VL1 form a first antigen binding domain and VH2 and VL2 form a second antigen binding domain. One of the first or second antigen binding domains binds CD28 and the other antigen binding domain binds CD20.

Any suitable CD20 binding domain and CD28 binding domain can be included in anti-CD20 x anti-CD28 antibody in the non-heterodimeric bispecific antibody format, including any of the CD20 binding domains and CD28 binding domains provided herein or a variant thereof (see, e.g., FIGS. 15-24, 26, and 30).

### 11. One Armed Central-scFv

One heterodimeric antibody format that finds particular use in subject anti-CD20 x anti-CD28 antibodies provided herein is the one armed central-scFv format shown in FIG. 29K. In this embodiment, one monomer comprises just an Fc domain, while the other monomer includes a Fab domain (a first antigen binding domain), a scFv domain (a second antigen binding domain) and an Fc domain, where the scFv domain is inserted between the Fc domain and the Fc domain.

In this embodiment, one monomer comprises a first heavy chain comprising a first variable heavy domain, a CH1 domain and Fc domain, with a scFv comprising a scFv variable light domain, an scFv linker and a scFv variable heavy domain. The scFv is covalently attached between the C-terminus of the CH1 domain of the heavy constant domain and the N-terminus of the first Fc domain using domain linkers, in either orientation, VH1-CH1-[optional domain linker]-VH2-scFv linker-VL2-[optional domain linker]-CH2-CH3 or VH1-CH1-[optional domain linker]-VL2-scFv linker-VH2-[optional domain linker]-CH2-CH3. The second monomer comprises an Fc domain (CH2-CH3). This embodiment further utilizes a light chain comprising a variable light domain and a constant light domain, that associates with the heavy chain to form a Fab. As for many of the embodiments herein, these constructs include skew variants, pI variants, ablation variants, additional Fc variants, etc. as desired and described herein.

Any suitable CD20 binding domain and CD28 binding domain can be included in the one armed central-scFv format, including any of the CD20 binding domains and CD28 binding domains provided herein or a variant thereof (see, e.g., FIGS. 15-24, 26, and 30).

12. mAb-Fv Format

One heterodimeric antibody format that finds particular use in subject anti-CD20 x anti-CD28 antibodies provided herein is the mAb-Fv format (FIG. 29L). In this embodiment, the format relies on the use of a C-terminal attachment of an “extra” variable heavy domain to one monomer and the C-terminal attachment of an “extra” variable light domain to the other monomer, thus forming a third antigen binding domain (i.e. an “extra” Fv domain), wherein the Fab portions of the two monomers bind CD28 and the “extra” Fv domain binds CD20.

In this embodiment, the first monomer comprises a first heavy chain, comprising a first variable heavy domain and a first constant heavy domain comprising a first Fc domain, with a first variable light domain covalently attached to the C-terminus of the first Fc domain using a domain linker (vh1-CH1-hinge-CH2-CH3-[optional linker]-vl2). The second monomer comprises a second variable heavy domain, a second constant heavy domain comprising a second Fc domain, and a third variable heavy domain covalently attached to the C-terminus of the second Fc domain using a domain linker (vh1-CH1-hinge-CH2-CH3-[optional linker]-vh2. This embodiment further utilizes a common light chain comprising a variable light domain and a constant light domain, which associates with the heavy chains to form two identical Fabs that include two identical Fvs. The two C-terminally attached variable domains make up the “extra” third Fv. As for many of the embodiments herein, these constructs include skew variants, pI variants, ablation variants, additional Fc variants, etc. as desired and described herein.

In addition, the Fc domains of the mAb-Fv format comprise skew variants (e.g. a set of amino acid substitutions as shown in FIG. 3, with particularly useful skew variants being selected from the group consisting of S364K/E357Q:L368D/K370S; L368D/K370S:S364K; L368E/K370S:S364K; T411T/E360E/Q362E:D401K; L368D/K370S:S364K/E357L, K370S:S364K/E357Q, T366S/L368A/Y407V:T366W and T366S/L368A/Y407V/Y349C:T366W/S354C), optionally ablation variants (including those shown in FIG. 5), optionally charged scFv linkers (including those shown in FIG. 6) and the heavy chain comprises pI variants (including those shown in FIG. 4).

Any suitable CD20 binding domain and CD28 binding domain can be included in the mAb-Fv format, including any of the CD20 binding domains and CD28 binding domains provided herein or a variant thereof (see, e.g., FIGS. 15-24, 26, and 30).

### 13. Central-Fv Format

O One heterodimeric antibody format that finds particular use in subject anti-CD20 x anti-CD28 antibodies provided herein is the central-Fv format shown in FIG. 29M. In this embodiment, the format relies on the use of an inserted Fv domain thus forming an “extra” third antigen binding domain, wherein the Fab portions of the two monomers bind CD20 and the “extra” central-Fv domain binds CD28. The Fv domain is inserted between the Fc domain and the CH1-Fv region of the monomers, thus providing a third antigen binding domain, wherein each monomer contains a component of the Fv (e.g. one monomer comprises a variable heavy domain and the other a variable light domain of the “extra” central Fv domain).

In this embodiment, one monomer comprises a first heavy chain comprising a first variable heavy domain, a CH1 domain, and Fc domain and an additional variable light domain. The additional variable light domain is covalently attached between the C-terminus of the CH1 domain of the heavy constant domain and the N-terminus of the first Fc domain using domain linkers (vh1-CH1-[optional linker]-vl2-hinge-CH2-CH3). The other monomer comprises a first heavy chain comprising a first variable heavy domain, a CH1 domain and Fc domain and an additional variable heavy domain (vh1-CH1-[optional linker]-vh2-hinge-CH2-CH3). The additional variable heavy domain is covalently attached between the C-terminus of the CH1 domain of the heavy constant domain and the N-terminus of the first Fc domain using domain linkers. This embodiment utilizes a common light chain comprising a variable light domain and a constant light domain, that associates with the heavy chains to form two identical Fabs that each bind CD20. The additional variable heavy domain and additional variable light domain form an “extra” central Fv that binds CD28. As for many of the embodiments herein, these constructs include skew variants, pI variants, ablation variants, additional Fc variants, etc. as desired and described herein.

Any suitable CD20 binding domain and CD28 binding domain can be included in the central-Fv format, including any of the CD20 binding domains and CD28 binding domains provided herein or a variant thereof (see, e.g., FIGS. 15-24, 26, and 30).

### 14. Trident Format

In some embodiments, the anti-CD20 x anti-CD28 antibodies provided herein are in the “Trident” format as generally described in WO2015/184203, hereby expressly incorporated by reference in its entirety and in particular for the Figures, Legends, definitions and sequences of “Heterodimer-Promoting Domains” or “HPDs”, including “K-coil” and “E-coil” sequences. Tridents rely on using two different HPDs that associate to form a heterodimeric structure as a component of the structure, see FIG. 29N. In this embodiment, the Trident format include a “traditional” heavy and light chain (e.g. VH1-CH1-hinge-CH2-CH3 and VL1-CL), a third chain comprising a first “diabody-type binding domain” or “DART®”, VH2-(linker)-VL3-HPD1 and a fourth chain comprising a second DART*, VH3-(linker)-(linker)-VL2-HPD2. The VH1 and VL1 form a first ABD, the VH2 and VL2 form a second ABD, and the VH3 and VL3 form a third ABD. In some cases, as is shown in FIG. 29N, the second and third ABDs bind the same antigen.

Any suitable CD20 binding domain and CD28 binding domain can be included in the trident format, including any of the CD20 binding domains and CD28 binding domains provided herein or a variant thereof (see, e.g., FIGS. 15-24, 26, and 30).

### V. Nucleic Acids

In another embodiment, provided herein are nucleic acid compositions encoding the anti-CD20 x anti-CD28 antibodies provided herein. A nucleic acid composition may refer to one or multiple polynucleotides.

As will be appreciated by those in the art, the nucleic acid compositions will depend on the format and scaffold of the heterodimeric protein. Thus, for example, when the format requires three amino acid sequences, such as for the 1+1 Fab-scFv-Fc or 2+1 Fab₂-scFv-Fc formats, three polynucleotides can be incorporated into one or more expression vectors for expression. In exemplary embodiments, each polynucleotide is incorporated into a different expression vector.

As is known in the art, the nucleic acids encoding the components of the binding domains and antibodies disclosed herein can be incorporated into expression vectors as is known in the art, and depending on the host cells used to produce the heterodimeric antibodies of the invention. Generally the nucleic acids are operably linked to any number of regulatory elements (promoters, origin of replication, selectable markers, ribosomal binding sites, inducers, etc.). The expression vectors can be extra-chromosomal or integrating vectors.

The polynucleotides and/or expression vectors of the invention are then transformed into any number of different types of host cells as is well known in the art, including mammalian, bacterial, yeast, insect and/or fungal cells, with mammalian cells (e.g., CHO cells), finding use in many embodiments.

In some embodiments, polynucleotides encoding each monomer are each contained within a single expression vector, generally under different or the same promoter controls. In embodiments of particular use in the present invention, each of these polynucleotides are contained on different expression vectors. As shown herein and in U.S. 62/025,931, hereby incorporated by reference, different vector ratios can be used to drive heterodimer formation. That is, surprisingly, while the proteins comprise first monomer:second monomer:light chains (in the case of many of the embodiments herein that have three polypeptides comprising the heterodimeric antibody) in a 1:1:2 ratio, these are not the ratios that give the best results.

The antibodies provided herein are made by culturing host cells comprising the expression vector(s) as is well known in the art. Once produced, traditional antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the “1+1 Fab-scFv-Fc” heterodimer (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

### VI. Biological and Biochemical Functionality of the Anti-CD28 x Anti-CD20 Antibodies

Generally the anti-CD20 x anti-CD28 antibodies described herein are administered to patients with a CD20-associated cancer (e.g., B cell malignancy), and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

### A. Antibody Compositions for In Vivo Administration

Formulations of the antibodies described herein are prepared for storage by mixing an antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

### VII. Treatments

Once made, the compositions of the invention find use in a number of oncology applications, by treating cancer, generally by enhancing immune responses (e.g., T cell activation and proliferation), particularly when used with anti-cancer therapies such as bispecific antibodies. In some embodiments, the antibodies provided herein enhance immune responses (e.g., T cell activation and proliferation) by providing agonistic co-stimulation of T cells in the microenvironment of cells overexpressing CD20.

In some embodiments, the anti-CD20 x anti-CD28 bispecific antibodies provided herein are administered with an anti-tumor therapy including, for example, anti-tumor-associated antigen (TAA) bispecific antibodies.

### A. Anti-CD20 x Anti-CD28 Combination Therapies

In some embodiments, the anti-CD20 x anti-CD28 antibodies provided herein are administered with is a T-cell engaging multispecific antibody, such as those that bind to human CD3, CD20, and CD79b. In some embodiments, the T-cell engaging multispecific antibody is the CD79b x CD20 X CD3 molecule shown in FIG. 32. Other anti-CD79b x anti-CD20 X anti-CD3 trispecific antibodies are disclosed in WO2022/200443 which is incorporated herein by reference.

In some embodiments, a method of treating a B-cell malignancy in a patient in need thereof, comprising administering to the patient a combination of an anti-CD20 x anti-CD28 antibody or an antibody fragment thereof and a T-cell engager, wherein:
(A) the anti-CD20 x anti-CD28 antibody or the fragment thereof comprises:
(a) a first antigen binding domain that binds to CD20 comprising:
(i) a vhCDR1 having an amino acid sequence of SEQ ID NO:440; a vhCDR2 having an amino acid sequence of SEQ ID NO:441; and a vhCDR3 having an amino acid sequence of SEQ ID NO:442; and a vICDR1 having an amino acid sequence of SEQ ID NO:444, a vICDR2 having an amino acid sequence of SEQ ID NO: 445, and a vICDR3 having an amino acid sequence of SEQ ID NO:446;
(ii) a vhCDR1 having an amino acid sequence of SEQ ID NO:440; a vhCDR2 having an amino acid sequence of SEQ ID NO:449; and a vhCDR3 having an amino acid sequence of SEQ ID NO:450; and a vICDR1 having an amino acid sequence of SEQ ID NO:452, a vICDR2 having an amino acid sequence of SEQ ID NO:445, and a vICDR3 having an amino acid sequence of SEQ ID NO:454;
(iii) a vhCDR1 having an amino acid sequence of SEQ ID NO:440; a vhCDR2 having an amino acid sequence of SEQ ID NO:457; and a vhCDR3 having an amino acid sequence of SEQ ID NO:458; and a vICDR1 having an amino acid sequence of SEQ ID NO:460, a vICDR2 having an amino acid sequence of SEQ ID NO:445, and a vICDR3 having an amino acid sequence of SEQ ID NO:462; or
(iv) a vhCDR1 having an amino acid sequence of SEQ ID NO:464; a vhCDR2 having an amino acid sequence of SEQ ID NO:465; and a vhCDR3 having an amino acid sequence of SEQ ID NO:466; and a vICDR1 having an amino acid sequence of SEQ ID NO:356, a vICDR2 having an amino acid sequence of SEQ ID NO:357, and a vICDR3 having an amino acid sequence of SEQ ID NO:470; and
(b) a second antigen binding domain that binds to CD28 comprising:
(i) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:8;
(ii) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:8;
(iii) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:22;
(iv) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:22;
(v) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:8; or
(vi) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:22; and
(B) the T cell engager is an anti-CD79b x anti-CD20 x anti-CD3 antibody or an antibody fragment thereof.

In some embodiments, the anti-CD20 x anti-CD28 antibodies provided herein are administered in combination with CD79b X CD20 X CD3 antibody comprising: (A) a CD79b binding domain comprising heavy chain CDRs of SEQ ID NOs: 524, 525, and 526; and light chain CDRs of SEQ ID NOs: 528, 529, and 530; (B) a CD20 binding domain comprising heavy chain CDRs of SEQ ID NOs: 540, 541 and 542; and light chain CDRs of SEQ ID NOs: 544, 545, and 546; and (C) a CD3 binding domain comprising heavy chain CDRs of SEQ ID NO: 532, 533, and 534; and light chain CDRs of SEQ ID NOs: 536, 537, and 538.

In some embodiments, a method of treating a B-cell malignancy in a patient in need thereof, comprises administering to the patient a combination of an anti-CD20 x anti-CD28 antibody or an antibody fragment thereof and a T-cell engager, wherein:
A) anti-CD20 x anti-CD28 antibody comprises: i) a first antigen binding domain having: (a) a VH1 comprising a vhCDR1 having an amino acid sequence of SEQ ID NO:440; a vhCDR2 having an amino acid sequence of SEQ ID NO:457; and a vhCDR3 having an amino acid sequence of SEQ ID NO:458; and (b) a VL1 comprising a vICDR1 having an amino acid sequence of SEQ ID NO:460, a vICDR2 having an amino acid sequence of SEQ ID NO:445, and a vICDR3 having an amino acid sequence of SEQ ID NO:462; and ii) a second antigen binding domain having: (a) a VH2 comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (b) a VL2 comprising a vICDR1 having an amino acid sequence of SEQ ID NO:6, a vICDR2 having an amino acid sequence of SEQ ID NO:7, and a vICDR3 having an amino acid sequence of SEQ ID NO:22; and
B) T cell engager is an anti-CD79b x anti-CD20 x anti-CD3 antibody or the antibody fragment thereof and comprises: (a) an antigen binding domain that binds to CD79b comprising a vhCDR1 having an amino acid sequence of SEQ ID NO:524, a vhCDR2 having an amino acid sequence of SEQ ID NO:525, and a vhCDR3 having an amino acid sequence of SEQ ID NO:526; and a vICDR1 having an amino acid sequence of SEQ ID NO:528, a vICDR2 having an amino acid sequence of SEQ ID NO:529, and a vICDR3 having an amino acid sequence of SEQ ID NO:530; (b) an antigen binding domain that binds to CD20 comprising a vhCDR1 having an amino acid sequence of SEQ ID NO:540, a vhCDR2 having an amino acid sequence of SEQ ID NO:541, and a vhCDR3 having an amino acid sequence of SEQ ID NO:542; and a vICDR1 having an amino acid sequence of SEQ ID NO:544, a vICDR2 having an amino acid sequence of SEQ ID NO:545, and a vICDR3 having an amino acid sequence of SEQ ID NO:546; and (c) an antigen binding domain that binds to CD3 comprising a vhCDR1 having an amino acid sequence of SEQ ID NO:532, a vhCDR2 having an amino acid sequence of SEQ ID NO:533, and a vhCDR3 having an amino acid sequence of SEQ ID NO:534; and a vICDR1 having an amino acid sequence of SEQ ID NO:536, a vICDR2 having an amino acid sequence of SEQ ID NO:537, and a vICDR3 having an amino acid sequence of SEQ ID NO:538.

In some embodiments, a method of enhancing T cell proliferation comprises contacting the cells with:
A) anti-CD20 x anti-CD28 antibody comprising: i) a first antigen binding domain having: (a) a VH1 comprising a vhCDR1 having an amino acid sequence of SEQ ID NO:440; a vhCDR2 having an amino acid sequence of SEQ ID NO:457; and a vhCDR3 having an amino acid sequence of SEQ ID NO:458; and (b) a VL1 comprising a vICDR1 having an amino acid sequence of SEQ ID NO:460, a vICDR2 having an amino acid sequence of SEQ ID NO:445, and a vICDR3 having an amino acid sequence of SEQ ID NO:462; and ii) a second antigen binding domain having: (a) a VH2 comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (b) a VL2 comprising a vICDR1 having an amino acid sequence of SEQ ID NO:6, a vICDR2 having an amino acid sequence of SEQ ID NO:7, and a vICDR3 having an amino acid sequence of SEQ ID NO:22; and
B) a T cell engager, wherein the T cell engager is an anti-CD79b x anti-CD20 x anti-CD3 antibody. In some embodiments, anti-CD79b x anti-CD20 x anti-CD3 antibody or the antibody fragment comprises: (a) an antigen binding domain that binds to CD79b comprising a vhCDR1 having an amino acid sequence of SEQ ID NO:524, a vhCDR2 having an amino acid sequence of SEQ ID NO:525, and a vhCDR3 having an amino acid sequence of SEQ ID NO:526; and a vICDR1 having an amino acid sequence of SEQ ID NO:528, a vICDR2 having an amino acid sequence of SEQ ID NO:529, and a vICDR3 having an amino acid sequence of SEQ ID NO:530; (b) an antigen binding domain that binds to CD20 comprising a vhCDR1 having an amino acid sequence of SEQ ID NO:540, a vhCDR2 having an amino acid sequence of SEQ ID NO:541, and a vhCDR3 having an amino acid sequence of SEQ ID NO:542; and a vICDR1 having an amino acid sequence of SEQ ID NO:544, a vICDR2 having an amino acid sequence of SEQ ID NO:545, and a vICDR3 having an amino acid sequence of SEQ ID NO:546; and (c) an antigen binding domain that binds to CD3 comprising a vhCDR1 having an amino acid sequence of SEQ ID NO:532, a vhCDR2 having an amino acid sequence of SEQ ID NO:533, and a vhCDR3 having an amino acid sequence of SEQ ID NO:534; and a vICDR1 having an amino acid sequence of SEQ ID NO:536, a vICDR2 having an amino acid sequence of SEQ ID NO:537, and a vICDR3 having an amino acid sequence of SEQ ID NO:538.

In some embodiments, a method of increasing central memory T cells in a patient suffering from a B-cell malignancy comprises administering to the patient a combination of an anti-CD20 x anti-CD28 antibody or an antibody fragment thereof and an anti-CD79b x anti-CD20 x anti-CD3 antibody or the antibody fragment, wherein:
A) anti-CD20 x anti-CD28 antibody comprises: i) a first antigen binding domain having: (a) a VH1 comprising a vhCDR1 having an amino acid sequence of SEQ ID NO:440; a vhCDR2 having an amino acid sequence of SEQ ID NO:457; and a vhCDR3 having an amino acid sequence of SEQ ID NO:458; and (b) a VL1 comprising a vICDR1 having an amino acid sequence of SEQ ID NO:460, a vICDR2 having an amino acid sequence of SEQ ID NO:445, and a vICDR3 having an amino acid sequence of SEQ ID NO:462; and ii) a second antigen binding domain having: (a) a VH2 comprising a vhCDR1 having an amino acid sequence of SEQ ID NO:2; a vhCDR2 having an amino acid sequence of SEQ ID NO:3; and a vhCDR3 having an amino acid sequence of SEQ ID NO:4; and (b) a VL2 comprising a vICDR1 having an amino acid sequence of SEQ ID NO:6, a vICDR2 having an amino acid sequence of SEQ ID NO:7, and a vICDR3 having an amino acid sequence of SEQ ID NO:22; and
B) anti-CD79b x anti-CD20 x anti-CD3 antibody or the antibody fragment comprises: (a) an antigen binding domain that binds to CD79b comprising a vhCDR1 having an amino acid sequence of SEQ ID NO:524, a vhCDR2 having an amino acid sequence of SEQ ID NO:525, and a vhCDR3 having an amino acid sequence of SEQ ID NO:526; and a vICDR1 having an amino acid sequence of SEQ ID NO:528, a vICDR2 having an amino acid sequence of SEQ ID NO:529, and a vICDR3 having an amino acid sequence of SEQ ID NO:530; (b) an antigen binding domain that binds to CD20 comprising a vhCDR1 having an amino acid sequence of SEQ ID NO:540, a vhCDR2 having an amino acid sequence of SEQ ID NO:541, and a vhCDR3 having an amino acid sequence of SEQ ID NO:542; and a vICDR1 having an amino acid sequence of SEQ ID NO:544, a vICDR2 having an amino acid sequence of SEQ ID NO:545, and a vICDR3 having an amino acid sequence of SEQ ID NO:546; and (c) an antigen binding domain that binds to CD3 comprising a vhCDR1 having an amino acid sequence of SEQ ID NO:532, a vhCDR2 having an amino acid sequence of SEQ ID NO:533, and a vhCDR3 having an amino acid sequence of SEQ ID NO:534; and a vICDR1 having an amino acid sequence of SEQ ID NO:536, a vICDR2 having an amino acid sequence of SEQ ID NO:537, and a vICDR3 having an amino acid sequence of SEQ ID NO:538.

In some embodiments, the anti-CD20 x anti-CD28 antibodies provided herein are administered in combination with CD79b X CD20 X CD3 antibody comprising: (A) a CD79b binding domain comprising a VH having an amino acid sequence of SEQ ID NO:523, and a VL having an amino acid sequence of SEQ ID NO: 527; (B) a CD20 binding domain comprising a VH having an amino acid sequence of SEQ ID NO: 539, and a VL having an amino acid sequence of SEQ ID NO:543; and (C) a CD3 binding domain comprising a VH having an amino acid sequence of SEQ ID NO:531, and a VL having an amino acid sequence of SEQ ID NO:535.

In some embodiments, a method of treating a B-cell malignancy in a patient in need thereof, comprising administering to the patient a combination of an anti-CD20 x anti-CD28 antibody or an antibody fragment thereof and an anti-CD79b x anti-CD20 x anti-CD3 antibody or the antibody fragment, wherein:
(a) the anti-CD20 x anti-CD28 antibody or the antibody fragment comprises:
   (i) the CD20 antigen binding domain comprising a VH1 having an amino acid sequence of SEQ ID NO:455; and a VL1 having an amino acid sequence of SEQ ID NO:459; and
   (ii) the CD28 antigen binding domain comprising a VH2 having an amino acid sequence of SEQ ID NO:63; and a VL2 having an amino acid sequence of SEQ ID NO:79; and
(b) the anti-CD79b x anti-CD20 x anti-CD3 antibody or the antibody fragment comprises:
   (i) the CD79b antigen binding domain comprising a VH1 having an amino acid sequence of SEQ ID NO:523; and a VL1 having an amino acid sequence of SEQ ID NO:527;
   (ii) the CD20 antigen binding domain comprising a VH2 having an amino acid sequence of SEQ ID NO:539; and a VL2 having an amino acid sequence of SEQ ID NO:543; and
   (iii) the CD3 antigen binding domain comprising a VH2 having an amino acid sequence of SEQ ID NO:531; and a VL2 having an amino acid sequence of SEQ ID NO:535.

In some embodiments, the anti-CD20 x anti-CD28 antibodies provided herein are administered in combination with CD79b X CD20 X CD3 antibody comprising: (i) a first monomer having an amino acid sequence of SEQ ID NO:520; (ii) a second monomer having an amino acid sequence of SEQ ID NO:521; and (iii) a light chain having an amino acid sequence of SEQ ID NO: 522.

In some embodiments, a method of treating a B-cell malignancy in a patient in need thereof, comprising administering to the patient a combination of an anti-CD20 x anti-CD28 antibody or an antibody fragment thereof and an anti-CD79b x anti-CD20 x anti-CD3 antibody or the antibody fragment, wherein:
(a) the anti-CD20 x anti-CD28 antibody or the antibody fragment comprises:
   (i) a first monomer polypeptide having an amino acid sequence of SEQ ID NO:461;
   (ii) a second monomer polypeptide having an amino acid sequence of SEQ ID NO:469;
   (iii) a first light chain polypeptide having an amino acid sequence of SEQ ID NO:429; and
(b) the anti-CD79b x anti-CD20 x anti-CD3 antibody or the antibody fragment comprises:
   (i) a fourth monomer polypeptide having an amino acid sequence of SEQ ID NO:520;
   (ii) a fifth monomer polypeptide having an amino acid sequence of SEQ ID NO:521; and
   (iii) a second light chain polypeptide having an amino acid sequence of SEQ ID NO:522.

In classic T cell/APC interaction, there is a first signal provided by TCR reactivity with peptide-MHC (Signal 1) and a second signal provided by CD28 crosslinking by CD80/CD86 being expressed on APCs (Signal 2) which together fully activate T cells (see FIG. 31A). In contrast, only the first signal is provided in treatment with CD3 T cell engaging antibodies that target a tumor-associated antigen (TAA)(i.e., anti-CD3 x anti-CD20 x anti-CD79b trispecific antibodies)(see FIG. 31B).

Without being bound by any particular theory of operation, it is believed that the anti-CD20 x anti-CD28 bispecific antibodies provided herein can enhance the anti-tumor response of an anti-CD79b x anti-CD20 x anti-CD3 trispecific antibody by enhancing T-cell activation, potency and persistence as well as overcoming immunosuppression within the tumor microenvironment (see Example 4). Thus, in one embodiment, provided herein are methods of treating a cancer-cell malignancies in a patient by administering the patient an anti-CD79b x anti-CD20 x anti-CD3, and an anti-CD20 x anti-CD28 bispecific antibody provided herein.

Accordingly, the combination of an anti-CD79b x anti-CD20 x anti-CD3 and an anti-CD20 x anti-CD28 bispecific antibody are useful in the treatment or prevention of a variety of B-cell malignancies, including (but not limited to) the following: B-cell lymphoma, non-Hodgkin lymphoma, diffuse large B-cell lymphoma (DLBCL), a mantle cell lymphoma (MCL), a follicular lymphoma (FL), acute lymphoblastic leukemia (ALL), chronic lymphocytic leukemia (CLL), small lymphocytic lymphoma (SLL), Waldenström macroglobulinemia (WM), multiple myeloma (MM), mucosa-associated lymphoid tissue (MALT) lymphoma, Hodgkin's lymphoma, Burkitt's lymphoma, hairy-cell leukemia, or Plasmacytoma cells. In some embodiments, the B-cell malignancy disclosed herein is a relapsed or refractory form of the cancer.

### B. Administrative Modalities

The antibodies provided herein administered to a subject, in accord with known methods, such as subcutaneous, intravenous administration as a bolus or by continuous infusion over a period of time. In some embodiments, anti-CD20 x anti-CD28 bispecific antibodies and the anti-CD79b x anti-CD20 x anti-CD3 antibodies are administered concurrently or sequentially.

### C. Treatment Modalities

In the methods of the invention, therapy is used to provide a positive therapeutic response with respect to a disease or condition.

By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the bispecific antibodies used in the present invention depend on the disease or condition to be treated and may be determined by the persons skilled in the art. An exemplary, non-limiting range for a therapeutically effective amount of anti-CD20 X anti-CD28 bispecific antibodies or anti-CD20X anti-CD3 bispecific antibodies that are disclosed herein is about 0.001-10 mg/kg, such as about 0.001-5 mg/kg, for example about 0.001-2 mg/kg, such as about 0.001-1 mg/kg, for instance about 0.001, about 0.01, about 0.1, about 1 or about 10 mg/kg. In some embodiments, each of the anti-CD20 X anti-CD28 bispecific antibodies or anti-CD20X anti-CD3 bispecific antibodies disclosed herein may be administered from about 0.1 mg to about 100 mg per dose.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### EXAMPLES

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are outlined in US Publications 2015/0307629, 2014/0288275 and WO2014/145806, all of which are expressly incorporated by reference in their entirety and particularly for the techniques outlined therein.

### Example 1: CD28 Binding Domains

Sequences for human, mouse, and cynomolgus CD28 are depicted in FIG. 1 and are useful for the development of cross-reactive CD28 antigen binding domains for ease of clinical development.

### 1A: Novel CD28 Binding Domains

An approach considered to avoid the superagonism associated with TGN1412 was to generate novel CD28 binding domains having lower affinity binding to CD28 and/or binding to a different CD28 epitope than TGN1412. In one campaign to generate such novel CD28 binding domains, in-house de novo phage libraries were panned against CD28.

### 1A(a): Phage-Derived Clone 1A7

It should be noted that this phage library utilized a human germline VL with diversity introduced into the LCDR3. The amino acid sequences for exemplary phage-derived clone 1A7 are depicted in FIG. 15.

The phage-derived clones were formatted as bivalent mAbs to investigate their binding characteristics. Plasmids containing the variable heavy and variable light domains of select clones were constructed by Gibson assembly and subcloned into a pTT5 expression vector containing the coding sequence for the IgG1 constant regions (with E233P/L234V/L235A/G236del/S67K ablation variants). DNA was transfected in HEK293E for expression and resulting bivalent mAbs were purified from the supernatant using protein A chromatography.

Affinity of the phage-derived bivalent mAbs for CD28 was screened using Octet, a BioLayer Interferometry (BLI)-based method. Experimental steps for Octet generally include the following: Immobilization (capture of ligand to a biosensor); Association (dipping of ligand-coated biosensors into wells containing the analyte); and Dissociation (returning of biosensors to well containing buffer). The resulting apparent dissociation constant (K_{Dapp}) are depicted in FIG. 28 for XENP28428 (based on clone 1A7) and additional phage-derived comparators.

Binding of the phage-derived bivalent mAbs to cell-surface CD28 was investigated. Human PBMCs were incubated with indicated concentrations of XENP28428 or comparator phage-derived mAbs for 1 hour at 4° C. Cells were then then stained with Alexa Fluor® 647 AffiniPure F(ab′)₂ Fragment Goat Anti-Human IgG, Fcγ fragment specific secondary antibody (Jackson ImmunoResearch, West Grove, Penn.) for 1 hour at 4° C. and analyzed by flow cytometry. The data (FIG. 28) show that the phage-derived mAbs were able to bind human PBMCs, although with much weaker maximum binding than prior art anti-CD28 mAb HuTN228 (XENP27181, sequences for which are depicted in FIG. 27).

### 1A(b): 1A7 is not Superagonistic

Potential superagonism of XENP28428 was assessed by air-drying per the Stebbings protocol (Stebbings R. et al. 2007). Air-drying of test articles was achieved by drying in a SpeedVac™ for 2 hours at room temperature. Human PBMCs were treated for 24 hours with 10 μg of air-dried XENP28428 (parental αCD28 mAb 1A7), and activity was compared to the superagonist TGN1412 (XENP29154; sequences for which are depicted in FIG. 27) or PBS control. Air-dried TGN1412 promoted IFNγ secretion from unstimulated human PBMC. In comparison, IFNγ level in PBMCs treated with air-dried XENP28428 remained similar to the negative control of PBS (data shown in FIG. 37).

### 1A(c): Engineering 1A7 Affinity Variants

Towards optimization of CD20 x CD28 bsAbs as described in Example 5, numerous 1A7 affinity variants were developed by engineering VH variants (illustrative sequences as depicted in FIG. 16 and additional sequences depicted in FIG. 22), VL variants (illustrative sequences as depicted in FIG. 17 and additional sequences depicted in FIG. 23), consensus sequences for which are depicted in FIG. 24, and combinations thereof (illustrative sequences for which are depicted in FIG. 18). Monovalent affinity of illustrative variants, in the context of scFvs, are depicted in FIG. 25. These sequences may further include “staple” modifications so that they may be used in a “stapled” scFv. Illustrative such VH and VL variants (and VH/VL combinations) with “staple” modifications are depicted in FIGS. 19-21.

### 1B: Additional CD28 Binding Domains

Sequences for additional CD28 binding domains which may find use in the CD20 x CD28 bsAbs of the invention are depicted in FIG. 26.

### Example 2: CD20 Binding Domains

Sequences for human, mouse, and cynomolgus CD20 are depicted in FIG. 2 and are useful for the development of cross-reactive CD20 antigen binding domains for ease of clinical development.

Sequences for CD20 binding domains which may find use in the CD20 x CD28 bsAbs of the invention are depicted in FIG. 30.

### Example 3: Engineering CD20 x CD28 bsAbs

T cells require multiple signals for complete activation and differentiation. Signal 1, promoted by recognition of a peptide-MHC (pMHC) complex by the T cell receptor (TCR), is absolutely required for T cell activation. Signal 2, which synergizes with, and amplifies Signal 1, is typically provided by the interaction of the CD28 ligands CD80 and CD86 with CD28 itself. Although CD28 engagement alone is inert, when combined with Signal 1 activation, it enhances T-cell activation, survival, and proliferative signals, including IL2 secretion (see FIG. 31). As CD80 and CD86 are naturally expressed by professional antigen-presenting cells (APC), the extent of CD28 costimulation in the tumor setting can be highly variable. By creating this novel class of tumor-targeted CD28 bispecific antibodies, CD80/CD86 engagement of CD28 can be mimicked, providing a source of Signal 2. Notably, signal can either be provided by the natural TCR:pMHC recognition of tumor cells, or it can be provided by combination of the CD28 bispecific with a CD3 T cell engager (which can mimic Signal 1). As there are an increasing number of CD3 T cell engagers undergoing clinical development for B-cell malignancies, it would be useful to develop CD20 x CD28 bsAbs to synergize with such molecules.

### 3A: Formats

### 3A(a): 1+1 Fab-scFv-Fc

The 1+1 Fab-scFv-Fc format (depicted schematically in FIG. 29A) comprises a first monomer comprising a single-chain Fv (“scFv”) with a first antigen binding specificity covalently attached to a first heterodimeric Fc domain i.e., scFv-domain linker-CH2-CH3, a second monomer comprising a heavy chain i.e., VH-CH1-hinge-CH2-CH3, wherein the CH2-CH3 is a second heterodimeric Fc domain complementary to the first heterodimeric Fc domain, and a light chain (LC) transfected separately so that a Fab domain having a second antigen binding specificity is formed with the variable heavy domain. Sequences for illustrative CD20 x CD28 bsAbs (based on binding domains as described in Examples 1 and 2) in the 1+1 Fab-scFv-Fc format are depicted in FIG. 33.

### 3A(b): 2+1 Fab2-scFv-Fc

The 2+1 Fab₂-scFv-Fc format (depicted schematically in FIG. 29B) comprises a first monomer comprising a first VH domain covalently attached to an scFv (having a first antigen binding specificity) covalently attached to a first heterodimeric Fc domain i.e., VH1-CH1-domain linker-scFv-domain linker-CH2-CH3, a second monomer comprising a second VH domain covalently attached to a complementary second heterodimeric Fc domain i.e., VH2-CH1-hinge-CH2-CH3, and a LC transfected separately so that Fab domains having a second antigen binding specificity are formed with the VH domains. Sequences for illustrative CD20 x CD28 bsAbs (based on binding domains as described in Examples 1 and 2) in the 2+1 Fab₂-scFv-Fc format are depicted in FIG. 34.

3A(c): 2+1 stackFab2-scFv-Fc

The 2+1 stackFab₂-scFv-Fc format (depicted schematically in FIG. 29F) comprises a first monomer comprising a first VH domain covalently attached to a second VH domain covalently attached to a first heterodimeric Fc domain i.e. VH1-CH1-domain-linker-VH2-CH1-hinge-CH2-CH3, a second monomer comprising an scFv (having a first antigen binding specificity) covalently attached to a complementary second heterodimeric Fc domain i.e., scFv-domain linker-CH2-CH3, and a LC transfected separately so that Fab domains having a second antigen binding specificity are formed with the VH domains. Sequences for illustrative CD20 x CD28 bsAbs (based on binding domains as described in Examples 1 and 2) in the 2+1 stackFab₂-scFv-Fc format are depicted in FIG. 35.

3A(d): 2+1 mAb-scFv

The 2+1 mAb-scFv format (depicted schematically in FIG. 29E) comprises a first monomer comprising a VH domain covalently attached to a first heterodimeric Fc domain covalently attached to an scFv (having a first antigen binding specificity) i.e. VH-CH1-hinge-CH2-CH3-domain linker-scFv, a second monomer comprising a VH domain covalently attached to a complementary second heterodimeric Fc domain i.e. VH-CH1-hinge-CH2-CH3, and a LC transfected separately so that Fab domains having a second antigen specificity are formed with the VH domains. Sequences for illustrative CD20 x CD28 bsAbs (based on binding domains as described in Examples 1 and 2) in the 2+1 mAb-scFv format are depicted in Figure.

### 3B: Platforms

Any number of heterodimerization approaches as is known in the art could find use in the bispecific formats, in combination with any number of approaches for purifying heterodimers from contaminating homodimers, including those depicted in FIGS. 3-4. Regardless of bsAb format, the CD28 bispecific antibodies are monovalent for CD28 and incorporate Fc variants engineered to ablate FcγR binding to avoid potential superagonism. Such Fc variants include those depicted in FIG. 5. Any of the number of linkers as is known in the art may find use in linking the VH and VL domains of the scFv. Finally, it may be useful to maximize serum half-life of the bsAbs, and any of the number of half-life extending variants as is known in the art may find use in these bsAbs.

Two platforms were used, herein referred to as Platform X and Platform Y.

### 3B(a): Platform X

Platform X (as described in FIG. 8) utilizes Backbones 1 or 11 in FIG. 10 or the Platform X backbone in FIG. 11. Specifically, this Platform utilizes the L368D/K370S (on a first monomer) S364K/E357Q (on a second monomer; typically the monomer with an scFv) heterodimeric Fc variants. One monomer (typically the monomer comprising L368D/K370S or the monomer that does not include an scFv) further includes pI variants N208D/Q295E/N384D/Q418E/N421D to increase negative charge of the chain. The scFv utilizes a positively charged (GKPGS)₄ linker (SEQ ID NO:42) between the VH and VL domains to increase positive charge of the scFv-comprising chain. Collectively, these two approaches enable easy purification of heterodimers from contaminating homodimers. The FcγR ablation variants utilized in this platform are the E233P/L234V/L235A/G236_/S267K substitutions on both the first and second monomers. In some cases, this platform includes the M428L/N434S half-life extension variants.

### 3B(b): Platform Y

Platform Y (as described in FIG. 8) utilizes Backbones 13 or 14 in FIG. 10 or the Platform J backbone in FIG. 11. Specifically, this Platform utilizes the T366S/L368A/Y407V (on a first monomer):T366W (on a second monomer) heterodimeric Fc variants. One monomer (typically the monomer comprising Fab) further includes H435R/Y436F to facilitate purification. The scFv may utilize a GGSEGKSSGSGSESKSTGGS (SEQ ID NO:55) (unstapled) or a GGGSGGSGGCPPCGGSGG (SEQ ID NO:56) (stapled) linker between the VH and VL domains. When using the latter stapled scFv linker, the VH and VL further include “staple” modifications/cysteine substitutions to form disulfide bridges with the linker. The FcγR ablation variants utilized in this platform are the L234A/L235A/D265S substitutions on both the first and second monomers. In some cases, this platform includes the M252Y/S254T/T256E half-life extension variants. Sequences for illustrative CD20 x CD28 bsAbs in Platform J are depicted in FIG. 33 as XENP42156, XENP42157, XENP42159, and XENP42160 and FIG. 36 as XENP42162 and XENP42163.

It should be noted that components from Platform X and Platform Y can be mix and matched. For example, Platform Y may utilize the (GKPGS)₄ linker (SEQ ID NO:42) of Platform X, for example, XENP42158 and XENP42161, sequences as depicted in FIG. 33. Additionally, each of Platform X and Platform Y may use alternative variants (e.g., alternative scFv linkers, alternative half-life extension variants, alternative FcγR ablation variants, etc.).

### Example 4: In Vitro Characterization of CD20 x CD28 bsAbs

Unless specified otherwise, illustrative CD79b x CD20 x CD3 trispecific antibody (triAb) used in the experiments herein is the CD79b x CD20 x CD3 bsAb sequences depicted in Figure.

Cancer cell lines used were OCI-Ly-10, Carnaval, Daudi, Ramos, Jeko, and WIL2-S. Cell surface antigen density levels were estimated by FACS using fluorescently-labeled beads as advised by the QuickCal protocol (Bangs Laboratories, Inc., Fishers, IN). 50,000 cells per well and MESF beads were combined with labeled mAbs for 30 minutes at 4° C. Cells were then washed and fixed in 1% PFA. Flow cytometry was performed to determine antibody binding, and antigen density calculations were carried out using QuickCal® V.2.3. software (Bangs Laboratories, Inc., Fishers, IN). Data depicting antigen density on the various cell lines are depicted in Table 3.

TABLE 3
Cancer Cell Line Antigen Density

| | Jeko | Ramos | Daudi | OCI-Ly-10 | Carnaval | WIL2-S |
|---|---|---|---|---|---|---|
| CD86 | 27K | 16K | 97K | 24K | 282K | 90K |
| CD80 | 1K | 0 | 14K | 8K | 14K | 11K |
| CD79b | 1.2M | 466K | 304K | 153K | 264K | 10K |
| CD20 | 1.2M | 900K | 726K | 1M | 1.2M | 275K |

4A: CD20 X CD28 bsAbs Combine Productively with CD79B X CD20 X CD3 triAb

CD20 x CD28 bsAbs were engineered and produced with various CD20 binding domains (including high affinity CD20-A_H1.202_L1.113, low affinity CD20-A_H1L1, CD20-B, and CD20-C, sequences as depicted in Figure) and CD28 binding domains of varying affinities (1A7_H1.14_L1.71, 1A7_H1_L1.71, and 1A7_H1_L1.71 respectively having 37 nM, 96 nM, and 180 nM CD28 binding affinities, sequences as depicted in Figure) in the 1+1 Fab-scFv-Fc and 2+1 mAb-scFv formats (in Platform X and Platform J) (sequences depicted in Figures and). To investigate their activity, the CD20 x CD28 bsAbs were paired with an illustrative CD79b x CD20 x CD3 T cell engager.

Carnaval, OCI-Ly-10, and Ramos cancer cells were incubated with T cells (1:1 effector:target ratio) in the presence of XENP42157 or XENP42160 and a CD79B x CD20 x CD3 triAb. In a first set of experiments, 1 μg/mL T cell engagers were combined with dose titration of CD20 x CD28 bsAb. In another set of experiments, 1 μg/mL CD20 x CD28 bsAb were combined with dose titration of CD3 T cell engagers. IL2 secretion by T cells was assessed after 24 hours, data for which are depicted in FIGS. 38-39.

### Example 5: CD20 X CD28 Enhancement of CD79bXCD20XCD3-Induced T-Cell Mediated Cytotoxicity

The ability of CD20 X CD28 bsAb to enhance T cell-mediated cytotoxicity was evaluated using co-culture assays of healthy donor-derived pan T cells at an E:T ratio of 1:1 in CD79b/CD20-expressing CARNAVAL and OCI-Ly10 cell lines. T cells from n=3 healthy donors were tested in T-cell/tumor co-culture cytotoxicity assays with the indicated antibodies and target cell lines expressing mKate2. Cytotoxicity was measured over 6 days. In a combination treatment, CD20 X CD28 bsAb enhanced CD79bXCD20XCD3 induced T-cell-mediated cytotoxicity of tumor cells in a concentration-dependent manner at an E:T ratio of 1:1 in the CD79b⁺CD20⁺ cell lines, OCI-Ly10, and CARNAVAL, but not in the K562 cell line (see FIG. 40). Compared to monotherapy treatment with the CD79bXCD20XCD3, the combination treatment of CD79bXCD20XCD3 with 10 nM CD20 X CD28 bsAb shifted the cytotoxicity EC₅₀ values in CARNAVAL and OCI-Ly10 cells by 31.8 to 40.3-fold, respectively. There was no enhancement of CD79bXCD20XCD3-induced cytotoxicity in any of the cell lines tested with the NullxCD28 control antibody, demonstrating that CD20 is required for CD20 X CD28 bsAb-mediated enhancement of T-cell-mediated cytotoxicity. In K562 cells, neither CD79bXCD20XCD3, CD20 X CD28 bsAb, nor their combination showed an effect on T-cell mediated cytotoxicity (see FIG. 40A).

The ability of CD20 X CD28 bsAb to enhance T cell activation was evaluated using CD25, measured as CD25-FITC area/well over 6 days. In a combination treatment, CD20 X CD28 bsAb enhanced CD79bXCD20XCD3 induced T-cell activation in a concentration-dependent manner at an E:T ratio of 1:1 in the CD79b+CD20+ cell lines, OCI-Ly10, and CARNAVAL, but not in the K562 cell line (see FIG. 40B). Compared to monotherapy treatment with the CD79bXCD20XCD3, the combination treatment of CD79bXCD20XCD3 with 10 nM CD20 X CD28 bsAb shifted the T-cell activation EC50 from 4.71 nM to 0.092 nM in OCI-Ly10 (51.2-fold) and from 0.76 nM to 0.041 nM in CARNAVAL (18.4-fold). There was no enhancement of CD79bXCD20XCD3-induced cytotoxicity in any of the cell lines tested with the NullxCD28 control antibody, demonstrating that CD20 is required for CD20 X CD28 bsAb-mediated enhancement of T-cell activation (see FIG. 40B).

Synergy was evaluated independently for each individual T cell donor (n=3) using a 4PL model with comparable results for the most highly synergistic areas. Strong synergy was observed in conditions of medium to high concentrations of CD20XCD28 bsAb and CD79bXCD20XCD3 in both CARNAVAL and OCI-Ly10 cell lines (see FIG. 40C).

### Example 6: In Vivo Anti-Tumor Activity

The effect of CD20XCD28 bsAb in combination with CD79bXCD20XCD3 was tested in subcutaneous (SC) OCI-Ly10 DLBCL xenografts in NSG mice. Tumor cells were implanted on Day 0, T cells were injected on Day 13, and dosing occurred on Days 14, 17, 20, 24, 27, 31, 34, 38, 41, 45 and 48 (dosing period indicated by the bar under the X-axis). For combination treatments, the indicated dose of CD20 X CD28 bsAb was combined with CD79bXCD20XCD3 at 1 mg/kg.

TGI (Tumor Growth Inhibition) of SC OCI-Ly10 DLBCL xenografts was calculated on Day 31, when at least two thirds of mice per group remained on study. Treatment with CD20 X CD28 bsAb at 0.5 mg/kg did not result in antitumor efficacy (2% TGI, ns). Treatment with CD79bxCD20xCD3 antibody at 0.3 mg/kg alone or in combination with CD20 X CD28 bsAb at any doses tested did not affect OCI-Ly10 tumor growth as compared to DPBS control treatment (data not shown). Treatment with CD79bXCD20XCD3 at 1 mg/kg resulted in suboptimal, statistically but not biologically significant TGI of 44%, as compared to DPBS control treatment. Combination of CD79bXCD20XCD3 at 1 mg/kg with CD20 X CD28 bsAb at 0.02, 0.1, and 0.5 mg/kg resulted in significant TGI of 50%, 49% and 75%, respectively, as compared to DPBS control treatment.

On Day 38, combination of CD79bXCD20XCD3 at 1 mg/kg with CD20 X CD28 bsAb at 0.5 mg/kg statistically enhanced antitumor efficacy compared to CD79bXCD20XCD3 monotherapy treatment. Lower dose combination treatments did not result in additional tumor growth inhibition compared to CD79bXCD20XCD3 at 1 mg/kg monotherapy. Combination treatment of CD79bXCD20XCD3 at 1 mg/kg with CD20 X CD28 bsAb at 0.5 mg/kg resulted in 97% TR with 2 of 8 partial response and 6 of 8 complete response by Day 62 (see FIG. 41).

### Example 7: CD20 X CD28 Enhancement of CD79bXCD20XCD3-Induced T-Cell Mediated Cytotoxicity in Cell Lines Expressing Low Levels of CD20 and CD79b

Cancer cell lines used were WILL-2 and NALM6. Cell surface antigen density levels were estimated by FACS using fluorescently-labeled beads as advised by the QuickCal protocol (Bangs Laboratories, Inc., Fishers, IN). 50,000 cells per well and MESF beads were combined with labeled mAbs for 30 minutes at 4° C. Cells were then washed and fixed in 1% PFA. Flow cytometry was performed to determine antibody binding, and antigen density calculations were carried out using QuickCal® V.2.3. software (Bangs Laboratories, Inc., Fishers, IN). Data depicting antigen density on the various cell lines are depicted in Table 4.

TABLE 4
Cancer cell line antigen density

| | WILL-2 | NALM6 |
|---|---|---|
| CD20 | 4,000 | Below limit |
| | | detection |
| CD79b | 2,000 | ~130 |

The ability of CD20 X CD28 bsAb to enhance T cell-mediated cytotoxicity was evaluated using co-culture assays of healthy donor-derived pan T cells at an E:T ratio of 5:1 in low CD79b/low CD20-expressing WILL-2 and NALM6 cell lines. T cells from n=3-6 healthy donors were tested in T-cell/tumor co-culture cytotoxicity assays with the indicated antibodies and target cell lines expressing mKate2. Cytotoxicity was measured over 6 days. In a combination treatment, CD20 X CD28 bsAb enhanced CD79bXCD20XCD3 induced T-cell-mediated cytotoxicity of tumor cells in a concentration-dependent manner at an E:T ratio of 5:1 in the CD79b+CD20+ cell lines, WILL-2 and NALM6 (see FIG. 43 A). Compared to monotherapy treatment with the CD79bXCD20XCD3, the combination treatment of CD79bXCD20XCD3 with 10 nM CD20 X CD28 bsAb shifted the maximum % cytotoxicity from 23.6% to 68.4% in WILL-2 (EC50 shifted by 9.2-fold) and from 42.1% to 74.1% in NALM6 cells (EC50 shifted by 6.5-fold). There was no enhancement of CD79bXCD20XCD3-induced cytotoxicity in any of the cell lines tested with the NullxCD28 control antibody, demonstrating that CD20 is required for CD20 X CD28 bsAb-mediated enhancement of T-cell-mediated cytotoxicity (see FIG. 42A).

CD20 X CD28 bsAb enhanced CD79bXCD20XCD3 induced T-cell activation was also measured by CD25 expression in a concentration-dependent manner at an E:T ratio of 5:1 in the CD79b+CD20+ cell lines, WILL-2 and NALM6 (see FIG. 43B). Compared to monotherapy treatment with the CD79bXCD20XCD3, the combination treatment of CD79bXCD20XCD3 with 10 nM CD20 X CD28 bsAb shifted the T-cell activation EC50 values in WILL-2 and NALM6 cells by 69.7 to 4.9-fold, respectively. There was no enhancement of CD79bXCD20XCD3-induced T-cell activation in any of the cell lines tested with the NullxCD28 control antibody, demonstrating that CD20 is required for CD20 X CD28 bsAb-mediated enhancement of T-cell-mediated cytotoxicity (see FIG. 42B).

Synergy was evaluated independently for each individual T cell donor (n=3-6) using a 4PL model with comparable results for the most highly synergistic areas. Strong synergy was observed in conditions of high concentrations of CD20XCD28 bsAb and CD79bXCD20XCD3 in both WILL-2 and NALM6 cell lines (see FIGS. 42C and 42D).

### Example 8: CD20 X CD28 Enhancement of CD79bXCD20XCD3-Induced T-Cell Activation, Proliferation and Cytokine Release

The ability of CD20 X CD28 bsAb to enhance T cell activation and proliferation was evaluated using co-culture assays of healthy donor-derived pan T cells at an E:T ratio of 1:1 in CD79b/CD20-expressing CARNAVAL and OCI-Ly10 cell lines. T cells from n=3 healthy donors were tested in T-cell/tumor co-culture cytotoxicity assays with the indicated antibodies and target cell lines for 72 hours. T-cell activation was measured as % CD25 and T-cell proliferation was measured as the % of cells that showed CellTrace Violet dilution compared to non stimulated T-cells.

In a combination treatment, CD20 X CD28 bsAb enhanced CD79bXCD20XCD3 induced T-cell activation and proliferation in a concentration-dependent manner at an E:T ratio of 1:1 in the CD79b+CD20+ cell lines, OCI-Ly10, and CARNAVAL (see FIG. 43A). The ability of CD20 X CD28 bsAb to enhance cytokine release was evaluated using co-culture assays of healthy donor-derived pan T cells at an E:T ratio of 1:1 in CD79b/CD20-expressing CARNAVAL and OCI-Ly10 cell lines. T cells from n=3 healthy donors were tested in T-cell/tumor co-culture cytotoxicity assays with the indicated antibodies and target cell lines for 72 hours. Cytokines were measured using the iQue Qbeads®Human PlexScreen (Sartorius) to measure interferon (IFN)-γ, IL-2 and tumor necrosis factor (TNF) a, according to the manufacturer's recommendations. In a combination treatment, CD20 X CD28 bsAb enhanced CD79bXCD20XCD3 induced increased cytokine release of IFN-γ, IL-2 and TNF-α in a concentration-dependent manner at an E:T ratio of 1:1 in the CD79b+CD20+ cell lines, OCI-Ly10, and CARNAVAL (see FIG. 43B).

### Example 9: CD20 X CD28 Enhancement of CD79bXCD20XCD3-Induced T-Cell Proliferation, Activation, Differentiation and Cytokine Release

The ability of CD20 X CD28 bsAb to enhance TCE-mediated T-cell proliferation, activation and differentiation was evaluated using co-culture assays of healthy donor-derived pan T cells and lymphoma cell lines, CARNAVAL or OCI-Ly10, at an E:T ratio of 1:1. T cells from n=2 healthy donors were tested in T-cell/tumor co-culture cytotoxicity assays with the indicated therapeutic antibodies and target cell lines for 72 hours. Supernatants were collected to measure secreted cytokines. Cytokines were measured using the Human Pro-inflammatory Panel 1 kit from Meso Scale Discovery (MSD) to measure interferon (IFN)-γ, IL-2 and tumor necrosis factor (TNF) a, according to the manufacturer's recommendations. Cells were stained using anti-CD4/PE-Cy7, anti-CD8/PerCP-Cy5.5, anti-CD69/PE, anti-CD25/APC, anti-CD45RA/Alexa Fluor 700, anti-CD45RO/BV605, anti-CD197 (CCR7)/AF488 and anti-CD19/BV786 antibodies and analyzed by flow cytometry. To measure T-cell proliferation, CTV dilution was evaluated. CTV fluorescence intensity is reduced through subsequent cell divisions (to approximately half of the fluorescent label of the parent cells) with each cell division and appears as a discrete peak in a histogram graph. To measure the level of T-cell activation, the absolute percentage of CD25 expressing cells for either CD4+ or CD8+ T cells was measured. To evaluate T-cell differentiation, the absolute percentage of T naïve (CCR7+CD45RO−), central memory T (TCM; CCR7+CD45RO+), effector memory T (TEM; CCR7-CD45RO+), and effector T (TE; CCR7-CD45RO−) cells for either CD4+ or CD8+ T cells was determined.

In a combination treatment, CD20 X CD28 bsAb enhanced CD79b X CD20 X CD3 induced T-cell proliferation. Compared to monotherapy treatment with the CD79b X CD20 X CD3, the combination treatment of CD79b X CD20 X CD3 with 10 nM CD20 X CD28 bsAb had a stronger effect on T-cell proliferation of pan T-cells, CD8+ and CD4+ cells. CD20 X CD28 bsAb had no effect on T-cell proliferation in the absence of TCE. Likewise, there was no enhancement of CD79b X CD20 X CD3-induced cytotoxicity in any of the cell lines tested with the CD20xNull or NullxCD28 control antibody, demonstrating that CD20 and CD28 binding is required for CD20 X CD28 bsAb-mediated enhancement of T-cell proliferation (see FIGS. 44A and 44B). Representative data for 1 T-cell donor of 2 donors tested in duplicate.

In a combination treatment, CD20 X CD28 bsAb enhanced CD79bXCD20XCD3 induced T-cell activation as measured by CD25 expression in a co-culture of pan-T cells and lymphoma cells, CARNAVAL or OCI-Ly10, at an E:T ratio of 1:1. Compared to monotherapy treatment with the CD79b X CD20 X CD3, the combination treatment of CD79b X CD20 X CD3 with 10 nM CD20 X CD28 bsAb increased the T-cell activation in pan-T and CARNAVAL or OCI-Ly10 co-cultures by 31 or 44% on CD4+ T-cells and 28 or 19% on CD8+ cells, respectively. There was no enhancement of CD79b X CD20 X CD3-induced T-cell activation with the CD20xNull or NullxCD28 control antibody, demonstrating that CD20 and CD28 crosslinking is required for CD20 X CD28 bsAb-mediated enhancement of T-cell activation (see FIG. 44C). Data is average+/−SD of 2 donors tested in duplicate.

In a combination treatment, CD20 X CD28 bsAb enhanced CD79b X CD20 X CD3-induced decrease in T naïve cells and increase in central memory T (TCM) cells. The combination of CD79b X CD20 X CD3 with 10 nM CD20 X CD28 bsAb further increased the percentage of TCM cells compared to CD79b X CD20 X CD3 monotherapy treatment, with a concurrent decrease in T naïve, effector memory T (TEM) cells, and effector T (TE) cells. CD20 X CD28 bsAb had no effect on T-cell differentiation in the absence of TCE. There was no enhancement of CD79b X CD20 X CD3-induced T-cell differentiation with the CD20xNull or NullxCD28 control antibody, demonstrating that CD20 and CD28 binding is required for CD20 X CD28 bsAb-mediated enhancement of T-cell differentiation (see FIGS. 44D, 44E, and 44F). Data is average+/−SD of 2 donors tested in duplicate. T naïve (CCR7+CD45RO−), central memory T (TCM; CCR7+CD45RO+), effector memory T (TEM; CCR7-CD45RO+), and effector T (TE; CCR7-CD45RO−).

In a combination treatment, CD20 X CD28 bsAb enhanced CD79b X CD20 X CD3-induced increase of IFN-γ, IL-2 and TNF-α in the supernatant of co-cultures of pan-T cells and CARNAVAL or OCI-Ly10 cells at two different doses of CD79b X CD20 X CD3 (see FIG. 44G). CARNAVAL: CD79bXCD20XCD3 Dose 1: 0.1 nM, dose 2: 0.5 nM. OCI-Ly10: CD79bXCD20XCD3 Dose 1: 1 nM, dose 2: 5 nM.

### Embodiments

The disclosure is also directed to the following aspects:

Embodiment 1. An antibody or an antibody fragment thereof comprising:
(a) a first antigen binding domain that binds to CD20, wherein the first antigen binding domain comprises:
   (i) a heavy chain complimentarity determining region 1 (vhCDR1), a vhCDR2 and a vhCDR3 of a variable heavy chain domain (VH1) having an amino acid sequence of SEQ ID NO:439, and a light chain complimentarity determining region 1 (vICDR1), a vlCDR2 and a vlCDR3 of a variable light chain domain (VL1) having an amino acid sequence of SEQ ID NO:443;
   (ii) a vhCDR1, a vhCDR2 and a vhCDR3 of a variable heavy chain domain (VH1) having an amino acid sequence of SEQ ID NO:447, and a light chain complimentarity determining region (vICDR1), a vlCDR2 and a vlCDR3 of a variable light chain domain (VL1) having an amino acid sequence of SEQ ID NO:451;
   (iii) a vhCDR1, a vhCDR2 and a vhCDR3 of a variable heavy chain domain (VH1) having an amino acid sequence of SEQ ID NO:455, and a light chain complimentarity determining region (vICDR1), a vlCDR2 and a vlCDR3 of a variable light chain domain (VL1) having an amino acid sequence of SEQ ID NO:459;
   (iv) a vhCDR1, a vhCDR2 and a vhCDR3 of a variable heavy chain domain (VH1) having an amino acid sequence of SEQ ID NO:463, and a light chain complimentarity determining region (vICDR1), a vlCDR2 and a vlCDR3 of a variable light chain domain (VL1) having an amino acid sequence of SEQ ID NO:467;
   (v) a vhCDR1, a vhCDR2 and a vhCDR3 of a variable heavy chain domain (VH1) having an amino acid sequence of SEQ ID NO:471, and a light chain complimentarity determining region (vICDR1), a vlCDR2 and a vlCDR3 of a variable light chain domain (VL1) having an amino acid sequence of SEQ ID NO:472;
   (vi) a vhCDR1, a vhCDR2 and a vhCDR3 of a variable heavy chain domain (VH1) having an amino acid sequence of SEQ ID NO:473, and a light chain complimentarity determining region (vICDR1), a vlCDR2 and a vlCDR3 of a variable light chain domain (VL1) having an amino acid sequence of SEQ ID NO:474;
   (vii) a vhCDR1, a vhCDR2 and a vhCDR3 of a variable heavy chain domain (VH1) having an amino acid sequence of SEQ ID NO:475, and a light chain complimentarity determining region (vICDR1), a vlCDR2 and a vlCDR3 of a variable light chain domain (VL1) having an amino acid sequence of SEQ ID NO:472;
   (viii) a vhCDR1, a vhCDR2 and a vhCDR3 of a variable heavy chain domain (VH1) having an amino acid sequence of SEQ ID NO:477, and a light chain complimentarity determining region (vICDR1), a vlCDR2 and a vlCDR3 of a variable light chain domain (VL1) having an amino acid sequence of SEQ ID NO:478;
   (ix) a vhCDR1, a vhCDR2 and a vhCDR3 of a variable heavy chain domain (VH1) having an amino acid sequence of SEQ ID NO:479, and a light chain complimentarity determining region (vICDR1), a vlCDR2 and a vlCDR3 of a variable light chain domain (VL1) having an amino acid sequence of SEQ ID NO:480;
   (x) a vhCDR1, a vhCDR2 and a vhCDR3 of a variable heavy chain domain (VH1) having an amino acid sequence of SEQ ID NO:481, and a light chain complimentarity determining region (vICDR1), a vlCDR2 and a vlCDR3 of a variable light chain domain (VL1) having an amino acid sequence of SEQ ID NO:482;
   (xi) a vhCDR1, a vhCDR2 and a vhCDR3 of a variable heavy chain domain (VH1) having an amino acid sequence of SEQ ID NO:483, and a light chain complimentarity determining region (vICDR1), a vlCDR2 and a vlCDR3 of a variable light chain domain (VL1) having an amino acid sequence of SEQ ID NO:484;
   (xii) a vhCDR1, a vhCDR2 and a vhCDR3 of a variable heavy chain domain (VH1) having an amino acid sequence of SEQ ID NO:487, and a light chain complimentarity determining region (vICDR1), a vlCDR2 and a vlCDR3 of a variable light chain domain (VL1) having an amino acid sequence of SEQ ID NO:467;
   (xiii) a vhCDR1, a vhCDR2 and a vhCDR3 of a variable heavy chain domain (VH1) having an amino acid sequence of SEQ ID NO:489, and a light chain complimentarity determining region (vICDR1), a vlCDR2 and a vlCDR3 of a variable light chain domain (VL1) having an amino acid sequence of SEQ ID NO:490;
   (xiv) a vhCDR1, a vhCDR2 and a vhCDR3 of a variable heavy chain domain (VH1) having an amino acid sequence of SEQ ID NO:491, and a light chain complimentarity determining region (vICDR1), a vlCDR2 and a vlCDR3 of a variable light chain domain (VL1) having an amino acid sequence of SEQ ID NO:492;
   (xv) a vhCDR1, a vhCDR2 and a vhCDR3 of a variable heavy chain domain (VH1) having an amino acid sequence of SEQ ID NO:495, and a light chain complimentarity determining region (vICDR1), a vlCDR2 and a vlCDR3 of a variable light chain domain (VL1) having an amino acid sequence of SEQ ID NO:496; or
   (xvi) a vhCDR1, a vhCDR2 and a vhCDR3 of a variable heavy chain domain (VH1) having an amino acid sequence of SEQ ID NO:497, and a light chain complimentarity determining region (vICDR1), a vlCDR2 and a vlCDR3 of a variable light chain domain (VL1) having an amino acid sequence of SEQ ID NO:498; and
(b) a second antigen binding domain that binds to CD28, wherein the second antigen binding domain comprises:
   (i) a vhCDR1, a vhCDR2 and a vhCDR3 of a variable heavy chain domain (VH2) having an amino acid sequence of SEQ ID NO:1, and a light chain complimentarity determining region (vICDR1), a vlCDR2 and a vlCDR3 of a variable light chain domain (VL2) having an amino acid sequence of SEQ ID NO:5;
   (ii) a vhCDR1, a vhCDR2 and a vhCDR3 of a variable heavy chain domain (VH2) having an amino acid sequence of SEQ ID NO:11, and a light chain complimentarity determining region (vICDR1), a vlCDR2 and a vlCDR3 of a variable light chain domain (VL2) having an amino acid sequence of SEQ ID NO:5;
   (iii) a vhCDR1, a vhCDR2 and a vhCDR3 of a variable heavy chain domain (VH2) having an amino acid sequence of SEQ ID NO:1, and a light chain complimentarity determining region (vICDR1), a vlCDR2 and a vlCDR3 of a variable light chain domain (VL2) having an amino acid sequence of SEQ ID NO:19;
   (iv) a vhCDR1, a vhCDR2 and a vhCDR3 of a variable heavy chain domain (VH2) having an amino acid sequence of SEQ ID NO:11, and a light chain complimentarity determining region (vICDR1), a vlCDR2 and a vlCDR3 of a variable light chain domain (VL2) having an amino acid sequence of SEQ ID NO:19;
   (v) a vhCDR1, a vhCDR2 and a vhCDR3 of a variable heavy chain domain (VH2) having an amino acid sequence of SEQ ID NO:15, and a light chain complimentarity determining region (vICDR1), a vlCDR2 and a vlCDR3 of a variable light chain domain (VL2) having an amino acid sequence of SEQ ID NO:5;
   (vi) a vhCDR1, a vhCDR2 and a vhCDR3 of a variable heavy chain domain (VH2) having an amino acid sequence of SEQ ID NO:15, and a light chain complimentarity determining region (vICDR1), a vlCDR2 and a vlCDR3 of a variable light chain domain (VL2) having an amino acid sequence of SEQ ID NO:19;
   (vii) a vhCDR1, a vhCDR2 and a vhCDR3 of a variable heavy chain domain (VH2) having an amino acid sequence of SEQ ID NO:63, and a light chain complimentarity determining region (vICDR1), a vlCDR2 and a vlCDR3 of a variable light chain domain (VL2) having an amino acid sequence of SEQ ID NO:75;
   (viii) a vhCDR1, a vhCDR2 and a vhCDR3 of a variable heavy chain domain (VH2) having an amino acid sequence of SEQ ID NO:67, and a light chain complimentarity determining region (vICDR1), a vlCDR2 and a vlCDR3 of a variable light chain domain (VL2) having an amino acid sequence of SEQ ID NO:75;
   (ix) a vhCDR1, a vhCDR2 and a vhCDR3 of a variable heavy chain domain (VH2) having an amino acid sequence of SEQ ID NO:63, and a light chain complimentarity determining region (vICDR1), a vlCDR2 and a vlCDR3 of a variable light chain domain (VL2) having an amino acid sequence of SEQ ID NO:79;
   (x) a vhCDR1, a vhCDR2 and a vhCDR3 of a variable heavy chain domain (VH2) having an amino acid sequence of SEQ ID NO:67, and a light chain complimentarity determining region (vICDR1), a vlCDR2 and a vlCDR3 of a variable light chain domain (VL2) having an amino acid sequence of SEQ ID NO:111;
   (xi) a vhCDR1, a vhCDR2 and a vhCDR3 of a variable heavy chain domain (VH2) having an amino acid sequence of SEQ ID NO:71, and a light chain complimentarity determining region (vICDR1), a vlCDR2 and a vlCDR3 of a variable light chain domain (VL2) having an amino acid sequence of SEQ ID NO:75;
   (xii) a vhCDR1, a vhCDR2 and a vhCDR3 of a variable heavy chain domain (VH2) having an amino acid sequence of SEQ ID NO:71, and a light chain complimentarity determining region (vICDR1), a vlCDR2 and a vlCDR3 of a variable light chain domain (VL2) having an amino acid sequence of SEQ ID NO:79;
   (xiii) a vhCDR1, a vhCDR2 and a vhCDR3 of a variable heavy chain domain (VH2) having an amino acid sequence of SEQ ID NO:320, and a light chain complimentarity determining region (vICDR1), a vlCDR2 and a vlCDR3 of a variable light chain domain (VL2) having an amino acid sequence of SEQ ID NO:324;
   (xiv) a vhCDR1, a vhCDR2 and a vhCDR3 of a variable heavy chain domain (VH2) having an amino acid sequence of SEQ ID NO:327, and a light chain complimentarity determining region (vICDR1), a vlCDR2 and a vlCDR3 of a variable light chain domain (VL2) having an amino acid sequence of SEQ ID NO:331;
   (xv) a vhCDR1, a vhCDR2 and a vhCDR3 of a variable heavy chain domain (VH2) having an amino acid sequence of SEQ ID NO:335, and a light chain complimentarity determining region (vICDR1), a vlCDR2 and a vlCDR3 of a variable light chain domain (VL2) having an amino acid sequence of SEQ ID NO:339;
   (xvi) a vhCDR1, a vhCDR2 and a vhCDR3 of a variable heavy chain domain (VH2) having an amino acid sequence of SEQ ID NO:343, and a light chain complimentarity determining region (vICDR1), a vlCDR2 and a vlCDR3 of a variable light chain domain (VL2) having an amino acid sequence of SEQ ID NO:347;
   (xvii) a vhCDR1, a vhCDR2 and a vhCDR3 of a variable heavy chain domain (VH2) having an amino acid sequence of SEQ ID NO:351, and a light chain complimentarity determining region (vICDR1), a vlCDR2 and a vlCDR3 of a variable light chain domain (VL2) having an amino acid sequence of SEQ ID NO:355;
   (xviii) a vhCDR1, a vhCDR2 and a vhCDR3 of a variable heavy chain domain (VH2) having an amino acid sequence of SEQ ID NO:359, and a light chain complimentarity determining region (vICDR1), a vlCDR2 and a vlCDR3 of a variable light chain domain (VL2) having an amino acid sequence of SEQ ID NO:355;
   (xix) a vhCDR1, a vhCDR2 and a vhCDR3 of a variable heavy chain domain (VH2) having an amino acid sequence of SEQ ID NO:367, and a light chain complimentarity determining region (vICDR1), a vlCDR2 and a vlCDR3 of a variable light chain domain (VL2) having an amino acid sequence of SEQ ID NO:371;
   (xx) a vhCDR1, a vhCDR2 and a vhCDR3 of a variable heavy chain domain (VH2) having an amino acid sequence of SEQ ID NO:375, and a light chain complimentarity determining region (vICDR1), a vlCDR2 and a vlCDR3 of a variable light chain domain (VL2) having an amino acid sequence of SEQ ID NO:380;
   (xxi) a vhCDR1, a vhCDR2 and a vhCDR3 of a variable heavy chain domain (VH2) having an amino acid sequence of SEQ ID NO:391, and a light chain complimentarity determining region (vICDR1), a vlCDR2 and a vlCDR3 of a variable light chain domain (VL2) having an amino acid sequence of SEQ ID NO:395;
   (xxii) a vhCDR1, a vhCDR2 and a vhCDR3 of a variable heavy chain domain (VH2) having an amino acid sequence of SEQ ID NO:399, and a light chain complimentarity determining region (vICDR1), a vlCDR2 and a vlCDR3 of a variable light chain domain (VL2) having an amino acid sequence of SEQ ID NO:403;
   (xxiii) a vhCDR1, a vhCDR2 and a vhCDR3 of a variable heavy chain domain (VH2) having an amino acid sequence of SEQ ID NO:407, and a light chain complimentarity determining region (vICDR1), a vlCDR2 and a vlCDR3 of a variable light chain domain (VL2) having an amino acid sequence of SEQ ID NO:411;
   (xxiv) a vhCDR1, a vhCDR2 and a vhCDR3 of a variable heavy chain domain (VH2) having an amino acid sequence of SEQ ID NO:415, and a light chain complimentarity determining region (vICDR1), a vlCDR2 and a vlCDR3 of a variable light chain domain (VL2) having an amino acid sequence of SEQ ID NO:419;
   (xxv) a vhCDR1, a vhCDR2 and a vhCDR3 of a variable heavy chain domain (VH2) having an amino acid sequence of SEQ ID NO:423, and a light chain complimentarity determining region (vICDR1), a vlCDR2 and a vlCDR3 of a variable light chain domain (VL2) having an amino acid sequence of SEQ ID NO:5; or
   (xxvi) a vhCDR1, a vhCDR2 and a vhCDR3 of a variable heavy chain domain (VH2) having an amino acid sequence of SEQ ID NO:431, and a light chain complimentarity determining region (vICDR1), a vlCDR2 and a vlCDR3 of a variable light chain domain (VL2) having an amino acid sequence of SEQ ID NO:435.

Embodiment 2. The antibody or the antibody fragment of embodiment 1, wherein
(a) the first antigen binding domain comprises:
   (i) a vhCDR1 having an amino acid sequence of SEQ ID NO:440; a vhCDR2 having an amino acid sequence of SEQ ID NO:441; and a vhCDR3 having an amino acid sequence of SEQ ID NO:442; and a vlCDR1 having an amino acid sequence of SEQ ID NO:444, a vlCDR2 having an amino acid sequence of SEQ ID NO: 445, and a vlCDR3 having an amino acid sequence of SEQ ID NO:446;
   (ii) a vhCDR1 having an amino acid sequence of SEQ ID NO:440; a vhCDR2 having an amino acid sequence of SEQ ID NO:449; and a vhCDR3 having an amino acid sequence of SEQ ID NO:450; and a vlCDR1 having an amino acid sequence of SEQ ID NO:452, a vlCDR2 having an amino acid sequence of SEQ ID NO:445, and a vlCDR3 having an amino acid sequence of SEQ ID NO:454;
   (iii) a vhCDR1 having an amino acid sequence of SEQ ID NO:440; a vhCDR2 having an amino acid sequence of SEQ ID NO:457; and a vhCDR3 having an amino acid sequence of SEQ ID NO:458; and a vlCDR1 having an amino acid sequence of SEQ ID NO:460, a vlCDR2 having an amino acid sequence of SEQ ID NO:445, and a vlCDR3 having an amino acid sequence of SEQ ID NO:462; or
   (iv) a vhCDR1 having an amino acid sequence of SEQ ID NO:464; a vhCDR2 having an amino acid sequence of SEQ ID NO:465; and a vhCDR3 having an amino acid sequence of SEQ ID NO:466; and a vlCDR1 having an amino acid sequence of SEQ ID NO:356, a vlCDR2 having an amino acid sequence of SEQ ID NO:357, and a vlCDR3 having an amino acid sequence of SEQ ID NO:470; and
(b) the second antigen binding domain comprises:
   (i) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:8;
   (ii) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vICDR2 having an amino acid sequence of SEQ ID NO:7, and a vICDR3 having an amino acid sequence of SEQ ID NO:8;
   (iii) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:22;
   (iv) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:22;
   (v) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:8; or
   (vi) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:22.

Embodiment 3. The antibody or the antibody fragment of embodiment 1 or 2, wherein:
(a) the first antigen binding domain comprises:
   (i) a VH1 having an amino acid sequence of SEQ ID NO:439; and a VL1 having an amino acid sequence of SEQ ID NO:443;
   (ii) a VH1 having an amino acid sequence of SEQ ID NO:447; and a VL1 having an amino acid sequence of SEQ ID NO:451;
   (iii) a VH1 having an amino acid sequence of SEQ ID NO:455; and a VL1 having an amino acid sequence of SEQ ID NO:459;
   (iv) a VH1 having an amino acid sequence of SEQ ID NO:463; and a VL1 having an amino acid sequence of SEQ ID NO:467;
   (v) a VH1 having an amino acid sequence of SEQ ID NO:471; and a VL1 having an amino acid sequence of SEQ ID NO:472;
   (vi) a VH1 having an amino acid sequence of SEQ ID NO:473; and a VL1 having an amino acid sequence of SEQ ID NO:474;
   (vii) a VH1 having an amino acid sequence of SEQ ID NO:475; and a VL1 having an amino acid sequence of SEQ ID NO:472;
   (viii) a VH1 having an amino acid sequence of SEQ ID NO:477; and a VL1 having an amino acid sequence of SEQ ID NO:478;
   (ix) a VH1 having an amino acid sequence of SEQ ID NO:479; and a VL1 having an amino acid sequence of SEQ ID NO:480;
   (x) a VH1 having an amino acid sequence of SEQ ID NO:481; and a VL1 having an amino acid sequence of SEQ ID NO:482;
   (xi) a VH1 having an amino acid sequence of SEQ ID NO:483; and a VL1 having an amino acid sequence of SEQ ID NO:484;
   (xii) a VH1 having an amino acid sequence of SEQ ID NO:487; and a VL1 having an amino acid sequence of SEQ ID NO:467;
   (xiii) a VH1 having an amino acid sequence of SEQ ID NO:489; and a VL1 having an amino acid sequence of SEQ ID NO:490;
   (xiv) a VH1 having an amino acid sequence of SEQ ID NO:491; and a VL1 having an amino acid sequence of SEQ ID NO:492;
   (xv) a VH1 having an amino acid sequence of SEQ ID NO:495; and a VL1 having an amino acid sequence of SEQ ID NO:496; or
   (xvi) a VH1 having an amino acid sequence of SEQ ID NO:497; and a VL1 having an amino acid sequence of SEQ ID NO:498; and
(b) the second antigen binding domain comprises:
   (i) a VH2 having an amino acid sequence of SEQ ID NO:1; and a VL2 having an amino acid sequence of SEQ ID NO:5;
   (ii) a VH2 having an amino acid sequence of SEQ ID NO:11; and a VL2 having an amino acid sequence of SEQ ID NO:5;
   (iii) a VH2 having an amino acid sequence of SEQ ID NO:1; and a VL2 having an amino acid sequence of SEQ ID NO:19;
   (iv) a VH2 having an amino acid sequence of SEQ ID NO:11; and a VL2 having an amino acid sequence of SEQ ID NO:19;
   (v) a VH2 having an amino acid sequence of SEQ ID NO:15; and a VL2 having an amino acid sequence of SEQ ID NO:5;
   (vi) a VH2 having an amino acid sequence of SEQ ID NO:15; and a VL2 having an amino acid sequence of SEQ ID NO:19;
   (vii) a VH2 having an amino acid sequence of SEQ ID NO:63; and a VL2 having an amino acid sequence of SEQ ID NO:75;
   (viii) a VH2 having an amino acid sequence of SEQ ID NO:67; and a VL2 having an amino acid sequence of SEQ ID NO:75;
   (ix) a VH2 having an amino acid sequence of SEQ ID NO:63; and a VL2 having an amino acid sequence of SEQ ID NO:79;
   (x) a VH2 having an amino acid sequence of SEQ ID NO:67; and a VL2 having an amino acid sequence of SEQ ID NO:111;
   (xi) a VH2 having an amino acid sequence of SEQ ID NO:71; and a VL2 having an amino acid sequence of SEQ ID NO:75;
   (xii) a VH2 having an amino acid sequence of SEQ ID NO:71; and a VL2 having an amino acid sequence of SEQ ID NO:79;
   (xiii) a VH2 having an amino acid sequence of SEQ ID NO:320; and a VL2 having an amino acid sequence of SEQ ID NO:324;
   (xiv) a VH2 having an amino acid sequence of SEQ ID NO:327; and a VL2 having an amino acid sequence of SEQ ID NO:331;
   (xv) a VH2 having an amino acid sequence of SEQ ID NO:335; and a VL2 having an amino acid sequence of SEQ ID NO:339;
   (xvi) a VH2 having an amino acid sequence of SEQ ID NO:343; and a VL2 having an amino acid sequence of SEQ ID NO:347;
   (xvii) a VH2 having an amino acid sequence of SEQ ID NO:351; and a VL2 having an amino acid sequence of SEQ ID NO:355;
   (xviii) a VH2 having an amino acid sequence of SEQ ID NO:359; and a VL2 having an amino acid sequence of SEQ ID NO:355;
   (xix) a VH2 having an amino acid sequence of SEQ ID NO:367; and a VL2 having an amino acid sequence of SEQ ID NO:371;
   (xx) a VH2 having an amino acid sequence of SEQ ID NO:375; and a VL2 having an amino acid sequence of SEQ ID NO:380; or
   (xxi) a VH2 having an amino acid sequence of SEQ ID NO:391; and a VL2 having an amino acid sequence of SEQ ID NO:395;
   (xxii) a VH2 having an amino acid sequence of SEQ ID NO:399; and a VL2 having an amino acid sequence of SEQ ID NO:403;
   (xxiii) a VH2 having an amino acid sequence of SEQ ID NO:407; and a VL2 having an amino acid sequence of SEQ ID NO:411;
   (xxiv) a VH2 having an amino acid sequence of SEQ ID NO:415; and a VL2 having an amino acid sequence of SEQ ID NO:419;
   (xxv) a VH2 having an amino acid sequence of SEQ ID NO:423; and a VL2 having an amino acid sequence of SEQ ID NO:5; or
   (xxvi) a VH2 having an amino acid sequence of SEQ ID NO:431; and a VL2 having an amino acid sequence of SEQ ID NO:435.

Embodiment 4. The antibody or the antibody fragment of any one of embodiments 1-3, wherein:
(a) the first antigen binding domain comprises:
   (i) a VH1 having an amino acid sequence that is at least 95% identical to SEQ ID NO:439; and a VL1 having an amino acid sequence that is at least 95% identical to SEQ ID NO:443;
   (ii) a VH1 having an amino acid sequence that is at least 95% identical to SEQ ID NO:447; and a VL1 having an amino acid sequence that is at least 95% identical to SEQ ID NO:451;
   (iii) a VH1 having an amino acid sequence that is at least 95% identical to SEQ ID NO:455; and a VL1 having an amino acid sequence that is at least 95% identical to SEQ ID NO:459;
   (iv) a VH1 having an amino acid sequence that is at least 95% identical to SEQ ID NO:463; and a VL1 having an amino acid sequence that is at least 95% identical to SEQ ID NO:467;
   (v) a VH1 having an amino acid sequence that is at least 95% identical to SEQ ID NO:471; and a VL1 having an amino acid sequence that is at least 95% identical to SEQ ID NO:472;
   (vi) a VH1 having an amino acid sequence that is at least 95% identical to SEQ ID NO:473; and a VL1 having an amino acid sequence that is at least 95% identical to SEQ ID NO:474;
   (vii) a VH1 having an amino acid sequence that is at least 95% identical to SEQ ID NO:475; and a VL1 having an amino acid sequence that is at least 95% identical to SEQ ID NO:472;
   (viii) a VH1 having an amino acid sequence that is at least 95% identical to SEQ ID NO:477; and a VL1 having an amino acid sequence that is at least 95% identical to SEQ ID NO:478;
   (ix) a VH1 having an amino acid sequence that is at least 95% identical to SEQ ID NO:479; and a VL1 having an amino acid sequence that is at least 95% identical to SEQ ID NO:480;
   (x) a VH1 having an amino acid sequence that is at least 95% identical to SEQ ID NO:481; and a VL1 having an amino acid sequence that is at least 95% identical to SEQ ID NO:482;
   (xi) a VH1 having an amino acid sequence that is at least 95% identical to SEQ ID NO:483; and a VL1 having an amino acid sequence that is at least 95% identical to SEQ ID NO:484;
   (xii) a VH1 having an amino acid sequence that is at least 95% identical to SEQ ID NO:487; and a VL1 having an amino acid sequence that is at least 95% identical to SEQ ID NO:467;
   (xiii) a VH1 having an amino acid sequence that is at least 95% identical to SEQ ID NO:489; and a VL1 having an amino acid sequence that is at least 95% identical to SEQ ID NO:490;
   (xiv) a VH1 having an amino acid sequence that is at least 95% identical to SEQ ID NO:491; and a VL1 having an amino acid sequence that is at least 95% identical to SEQ ID NO:492;
   (xv) a VH1 having an amino acid sequence that is at least 95% identical to SEQ ID NO:495; and a VL1 having an amino acid sequence that is at least 95% identical to SEQ ID NO:496; or
   (xvi) a VH1 having an amino acid sequence that is at least 95% identical to SEQ ID NO:497; and a VL1 having an amino acid sequence that is at least 95% identical to SEQ ID NO:498; and
(b) the second antigen binding domain comprises:
   (i) a VH2 having an amino acid sequence that is at least 95% identical to SEQ ID NO:1; and a VL2 having an amino acid sequence that is at least 95% identical to SEQ ID NO:5;
   (ii) a VH2 having an amino acid sequence that is at least 95% identical to SEQ ID NO:11; and a VL2 having an amino acid sequence that is at least 95% identical to SEQ ID NO:5;
   (iii) a VH2 having an amino acid sequence that is at least 95% identical to SEQ ID NO:1; and a VL2 having an amino acid sequence that is at least 95% identical to SEQ ID NO:19;
   (iv) a VH2 having an amino acid sequence that is at least 95% identical to SEQ ID NO:11; and a VL2 having an amino acid sequence that is at least 95% identical to SEQ ID NO:19;
   (v) a VH2 having an amino acid sequence that is at least 95% identical to SEQ ID NO:15; and a VL2 having an amino acid sequence that is at least 95% identical to SEQ ID NO:5;
   (vi) a VH2 having an amino acid sequence that is at least 95% identical to SEQ ID NO:15; and a VL2 having an amino acid sequence that is at least 95% identical to SEQ ID NO:19;
   (vii) a VH2 having an amino acid sequence that is at least 95% identical to SEQ ID NO:63; and a VL2 having an amino acid sequence that is at least 95% identical to SEQ ID NO:75;
   (viii) a VH2 having an amino acid sequence that is at least 95% identical to SEQ ID NO:67; and a VL2 having an amino acid sequence that is at least 95% identical to SEQ ID NO:75;
   (ix) a VH2 having an amino acid sequence that is at least 95% identical to SEQ ID NO:63; and a VL2 having an amino acid sequence that is at least 95% identical to SEQ ID NO:79;
   (x) a VH2 having an amino acid sequence that is at least 95% identical to SEQ ID NO:67; and a VL2 having an amino acid sequence that is at least 95% identical to SEQ ID NO:111;
   (xi) a VH2 having an amino acid sequence that is at least 95% identical to SEQ ID NO:71; and a VL2 having an amino acid sequence that is at least 95% identical to SEQ ID NO:75;
   (xii) a VH2 having an amino acid sequence that is at least 95% identical to SEQ ID NO:71; and a VL2 having an amino acid sequence that is at least 95% identical to SEQ ID NO:79;
   (xiii) a VH2 having an amino acid sequence that is at least 95% identical to SEQ ID NO:320; and a VL2 having an amino acid sequence that is at least 95% identical to SEQ ID NO:324;
   (xiv) a VH2 having an amino acid sequence that is at least 95% identical to SEQ ID NO:327; and a VL2 having an amino acid sequence that is at least 95% identical to SEQ ID NO:331;
   (xv) a VH2 having an amino acid sequence that is at least 95% identical to SEQ ID NO:335; and a VL2 having an amino acid sequence that is at least 95% identical to SEQ ID NO:339;
   (xvi) a VH2 having an amino acid sequence that is at least 95% identical to SEQ ID NO:343; and a VL2 having an amino acid sequence that is at least 95% identical to SEQ ID NO:347;
   (xvii) a VH2 having an amino acid sequence that is at least 95% identical to SEQ ID NO:351; and a VL2 having an amino acid sequence that is at least 95% identical to SEQ ID NO:355;
   (xviii) a VH2 having an amino acid sequence that is at least 95% identical to SEQ ID NO:359; and a VL2 having an amino acid sequence that is at least 95% identical to SEQ ID NO:355;
   (xix) a VH2 having an amino acid sequence that is at least 95% identical to SEQ ID NO:367; and a VL2 having an amino acid sequence that is at least 95% identical to SEQ ID NO:371;
   (xx) a VH2 having an amino acid sequence that is at least 95% identical to SEQ ID NO:375; and a VL2 having an amino acid sequence that is at least 95% identical to SEQ ID NO:380; or
   (xxi) a VH2 having an amino acid sequence that is at least 95% identical to SEQ ID NO:391; and a VL2 having an amino acid sequence that is at least 95% identical to SEQ ID NO:395;
   (xxii) a VH2 having an amino acid sequence that is at least 95% identical to SEQ ID NO:399; and a VL2 having an amino acid sequence that is at least 95% identical to SEQ ID NO:403;
   (xxiii) a VH2 having an amino acid sequence that is at least 95% identical to SEQ ID NO:407; and a VL2 having an amino acid sequence that is at least 95% identical to SEQ ID NO:411;
   (xxiv) a VH2 having an amino acid sequence that is at least 95% identical to SEQ ID NO:415; and a VL2 having an amino acid sequence that is at least 95% identical to SEQ ID NO:419;
   (xxv) a VH2 having an amino acid sequence that is at least 95% identical to SEQ ID NO:423; and a VL2 having an amino acid sequence that is at least 95% identical to SEQ ID NO:5; or
   (xxvi) a VH2 having an amino acid sequence that is at least 95% identical to SEQ ID NO:431; and a VL2 having an amino acid sequence that is at least 95% identical to SEQ ID NO:435.

Embodiment 5. The antibody or the antibody fragment of any one of embodiments 1-4, wherein:
(a) the first antigen binding domain comprises:
   (i) a VH1 having an amino acid sequence of SEQ ID NO:439; and a VL1 having an amino acid sequence of SEQ ID NO:443;
   (ii) a VH1 having an amino acid sequence of SEQ ID NO:447; and a VL1 having an amino acid sequence of SEQ ID NO:451;
   (iii) a VH1 having an amino acid sequence of SEQ ID NO:455; and a VL1 having an amino acid sequence of SEQ ID NO:459; or
   (iv) a VH1 having an amino acid sequence of SEQ ID NO:463; and a VL1 having an amino acid sequence of SEQ ID NO:467; and
(b) the second antigen binding domain comprises:
   (i) a VH2 having an amino acid sequence of SEQ ID NO:1; and a VL2 having an amino acid sequence of SEQ ID NO:5;
   (ii) a VH2 having an amino acid sequence of SEQ ID NO:11; and a VL2 having an amino acid sequence of SEQ ID NO:5;
   (iii) a VH2 having an amino acid sequence of SEQ ID NO:1; and a VL2 having an amino acid sequence of SEQ ID NO:19;
   (iv) a VH2 having an amino acid sequence of SEQ ID NO:11; and a VL2 having an amino acid sequence of SEQ ID NO:19;
   (v) a VH2 having an amino acid sequence of SEQ ID NO:15; and a VL2 having an amino acid sequence of SEQ ID NO:5;
   (vi) a VH2 having an amino acid sequence of SEQ ID NO:15; and a VL2 having an amino acid sequence of SEQ ID NO:19;
   (vii) a VH2 having an amino acid sequence of SEQ ID NO:63; and a VL2 having an amino acid sequence of SEQ ID NO:75;
   (viii) a VH2 having an amino acid sequence of SEQ ID NO:67; and a VL2 having an amino acid sequence of SEQ ID NO:75;
   (ix) a VH2 having an amino acid sequence of SEQ ID NO:63; and a VL2 having an amino acid sequence of SEQ ID NO:79;
   (x) a VH2 having an amino acid sequence of SEQ ID NO:67; and a VL2 having an amino acid sequence of SEQ ID NO:79;
   (xi) a VH2 having an amino acid sequence of SEQ ID NO:71; and a VL2 having an amino acid sequence of SEQ ID NO:75; or
   (xii) a VH2 having an amino acid sequence of SEQ ID NO:71; and a VL2 having an amino acid sequence of SEQ ID NO:79.

Embodiment 6. The antibody or the antibody fragment of any one of embodiments 1-5, wherein the first antigen binding domain is a diabody, a Fab, a F(ab′)2, a Fc, a Fabc, a Fv, a scFv, or a protein scaffold.

Embodiment 7. The antibody or the antibody fragment of any one of embodiments 1-6, wherein the second antigen binding domain is a diabody, a Fab, a F(ab′)2, a Fc, a Fabc, a Fv, a scFv, or a protein scaffold.

Embodiment 8. The antibody or the antibody fragment of any one of embodiments 1-7, wherein the antibody is a humanized antibody or an antibody fragment.

Embodiment 9. The antibody or the antibody fragment of any one of embodiments 1-8, wherein the antibody is an IgG antibody or an antibody fragment.

Embodiment 10. The antibody or the antibody fragment of any one of embodiments 1-9, wherein the antibody comprises a first Fc domain and a second Fc domain.

Embodiment 11. The antibody or the antibody fragment of embodiment 10, wherein one of the first and the second Fc domains comprises an amino acid substitution T366W, and the other of the first and the second Fc domains comprises one or more amino acid substitutions T366S/L368A/Y407V, wherein numbering is according to EU numbering.

Embodiment 12. The antibody or the antibody fragment of embodiment 10, wherein the first and the second Fc domains comprise one or more amino acid substitutions L234A/L235A/D265S, wherein numbering is according to EU numbering.

Embodiment 13. The antibody or the antibody fragment of embodiment 10, wherein the first or the second Fc domain comprises one or more amino acid substitutions H435R/Y436F, wherein numbering is according to EU numbering.

Embodiment 14. The antibody or the antibody fragment of embodiment 10, wherein the first and the second Fc domains comprise amino acid substitutions selected from the following: S364K/E357Q; L368D/K370S; S364K; L368D/K370S; S364K; L368E/K370S; D401K; T411E/K360E/Q362E; T366W; and T366S/L368A/Y407V, wherein numbering is according to EU numbering.

Embodiment 15. The antibody or the antibody fragment of any one of embodiments 1-14, wherein the first antigen binding domain is a Fab and the second antigen binding domain is a stapled scFv.

Embodiment 16. The antibody or the antibody fragment of any one of embodiments 1-15, wherein:
(a) the first antigen binding domain comprises a vhCDR1 having an amino acid sequence of SEQ ID NO:440; a vhCDR2 having an amino acid sequence of SEQ ID NO:457; and a vhCDR3 having an amino acid sequence of SEQ ID NO:458; and a vlCDR1 having an amino acid sequence of SEQ ID NO:460, a vlCDR2 having an amino acid sequence of SEQ ID NO:445, and a vlCDR3 having an amino acid sequence of SEQ ID NO:462; and
(b) the second antigen binding domain comprises a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:22.

Embodiment 17. The antibody or the antibody fragment of any one of embodiments 1-16, wherein:
(a) the first antigen binding domain comprises a VH1 having an amino acid sequence of SEQ ID NO:455; and a VL1 having an amino acid sequence of SEQ ID NO:459; and
(b) the second antigen binding domain comprises a VH2 having an amino acid sequence of SEQ ID NO:63; and a VL2 having an amino acid sequence of SEQ ID NO:79.

Embodiment 18. A nucleic acid sequence encoding an antibody or an antibody fragment of embodiment 2.

Embodiment 19. An expression vector comprising a nucleic acid sequence of embodiment 18.

Embodiment 20. A host cell comprising an expression vector of embodiment 19.

Embodiment 21. An antibody or an antibody fragment thereof comprising:
a) a first monomer comprising, from N-terminal to C-terminal, a VH1-CH1-hinge-CH2-CH3, wherein the VH1 is the first variable heavy chain domain and CH2-CH3 is a first Fc domain; and
b) a second monomer comprising:
   i) a single chain variable fragment (scFv) comprising a second variable heavy chain domain (VH2), a linker, and a second variable light chain domain (VL2), and
   ii) a second Fc domain, wherein the scFv is covalently attached to the N-terminus of the second Fc domain using a domain linker;
c) a light chain comprising, from N-terminal to C-terminal, VL1-CL, wherein the VL1 is the first variable light chain domain and CL is a constant light chain domain,
wherein the VH1 and the VL1 together form a first antigen binding domain and the VH2 and the VL2 together form a second binding domain, and
wherein the first antigen binding domain binds to CD20 and the second antigen binding domain binds to CD28.

Embodiment 22. The antibody or the antibody fragment of embodiment 21, wherein:
(a) the first antigen binding domain comprises:
   (i) a vhCDR1 having an amino acid sequence of SEQ ID NO:440; a vhCDR2 having an amino acid sequence of SEQ ID NO:441; and a vhCDR3 having an amino acid sequence of SEQ ID NO:442; and a vlCDR1 having an amino acid sequence of SEQ ID NO:444, a vlCDR2 having an amino acid sequence of SEQ ID NO: 445, and a vlCDR3 having an amino acid sequence of SEQ ID NO:446;
   (ii) a vhCDR1 having an amino acid sequence of SEQ ID NO:440; a vhCDR2 having an amino acid sequence of SEQ ID NO:449; and a vhCDR3 having an amino acid sequence of SEQ ID NO:450; and a vlCDR1 having an amino acid sequence of SEQ ID NO:452, a vlCDR2 having an amino acid sequence of SEQ ID NO:445, and a vlCDR3 having an amino acid sequence of SEQ ID NO:454;
   (iii) a vhCDR1 having an amino acid sequence of SEQ ID NO:440; a vhCDR2 having an amino acid sequence of SEQ ID NO:457; and a vhCDR3 having an amino acid sequence of SEQ ID NO:458; and a vICDR1 having an amino acid sequence of SEQ ID NO:460, a vICDR2 having an amino acid sequence of SEQ ID NO:445, and a vICDR3 having an amino acid sequence of SEQ ID NO:462; or
   (iv) a vhCDR1 having an amino acid sequence of SEQ ID NO:464; a vhCDR2 having an amino acid sequence of SEQ ID NO:465; and a vhCDR3 having an amino acid sequence of SEQ ID NO:466; and a vICDR1 having an amino acid sequence of SEQ ID NO:356, a vICDR2 having an amino acid sequence of SEQ ID NO:357, and a vICDR3 having an amino acid sequence of SEQ ID NO:470; and
(b) the second antigen binding domain comprises:
   (i) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:8;
   (ii) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:8;
   (iii) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:22;
   (iv) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:22;
   (v) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:8; or
   (vi) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:22.

Embodiment 23. The antibody or the antibody fragment of embodiment 21 or 22, wherein:
(a) the first antigen binding domain comprises:
   (i) a VH1 having an amino acid sequence of SEQ ID NO:439; and a VL1 having an amino acid sequence of SEQ ID NO:443;
   (ii) a VH1 having an amino acid sequence of SEQ ID NO:447; and a VL1 having an amino acid sequence of SEQ ID NO:451;
   (iii) a VH1 having an amino acid sequence of SEQ ID NO:455; and a VL1 having an amino acid sequence of SEQ ID NO:459;
   (iv) a VH1 having an amino acid sequence of SEQ ID NO:463; and a VL1 having an amino acid sequence of SEQ ID NO:467;
   (v) a VH1 having an amino acid sequence of SEQ ID NO:471; and a VL1 having an amino acid sequence of SEQ ID NO:472;
   (vi) a VH1 having an amino acid sequence of SEQ ID NO:473; and a VL1 having an amino acid sequence of SEQ ID NO:474;
   (vii) a VH1 having an amino acid sequence of SEQ ID NO:475; and a VL1 having an amino acid sequence of SEQ ID NO:472;
   (viii) a VH1 having an amino acid sequence of SEQ ID NO:477; and a VL1 having an amino acid sequence of SEQ ID NO:478;
   (ix) a VH1 having an amino acid sequence of SEQ ID NO:479; and a VL1 having an amino acid sequence of SEQ ID NO:480;
   (x) a VH1 having an amino acid sequence of SEQ ID NO:481; and a VL1 having an amino acid sequence of SEQ ID NO:482;
   (xi) a VH1 having an amino acid sequence of SEQ ID NO:483; and a VL1 having an amino acid sequence of SEQ ID NO:484;
   (xii) a VH1 having an amino acid sequence of SEQ ID NO:487; and a VL1 having an amino acid sequence of SEQ ID NO:467;
   (xiii) a VH1 having an amino acid sequence of SEQ ID NO:489; and a VL1 having an amino acid sequence of SEQ ID NO:490;
   (xiv) a VH1 having an amino acid sequence of SEQ ID NO:491; and a VL1 having an amino acid sequence of SEQ ID NO:492;
   (xv) a VH1 having an amino acid sequence of SEQ ID NO:495; and a VL1 having an amino acid sequence of SEQ ID NO:496; or
   (xvi) a VH1 having an amino acid sequence of SEQ ID NO:497; and a VL1 having an amino acid sequence of SEQ ID NO:498; and
(b) the second antigen binding domain comprises:
   (i) a VH2 having an amino acid sequence of SEQ ID NO:1; and a VL2 having an amino acid sequence of SEQ ID NO:5;
   (ii) a VH2 having an amino acid sequence of SEQ ID NO:11; and a VL2 having an amino acid sequence of SEQ ID NO:5;
   (iii) a VH2 having an amino acid sequence of SEQ ID NO:1; and a VL2 having an amino acid sequence of SEQ ID NO:19;
   (iv) a VH2 having an amino acid sequence of SEQ ID NO:11; and a VL2 having an amino acid sequence of SEQ ID NO:19;
   (v) a VH2 having an amino acid sequence of SEQ ID NO:15; and a VL2 having an amino acid sequence of SEQ ID NO:5;
   (vi) a VH2 having an amino acid sequence of SEQ ID NO:15; and a VL2 having an amino acid sequence of SEQ ID NO:19;
   (vii) a VH2 having an amino acid sequence of SEQ ID NO:63; and a VL2 having an amino acid sequence of SEQ ID NO:75;
   (viii) a VH2 having an amino acid sequence of SEQ ID NO:67; and a VL2 having an amino acid sequence of SEQ ID NO:75;
   (ix) a VH2 having an amino acid sequence of SEQ ID NO:63; and a VL2 having an amino acid sequence of SEQ ID NO:79;
   (x) a VH2 having an amino acid sequence of SEQ ID NO:67; and a VL2 having an amino acid sequence of SEQ ID NO:111;
   (xi) a VH2 having an amino acid sequence of SEQ ID NO:71; and a VL2 having an amino acid sequence of SEQ ID NO:75;
   (xii) a VH2 having an amino acid sequence of SEQ ID NO:71; and a VL2 having an amino acid sequence of SEQ ID NO:79;
   (xiii) a VH2 having an amino acid sequence of SEQ ID NO:320; and a VL2 having an amino acid sequence of SEQ ID NO:324;
   (xiv) a VH2 having an amino acid sequence of SEQ ID NO:327; and a VL2 having an amino acid sequence of SEQ ID NO:331;
   (xv) a VH2 having an amino acid sequence of SEQ ID NO:335; and a VL2 having an amino acid sequence of SEQ ID NO:339;
   (xvi) a VH2 having an amino acid sequence of SEQ ID NO:343; and a VL2 having an amino acid sequence of SEQ ID NO:347;
   (xvii) a VH2 having an amino acid sequence of SEQ ID NO:351; and a VL2 having an amino acid sequence of SEQ ID NO:355;
   (xviii) a VH2 having an amino acid sequence of SEQ ID NO:359; and a VL2 having an amino acid sequence of SEQ ID NO:355;
   (xix) a VH2 having an amino acid sequence of SEQ ID NO:367; and a VL2 having an amino acid sequence of SEQ ID NO:371;
   (xx) a VH2 having an amino acid sequence of SEQ ID NO:375; and a VL2 having an amino acid sequence of SEQ ID NO:380; or
   (xxi) a VH2 having an amino acid sequence of SEQ ID NO:391; and a VL2 having an amino acid sequence of SEQ ID NO:395;
   (xxii) a VH2 having an amino acid sequence of SEQ ID NO:399; and a VL2 having an amino acid sequence of SEQ ID NO:403;
   (xxiii) a VH2 having an amino acid sequence of SEQ ID NO:407; and a VL2 having an amino acid sequence of SEQ ID NO:411;
   (xxiv) a VH2 having an amino acid sequence of SEQ ID NO:415; and a VL2 having an amino acid sequence of SEQ ID NO:419;
   (xxv) a VH2 having an amino acid sequence of SEQ ID NO:423; and a VL2 having an amino acid sequence of SEQ ID NO:5; or
   (xxvi) a VH2 having an amino acid sequence of SEQ ID NO:431; and a VL2 having an amino acid sequence of SEQ ID NO:435.

Embodiment 24. The antibody or the antibody fragment of any one of embodiments 21-23, wherein:
(a) the first antigen binding domain comprises:
   (i) a VH1 having an amino acid sequence that is at least 95% identical to SEQ ID NO:439; and a VL1 having an amino acid sequence that is at least 95% identical to SEQ ID NO:443;
   (ii) a VH1 having an amino acid sequence that is at least 95% identical to SEQ ID NO:447; and a VL1 having an amino acid sequence that is at least 95% identical to SEQ ID NO:451;
   (iii) a VH1 having an amino acid sequence that is at least 95% identical to SEQ ID NO:455; and a VL1 having an amino acid sequence that is at least 95% identical to SEQ ID NO:459;
   (iv) a VH1 having an amino acid sequence that is at least 95% identical to SEQ ID NO:463; and a VL1 having an amino acid sequence that is at least 95% identical to SEQ ID NO:467;
   (v) a VH1 having an amino acid sequence that is at least 95% identical to SEQ ID NO:471; and a VL1 having an amino acid sequence that is at least 95% identical to SEQ ID NO:472;
   (vi) a VH1 having an amino acid sequence that is at least 95% identical to SEQ ID NO:473; and a VL1 having an amino acid sequence that is at least 95% identical to SEQ ID NO:474;
   (vii) a VH1 having an amino acid sequence that is at least 95% identical to SEQ ID NO:475; and a VL1 having an amino acid sequence that is at least 95% identical to SEQ ID NO:472;
   (viii) a VH1 having an amino acid sequence that is at least 95% identical to SEQ ID NO:477; and a VL1 having an amino acid sequence that is at least 95% identical to SEQ ID NO:478;
   (ix) a VH1 having an amino acid sequence that is at least 95% identical to SEQ ID NO:479; and a VL1 having an amino acid sequence that is at least 95% identical to SEQ ID NO:480;
   (x) a VH1 having an amino acid sequence that is at least 95% identical to SEQ ID NO:481; and a VL1 having an amino acid sequence that is at least 95% identical to SEQ ID NO:482;
   (xi) a VH1 having an amino acid sequence that is at least 95% identical to SEQ ID NO:483; and a VL1 having an amino acid sequence that is at least 95% identical to SEQ ID NO:484;
   (xii) a VH1 having an amino acid sequence that is at least 95% identical to SEQ ID NO:487; and a VL1 having an amino acid sequence that is at least 95% identical to SEQ ID NO:467;
   (xiii) a VH1 having an amino acid sequence that is at least 95% identical to SEQ ID NO:489; and a VL1 having an amino acid sequence that is at least 95% identical to SEQ ID NO:490;
   (xiv) a VH1 having an amino acid sequence that is at least 95% identical to SEQ ID NO:491; and a VL1 having an amino acid sequence that is at least 95% identical to SEQ ID NO:492;
   (xv) a VH1 having an amino acid sequence that is at least 95% identical to SEQ ID NO:495; and a VL1 having an amino acid sequence that is at least 95% identical to SEQ ID NO:496; or
   (xvi) a VH1 having an amino acid sequence that is at least 95% identical to SEQ ID NO:497; and a VL1 having an amino acid sequence that is at least 95% identical to SEQ ID NO:498; and
(b) the second antigen binding domain comprises:
   (i) a VH2 having an amino acid sequence that is at least 95% identical to SEQ ID NO:1; and a VL2 having an amino acid sequence that is at least 95% identical to SEQ ID NO:5;
   (ii) a VH2 having an amino acid sequence that is at least 95% identical to SEQ ID NO:11; and a VL2 having an amino acid sequence that is at least 95% identical to SEQ ID NO:5;
   (iii) a VH2 having an amino acid sequence that is at least 95% identical to SEQ ID NO:1; and a VL2 having an amino acid sequence that is at least 95% identical to SEQ ID NO:19;
   (iv) a VH2 having an amino acid sequence that is at least 95% identical to SEQ ID NO:11; and a VL2 having an amino acid sequence that is at least 95% identical to SEQ ID NO:19;
   (v) a VH2 having an amino acid sequence that is at least 95% identical to SEQ ID NO:15; and a VL2 having an amino acid sequence that is at least 95% identical to SEQ ID NO:5;
   (vi) a VH2 having an amino acid sequence that is at least 95% identical to SEQ ID NO:15; and a VL2 having an amino acid sequence that is at least 95% identical to SEQ ID NO:19;
   (vii) a VH2 having an amino acid sequence that is at least 95% identical to SEQ ID NO:63; and a VL2 having an amino acid sequence that is at least 95% identical to SEQ ID NO:75;
   (viii) a VH2 having an amino acid sequence that is at least 95% identical to SEQ ID NO:67; and a VL2 having an amino acid sequence that is at least 95% identical to SEQ ID NO:75;
   (ix) a VH2 having an amino acid sequence that is at least 95% identical to SEQ ID NO:63; and a VL2 having an amino acid sequence that is at least 95% identical to SEQ ID NO:79;
   (x) a VH2 having an amino acid sequence that is at least 95% identical to SEQ ID NO:67; and a VL2 having an amino acid sequence that is at least 95% identical to SEQ ID NO:111;
   (xi) a VH2 having an amino acid sequence that is at least 95% identical to SEQ ID NO:71; and a VL2 having an amino acid sequence that is at least 95% identical to SEQ ID NO:75;
   (xii) a VH2 having an amino acid sequence that is at least 95% identical to SEQ ID NO:71; and a VL2 having an amino acid sequence that is at least 95% identical to SEQ ID NO:79;
   (xiii) a VH2 having an amino acid sequence that is at least 95% identical to SEQ ID NO:320; and a VL2 having an amino acid sequence that is at least 95% identical to SEQ ID NO:324;
   (xiv) a VH2 having an amino acid sequence that is at least 95% identical to SEQ ID NO:327; and a VL2 having an amino acid sequence that is at least 95% identical to SEQ ID NO:331;
   (xv) a VH2 having an amino acid sequence that is at least 95% identical to SEQ ID NO:335; and a VL2 having an amino acid sequence that is at least 95% identical to SEQ ID NO:339;
   (xvi) a VH2 having an amino acid sequence that is at least 95% identical to SEQ ID NO:343; and a VL2 having an amino acid sequence that is at least 95% identical to SEQ ID NO:347;
   (xvii) a VH2 having an amino acid sequence that is at least 95% identical to SEQ ID NO:351; and a VL2 having an amino acid sequence that is at least 95% identical to SEQ ID NO:355;
   (xviii) a VH2 having an amino acid sequence that is at least 95% identical to SEQ ID NO:359; and a VL2 having an amino acid sequence that is at least 95% identical to SEQ ID NO:355;
   (xix) a VH2 having an amino acid sequence that is at least 95% identical to SEQ ID NO:367; and a VL2 having an amino acid sequence that is at least 95% identical to SEQ ID NO:371;
   (xx) a VH2 having an amino acid sequence that is at least 95% identical to SEQ ID NO:375; and a VL2 having an amino acid sequence that is at least 95% identical to SEQ ID NO:380; or
   (xxi) a VH2 having an amino acid sequence that is at least 95% identical to SEQ ID NO:391; and a VL2 having an amino acid sequence that is at least 95% identical to SEQ ID NO:395;
   (xxii) a VH2 having an amino acid sequence that is at least 95% identical to SEQ ID NO:399; and a VL2 having an amino acid sequence that is at least 95% identical to SEQ ID NO:403;
   (xxiii) a VH2 having an amino acid sequence that is at least 95% identical to SEQ ID NO:407; and a VL2 having an amino acid sequence that is at least 95% identical to SEQ ID NO:411;
   (xxiv) a VH2 having an amino acid sequence that is at least 95% identical to SEQ ID NO:415; and a VL2 having an amino acid sequence that is at least 95% identical to SEQ ID NO:419;
   (xxv) a VH2 having an amino acid sequence that is at least 95% identical to SEQ ID NO:423; and a VL2 having an amino acid sequence that is at least 95% identical to SEQ ID NO:5; or
   (xxvi) a VH2 having an amino acid sequence that is at least 95% identical to SEQ ID NO:431; and a VL2 having an amino acid sequence that is at least 95% identical to SEQ ID NO:435.

Embodiment 25. The antibody or the antibody fragment of any one of embodiments 21-24, wherein:
(a) the first antigen binding domain comprises:
   (i) a VH1 having an amino acid sequence of SEQ ID NO:439; and a VL1 having an amino acid sequence of SEQ ID NO:443;
   (ii) a VH1 having an amino acid sequence of SEQ ID NO:447; and a VL1 having an amino acid sequence of SEQ ID NO:451;
   (iii) a VH1 having an amino acid sequence of SEQ ID NO:455; and a VL1 having an amino acid sequence of SEQ ID NO:459; or
   (iv) a VH1 having an amino acid sequence of SEQ ID NO:463; and a VL1 having an amino acid sequence of SEQ ID NO:467; and
(b) the second antigen binding domain comprises:
   (i) a VH2 having an amino acid sequence of SEQ ID NO:1; and a VL2 having an amino acid sequence of SEQ ID NO:5;
   (ii) a VH2 having an amino acid sequence of SEQ ID NO:11; and a VL2 having an amino acid sequence of SEQ ID NO:5;
   (iii) a VH2 having an amino acid sequence of SEQ ID NO:1; and a VL2 having an amino acid sequence of SEQ ID NO:19;
   (iv) a VH2 having an amino acid sequence of SEQ ID NO:11; and a VL2 having an amino acid sequence of SEQ ID NO:19;
   (v) a VH2 having an amino acid sequence of SEQ ID NO:15; and a VL2 having an amino acid sequence of SEQ ID NO:5;
   (vi) a VH2 having an amino acid sequence of SEQ ID NO:15; and a VL2 having an amino acid sequence of SEQ ID NO:19;
   (vii) a VH2 having an amino acid sequence of SEQ ID NO:63; and a VL2 having an amino acid sequence of SEQ ID NO:75;
   (viii) a VH2 having an amino acid sequence of SEQ ID NO:67; and a VL2 having an amino acid sequence of SEQ ID NO:75;
   (ix) a VH2 having an amino acid sequence of SEQ ID NO:63; and a VL2 having an amino acid sequence of SEQ ID NO:79;
   (x) a VH2 having an amino acid sequence of SEQ ID NO:67; and a VL2 having an amino acid sequence of SEQ ID NO:79;
   (xi) a VH2 having an amino acid sequence of SEQ ID NO:71; and a VL2 having an amino acid sequence of SEQ ID NO:75; or
   (xii) a VH2 having an amino acid sequence of SEQ ID NO:71; and a VL2 having an amino acid sequence of SEQ ID NO:79.

Embodiment 26. The antibody or the antibody fragment of any one of embodiments 21-25, wherein:
(a) a first monomer is selected from the amino acid sequences represented by SEQ ID NOs: 405, 422, 427, 436, 453, 456. 461, and 485;
b) a second monomer is selected from the amino acid sequences represented by SEQ ID NOs: 406, 417, 418, 421, 428, 430, 432, 434, 438, 448, 468, 469, and 486; and
c) a light chain is selected from the amino acid sequences represented by SEQ ID NOs: 334, 366, 426, and 429.

Embodiment 27. The antibody or the antibody fragment of any one of embodiments 21-26, wherein the antibody is a humanized antibody or an antibody fragment.

Embodiment 28. The antibody or the antibody fragment of any one of embodiments 21-27, wherein the antibody is an IgG antibody or an antibody fragment.

Embodiment 29. The antibody or the antibody fragment of any one of embodiments 21-28, wherein one of the first and the second Fc domains comprises an amino acid substitution T366W, and the other of the first and the second Fc domains comprises one or more amino acid substitutions T366S/L368A/Y407V, wherein numbering is according to EU numbering.

Embodiment 30. The antibody or the antibody fragment of any one of embodiments 21-29, wherein the first and the second Fc domains comprise one or more amino acid substitutions L234A/L235A/D265S, wherein numbering is according to EU numbering.

Embodiment 31. The antibody or the antibody fragment of any one of embodiments 21-30, wherein the first or the second Fc domain comprises one or more amino acid substitutions H435R/Y436F, wherein numbering is according to EU numbering.

Embodiment 32. The antibody or the antibody fragment of any one of embodiments 21-31, wherein the first and the second Fc domains comprise amino acid substitutions selected from the following: S364K/E357Q; L368D/K370S; S364K; L368D/K370S; S364K; L368E/K370S; D401K; T411E/K360E/Q362E; T366W; and T366S/L368A/Y407V, wherein numbering is according to EU numbering.

Embodiment 33. The antibody or the antibody fragment of any one of embodiments 21-32, wherein:
(a) the first antigen binding domain comprises a vhCDR1 having an amino acid sequence of SEQ ID NO:440; a vhCDR2 having an amino acid sequence of SEQ ID NO:457; and a vhCDR3 having an amino acid sequence of SEQ ID NO:458; and a vlCDR1 having an amino acid sequence of SEQ ID NO:460, a vlCDR2 having an amino acid sequence of SEQ ID NO:445, and a vlCDR3 having an amino acid sequence of SEQ ID NO:462; and
(b) the second antigen binding domain comprises a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:22.

Embodiment 34. The antibody or the antibody fragment of any one of embodiments 21-33, wherein:
(a) the first antigen binding domain comprises a VH1 having an amino acid sequence of SEQ ID NO:455; and a VL1 having an amino acid sequence of SEQ ID NO:459; and
(b) the second antigen binding domain comprises a VH2 having an amino acid sequence of SEQ ID NO:63; and a VL2 having an amino acid sequence of SEQ ID NO:79.

Embodiment 35. The antibody or the antibody fragment of any one of embodiments 21-34, wherein the first monomer comprises the amino acid sequence of SEQ ID NO: 461, the second monomer comprises the amino acid sequence of SEQ ID NO: 469, and the light chain comprises the amino acid sequence of SEQ ID NO: 429.

Embodiment 36. A method of treating a B-cell malignancy in a patient in need thereof, comprising administering to the patient a combination of an anti-CD20 x anti-CD28 antibody or an antibody fragment thereof and a T-cell engager, wherein:
(A) the anti-CD20 x anti-CD28 antibody or the fragment thereof comprises:
(a) a first antigen binding domain that binds to CD20 comprising:
(i) a vhCDR1 having an amino acid sequence of SEQ ID NO:440; a vhCDR2 having an amino acid sequence of SEQ ID NO:441; and a vhCDR3 having an amino acid sequence of SEQ ID NO:442; and a vlCDR1 having an amino acid sequence of SEQ ID NO:444, a vlCDR2 having an amino acid sequence of SEQ ID NO: 445, and a vlCDR3 having an amino acid sequence of SEQ ID NO:446;
(ii) a vhCDR1 having an amino acid sequence of SEQ ID NO:440; a vhCDR2 having an amino acid sequence of SEQ ID NO:449; and a vhCDR3 having an amino acid sequence of SEQ ID NO:450; and a vlCDR1 having an amino acid sequence of SEQ ID NO:452, a vlCDR2 having an amino acid sequence of SEQ ID NO:445, and a vlCDR3 having an amino acid sequence of SEQ ID NO:454;
(iii) a vhCDR1 having an amino acid sequence of SEQ ID NO:440; a vhCDR2 having an amino acid sequence of SEQ ID NO:457; and a vhCDR3 having an amino acid sequence of SEQ ID NO:458; and a vlCDR1 having an amino acid sequence of SEQ ID NO:460, a vlCDR2 having an amino acid sequence of SEQ ID NO:445, and a vlCDR3 having an amino acid sequence of SEQ ID NO:462; or
(iv) a vhCDR1 having an amino acid sequence of SEQ ID NO:464; a vhCDR2 having an amino acid sequence of SEQ ID NO:465; and a vhCDR3 having an amino acid sequence of SEQ ID NO:466; and a vlCDR1 having an amino acid sequence of SEQ ID NO:356, a vlCDR2 having an amino acid sequence of SEQ ID NO:357, and a vlCDR3 having an amino acid sequence of SEQ ID NO:470; and
(b) a second antigen binding domain that binds to CD28 comprising:
(i) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:8;
(ii) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:8;
(iii) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:22;
(iv) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:22;
(v) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:8; or
(vi) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:22; and
(B) the T cell engager is an anti-CD79b x anti-CD20 x anti-CD3 antibody or an antibody fragment thereof.

Embodiment 37. The method of embodiment 36, wherein the anti-CD20 x anti-CD28 antibody or the antibody fragment comprises:
(i) the first antigen binding domain that binds to CD20 comprises a vhCDR1 having an amino acid sequence of SEQ ID NO:440, a vhCDR2 having an amino acid sequence of SEQ ID NO:457, and a vhCDR3 having an amino acid sequence of SEQ ID NO:458; and a vICDR1 having an amino acid sequence of SEQ ID NO:460, a vICDR2 having an amino acid sequence of SEQ ID NO:445, and a vICDR3 having an amino acid sequence of SEQ ID NO:462; and
(ii) the second antigen binding domain that binds to CD28 comprises a vhCDR1 having an amino acid sequence of SEQ ID NO:2, a vhCDR2 having an amino acid sequence of SEQ ID NO:3, and a vhCDR3 having an amino acid sequence of SEQ ID NO:4; and a vICDR1 having an amino acid sequence of SEQ ID NO:6, a vICDR2 having an amino acid sequence of SEQ ID NO:7, and a vICDR3 having an amino acid sequence of SEQ ID NO:22.

Embodiment 38. The method of embodiment 36 or 37, wherein the anti-CD79b x anti-CD20 x anti-CD3 antibody or the antibody fragment comprises:
(a) an antigen binding domain that binds to CD79b comprising a vhCDR1 having an amino acid sequence of SEQ ID NO:524, a vhCDR2 having an amino acid sequence of SEQ ID NO:525, and a vhCDR3 having an amino acid sequence of SEQ ID NO:526; and a vICDR1 having an amino acid sequence of SEQ ID NO:528, a vICDR2 having an amino acid sequence of SEQ ID NO:529, and a vICDR3 having an amino acid sequence of SEQ ID NO:530;
(b) an antigen binding domain that binds to CD20 comprising a vhCDR1 having an amino acid sequence of SEQ ID NO:540, a vhCDR2 having an amino acid sequence of SEQ ID NO:541, and a vhCDR3 having an amino acid sequence of SEQ ID NO:542; and a vICDR1 having an amino acid sequence of SEQ ID NO:544, a vICDR2 having an amino acid sequence of SEQ ID NO:545, and a vICDR3 having an amino acid sequence of SEQ ID NO:546; and
(c) an antigen binding domain that binds to CD3 comprising a vhCDR1 having an amino acid sequence of SEQ ID NO:532, a vhCDR2 having an amino acid sequence of SEQ ID NO:533, and a vhCDR3 having an amino acid sequence of SEQ ID NO:534; and a vICDR1 having an amino acid sequence of SEQ ID NO:536, a vICDR2 having an amino acid sequence of SEQ ID NO:537, and a vICDR3 having an amino acid sequence of SEQ ID NO:538.

Embodiment 39. The method of any one of embodiments 36-38, wherein the anti-CD20 x anti-CD28 antibody or the antibody fragment comprises:
(a) the CD20 antigen binding domain comprising a VH1 having an amino acid sequence of SEQ ID NO:455; and a VL1 having an amino acid sequence of SEQ ID NO:459; and
(b) the CD28 antigen binding domain comprising a VH2 having an amino acid sequence of SEQ ID NO:63; and a VL2 having an amino acid sequence of SEQ ID NO:79.

Embodiment 40. The method of any one of embodiments 36-39, wherein the anti-CD79b x anti-CD20 x anti-CD3 antibody or the antibody fragment comprises:
(a) the CD79b antigen binding domain comprising a VH1 having an amino acid sequence of SEQ ID NO:523; and a VL1 having an amino acid sequence of SEQ ID NO:527;
(b) the CD20 antigen binding domain comprising a VH2 having an amino acid sequence of SEQ ID NO:539; and a VL2 having an amino acid sequence of SEQ ID NO:543; and
(c) the CD3 antigen binding domain comprising a VH2 having an amino acid sequence of SEQ ID NO:531; and a VL2 having an amino acid sequence of SEQ ID NO:535.

Embodiment 41. The method of any one of embodiments 36-40, wherein:
(a) the anti-CD20 x anti-CD28 antibody or the antibody fragment comprises:
   (i) the CD20 antigen binding domain comprising a VH1 having an amino acid sequence of SEQ ID NO:455; and a VL1 having an amino acid sequence of SEQ ID NO:459; and
   (ii) the CD28 antigen binding domain comprising a VH2 having an amino acid sequence of SEQ ID NO:63; and a VL2 having an amino acid sequence of SEQ ID NO:79; and
(b) the anti-CD79b x anti-CD20 x anti-CD3 antibody or the antibody fragment comprises:
   (i) the CD79b antigen binding domain comprising a VH1 having an amino acid sequence of SEQ ID NO:523; and a VL1 having an amino acid sequence of SEQ ID NO:527;
   (ii) the CD20 antigen binding domain comprising a VH2 having an amino acid sequence of SEQ ID NO:539; and a VL2 having an amino acid sequence of SEQ ID NO:543; and
   (iii) the CD3 antigen binding domain comprising a VH2 having an amino acid sequence of SEQ ID NO:531; and a VL2 having an amino acid sequence of SEQ ID NO:535.

Embodiment 42. The method of any one of embodiments 36-41, wherein the anti-CD20 x anti-CD28 antibody or the antibody fragment comprises:
(a) a first monomer polypeptide having an amino acid sequence of SEQ ID NO:461;
(b) a second monomer polypeptide having an amino acid sequence of SEQ ID NO:469; and
(c) a light chain polypeptide having an amino acid sequence of SEQ ID NO:429.

Embodiment 43. The method of any one of embodiments 36-42, wherein the anti-CD79b x anti-CD20 x anti-CD3 antibody or the antibody fragment comprises:
(a) a first monomer polypeptide having an amino acid sequence of SEQ ID NO:520;
(b) a second monomer polypeptide having an amino acid sequence of SEQ ID NO:521; and
(c) a light chain polypeptide having an amino acid sequence of SEQ ID NO:522.

Embodiment 44. The method of any one of embodiments 36-43, wherein:
(a) the anti-CD20 x anti-CD28 antibody or the antibody fragment comprises:
   (i) a first monomer polypeptide having an amino acid sequence of SEQ ID NO:461;
   (ii) a second monomer polypeptide having an amino acid sequence of SEQ ID NO:469;
   (iii) a first light chain polypeptide having an amino acid sequence of SEQ ID NO:429; and
(b) the anti-CD79b x anti-CD20 x anti-CD3 antibody or the antibody fragment comprises:
   (i) a fourth monomer polypeptide having an amino acid sequence of SEQ ID NO:520;
   (ii) a fifth monomer polypeptide having an amino acid sequence of SEQ ID NO:521; and
   (iii) a second light chain polypeptide having an amino acid sequence of SEQ ID NO:522.

Embodiment 45. The method of any one of embodiments 36-44, wherein the anti-CD20 x anti-CD28 antibody or the antibody fragment thereof and the anti-CD79b x anti-CD20 x anti-CD3 antibody or the antibody fragment thereof are administered together in a single composition or administered separately in two or more different compositions.

Embodiment 46. The method of any one of embodiments 36-45, wherein the anti-CD20 x anti-CD28 antibody or the antibody fragment thereof and the anti-CD79b x anti-CD20 x anti-CD3 antibody or the antibody fragment thereof are administered subcutaneously or intravenously.

Embodiment 47. The method of any one of embodiments 36-46, wherein the anti-CD20 x anti-CD28 antibody or the antibody fragment thereof and the anti-CD79b x anti-CD20 x anti-CD3 antibody or the antibody fragment thereof are administered concurrently or sequentially.

Embodiment 48. The method of any one of embodiments 36-47, wherein the B-cell malignancy is B-cell lymphoma, non-Hodgkin lymphoma (NHL), diffuse large B-cell lymphoma (DLBCL), a mantle cell lymphoma (MCL), a follicular lymphoma (FL), acute lymphoblastic leukemia (ALL), chronic lymphocytic leukemia (CLL), small lymphocytic lymphoma (SLL), Waldenström macroglobulinemia (WM), multiple myeloma (MM), mucosa-associated lymphoid tissue (MALT) lymphoma, Hodgkin's lymphoma, Burkitt's lymphoma, hairy-cell leukemia, or Plasmacytoma.

1. An antibody or an antibody fragment thereof comprising:
(a) a first antigen binding domain that binds to CD20, wherein the first antigen binding domain comprises:
(i) a heavy chain complementarity determining region 1 (vhCDR1), a heavy chain-complementarity determining region 2 (vhCDR2) and a heavy chain complementarity determining region 3 (vhCDR3) of a variable heavy chain domain (VH1) having an amino acid sequence of SEQ ID NO:439, and a light chain complementarity determining region 1 (vlCDR1), a light chain complementarity determining region 2 (vlCDR2) and a light chain complementarity determining region 3 (vlCDR3) of a variable light chain domain (VL1) having an amino acid sequence of SEQ ID NO:443;
(ii) a vhCDR1, a vhCDR2 and a vhCDR3 of a variable heavy chain domain (VH1) having an amino acid sequence of SEQ ID NO:447, and a vlCDR1, a vlCDR2 and a vlCDR3 of a variable light chain domain (VL1) having an amino acid sequence of SEQ ID NO:451;
(iii) a vhCDR1, a vhCDR2 and a vhCDR3 of a variable heavy chain domain (VH1) having an amino acid sequence of SEQ ID NO:455, and a vlCDR1, a vlCDR2 and a vlCDR3 of a variable light chain domain (VL1) having an amino acid sequence of SEQ ID NO:459;
(iv) a vhCDR1, a vhCDR2 and a vhCDR3 of a variable heavy chain domain (VH1) having an amino acid sequence of SEQ ID NO:463, and a vlCDR1, a vlCDR2 and a vlCDR3 of a variable light chain domain (VL1) having an amino acid sequence of SEQ ID NO:467;
(v) a vhCDR1, a vhCDR2 and a vhCDR3 of a variable heavy chain domain (VH1) having an amino acid sequence of SEQ ID NO:471, and a vlCDR1, a vlCDR2 and a vlCDR3 of a variable light chain domain (VL1) having an amino acid sequence of SEQ ID NO:472;
(vi) a vhCDR1, a vhCDR2 and a vhCDR3 of a variable heavy chain domain (VH1) having an amino acid sequence of SEQ ID NO:473, and a vlCDR1, a vlCDR2 and a vlCDR3 of a variable light chain domain (VL1) having an amino acid sequence of SEQ ID NO:474;
(vii) a vhCDR1, a vhCDR2 and a vhCDR3 of a variable heavy chain domain (VH1) having an amino acid sequence of SEQ ID NO:475, and a vlCDR1, a vlCDR2 and a vlCDR3 of a variable light chain domain (VL1) having an amino acid sequence of SEQ ID NO:472;
(viii) a vhCDR1, a vhCDR2 and a vhCDR3 of a variable heavy chain domain (VH1) having an amino acid sequence of SEQ ID NO:477, and a vlCDR1, a vlCDR2 and a vlCDR3 of a variable light chain domain (VL1) having an amino acid sequence of SEQ ID NO:478;
(ix) a vhCDR1, a vhCDR2 and a vhCDR3 of a variable heavy chain domain (VH1) having an amino acid sequence of SEQ ID NO:479, and a vlCDR1, a vlCDR2 and a vlCDR3 of a variable light chain domain (VL1) having an amino acid sequence of SEQ ID NO:480;
(x) a vhCDR1, a vhCDR2 and a vhCDR3 of a variable heavy chain domain (VH1) having an amino acid sequence of SEQ ID NO:481, and a vlCDR1, a vlCDR2 and a vlCDR3 of a variable light chain domain (VL1) having an amino acid sequence of SEQ ID NO:482;
(xi) a vhCDR1, a vhCDR2 and a vhCDR3 of a variable heavy chain domain (VH1) having an amino acid sequence of SEQ ID NO:483, and a vlCDR1, a vlCDR2 and a vlCDR3 of a variable light chain domain (VL1) having an amino acid sequence of SEQ ID NO:484;
(xii) a vhCDR1, a vhCDR2 and a vhCDR3 of a variable heavy chain domain (VH1) having an amino acid sequence of SEQ ID NO:487, and a vlCDR1, a vlCDR2 and a vlCDR3 of a variable light chain domain (VL1) having an amino acid sequence of SEQ ID NO:467;
(xiii) a vhCDR1, a vhCDR2 and a vhCDR3 of a variable heavy chain domain (VH1) having an amino acid sequence of SEQ ID NO:489, and a vlCDR1, a vlCDR2 and a vlCDR3 of a variable light chain domain (VL1) having an amino acid sequence of SEQ ID NO:490;
(xiv) a vhCDR1, a vhCDR2 and a vhCDR3 of a variable heavy chain domain (VH1) having an amino acid sequence of SEQ ID NO:491, and a vlCDR1, a vlCDR2 and a vlCDR3 of a variable light chain domain (VL1) having an amino acid sequence of SEQ ID NO:492;
(xv) a vhCDR1, a vhCDR2 and a vhCDR3 of a variable heavy chain domain (VH1) having an amino acid sequence of SEQ ID NO:495, and a vlCDR1, a vlCDR2 and a vlCDR3 of a variable light chain domain (VL1) having an amino acid sequence of SEQ ID NO:496; or
(xvi) a vhCDR1, a vhCDR2 and a vhCDR3 of a variable heavy chain domain (VH1) having an amino acid sequence of SEQ ID NO:497, and a vlCDR1, a vlCDR2 and a vlCDR3 of a variable light chain domain (VL1) having an amino acid sequence of SEQ ID NO:498; and
(b) a second antigen binding domain that binds to CD28, wherein the second antigen binding domain comprises:
(i) a vhCDR1, a vhCDR2 and a vhCDR3 of a variable heavy chain domain (VH2) having an amino acid sequence of SEQ ID NO:1, and a vlCDR1, a vlCDR2 and a vlCDR3 of a variable light chain domain (VL2) having an amino acid sequence of SEQ ID NO:5;
(ii) a vhCDR1, a vhCDR2 and a vhCDR3 of a variable heavy chain domain (VH2) having an amino acid sequence of SEQ ID NO:11, and a vlCDR1, a vlCDR2 and a vlCDR3 of a variable light chain domain (VL2) having an amino acid sequence of SEQ ID NO:5;
(iii) a vhCDR1, a vhCDR2 and a vhCDR3 of a variable heavy chain domain (VH2) having an amino acid sequence of SEQ ID NO:1, and a vlCDR1, a vlCDR2 and a vlCDR3 of a variable light chain domain (VL2) having an amino acid sequence of SEQ ID NO:19;
(iv) a vhCDR1, a vhCDR2 and a vhCDR3 of a variable heavy chain domain (VH2) having an amino acid sequence of SEQ ID NO:11, and a vlCDR1, a vlCDR2 and a vlCDR3 of a variable light chain domain (VL2) having an amino acid sequence of SEQ ID NO:19;
(v) a vhCDR1, a vhCDR2 and a vhCDR3 of a variable heavy chain domain (VH2) having an amino acid sequence of SEQ ID NO:15, and a vlCDR1, a vlCDR2 and a vlCDR3 of a variable light chain domain (VL2) having an amino acid sequence of SEQ ID NO:5;
(vi) a vhCDR1, a vhCDR2 and a vhCDR3 of a variable heavy chain domain (VH2) having an amino acid sequence of SEQ ID NO:15, and a vlCDR1, a vlCDR2 and a vlCDR3 of a variable light chain domain (VL2) having an amino acid sequence of SEQ ID NO:19;
(vii) a vhCDR1, a vhCDR2 and a vhCDR3 of a variable heavy chain domain (VH2) having an amino acid sequence of SEQ ID NO:63, and a vlCDR1, a vlCDR2 and a vlCDR3 of a variable light chain domain (VL2) having an amino acid sequence of SEQ ID NO:75;
(viii) a vhCDR1, a vhCDR2 and a vhCDR3 of a variable heavy chain domain (VH2) having an amino acid sequence of SEQ ID NO:67, and a vlCDR1, a vlCDR2 and a vlCDR3 of a variable light chain domain (VL2) having an amino acid sequence of SEQ ID NO:75;
(ix) a vhCDR1, a vhCDR2 and a vhCDR3 of a variable heavy chain domain (VH2) having an amino acid sequence of SEQ ID NO:63, and a vlCDR1, a vlCDR2 and a vlCDR3 of a variable light chain domain (VL2) having an amino acid sequence of SEQ ID NO:79;
(x) a vhCDR1, a vhCDR2 and a vhCDR3 of a variable heavy chain domain (VH2) having an amino acid sequence of SEQ ID NO:67, and a vlCDR1, a vlCDR2 and a vlCDR3 of a variable light chain domain (VL2) having an amino acid sequence of SEQ ID NO:111;
(xi) a vhCDR1, a vhCDR2 and a vhCDR3 of a variable heavy chain domain (VH2) having an amino acid sequence of SEQ ID NO:71, and a vlCDR1, a vlCDR2 and a vlCDR3 of a variable light chain domain (VL2) having an amino acid sequence of SEQ ID NO:75;
(xii) a vhCDR1, a vhCDR2 and a vhCDR3 of a variable heavy chain domain (VH2) having an amino acid sequence of SEQ ID NO:71, and a vlCDR1, a vlCDR2 and a vlCDR3 of a variable light chain domain (VL2) having an amino acid sequence of SEQ ID NO:79;
(xiii) a vhCDR1, a vhCDR2 and a vhCDR3 of a variable heavy chain domain (VH2) having an amino acid sequence of SEQ ID NO:320, and a vlCDR1, a vlCDR2 and a vlCDR3 of a variable light chain domain (VL2) having an amino acid sequence of SEQ ID NO:324;
(xiv) a vhCDR1, a vhCDR2 and a vhCDR3 of a variable heavy chain domain (VH2) having an amino acid sequence of SEQ ID NO:327, and a vlCDR1, a vlCDR2 and a vlCDR3 of a variable light chain domain (VL2) having an amino acid sequence of SEQ ID NO:331;
(xv) a vhCDR1, a vhCDR2 and a vhCDR3 of a variable heavy chain domain (VH2) having an amino acid sequence of SEQ ID NO:335, and a vlCDR1, a vlCDR2 and a vlCDR3 of a variable light chain domain (VL2) having an amino acid sequence of SEQ ID NO:339;
(xvi) a vhCDR1, a vhCDR2 and a vhCDR3 of a variable heavy chain domain (VH2) having an amino acid sequence of SEQ ID NO:343, and a vlCDR1, a vlCDR2 and a vlCDR3 of a variable light chain domain (VL2) having an amino acid sequence of SEQ ID NO:347;
(xvii) a vhCDR1, a vhCDR2 and a vhCDR3 of a variable heavy chain domain (VH2) having an amino acid sequence of SEQ ID NO:351, and a vlCDR1, a vlCDR2 and a vlCDR3 of a variable light chain domain (VL2) having an amino acid sequence of SEQ ID NO:355;
(xviii) a vhCDR1, a vhCDR2 and a vhCDR3 of a variable heavy chain domain (VH2) having an amino acid sequence of SEQ ID NO:359, and a vlCDR1, a vlCDR2 and a vlCDR3 of a variable light chain domain (VL2) having an amino acid sequence of SEQ ID NO:355;
(xix) a vhCDR1, a vhCDR2 and a vhCDR3 of a variable heavy chain domain (VH2) having an amino acid sequence of SEQ ID NO:367, and a vlCDR1, a vlCDR2 and a vlCDR3 of a variable light chain domain (VL2) having an amino acid sequence of SEQ ID NO:371;
(xx) a vhCDR1, a vhCDR2 and a vhCDR3 of a variable heavy chain domain (VH2) having an amino acid sequence of SEQ ID NO:375, and a vlCDR1, a vlCDR2 and a vlCDR3 of a variable light chain domain (VL2) having an amino acid sequence of SEQ ID NO:380;
(xxi) a vhCDR1, a vhCDR2 and a vhCDR3 of a variable heavy chain domain (VH2) having an amino acid sequence of SEQ ID NO:391, and a vlCDR1, a vlCDR2 and a vlCDR3 of a variable light chain domain (VL2) having an amino acid sequence of SEQ ID NO:395;
(xxii) a vhCDR1, a vhCDR2 and a vhCDR3 of a variable heavy chain domain (VH2) having an amino acid sequence of SEQ ID NO:399, and a vlCDR1, a vlCDR2 and a vlCDR3 of a variable light chain domain (VL2) having an amino acid sequence of SEQ ID NO:403;
(xxiii) a vhCDR1, a vhCDR2 and a vhCDR3 of a variable heavy chain domain (VH2) having an amino acid sequence of SEQ ID NO:407, and a vlCDR1, a vlCDR2 and a vlCDR3 of a variable light chain domain (VL2) having an amino acid sequence of SEQ ID NO:411;
(xxiv) a vhCDR1, a vhCDR2 and a vhCDR3 of a variable heavy chain domain (VH2) having an amino acid sequence of SEQ ID NO:415, and a vlCDR1, a vlCDR2 and a vlCDR3 of a variable light chain domain (VL2) having an amino acid sequence of SEQ ID NO:419;
(xxv) a vhCDR1, a vhCDR2 and a vhCDR3 of a variable heavy chain domain (VH2) having an amino acid sequence of SEQ ID NO:423, and a vlCDR1, a vlCDR2 and a vlCDR3 of a variable light chain domain (VL2) having an amino acid sequence of SEQ ID NO:5; or
(xxvi) a vhCDR1, a vhCDR2 and a vhCDR3 of a variable heavy chain domain (VH2) having an amino acid sequence of SEQ ID NO:431, and a vlCDR1, a vlCDR2 and a vlCDR3 of a variable light chain domain (VL2) having an amino acid sequence of SEQ ID NO:435.

2. The antibody or the antibody fragment of claim 1, wherein
(a) the first antigen binding domain comprises:
(i) a vhCDR1 having an amino acid sequence of SEQ ID NO:440; a vhCDR2 having an amino acid sequence of SEQ ID NO:441; and a vhCDR3 having an amino acid sequence of SEQ ID NO:442; and a vlCDR1 having an amino acid sequence of SEQ ID NO:444, a vlCDR2 having an amino acid sequence of SEQ ID NO: 445, and a vlCDR3 having an amino acid sequence of SEQ ID NO:446;
(ii) a vhCDR1 having an amino acid sequence of SEQ ID NO:440; a vhCDR2 having an amino acid sequence of SEQ ID NO:449; and a vhCDR3 having an amino acid sequence of SEQ ID NO:450; and a vlCDR1 having an amino acid sequence of SEQ ID NO:452, a vlCDR2 having an amino acid sequence of SEQ ID NO:445, and a vlCDR3 having an amino acid sequence of SEQ ID NO:454;
(iii) a vhCDR1 having an amino acid sequence of SEQ ID NO:440; a vhCDR2 having an amino acid sequence of SEQ ID NO:457; and a vhCDR3 having an amino acid sequence of SEQ ID NO:458; and a vlCDR1 having an amino acid sequence of SEQ ID NO:460, a vlCDR2 having an amino acid sequence of SEQ ID NO:445, and a vlCDR3 having an amino acid sequence of SEQ ID NO:462; or
(iv) a vhCDR1 having an amino acid sequence of SEQ ID NO:464; a vhCDR2 having an amino acid sequence of SEQ ID NO:465; and a vhCDR3 having an amino acid sequence of SEQ ID NO:466; and a vlCDR1 having an amino acid sequence of SEQ ID NO:356, a vlCDR2 having an amino acid sequence of SEQ ID NO:357, and a vlCDR3 having an amino acid sequence of SEQ ID NO:470; and
(b) the second antigen binding domain comprises:
(i) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:8;
(ii) a vhCDR1 having an amino acid sequence of SEQ ID NO: 12; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:8;
(iii) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:22;
(iv) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:22;
(v) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:8; or
(vi) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:22.

3. The antibody or the antibody fragment of claim 1, wherein:
(a) the first antigen binding domain comprises:
(i) a VH1 having an amino acid sequence of SEQ ID NO:439; and a VL1 having an amino acid sequence of SEQ ID NO:443;
(ii) a VH1 having an amino acid sequence of SEQ ID NO:447; and a VL1 having an amino acid sequence of SEQ ID NO:451;
(iii) a VH1 having an amino acid sequence of SEQ ID NO:455; and a VL1 having an amino acid sequence of SEQ ID NO:459;
(iv) a VH1 having an amino acid sequence of SEQ ID NO:463; and a VL1 having an amino acid sequence of SEQ ID NO:467;
(v) a VH1 having an amino acid sequence of SEQ ID NO:471; and a VL1 having an amino acid sequence of SEQ ID NO:472;
(vi) a VH1 having an amino acid sequence of SEQ ID NO:473; and a VL1 having an amino acid sequence of SEQ ID NO:474;
(vii) a VH1 having an amino acid sequence of SEQ ID NO:475; and a VL1 having an amino acid sequence of SEQ ID NO:472;
(viii) a VH1 having an amino acid sequence of SEQ ID NO:477; and a VL1 having an amino acid sequence of SEQ ID NO:478;
(ix) a VH1 having an amino acid sequence of SEQ ID NO 479; and a VL1 having an amino acid sequence of SEQ ID NO:480;
(x) a VH1 having an amino acid sequence of SEQ ID NO:481; and a VL1 having an amino acid sequence of SEQ ID NO:482;
(xi) a VH1 having an amino acid sequence of SEQ ID NO:483; and a VL1 having an amino acid sequence of SEQ ID NO:484;
(xii) a VH1 having an amino acid sequence of SEQ ID NO:487; and a VL1 having an amino acid sequence of SEQ ID NO:467;
(xiii) a VH1 having an amino acid sequence of SEQ ID NO:489; and a VL1 having an amino acid sequence of SEQ ID NO:490;
(xiv) a VH1 having an amino acid sequence of SEQ ID NO:491; and a VL1 having an amino acid sequence of SEQ ID NO:492;
(xv) a VH1 having an amino acid sequence of SEQ ID NO:495; and a VL1 having an amino acid sequence of SEQ ID NO:496; or
(xvi) a VH1 having an amino acid sequence of SEQ ID NO:497; and a VL1 having an amino acid sequence of SEQ ID NO:498; and
(b) the second antigen binding domain comprises:
(i) a VH2 having an amino acid sequence of SEQ ID NO:1; and a VL2 having an amino acid sequence of SEQ ID NO:5;
(ii) a VH2 having an amino acid sequence of SEQ ID NO:11; and a VL2 having an amino acid sequence of SEQ ID NO:5;
(iii) a VH2 having an amino acid sequence of SEQ ID NO:1; and a VL2 having an amino acid sequence of SEQ ID NO:19;
(iv) a VH2 having an amino acid sequence of SEQ ID NO:11; and a VL2 having an amino acid sequence of SEQ ID NO:19;
(v) a VH2 having an amino acid sequence of SEQ ID NO:15; and a VL2 having an amino acid sequence of SEQ ID NO:5;
(vi) a VH2 having an amino acid sequence of SEQ ID NO:15; and a VL2 having an amino acid sequence of SEQ ID NO:19;
(vii) a VH2 having an amino acid sequence of SEQ ID NO:63; and a VL2 having an amino acid sequence of SEQ ID NO:75;
(viii) a VH2 having an amino acid sequence of SEQ ID NO:67; and a VL2 having an amino acid sequence of SEQ ID NO:75;
(ix) a VH2 having an amino acid sequence of SEQ ID NO:63; and a VL2 having an amino acid sequence of SEQ ID NO:79;
(x) a VH2 having an amino acid sequence of SEQ ID NO:67; and a VL2 having an amino acid sequence of SEQ ID NO:111;
(xi) a VH2 having an amino acid sequence of SEQ ID NO:71; and a VL2 having an amino acid sequence of SEQ ID NO:75;
(xii) a VH2 having an amino acid sequence of SEQ ID NO:71; and a VL2 having an amino acid sequence of SEQ ID NO:79;
(xiii) a VH2 having an amino acid sequence of SEQ ID NO:320; and a VL2 having an amino acid sequence of SEQ ID NO:324;
(xiv) a VH2 having an amino acid sequence of SEQ ID NO:327; and a VL2 having an amino acid sequence of SEQ ID NO:331;
(xv) a VH2 having an amino acid sequence of SEQ ID NO: 335; and a VL2 having an amino acid sequence of SEQ ID NO: 339;
(xvi) a VH2 having an amino acid sequence of SEQ ID NO:343; and a VL2 having an amino acid sequence of SEQ ID NO:347;
(xvii) a VH2 having an amino acid sequence of SEQ ID NO:351; and a VL2 having an amino acid sequence of SEQ ID NO:355;
(xviii) a VH2 having an amino acid sequence of SEQ ID NO:359; and a VL2 having an amino acid sequence of SEQ ID NO:355;
(xix) a VH2 having an amino acid sequence of SEQ ID NO:367; and a VL2 having an amino acid sequence of SEQ ID NO: 371;
(xx) a VH2 having an amino acid sequence of SEQ ID NO:375; and a VL2 having an amino acid sequence of SEQ ID NO:380; or
(xxi) a VH2 having an amino acid sequence of SEQ ID NO:391; and a VL2 having an amino acid sequence of SEQ ID NO:395;
(xxii) a VH2 having an amino acid sequence of SEQ ID NO:399; and a VL2 having an amino acid sequence of SEQ ID NO:403;
(xxiii) a VH2 having an amino acid sequence of SEQ ID NO:407; and a VL2 having an amino acid sequence of SEQ ID NO:411;
(xxiv) a VH2 having an amino acid sequence of SEQ ID NO:415; and a VL2 having an amino acid sequence of SEQ ID NO:419;
(xxv) a VH2 having an amino acid sequence of SEQ ID NO:423; and a VL2 having an amino acid sequence of SEQ ID NO:5; or
(xxvi) a VH2 having an amino acid sequence of SEQ ID NO:431; and a VL2 having an amino acid sequence of SEQ ID NO:435.

4. (canceled)

5. The antibody or the antibody fragment of claim 1, wherein:
(a) the first antigen binding domain comprises:
(i) a VH1 having an amino acid sequence of SEQ ID NO:439; and a VL1 having an amino acid sequence of SEQ ID NO:443;
(ii) a VH1 having an amino acid sequence of SEQ ID NO:447; and a VL1 having an amino acid sequence of SEQ ID NO:451;
(iii) a VH1 having an amino acid sequence of SEQ ID NO:455; and a VL1 having an amino acid sequence of SEQ ID NO:459; or
(iv) a VH1 having an amino acid sequence of SEQ ID NO:463; and a VL1 having an amino acid sequence of SEQ ID NO:467; and
(b) the second antigen binding domain comprises:
(i) a VH2 having an amino acid sequence of SEQ ID NO:1; and a VL2 having an amino acid sequence of SEQ ID NO:5;
(ii) a VH2 having an amino acid sequence of SEQ ID NO:11; and a VL2 having an amino acid sequence of SEQ ID NO:5;
(iii) a VH2 having an amino acid sequence of SEQ ID NO:1; and a VL2 having an amino acid sequence of SEQ ID NO:19;
(iv) a VH2 having an amino acid sequence of SEQ ID NO:11; and a VL2 having an amino acid sequence of SEQ ID NO:19;
(v) a VH2 having an amino acid sequence of SEQ ID NO:15; and a VL2 having an amino acid sequence of SEQ ID NO:5;
(vi) a VH2 having an amino acid sequence of SEQ ID NO:15; and a VL2 having an amino acid sequence of SEQ ID NO:19;
(vii) a VH2 having an amino acid sequence of SEQ ID NO:63; and a VL2 having an amino acid sequence of SEQ ID NO:75;
(viii) a VH2 having an amino acid sequence of SEQ ID NO:67; and a VL2 having an amino acid sequence of SEQ ID NO:75;
(ix) a VH2 having an amino acid sequence of SEQ ID NO:63; and a VL2 having an amino acid sequence of SEQ ID NO:79;
(x) a VH2 having an amino acid sequence of SEQ ID NO:67; and a VL2 having an amino acid sequence of SEQ ID NO:79;
(xi) a VH2 having an amino acid sequence of SEQ ID NO:71; and a VL2 having an amino acid sequence of SEQ ID NO:75; or
(xii) a VH2 having an amino acid sequence of SEQ ID NO:71; and a VL2 having an amino acid sequence of SEQ ID NO:79.

6. The antibody or the antibody fragment of claim 1, wherein the first antigen binding domain is a diabody, a Fab, a F(ab′)2, a Fc, a Fabc, a Fv, a scFv, or a protein scaffold.

7. The antibody or the antibody fragment of claim 1, wherein the second antigen binding domain is a diabody, a Fab, a F(ab′)2, a Fc, a Fabc, a Fv, a scFv, or a protein scaffold.

8. (canceled)

9. (canceled)

10. (canceled)

11. (canceled)

12. (canceled)

13. (canceled)

14. (canceled)

15. (canceled)

16. The antibody or the antibody fragment of claim 1, wherein:
(a) the first antigen binding domain comprises a vhCDR1 having an amino acid sequence of SEQ ID NO:440; a vhCDR2 having an amino acid sequence of SEQ ID NO:457; and a vhCDR3 having an amino acid sequence of SEQ ID NO:458; and a vlCDR1 having an amino acid sequence of SEQ ID NO:460, a vlCDR2 having an amino acid sequence of SEQ ID NO:445, and a vlCDR3 having an amino acid sequence of SEQ ID NO:462; and
(b) the second antigen binding domain comprises a vhCDR1 having an amino acid sequence of SEQ ID NO:2; a vhCDR2 having an amino acid sequence of SEQ ID NO:3;
and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:22.

17. The antibody or the antibody fragment of claim 1, wherein:
(a) the first antigen binding domain comprises a VH1 having an amino acid sequence of SEQ ID NO:455; and a VL1 having an amino acid sequence of SEQ ID NO:459; and
(b) the second antigen binding domain comprises a VH2 having an amino acid sequence of SEQ ID NO:63; and a VL2 having an amino acid sequence of SEQ ID NO:79.

18. A nucleic acid sequence encoding an antibody or an antibody fragment of claim 2.

19. An expression vector comprising a nucleic acid sequence of claim 18.

20. A host cell comprising an expression vector of claim 19.

21. An antibody or an antibody fragment thereof comprising:
a) a first monomer comprising, from N-terminal to C-terminal, a VH1-CH1-hinge-CH2-CH3, wherein the VH1 is the first variable heavy chain domain and CH2-CH3 is a first Fc domain; and
b) a second monomer comprising:
i) a single chain variable fragment (scFv) comprising a second variable heavy chain domain (VH2), a linker, and a second variable light chain domain (VL2), and
ii) a second Fc domain, wherein the scFv is covalently attached to the N-terminus of the second Fc domain using a domain linker;
c) a light chain comprising, from N-terminal to C-terminal, VL1-CL, wherein the VL1 is the first variable light chain domain and CL is a constant light chain domain,
wherein the VH1 and the VL1 together form a first antigen binding domain and the VH2 and the VL2 together form a second binding domain, and
wherein the first antigen binding domain binds to CD20 and the second antigen binding domain binds to CD28.

22. The antibody or the antibody fragment of claim 21, wherein:
(a) the first antigen binding domain comprises:
(i) a vhCDR1 having an amino acid sequence of SEQ ID NO:440; a vhCDR2 having an amino acid sequence of SEQ ID NO:441; and a vhCDR3 having an amino acid sequence of SEQ ID NO:442; and a vlCDR1 having an amino acid sequence of SEQ ID NO:444, a vlCDR2 having an amino acid sequence of SEQ ID NO: 445, and a vlCDR3 having an amino acid sequence of SEQ ID NO:446;
(ii) a vhCDR1 having an amino acid sequence of SEQ ID NO:440; a vhCDR2 having an amino acid sequence of SEQ ID NO:449; and a vhCDR3 having an amino acid sequence of SEQ ID NO:450; and a vlCDR1 having an amino acid sequence of SEQ ID NO:452, a vlCDR2 having an amino acid sequence of SEQ ID NO:445, and a vlCDR3 having an amino acid sequence of SEQ ID NO:454;
(iii) a vhCDR1 having an amino acid sequence of SEQ ID NO:440; a vhCDR2 having an amino acid sequence of SEQ ID NO:457; and a vhCDR3 having an amino acid sequence of SEQ ID NO:458; and a vlCDR1 having an amino acid sequence of SEQ ID NO:460, a vlCDR2 having an amino acid sequence of SEQ ID NO:445, and a vlCDR3 having an amino acid sequence of SEQ ID NO:462; or
(iv) a vhCDR1 having an amino acid sequence of SEQ ID NO:464; a vhCDR2 having an amino acid sequence of SEQ ID NO:465; and a vhCDR3 having an amino acid sequence of SEQ ID NO:466; and a vlCDR1 having an amino acid sequence of SEQ ID NO:356, a vlCDR2 having an amino acid sequence of SEQ ID NO:357, and a vlCDR3 having an amino acid sequence of SEQ ID NO:470; and
(b) the second antigen binding domain comprises:
(i) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:8;
(ii) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:8;
(iii) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:22;
(iv) a vhCDR1 having an amino acid sequence of SEQ ID NO: 12; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:22;
(v) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:8; or
(vi) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:22.

23. The antibody or the antibody fragment of claim 21, wherein:
(a) the first antigen binding domain comprises:
(i) a VH1 having an amino acid sequence of SEQ ID NO:439; and a VL1 having an amino acid sequence of SEQ ID NO:443;
(ii) a VH1 having an amino acid sequence of SEQ ID NO:447; and a VL1 having an amino acid sequence of SEQ ID NO:451;
(iii) a VH1 having an amino acid sequence of SEQ ID NO:455; and a VL1 having an amino acid sequence of SEQ ID NO:459;
(iv) a VH1 having an amino acid sequence of SEQ ID NO:463; and a VL1 having an amino acid sequence of SEQ ID NO:467;
(v) a VH1 having an amino acid sequence of SEQ ID NO:471; and a VL1 having an amino acid sequence of SEQ ID NO:472;
(vi) a VH1 having an amino acid sequence of SEQ ID NO:473; and a VL1 having an amino acid sequence of SEQ ID NO:474;
(vii) a VH1 having an amino acid sequence of SEQ ID NO:475; and a VL1 having an amino acid sequence of SEQ ID NO:472;
(viii) a VH1 having an amino acid sequence of SEQ ID NO:477; and a VL1 having an amino acid sequence of SEQ ID NO:478;
(ix) a VH1 having an amino acid sequence of SEQ ID NO 479; and a VL1 having an amino acid sequence of SEQ ID NO:480;
(x) a VH1 having an amino acid sequence of SEQ ID NO:481; and a VL1 having an amino acid sequence of SEQ ID NO:482;
(xi) a VH1 having an amino acid sequence of SEQ ID NO:483; and a VL1 having an amino acid sequence of SEQ ID NO:484;
(xii) a VH1 having an amino acid sequence of SEQ ID NO:487; and a VL1 having an amino acid sequence of SEQ ID NO:467;
(xiii) a VH1 having an amino acid sequence of SEQ ID NO:489; and a VL1 having an amino acid sequence of SEQ ID NO:490;
(xiv) a VH1 having an amino acid sequence of SEQ ID NO:491; and a VL1 having an amino acid sequence of SEQ ID NO:492;
(xv) a VH1 having an amino acid sequence of SEQ ID NO:495; and a VL1 having an amino acid sequence of SEQ ID NO:496; or
(xvi) a VH1 having an amino acid sequence of SEQ ID NO:497; and a VL1 having an amino acid sequence of SEQ ID NO:498; and
(b) the second antigen binding domain comprises:
(i) a VH2 having an amino acid sequence of SEQ ID NO:1; and a VL2 having an amino acid sequence of SEQ ID NO:5;
(ii) a VH2 having an amino acid sequence of SEQ ID NO:11; and a VL2 having an amino acid sequence of SEQ ID NO:5;
(iii) a VH2 having an amino acid sequence of SEQ ID NO:1; and a VL2 having an amino acid sequence of SEQ ID NO:19;
(iv) a VH2 having an amino acid sequence of SEQ ID NO:11; and a VL2 having an amino acid sequence of SEQ ID NO:19;
(v) a VH2 having an amino acid sequence of SEQ ID NO:15; and a VL2 having an amino acid sequence of SEQ ID NO:5;
(vi) a VH2 having an amino acid sequence of SEQ ID NO:15; and a VL2 having an amino acid sequence of SEQ ID NO:19;
(vii) a VH2 having an amino acid sequence of SEQ ID NO:63; and a VL2 having an amino acid sequence of SEQ ID NO:75;
(viii) a VH2 having an amino acid sequence of SEQ ID NO:67; and a VL2 having an amino acid sequence of SEQ ID NO:75;
(ix) a VH2 having an amino acid sequence of SEQ ID NO:63; and a VL2 having an amino acid sequence of SEQ ID NO:79;
(x) a VH2 having an amino acid sequence of SEQ ID NO:67; and a VL2 having an amino acid sequence of SEQ ID NO:111;
(xi) a VH2 having an amino acid sequence of SEQ ID NO:71; and a VL2 having an amino acid sequence of SEQ ID NO:75;
(xii) a VH2 having an amino acid sequence of SEQ ID NO:71; and a VL2 having an amino acid sequence of SEQ ID NO:79;
(xiii) a VH2 having an amino acid sequence of SEQ ID NO:320; and a VL2 having an amino acid sequence of SEQ ID NO:324;
(xiv) a VH2 having an amino acid sequence of SEQ ID NO:327; and a VL2 having an amino acid sequence of SEQ ID NO:331;
(xv) a VH2 having an amino acid sequence of SEQ ID NO: 335; and a VL2 having an amino acid sequence of SEQ ID NO: 339;
(xvi) a VH2 having an amino acid sequence of SEQ ID NO:343; and a VL2 having an amino acid sequence of SEQ ID NO:347;
(xvii) a VH2 having an amino acid sequence of SEQ ID NO:351; and a VL2 having an amino acid sequence of SEQ ID NO:355;
(xviii) a VH2 having an amino acid sequence of SEQ ID NO:359; and a VL2 having an amino acid sequence of SEQ ID NO:355;
(xix) a VH2 having an amino acid sequence of SEQ ID NO:367; and a VL2 having an amino acid sequence of SEQ ID NO: 371;
(xx) a VH2 having an amino acid sequence of SEQ ID NO:375; and a VL2 having an amino acid sequence of SEQ ID NO:380; or
(xxi) a VH2 having an amino acid sequence of SEQ ID NO:391; and a VL2 having an amino acid sequence of SEQ ID NO:395;
(xxii) a VH2 having an amino acid sequence of SEQ ID NO:399; and a VL2 having an amino acid sequence of SEQ ID NO:403;
(xxiii) a VH2 having an amino acid sequence of SEQ ID NO:407; and a VL2 having an amino acid sequence of SEQ ID NO:411;
(xxiv) a VH2 having an amino acid sequence of SEQ ID NO:415; and a VL2 having an amino acid sequence of SEQ ID NO:419;
(xxv) a VH2 having an amino acid sequence of SEQ ID NO:423; and a VL2 having an amino acid sequence of SEQ ID NO:5; or
(xxvi) a VH2 having an amino acid sequence of SEQ ID NO:431; and a VL2 having an amino acid sequence of SEQ ID NO:435.

24. The antibody or the antibody fragment of claim 21, wherein:
(a) the first antigen binding domain comprises:
(i) a VH1 having an amino acid sequence that is at least 95% identical to SEQ ID NO:439; and a VL1 having an amino acid sequence that is at least 95% identical to SEQ ID NO:443;
(ii) a VH1 having an amino acid sequence that is at least 95% identical to SEQ ID NO:447; and a VL1 having an amino acid sequence that is at least 95% identical to SEQ ID NO:451;
(iii) a VH1 having an amino acid sequence that is at least 95% identical to SEQ ID NO:455; and a VL1 having an amino acid sequence that is at least 95% identical to SEQ ID NO:459;
(iv) a VH1 having an amino acid sequence that is at least 95% identical to SEQ ID NO:463; and a VL1 having an amino acid sequence that is at least 95% identical to SEQ ID NO:467;
(v) a VH1 having an amino acid sequence that is at least 95% identical to SEQ ID NO:471; and a VL1 having an amino acid sequence that is at least 95% identical to SEQ ID NO:472;
(vi) a VH1 having an amino acid sequence that is at least 95% identical to SEQ ID NO:473; and a VL1 having an amino acid sequence that is at least 95% identical to SEQ ID NO:474;
(vii) a VH1 having an amino acid sequence that is at least 95% identical to SEQ ID NO:475; and a VL1 having an amino acid sequence that is at least 95% identical to SEQ ID NO:472;
(viii) a VH1 having an amino acid sequence that is at least 95% identical to SEQ ID NO:477; and a VL1 having an amino acid sequence that is at least 95% identical to SEQ ID NO:478;
(ix) a VH1 having an amino acid sequence that is at least 95% identical to SEQ ID NO:479; and a VL1 having an amino acid sequence that is at least 95% identical to SEQ ID NO:480;
(x) a VH1 having an amino acid sequence that is at least 95% identical to SEQ ID NO:481; and a VL1 having an amino acid sequence that is at least 95% identical to SEQ ID NO:482;
(xi) a VH1 having an amino acid sequence that is at least 95% identical to SEQ ID NO:483; and a VL1 having an amino acid sequence that is at least 95% identical to SEQ ID NO:484;
(xii) a VH1 having an amino acid sequence that is at least 95% identical to SEQ ID NO:487; and a VL1 having an amino acid sequence that is at least 95% identical to SEQ ID NO:467;
(xiii) a VH1 having an amino acid sequence that is at least 95% identical to SEQ ID NO:489; and a VL1 having an amino acid sequence that is at least 95% identical to SEQ ID NO:490;
(xiv) a VH1 having an amino acid sequence that is at least 95% identical to SEQ ID NO:491; and a VL1 having an amino acid sequence that is at least 95% identical to SEQ ID NO:492;
(xv) a VH1 having an amino acid sequence that is at least 95% identical to SEQ ID NO:495; and a VL1 having an amino acid sequence that is at least 95% identical to SEQ ID NO:496; or
(xvi) a VH1 having an amino acid sequence that is at least 95% identical to SEQ ID NO:497; and a VL1 having an amino acid sequence that is at least 95% identical to SEQ ID NO:498; and
(b) the second antigen binding domain comprises:
(i) a VH2 having an amino acid sequence that is at least 95% identical to SEQ ID NO:1; and a VL2 having an amino acid sequence that is at least 95% identical to SEQ ID NO: 5;
(ii) a VH2 having an amino acid sequence that is at least 95% identical to SEQ ID NO:11; and a VL2 having an amino acid sequence that is at least 95% identical to SEQ ID NO:5;
(iii) a VH2 having an amino acid sequence that is at least 95% identical to SEQ ID NO:1; and a VL2 having an amino acid sequence that is at least 95% identical to SEQ ID NO:19;
(iv) a VH2 having an amino acid sequence that is at least 95% identical to SEQ ID NO:11; and a VL2 having an amino acid sequence that is at least 95% identical to SEQ ID NO:19;
(v) a VH2 having an amino acid sequence that is at least 95% identical to SEQ ID NO:15; and a VL2 having an amino acid sequence that is at least 95% identical to SEQ ID NO:5;
(vi) a VH2 having an amino acid sequence that is at least 95% identical to SEQ ID NO:15; and a VL2 having an amino acid sequence that is at least 95% identical to SEQ ID NO:19;
(vii) a VH2 having an amino acid sequence that is at least 95% identical to SEQ ID NO:63; and a VL2 having an amino acid sequence that is at least 95% identical to SEQ ID NO:75;
(viii) a VH2 having an amino acid sequence that is at least 95% identical to SEQ ID NO:67; and a VL2 having an amino acid sequence that is at least 95% identical to SEQ ID NO:75;
(ix) a VH2 having an amino acid sequence that is at least 95% identical to SEQ ID NO:63; and a VL2 having an amino acid sequence that is at least 95% identical to SEQ ID NO:79;
(x) a VH2 having an amino acid sequence that is at least 95% identical to SEQ ID NO:67; and a VL2 having an amino acid sequence that is at least 95% identical to SEQ ID NO:111;
(xi) a VH2 having an amino acid sequence that is at least 95% identical to SEQ ID NO:71; and a VL2 having an amino acid sequence that is at least 95% identical to SEQ ID NO:75;
(xii) a VH2 having an amino acid sequence that is at least 95% identical to SEQ ID NO:71; and a VL2 having an amino acid sequence that is at least 95% identical to SEQ ID NO:79;
(xiii) a VH2 having an amino acid sequence that is at least 95% identical to SEQ ID NO:320; and a VL2 having an amino acid sequence that is at least 95% identical to SEQ ID NO:324;
(xiv) a VH2 having an amino acid sequence that is at least 95% identical to SEQ ID NO:327; and a VL2 having an amino acid sequence that is at least 95% identical to SEQ ID NO:331;
(xv) a VH2 having an amino acid sequence that is at least 95% identical to SEQ ID NO:335; and a VL2 having an amino acid sequence that is at least 95% identical to SEQ ID NO:339;
(xvi) a VH2 having an amino acid sequence that is at least 95% identical to SEQ ID NO:343; and a VL2 having an amino acid sequence that is at least 95% identical to SEQ ID NO:347;
(xvii) a VH2 having an amino acid sequence that is at least 95% identical to SEQ ID NO:351; and a VL2 having an amino acid sequence that is at least 95% identical to SEQ ID NO:355;
(xviii) a VH2 having an amino acid sequence that is at least 95% identical to SEQ ID NO:359; and a VL2 having an amino acid sequence that is at least 95% identical to SEQ ID NO:355;
(xix) a VH2 having an amino acid sequence that is at least 95% identical to SEQ ID NO:367; and a VL2 having an amino acid sequence that is at least 95% identical to SEQ ID NO:371;
(xx) a VH2 having an amino acid sequence that is at least 95% identical to SEQ ID NO:375; and a VL2 having an amino acid sequence that is at least 95% identical to SEQ ID NO:380; or
(xxi) a VH2 having an amino acid sequence that is at least 95% identical to SEQ ID NO:391; and a VL2 having an amino acid sequence that is at least 95% identical to SEQ ID NO:395;
(xxii) a VH2 having an amino acid sequence that is at least 95% identical to SEQ ID NO:399; and a VL2 having an amino acid sequence that is at least 95% identical to SEQ ID NO:403;
(xxiii) a VH2 having an amino acid sequence that is at least 95% identical to SEQ ID NO:407; and a VL2 having an amino acid sequence that is at least 95% identical to SEQ ID NO:411;
(xxiv) a VH2 having an amino acid sequence that is at least 95% identical to SEQ ID NO:415; and a VL2 having an amino acid sequence that is at least 95% identical to SEQ ID NO:419;
(xxv) a VH2 having an amino acid sequence that is at least 95% identical to SEQ ID NO:423; and a VL2 having an amino acid sequence that is at least 95% identical to SEQ ID NO:5; or
(xxvi) a VH2 having an amino acid sequence that is at least 95% identical to SEQ ID NO:431; and a VL2 having an amino acid sequence that is at least 95% identical to SEQ ID NO:435.

25. The antibody or the antibody fragment of claim 21, wherein:
(a) the first antigen binding domain comprises:
(i) a VH1 having an amino acid sequence of SEQ ID NO:439; and a VL1 having an amino acid sequence of SEQ ID NO:443;
(ii) a VH1 having an amino acid sequence of SEQ ID NO:447; and a VL1 having an amino acid sequence of SEQ ID NO:451;
(iii) a VH1 having an amino acid sequence of SEQ ID NO:455; and a VL1 having an amino acid sequence of SEQ ID NO:459; or
(iv) a VH1 having an amino acid sequence of SEQ ID NO:463; and a VL1 having an amino acid sequence of SEQ ID NO:467; and
(b) the second antigen binding domain comprises:
(i) a VH2 having an amino acid sequence of SEQ ID NO:1; and a VL2 having an amino acid sequence of SEQ ID NO:5;
(ii) a VH2 having an amino acid sequence of SEQ ID NO:11; and a VL2 having an amino acid sequence of SEQ ID NO:5;
(iii) a VH2 having an amino acid sequence of SEQ ID NO:1; and a VL2 having an amino acid sequence of SEQ ID NO:19;
(iv) a VH2 having an amino acid sequence of SEQ ID NO:11; and a VL2 having an amino acid sequence of SEQ ID NO:19;
(v) a VH2 having an amino acid sequence of SEQ ID NO:15; and a VL2 having an amino acid sequence of SEQ ID NO:5;
(vi) a VH2 having an amino acid sequence of SEQ ID NO:15; and a VL2 having an amino acid sequence of SEQ ID NO:19;
(vii) a VH2 having an amino acid sequence of SEQ ID NO:63; and a VL2 having an amino acid sequence of SEQ ID NO:75;
(viii) a VH2 having an amino acid sequence of SEQ ID NO:67; and a VL2 having an amino acid sequence of SEQ ID NO:75;
(ix) a VH2 having an amino acid sequence of SEQ ID NO:63; and a VL2 having an amino acid sequence of SEQ ID NO:79;
(x) a VH2 having an amino acid sequence of SEQ ID NO:67; and a VL2 having an amino acid sequence of SEQ ID NO:79;
(xi) a VH2 having an amino acid sequence of SEQ ID NO:71; and a VL2 having an amino acid sequence of SEQ ID NO:75; or
(xii) a VH2 having an amino acid sequence of SEQ ID NO:71; and a VL2 having an amino acid sequence of SEQ ID NO:79.

26. The antibody or the antibody fragment of claim 21, wherein:
(a) a first monomer is selected from the amino acid sequences represented by SEQ ID NOs: 405, 422, 427, 436, 453, 456. 461, and 485;
(b) a second monomer is selected from the amino acid sequences represented by SEQ ID NOs: 406, 417, 418, 421, 428, 430, 432, 434, 438, 448, 468, 469, and 486; and
(c) a light chain is selected from the amino acid sequences represented by SEQ ID NOs: 334, 366, 426, and 429.

27. (canceled)

28. (canceled)

29. (canceled)

30. (canceled)

31. (canceled)

32. (canceled)

33. The antibody or the antibody fragment of claim 21, wherein:
(a) the first antigen binding domain comprises a vhCDR1 having an amino acid sequence of SEQ ID NO:440; a vhCDR2 having an amino acid sequence of SEQ ID NO:457; and a vhCDR3 having an amino acid sequence of SEQ ID NO:458; and a vlCDR1 having an amino acid sequence of SEQ ID NO:460, a vlCDR2 having an amino acid sequence of SEQ ID NO:445, and a vlCDR3 having an amino acid sequence of SEQ ID NO:462; and
(b) the second antigen binding domain comprises a vhCDR1 having an amino acid sequence of SEQ ID NO:2; a vhCDR2 having an amino acid sequence of SEQ ID NO:3;
and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:22.

34. The antibody or the antibody fragment of claim 21, wherein:
(a) the first antigen binding domain comprises a VH1 having an amino acid sequence of SEQ ID NO:455; and a VL1 having an amino acid sequence of SEQ ID NO:459; and
(b) the second antigen binding domain comprises a VH2 having an amino acid sequence of SEQ ID NO:63; and a VL2 having an amino acid sequence of SEQ ID NO:79.

35. The antibody or the antibody fragment of claim 21, wherein the first monomer comprises the amino acid sequence of SEQ ID NO: 461, the second monomer comprises the amino acid sequence of SEQ ID NO: 469, and the light chain comprises the amino acid sequence of SEQ ID NO: 429.

36. A method of treating a B-cell malignancy in a patient in need thereof, comprising administering to the patient a combination of an anti-CD20 x anti-CD28 antibody or an antibody fragment thereof and a T-cell engager, wherein:
(A) the anti-CD20 x anti-CD28 antibody or the fragment thereof comprises:
(a) a first antigen binding domain that binds to CD20 comprising:
(i) a vhCDR1 having an amino acid sequence of SEQ ID NO:440; a vhCDR2 having an amino acid sequence of SEQ ID NO:441; and a vhCDR3 having an amino acid sequence of SEQ ID NO:442; and a vlCDR1 having an amino acid sequence of SEQ ID NO:444, a vlCDR2 having an amino acid sequence of SEQ ID NO: 445, and a vlCDR3 having an amino acid sequence of SEQ ID NO:446;
(ii) a vhCDR1 having an amino acid sequence of SEQ ID NO:440; a vhCDR2 having an amino acid sequence of SEQ ID NO:449; and a vhCDR3 having an amino acid sequence of SEQ ID NO:450; and a vlCDR1 having an amino acid sequence of SEQ ID NO:452, a vlCDR2 having an amino acid sequence of SEQ ID NO:445, and a vlCDR3 having an amino acid sequence of SEQ ID NO:454;
(iii) a vhCDR1 having an amino acid sequence of SEQ ID NO:440; a vhCDR2 having an amino acid sequence of SEQ ID NO:457; and a vhCDR3 having an amino acid sequence of SEQ ID NO:458; and a vlCDR1 having an amino acid sequence of SEQ ID NO:460, a vlCDR2 having an amino acid sequence of SEQ ID NO:445, and a vlCDR3 having an amino acid sequence of SEQ ID NO:462; or
(iv) a vhCDR1 having an amino acid sequence of SEQ ID NO:464; a vhCDR2 having an amino acid sequence of SEQ ID NO:465; and a vhCDR3 having an amino acid sequence of SEQ ID NO:466; and a vlCDR1 having an amino acid sequence of SEQ ID NO:356, a vlCDR2 having an amino acid sequence of SEQ ID NO:357, and a vlCDR3 having an amino acid sequence of SEQ ID NO:470; and
(b) a second antigen binding domain that binds to CD28 comprising:
(i) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:8;
(ii) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:8;
(iii) a vhCDR1 having an amino acid sequence of SEQ ID NO:2; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:22;
(iv) a vhCDR1 having an amino acid sequence of SEQ ID NO:12; a vhCDR2 having an amino acid sequence of SEQ ID NO:3; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:22;
(v) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:8; or
(vi) a vhCDR1 having an amino acid sequence of SEQ ID NO:12; a vhCDR2 having an amino acid sequence of SEQ ID NO:17; and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:22; and
(B) the T cell engager is an anti-CD79b x anti-CD20 x anti-CD3 antibody or an antibody fragment thereof.

37. The method of claim 36, wherein the anti-CD20 x anti-CD28 antibody or the antibody fragment comprises:
(i) the first antigen binding domain that binds to CD20 comprises a vhCDR1 having an amino acid sequence of SEQ ID NO:440, a vhCDR2 having an amino acid sequence of SEQ ID NO:457, and a vhCDR3 having an amino acid sequence of SEQ ID NO:458; and a vlCDR1 having an amino acid sequence of SEQ ID NO:460, a vlCDR2 having an amino acid sequence of SEQ ID NO:445, and a vlCDR3 having an amino acid sequence of SEQ ID NO:462; and
(ii) the second antigen binding domain that binds to CD28 comprises a vhCDR1 having an amino acid sequence of SEQ ID NO:2, a vhCDR2 having an amino acid sequence of SEQ ID NO:3, and a vhCDR3 having an amino acid sequence of SEQ ID NO:4; and a vlCDR1 having an amino acid sequence of SEQ ID NO:6, a vlCDR2 having an amino acid sequence of SEQ ID NO:7, and a vlCDR3 having an amino acid sequence of SEQ ID NO:22.

38. The method of claim 36, wherein the anti-CD79b x anti-CD20 x anti-CD3 antibody or the antibody fragment comprises:
(a) an antigen binding domain that binds to CD79b comprising a vhCDR1 having an amino acid sequence of SEQ ID NO:524, a vhCDR2 having an amino acid sequence of SEQ ID NO:525, and a vhCDR3 having an amino acid sequence of SEQ ID NO:526; and a vlCDR1 having an amino acid sequence of SEQ ID NO:528, a vlCDR2 having an amino acid sequence of SEQ ID NO:529, and a vlCDR3 having an amino acid sequence of SEQ ID NO:530;
(b) an antigen binding domain that binds to CD20 comprising a vhCDR1 having an amino acid sequence of SEQ ID NO:540, a vhCDR2 having an amino acid sequence of SEQ ID NO:541, and a vhCDR3 having an amino acid sequence of SEQ ID NO:542; and a vlCDR1 having an amino acid sequence of SEQ ID NO:544, a vlCDR2 having an amino acid sequence of SEQ ID NO:545, and a vlCDR3 having an amino acid sequence of SEQ ID NO:546; and
(c) an antigen binding domain that binds to CD3 comprising a vhCDR1 having an amino acid sequence of SEQ ID NO:532, a vhCDR2 having an amino acid sequence of SEQ ID NO:533, and a vhCDR3 having an amino acid sequence of SEQ ID NO:534; and a vlCDR1 having an amino acid sequence of SEQ ID NO:536, a vlCDR2 having an amino acid sequence of SEQ ID NO:537, and a vlCDR3 having an amino acid sequence of SEQ ID NO:538.

39. The method of claim 36, wherein the anti-CD20 x anti-CD28 antibody or the antibody fragment comprises:
(a) the CD20 antigen binding domain comprising a VH1 having an amino acid sequence of SEQ ID NO:455; and a VL1 having an amino acid sequence of SEQ ID NO:459; and
(b) the CD28 antigen binding domain comprising a VH2 having an amino acid sequence of SEQ ID NO:63; and a VL2 having an amino acid sequence of SEQ ID NO:79.

40. The method of claim 36, wherein the anti-CD79b x anti-CD20 x anti-CD3 antibody or the antibody fragment comprises:
(a) the CD79b antigen binding domain comprising a VH1 having an amino acid sequence of SEQ ID NO:523; and a VL1 having an amino acid sequence of SEQ ID NO:527;
(b) the CD20 antigen binding domain comprising a VH2 having an amino acid sequence of SEQ ID NO:539; and a VL2 having an amino acid sequence of SEQ ID NO:543; and
(c) the CD3 antigen binding domain comprising a VH2 having an amino acid sequence of SEQ ID NO:531; and a VL2 having an amino acid sequence of SEQ ID NO:535.

41. The method of claim 36, wherein:
(a) the anti-CD20 x anti-CD28 antibody or the antibody fragment comprises:
(i) the CD20 antigen binding domain comprising a VH1 having an amino acid sequence of SEQ ID NO:455; and a VL1 having an amino acid sequence of SEQ ID NO:459; and
(ii) the CD28 antigen binding domain comprising a VH2 having an amino acid sequence of SEQ ID NO:63; and a VL2 having an amino acid sequence of SEQ ID NO:79; and
(b) the anti-CD79b x anti-CD20 x anti-CD3 antibody or the antibody fragment comprises:
(i) the CD79b antigen binding domain comprising a VH1 having an amino acid sequence of SEQ ID NO:523; and a VL1 having an amino acid sequence of SEQ ID NO:527;
(ii) the CD20 antigen binding domain comprising a VH2 having an amino acid sequence of SEQ ID NO:539; and a VL2 having an amino acid sequence of SEQ ID NO:543; and
(iii) the CD3 antigen binding domain comprising a VH2 having an amino acid sequence of SEQ ID NO:531; and a VL2 having an amino acid sequence of SEQ ID NO:535.

42. The method of claim 36, wherein the anti-CD20 x anti-CD28 antibody or the antibody fragment comprises:
(a) a first monomer polypeptide having an amino acid sequence of SEQ ID NO:461;
(b) a second monomer polypeptide having an amino acid sequence of SEQ ID NO:469; and
(c) a light chain polypeptide having an amino acid sequence of SEQ ID NO:429.

43. The method of claim 36, wherein the anti-CD79b x anti-CD20 x anti-CD3 antibody or the antibody fragment comprises:
(a) a first monomer polypeptide having an amino acid sequence of SEQ ID NO:520;
(b) a second monomer polypeptide having an amino acid sequence of SEQ ID NO:521; and
(c) a light chain polypeptide having an amino acid sequence of SEQ ID NO:522.

44. The method of claim 36, wherein:
(a) the anti-CD20 x anti-CD28 antibody or the antibody fragment comprises:
(i) a first monomer polypeptide having an amino acid sequence of SEQ ID NO:461;
(ii) a second monomer polypeptide having an amino acid sequence of SEQ ID NO:469;
(iii) a first light chain polypeptide having an amino acid sequence of SEQ ID NO:429; and
(b) the anti-CD79b x anti-CD20 x anti-CD3 antibody or the antibody fragment comprises:
(i) a fourth monomer polypeptide having an amino acid sequence of SEQ ID NO:520;
(ii) a fifth monomer polypeptide having an amino acid sequence of SEQ ID NO:521; and
(iii) a second light chain polypeptide having an amino acid sequence of SEQ ID NO:522.

45. (canceled)

46. (canceled)

47. (canceled)

48. The method of claim 36, wherein the B-cell malignancy is B-cell lymphoma, non-Hodgkin lymphoma (NHL), diffuse large B-cell lymphoma (DLBCL), a mantle cell lymphoma (MCL), a follicular lymphoma (FL), acute lymphoblastic leukemia (ALL), chronic lymphocytic leukemia (CLL), small lymphocytic lymphoma (SLL), Waldenstrom macroglobulinemia (WM), multiple myeloma (MM), mucosa-associated lymphoid tissue (MALT) lymphoma, Hodgkin's lymphoma, Burkitt's lymphoma, hairy-cell leukemia, or Plasmacytoma.
